(19) United States
(12) Patent Application Publication
(10) Pub. No.: US 2025/0066379 A1
(43) Pub. Date: Feb. 27, 2025

(54) ANTIMALARIAL AGENTS

(71) Applicants: Merck Sharp & Dohme LLC, Rahway, NJ (US); The Walter and Eliza Hall Institute of Medical Research, Parkville, Victoria (AU); MSD R&D (China) Co., Ltd., Beijing (CN)

(72) Inventors: John A. McCauley, Maple Glen, PA (US); Manuel De Lera Ruiz, Perkasie, PA (US); Zhuyan Guo, Scotch Plains, NJ (US); Philippe Nantermet, Lansdale, PA (US); Michael J. Kelly, III, Paoli, PA (US); Alvaro Gutierrez Bonet, Lansdale, PA (US); Lianyun Zhao, Beijing (CN); Zhiyu Lei, Shanghai (CN); Bin Hu, Shanghai (CN); Dongmei Zhan, Shanghai (CN); Anthony Hodder, Parkville (AU)

(73) Assignees: Merck Sharp & Dohme LLC, Rahway, NJ (US); The Walter and Eliza Hall Institute of Medical Research, Parkville, Victoria (AU); MSD R&D (China) Co., Ltd., Beijing (CN)

(21) Appl. No.: 18/716,280

(22) PCT Filed: Dec. 5, 2022
(86) PCT No.: PCT/CN2022/051770

§ 371 Date: Jun. 4, 2024

Related U.S. Application Data
(60) Provisional application No. 63/397,614, filed on Aug. 12, 2022.

(30) Foreign Application Priority Data
Dec. 7, 2021 (WO) PCT/CN2021/136177

(51) Int. Cl. C07D 491/22; A61K 31/519; A61K 45/06; C07D 487/08; C07D 491/048; C07D 491/052; C07D 519/00

(52) U.S. Cl. CPC C07D 491/22; A61K 31/519; A61K 45/06; C07D 487/08; C07D 491/048; C07D 491/052; C07D 519/00

(57) ABSTRACT

Provided are methods of treating malaria comprising administration of compounds of Formula (I), or a pharmaceutically acceptable salt thereof, to a subject in need thereof, wherein the variables are as defined herein. Also provided are uses of the compounds of Formula (I), as defined herein, for inhibiting plasmepsin X, plasmepsin IX or plasmepsin X and IX activity, for treating a Plasmodium infection, and for treating malaria. Also provided are methods of treatment further comprising administration of one or more additional anti-malarial compounds.

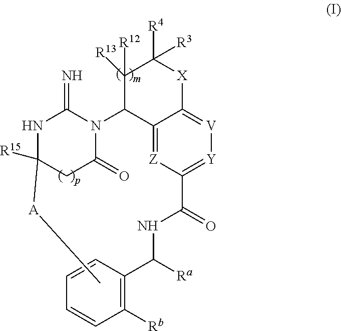

### FIELD OF THE INVENTION

The present invention relates to compounds of Formula (I), or pharmaceutically acceptable salts thereof, useful for the treatment of Plasmodium infections. More specifically, the present invention relates to compounds of Formula (I), or pharmaceutically acceptable salts thereof, useful for the treatment of malaria.

### BACKGROUND OF THE INVENTION

Malaria is a major disease in humans with several hundred million infections and over 450,000 deaths each year. The most lethal form of malaria is caused by Plasmodium falciparum. This protozoan parasite is responsible for almost all malarial deaths with most occurring in Africa. P. falciparum has a complex life cycle starting in the Anopheles mosquito vector when sporozoite forms are injected into the human host during a blood feed. These sporozoites migrate to the liver and invade hepatocytes in which they develop to form thousands of liver merozoites that egress into the blood where they invade erythrocytes to commence the asexual cycle of the parasite responsible for the symptoms of malaria. The parasite develops within the protected niche of the red cell to form 16-32 merozoites that, once mature, egress from the host cell to invade new red blood cells. Some of these parasites differentiate to form gametocytes, the sexual form of the parasite. These can be taken up by the mosquito where male and female gametes form, fuse and differentiate into oocysts on the mosquito midgut extracellular matrix. Sporozoites form within the oocyst and upon egress migrate to the salivary gland for delivery to the next host during blood feeding for perpetuation and survival of the parasite.

Other forms of malaria include a relapsing form of malaria caused by P. vivax which is responsible for significant morbidity, can cause virulent forms of this disease with some deaths and is mainly a problem outside Africa. P. knowlesi is found in South East Asia and is a zoonotic parasite that normally infects long-tailed macaques but has been shown to infect humans in Malaysian Borneo.

Artemisinin combined with partner drugs have become a mainstay in the treatment and control of malaria. However, due to the increasing threat of artemisinin-based combination therapy (ACT) drug resistance, the development of new antimalarials with novel targets that inhibit multiple steps in the parasite life cycle is an urgent priority for the malaria control field. Such novel antimalarials, as monotherapies or ACT partner drugs, could make strides towards malaria elimination as there is a reduced likelihood of parasites with preexisting resistance mutations being present in the parasite population.

Currently, aspartic acid proteases are prime targets for drug development: the HIV aspartic acid protease has been successfully targeted with a drug in clinical use; inhibitors that target human renin, BACE1 and gamma-secretase have been or are in clinical development. In the antimalarial drug space, P. falciparum aspartic acid proteases plasmepsin X and IX (PMX and PMIX) have been identified as potential targets since inhibitors block parasite egress and invasion of the host cell and prevent maturation of some rhoptry and micronemal proteins required for this process (Pino P, et al. A multistage antimalarial targets the plasmepsins IX and X essential for invasion and egress. Science. 2017; 358(6362):522-8.)

### SUMMARY OF THE INVENTION

The present invention is directed to compounds of Formula (I):

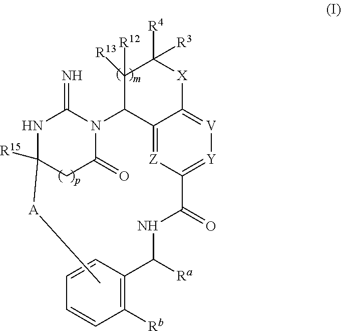

wherein A, X, V, Y, Z, R^{a}, R^{b}, R³, R⁴, R¹², R¹³, R¹⁵, m and p are described below.

Also described herein are methods of treatment of Plasmodium infections comprising administering to a subject in need thereof a compound of any of Formulas (I)-(VIII), or a pharmaceutically acceptable salt thereof. Also described herein are methods of treatment of Plasmodium infections comprising administering to a subject in need thereof a composition comprising a compound of any of Formulas (I)-(VIII), or a pharmaceutically acceptable salt thereof, and a pharmaceutically acceptable carrier.

Also described herein are methods of treatment of malaria comprising administering to a subject in need thereof a compound of any of Formulas (I)-(VIII), or a pharmaceutically acceptable salt thereof.

The present invention further provides the use of compositions, including pharmaceutical compositions, comprising one or more compounds of the invention (e.g., one compound of the invention), or a tautomer thereof, or a pharmaceutically acceptable salt or solvate of said compound(s) and/or said tautomer(s), optionally together with one or more additional therapeutic agents, optionally in an acceptable (e.g., pharmaceutically acceptable) carrier or diluent, for the treatment of malaria.

Moreover, the present invention provides methods for the use of pharmaceutical compositions comprising one or more of said compounds in the free form or in pharmaceutically acceptable salt form, together with one or more customary pharmaceutical excipient(s), for the treatment of Plasmodium infections, the treatment of malaria, the inhibition of plasmepsin X, or the dual inhibition of plasmepsin X and plasmepsin IX. Methods for the use of combinations of the compounds or salts of the invention together with one or more additional pharmaceutically active agents are also provided.

The present invention further provides methods for the inhibition of plasmepsin X, or the dual inhibition of plasmepsin X and plasmepsin IX activity and of treatment, prevention, amelioration and/or delaying onset of diseases or disorders in which the inhibition of plasmepsin X and/or plasmepsin IX has or may have a therapeutic effect, e.g. malaria.

The present invention further provides methods for the inhibition of P. falciparum aspartic acid proteases. The present invention further provides methods for blocking P. falciparum growth by inhibiting plasmepsin X. The present invention further provides methods for blocking P. falciparum growth by inhibiting both plasmepsin X and plasmepsin IX.

The present invention further provides methods for the treatment of malaria by inhibiting plasmepsin X. The present invention further provides methods for the treatment of malaria by inhibiting both plasmepsin X and plasmepsin IX.

These and other embodiments of the invention, which are described in detail below or will become clear to those of ordinary skill in the art, are included within the scope of the invention.

### DETAILED DESCRIPTION OF THE INVENTION

Described herein are compounds having the structural Formula (I):

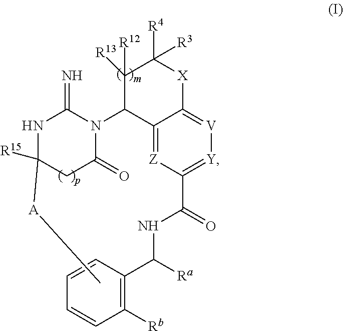

wherein A is a straight or branched, saturated or unsaturated (C₃-C₁₀)alkylene, phenyl(C₃-C₁₀)alkylene or cycloalkyl(C₃-C₁₀)alkylene comprising at least one —CH₂— group, wherein one or more additional —CH₂— groups in A are optionally and independently replaced with a moiety selected from the group consisting of O, S, NR, —CONR, NRCO, SO₂, and SO₂NR and wherein one or more of the hydrogens along A can be replaced with a halogen;
X is a bond, C(R¹⁴)₂, O, S, SO, SO₂ or NH;
Y is CR⁹ or N, wherein when Y is N, Z is CR¹¹ and V is CR¹⁰;
V is CR¹⁰ or N, wherein when V is N, Z is CR¹¹ and Y is CR⁹;
Z is CR¹¹ or N, wherein when Z is N, V is CR¹⁰ and Y is CR⁹;
each occurrence of R is independently hydrogen, C₁-C₆alkylCOOH, —COOH, C₃-C₆cycloalkyl, C₁-C₆alkyl, haloC₁-C₆alkyl, C₁-C₆alkylOH, —COC₁-C₆alkyl or —COOC₁-C₆alkyl;
R^{a} is hydrogen, halogen, —CN, —OH, C₁-C₆alkoxy, C₁-C₆alkylOC₁-C₆alkyl, C₁-C₆alkylCOOH, —COOH, oxo, —COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, C₁-C₆alkylOhaloC₁-C₆alkyl, haloC₁-C₆alkyl, C₁-C₆alkylOH, —CON(R⁷)(R⁸), —N(R⁷)(R⁸) or C₁-C₆alkylN(R⁷)(R⁸) or when taken with R^{b} forms a C₃-C₆cycloalkyl or heterocycloalkyl, wherein the C₃-C₆cycloalkyl or heterocycloalkyl is unsubstituted or substituted with one to four substituents independently selected from the group consisting of halogen, —CN, —OH, C₁-C₆alkoxy, C₁-C₆alkylOC₁-C₆alkyl, C₁-C₆alkylCOOH, —COOH, oxo, —COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, C₁-C₆alkylOhaloC₁-C₆alkyl, haloC₁-C₆alkyl, C₁-C₆alkylOH, —CON(R⁷)(R⁸), —N(R⁷)(R⁸) and C₁-C₆alkylN(R⁷)(R⁸);
R^{b} is hydrogen, halogen, —CN, —OH, C₁-C₆alkoxy, C₁-C₆alkylOC₁-C₆alkyl, C₁-C₆alkylCOOH, —COOH, oxo, —COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, C₁-C₆alkylOhaloC₁-C₆alkyl, haloC₁-C₆alkyl, C₁-C₆alkylOH, —CON(R⁷)(R⁸), —N(R⁷)(R⁸) or C₁-C₆alkylN(R⁷)(R⁸) or when taken with R^{a} forms a C₃-C₆cycloalkyl or heterocycloalkyl, wherein the C₃-C₆cycloalkyl or heterocycloalkyl is unsubstituted or substituted with one to four substituents independently selected from the group consisting of halogen, —CN, —OH, C₁-C₆alkoxy, C₁-C₆alkylOC₁-C₆alkyl, C₁-C₆alkylCOOH, —COOH, oxo, —COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, C₁-C₆alkylOhaloC₁-C₆alkyl, haloC₁-C₆alkyl, C₁-C₆alkylOH, —CON(R⁷)(R⁸), —N(R⁷)(R⁸) and C₁-C₆alkylN(R⁷)(R⁸);
R³ is hydrogen, halogen, —CN, —OH, C₁-C₆alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸), —N(R⁷)(R⁸), C₁-C₆alkylN(R⁷)(R⁸), C₁-C₆alkyl(OCH₂CH₂)ₙN(R⁷)(R⁸) or C₁-C₆alkylOhaloC₁-C₆alkyl or when taken with R⁴ forms a C₃-C₆cycloalkyl or C₃-C₆heterocycloalkyl;
R⁴ is hydrogen, halogen, —CN, —OH, C₁-C₆alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸), —N(R⁷)(R⁸), C₁-C₆alkylN(R⁷)(R⁸), C₁-C₆alkyl(OCH₂CH₂)ₙN(R⁷)(R⁸) or C₁-C₆alkylOhaloC₁-C₆alkyl or when taken with R³ forms a C₃-C₆cycloalkyl or C₃-C₆heterocycloalkyl;
each occurrence of R⁷ is independently hydrogen, C₁-C₆alkylCOOH, —COOH, C₃-C₆cycloalkyl, C₁-C₆alkyl, haloC₁-C₆alkyl, C₁-C₆alkylOH, —COC₁-C₆alkyl or —COOC₁-C₆alkyl;
each occurrence of R⁸ is independently hydrogen, C₁-C₆alkylCOOH, —COOH, C₃-C₆cycloalkyl, C₁-C₆alkyl, haloC₁-C₆alkyl, C₁-C₆alkylOH, —COC₁-C₆alkyl or —COOC₁-C₆alkyl;
R⁹ is hydrogen, halogen, —CN, —OH, C₁-C₆alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸), —N(R⁷)(R⁸) or C₁-C₆alkylN(R⁷)(R⁸);
R¹⁰ is hydrogen, halogen, —CN, —OH, C₁-C₆alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸), —N(R⁷)(R⁸) or C₁-C₆alkylN(R⁷)(R⁸);
R¹¹ is hydrogen, halogen, —CN, —OH, C₁-C₆alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸), —N(R⁷)(R⁸) or C₁-C₆alkylN(R⁷)(R⁸);
R¹² is hydrogen, halogen, —CN, —OH, C₁-C₆alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸), —N(R⁷)(R⁸) or C₁-C₆alkylN(R⁷)(R⁸);
R¹³ is hydrogen, halogen, —CN, —OH, C₁-C₆alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸), —N(R⁷)(R⁸) or C₁-C₆alkylN(R⁷)(R⁸);
each occurrence of R¹⁴ is independently selected from the group consisting of hydrogen, halogen, —CN, —OH, C₁-C₆alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸), —N(R⁷)(R⁸) and C₁-C₆alkylN(R⁷)(R⁸);
R¹⁵ is hydrogen, halogen, —CN, —OH, C₁-C₆alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸), —N(R⁷)(R⁸) or C₁-C₆alkylN(R⁷)(R⁸);
m is 0 or 1;
n is 1, 2, 3 or 4; and
p is 0 or 1.

In the embodiments described herein, X is a bond, C(R¹⁴)₂, O, S, SO, SO₂ or NH. In certain embodiments described herein X is a bond. In certain embodiments, X is C(R¹⁴)₂, wherein R¹⁴ is discussed in further detail below. In certain embodiments, X is a bond, CH₂, CH(CH₃), C(CH₃)₂, O, CH(OCH₃), SO₂ or CF₂. In other embodiments, X is CH₂, O, S, SO, SO₂ or NH. In certain embodiments, X is CH₂. In certain embodiments described herein, X is O. In certain embodiments described herein, X is S. In certain embodiments described herein, X is SO. In other embodiments described herein, X is SO₂. In certain embodiments described herein, X is NH.

In the embodiments described herein, Y is CR⁹ or N. In certain embodiments, Y is CR⁹, wherein R⁹ is discussed in detail below. In certain embodiments, Y is N. In certain embodiments, Y is CH. In certain embodiments, wherein when Y is N, Z is CR¹¹ and V is CR¹⁰.

In the embodiments described herein, V is CR¹⁰ or N. In certain embodiments, V is CR¹⁰, wherein R¹⁰ is discussed in detail below. In certain embodiments, V is N. In certain embodiments, V is CH. In certain embodiments, wherein when V is N, Z is CR¹¹ and Y is CR⁹.

In the embodiments described herein, Z is CR¹¹ or N. In certain embodiments, Z is CR¹¹, wherein R¹¹ is discussed in detail below. In certain embodiments, Z is CH. In certain embodiments, Z is N. In certain embodiments, wherein when Z is N, V is CR¹⁰ and Y is CR⁹.

In certain embodiments, X is O, Y and V are each CH and Z is N. In certain embodiments, X is O, Y and Z are each CH and V is N. In certain embodiments, X is O and V, Y and Z are all simultaneously CH.

In the compounds described herein, R^{a} is hydrogen, halogen, —CN, —OH, C₁-C₆alkoxy, C₁-C₆alkylOC₁-C₆alkyl, C₁-C₆alkylCOOH, —COOH, oxo, —COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, —C₁-C₆alkylOhaloC₁-C₆alkyl, haloC₁-C₆alkyl, C₁-C₆alkylOH, —CON(R⁷)(R⁸), —N(R⁷)(R⁸) or C₁-C₆alkylN(R⁷)(R⁸) or when taken with R^{b} forms a C₃-C₆cycloalkyl or heterocycloalkyl, wherein the C₃-C₆cycloalkyl or heterocycloalkyl is unsubstituted or substituted with one to four substituents independently selected from the group consisting of halogen, —CN, —OH, C₁-C₆alkoxy, C₁-C₆alkylOC₁-C₆alkyl, C₁-C₆alkylCOOH, —COOH, oxo, —COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, —C₁-C₆alkylOhaloC₁-C₆alkyl, haloC₁-C₆alkyl, C₁-C₆alkylOH, —CON(R⁷)(R⁸), —N(R⁷)(R⁸) and C₁-C₆alkylN(R⁷)(R⁸).

In certain embodiments described herein, R^{a} is hydrogen.

In certain embodiments, R^{a} is halogen. Examples of suitable halogens include chlorine, bromine, fluorine and iodine.

In certain embodiments, R^{a} is —CN.

In certain embodiments, R^{a} is —OH.

In certain embodiments, R^{a} is C₁-C₆alkoxy. Suitable alkoxys include, but are not limited to, methoxy, ethoxy, n-propoxy, isopropoxy and n-butoxy.

In certain embodiments, R^{a} is C₁-C₆alkylOC₁-C₆alkyl.

In certain embodiments, R^{a} is C₁-C₆alkylCOOH.

In certain embodiments, R^{a} is —COOH.

In certain embodiments, R^{a} is an oxo group.

In certain embodiments, R^{a} is —COOC₁-C₆alkyl.

In certain embodiments, R^{a} is C₁-C₆alkylCOOC₁-C₆alkyl.

In certain embodiments, R^{a} is C₃-C₆cycloalkyl. Suitable examples of cycloalkyls include, but are not limited to, cyclopropyl, cyclobutyl, cyclopentyl and cyclohexyl.

In certain embodiments, R^{a} is C₁-C₆alkylC₃-C₆cycloalkyl. Suitable examples of cycloalkyls include, but are not limited to

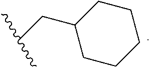 [SMILES: *CC1CCCCC1]

In certain embodiments, R^{a} is C₁-C₆alkyl. Examples of C₁-C₆alkyl groups include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R^{a} is methyl.

In certain embodiments, R^{a} is C₁-C₆alkylOhaloC₁-C₆alkyl. Suitable examples of C₁-C₆alkylOhaloC₁-C₆alkyls include, but are not limited to,

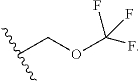 [SMILES: *COC(F)(F)F]

In certain embodiments, R^{a} is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl.

In certain embodiments, R^{a} is C₁-C₆alkylOH. Suitable alcohols include, but are not limited to, methanol, ethanol, propanol, butanol and iso-butanol.

In certain embodiments, R^{a} is —CON(R⁷)(R⁸). In certain embodiments, R^{a} is —N(R⁷)(R⁸). In certain embodiments, R^{a} is C₁-C₆alkylN(R⁷)(R⁸), wherein R⁷ and R⁸ will be described in detail below.

In certain embodiments, the compounds described herein have the Formula (II):

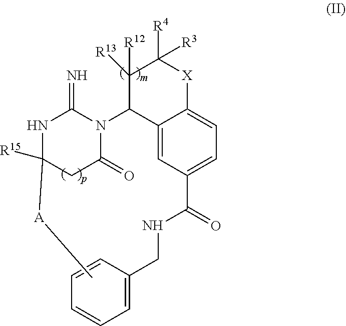

wherein A, R³, R⁴, R¹², R¹³, R¹⁵, m and p are as defined in Formula (I).

In certain embodiments, R^{a} is taken with R^{b} and forms a C₃-C₆cycloalkyl or heterocycloalkyl, wherein the C₃-C₆cycloalkyl or heterocycloalkyl is unsubstituted or substituted with one to four substituents independently selected from the group consisting of halogen, —CN, —OH, C₁-C₆alkoxy, C₁-C₆alkylOC₁-C₆alkyl, C₁-C₆alkylCOOH, —COOH, oxo, —COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, —C₁-C₆alkylOhaloC₁-C₆alkyl, haloC₁-C₆alkyl, C₁-C₆alkylOH, —CON(R⁷)(R⁸), —N(R⁷)(R⁸) and C₁-C₆alkylN(R⁷)(R⁸).

In certain embodiments, R^{a} is taken with R^{b} and forms a C₃-C₆cycloalkyl, wherein the cycloalkyl is unsubstituted or substituted with one to four substituents independently selected from the group consisting of halogen, —CN, —OH, C₁-C₆alkoxy, C₁-C₆alkylOC₁-C₆alkyl, C₁-C₆alkylCOOH, —COOH, oxo, —COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, —C₁-C₆alkylOhaloC₁-C₆alkyl, haloC₁-C₆alkyl, C₁-C₆alkylOH, —CON(R⁷)(R⁸), —N(R⁷)(R⁸) and C₁-C₆alkylN(R⁷)(R⁸).

Suitable examples of cycloalkyls include, but are not limited to, cyclopropyl, cyclobutyl, cyclopentyl and cyclohexyl.

In certain embodiments, the cycloalkyl is unsubstituted. In certain embodiments, the heterocycloalkyl is substituted with one to four substituents independently selected from the group consisting of halogen, —CN, —OH, C₁-C₆alkoxy, C₁-C₆alkylOC₁-C₆alkyl, C₁-C₆alkylCOOH, —COOH, oxo, —COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, —C₁-C₆alkylOhaloC₁-C₆alkyl, haloC₁-C₆alkyl, C₁-C₆alkylOH, —CON(R⁷)(R⁸), —N(R⁷)(R⁸) and C₁-C₆alkylN(R⁷)(R⁸).

In certain embodiments, the cycloalkyl is substituted with one substituent selected from the group consisting of halogen, —CN, —OH, C₁-C₆alkoxy, C₁-C₆alkylOC₁-C₆alkyl, C₁-C₆alkylCOOH, —COOH, oxo, —COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, —C₁-C₆alkylOhaloC₁-C₆alkyl, haloC₁-C₆alkyl, C₁-C₆alkylOH, —CON(R⁷)(R⁸), —N(R⁷)(R⁸) and C₁-C₆alkylN(R⁷)(R⁸).

In certain embodiments, the cycloalkyl is substituted with two substituents independently selected from the group consisting of halogen, —CN, —OH, C₁-C₆alkoxy, C₁-C₆alkylOC₁-C₆alkyl, C₁-C₆alkylCOOH, —COOH, oxo, —COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, —C₁-C₆alkylOhaloC₁-C₆alkyl, haloC₁-C₆alkyl, C₁-C₆alkylOH, —CON(R⁷)(R⁸), —N(R⁷)(R⁸) and C₁-C₆alkylN(R⁷)(R⁸).

In certain embodiments, the cycloalkyl is substituted with three substituents independently selected from the group consisting of halogen, —CN, —OH, C₁-C₆alkoxy, C₁-C₆alkylOC₁-C₆alkyl, C₁-C₆alkylCOOH, —COOH, oxo, —COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, —C₁-C₆alkylOhaloC₁-C₆alkyl, haloC₁-C₆alkyl, C₁-C₆alkylOH, —CON(R⁷)(R⁸), —N(R⁷)(R⁸) and C₁-C₆alkylN(R⁷)(R⁸).

In certain embodiments, the cycloalkyl is substituted with —OH.

In certain embodiments, the cycloalkyl is substituted with four substituents independently selected from the group consisting of halogen, —CN, —OH, C₁-C₆alkoxy, C₁-C₆alkylOC₁-C₆alkyl, C₁-C₆alkylCOOH, —COOH, oxo, —COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, —C₁-C₆alkylOhaloC₁-C₆alkyl, haloC₁-C₆alkyl, C₁-C₆alkylOH, —CON(R⁷)(R⁸), —N(R⁷)(R⁸) and C₁-C₆alkylN(R⁷)(R⁸).

In certain embodiments, R^{a} is taken with R^{b} and forms a heterocycloalkyl, wherein the heterocycloalkyl is unsubstituted or substituted with one to four substituents independently selected from the group consisting of halogen, —CN, —OH, C₁-C₆alkoxy, C₁-C₆alkylOC₁-C₆alkyl, C₁-C₆alkylCOOH, —COOH, oxo, —COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, —C₁-C₆alkylOhaloC₁-C₆alkyl, haloC₁-C₆alkyl, C₁-C₆alkylOH, —CON(R⁷)(R⁸), —N(R⁷)(R⁸) and C₁-C₆alkylN(R⁷)(R⁸).

Non-limiting examples of monocyclic heterocycloalkyl groups include piperidyl, oxetanyl, pyrrolyl, piperazinyl, morpholinyl, thiomorpholinyl, thiazolidinyl, 1,4-dioxanyl, tetrahydrofuranyl, tetrahydrothiophenyl, beta lactam, gamma lactam, delta lactam, beta lactone, gamma lactone, delta lactone, and pyrrolidinone, and oxides thereof. Non-limiting examples of heterocycloalkyl groups include, but are not limited to,

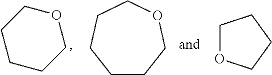

Non-limiting examples of bicyclic heterocycloalkyl groups include, but are not limited to,

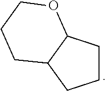

In certain embodiments, R^{a} is taken with R^{b} and forms:

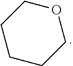

In certain embodiments, the heterocycloalkyl is unsubstituted. In certain embodiments, the heterocycloalkyl is substituted with one to four substituents independently selected from the group consisting of halogen, —CN, —OH, C₁-C₆alkoxy, C₁-C₆alkylOC₁-C₆alkyl, C₁-C₆alkylCOOH, —COOH, oxo, —COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, —C₁-C₆alkylOhaloC₁-C₆alkyl, haloC₁-C₆alkyl, C₁-C₆alkylOH, —CON(R⁷)(R⁸), —N(R⁷)(R⁸) and C₁-C₆alkylN(R⁷)(R⁸).

In certain embodiments, the heterocycloalkyl is substituted with one substituent selected from the group consisting of halogen, —CN, —OH, C₁-C₆alkoxy, C₁-C₆alkylOC₁-C₆alkyl, C₁-C₆alkylCOOH, —COOH, oxo, —COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, —C₁-C₆alkylOhaloC₁-C₆alkyl, haloC₁-C₆alkyl, C₁-C₆alkylOH, —CON(R⁷)(R⁸), —N(R⁷)(R⁸) and C₁-C₆alkylN(R⁷)(R⁸).

In certain embodiments, the heterocycloalkyl is substituted with two substituents independently selected from the group consisting of halogen, —CN, —OH, C₁-C₆alkoxy, C₁-C₆alkylOC₁-C₆alkyl, C₁-C₆alkylCOOH, —COOH, oxo, —COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, —C₁-C₆alkylOhaloC₁-C₆alkyl, haloC₁-C₆alkyl, C₁-C₆alkylOH, —CON(R⁷)(R⁸), —N(R⁷)(R⁸) and C₁-C₆alkylN(R⁷)(R⁸).

In certain embodiments, the heterocycloalkyl is substituted with two C₁-C₆alkyl substituents.

In certain embodiments, the heterocycloalkyl is substituted with three substituents independently selected from the group consisting of halogen, —CN, —OH, C₁-C₆alkoxy, C₁-C₆alkylOC₁-C₆alkyl, C₁-C₆alkylCOOH, —COOH, oxo, —COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, —C₁-C₆alkylOhaloC₁-C₆alkyl, haloC₁-C₆alkyl, C₁-C₆alkylOH, —CON(R⁷)(R⁸), —N(R⁷)(R⁸) and C₁-C₆alkylN(R⁷)(R⁸).

In certain embodiments, the heterocycloalkyl is substituted with three substituents independently selected from the group consisting of C₁-C₆alkyl and —OH.

In certain embodiments, the heterocycloalkyl is substituted with four substituents independently selected from the group consisting of halogen, —CN, —OH, C₁-C₆alkoxy, C₁-C₆alkylOC₁-C₆alkyl, C₁-C₆alkylCOOH, —COOH, oxo, —COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, —C₁-C₆alkylOhaloC₁-C₆alkyl, haloC₁-C₆alkyl, C₁-C₆alkylOH, —CON(R⁷)(R⁸), —N(R⁷)(R⁸) and C₁-C₆alkylN(R⁷)(R⁸).

In the compounds described herein, R^{b} is hydrogen, halogen, —CN, —OH, C₁-C₆alkoxy, C₁-C₆alkylOC₁-C₆alkyl, C₁-C₆alkylCOOH, —COOH, oxo, —COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, —C₁-C₆alkylOhaloC₁-C₆alkyl, haloC₁-C₆alkyl, C₁-C₆alkylOH, —CON(R⁷)(R⁸), —N(R⁷)(R⁸) or C₁-C₆alkylN(R⁷)(R⁸) or when taken with R^{a} forms a C₃-C₆cycloalkyl or heterocycloalkyl, wherein the C₃-C₆cycloalkyl or heterocycloalkyl is unsubstituted or substituted with one to four substituents independently selected from the group consisting of halogen, —CN, —OH, C₁-C₆alkoxy, C₁-C₆alkylOC₁-C₆alkyl, C₁-C₆alkylCOOH, —COOH, oxo, —COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, —C₁-C₆alkylOhaloC₁-C₆alkyl, haloC₁-C₆alkyl, C₁-C₆alkylOH, —CON(R⁷)(R⁸), —N(R⁷)(R⁸) and C₁-C₆alkylN(R⁷)(R⁸).

In certain embodiments described herein, R^{b} is hydrogen.

In certain embodiments, R^{b} is halogen. Examples of suitable halogens include chlorine, bromine, fluorine and iodine.

In certain embodiments, R^{b} is —CN.

In certain embodiments, R^{b} is —OH.

In certain embodiments, R^{b} is C₁-C₆alkoxy. Suitable alkoxys include, but are not limited to, methoxy, ethoxy, n-propoxy, isopropoxy and n-butoxy.

In certain embodiments, R^{b} is C₁-C₆alkylOC₁-C₆alkyl.

In certain embodiments, R^{b} is C₁-C₆alkylCOOH.

In certain embodiments, R^{b} is —COOH.

In certain embodiments, R^{b} is an oxo group.

In certain embodiments, R^{b} is —COOC₁-C₆alkyl.

In certain embodiments, R^{b} is C₁-C₆alkylCOOC₁-C₆alkyl.

In certain embodiments, R^{b} is C₃-C₆cycloalkyl. Suitable examples of cycloalkyls include, but are not limited to, cyclopropyl, cyclobutyl, cyclopentyl and cyclohexyl.

In certain embodiments, R^{b} is C₁-C₆alkylC₃-C₆cycloalkyl. Suitable examples of C₁-C₆alkylC₃-C₆cycloalkyls include, but are not limited to

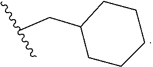 [SMILES: *CC1CCCCC1]

In certain embodiments, R^{b} is C₁-C₆alkyl. Examples of C₁-C₆alkyl groups include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R^{b} is methyl.

In certain embodiments, R^{b} is C₁-C₆alkylOhaloC₁-C₆alkyl. Suitable examples of C₁-C₆alkylOhaloC₁-C₆alkyls include, but are not limited to,

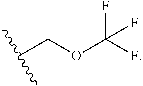 [SMILES: *COC(F)(F)F]

In certain embodiments, R^{b} is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl.

In certain embodiments, R^{b} is C₁-C₆alkylOH. Suitable alcohols include, but are not limited to, methanol, ethanol, propanol, butanol and iso-butanol.

In certain embodiments, R^{b} is —CON(R⁷)(R⁸). In certain embodiments, R^{b} is N(R⁷)(R⁸). In certain embodiments, R^{b} is C₁-C₆alkylN(R⁷)(R⁸), wherein R⁷ and R⁸ will be described in detail below.

In certain embodiments, R^{b} is taken with R^{a} and forms a cycloalkyl or heterocycloalkyl, wherein the cycloalkyl or heterocycloalkyl is unsubstituted or substituted with one to four substituents independently selected from the group consisting of halogen, —CN, —OH, C₁-C₆alkoxy, C₁-C₆alkylOC₁-C₆alkyl, C₁-C₆alkylCOOH, —COOH, oxo, —COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, —C₁-C₆alkylOhaloC₁-C₆alkyl, haloC₁-C₆alkyl, C₁-C₆alkylOH, —CON(R⁷)(R⁸), —N(R⁷)(R⁸) and C₁-C₆alkylN(R⁷)(R⁸).

In certain embodiments, R^{b} is taken with R^{a} and forms a cycloalkyl, wherein the cycloalkyl is unsubstituted or substituted with one to four substituents independently selected from the group consisting of halogen, —CN, —OH, C₁-C₆alkoxy, C₁-C₆alkylOC₁-C₆alkyl, C₁-C₆alkylCOOH, —COOH, oxo, —COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, —C₁-C₆alkylOhaloC₁-C₆alkyl, haloC₁-C₆alkyl, C₁-C₆alkylOH, —CON(R⁷)(R⁸), —N(R⁷)(R⁸) and C₁-C₆alkylN(R⁷)(R⁸).

Suitable examples of cycloalkyls include, but are not limited to, cyclopropyl, cyclobutyl, cyclopentyl and cyclohexyl.

In certain embodiments, the cycloalkyl is unsubstituted. In certain embodiments, the cycloalkyl is substituted with one to four substituents independently selected from the group consisting of halogen, —CN, —OH, C₁-C₆alkoxy, C₁-C₆alkylOC₁-C₆alkyl, C₁-C₆alkylCOOH, —COOH, heterooxo, —COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, —C₁-C₆alkylOhaloC₁-C₆alkyl, haloC₁-C₆alkyl, C₁-C₆alkylOH, —CON(R⁷)(R⁸), —N(R⁷)(R⁸) and C₁-C₆alkylN(R⁷)(R⁸).

In certain embodiments, the cycloalkyl is substituted with one substituent selected from the group consisting of halogen, —CN, —OH, C₁-C₆alkoxy, C₁-C₆alkylOC₁-C₆alkyl, C₁-C₆alkylCOOH, —COOH, oxo, —COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, —C₁-C₆alkylOhaloC₁-C₆alkyl, haloC₁-C₆alkyl, C₁-C₆alkylOH, —CON(R⁷)(R⁸), —N(R⁷)(R⁸) and C₁-C₆alkylN(R⁷)(R⁸).

In certain embodiments, the cycloalkyl is substituted with two substituents independently selected from the group consisting of halogen, —CN, —OH, C₁-C₆alkoxy, C₁-C₆alkylOC₁-C₆alkyl, C₁-C₆alkylCOOH, —COOH, oxo, —COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, —C₁-C₆alkylOhaloC₁-C₆alkyl, haloC₁-C₆alkyl, C₁-C₆alkylOH, —CON(R⁷)(R⁸), —N(R⁷)(R⁸) and C₁-C₆alkylN(R⁷)(R⁸).

In certain embodiments, the cycloalkyl is substituted with three substituents independently selected from the group consisting of halogen, —CN, —OH, C₁-C₆alkoxy, C₁-C₆alkylOC₁-C₆alkyl, C₁-C₆alkylCOOH, —COOH, oxo, —COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, —C₁-C₆alkylOhaloC₁-C₆alkyl, haloC₁-C₆alkyl, C₁-C₆alkylOH, —CON(R⁷)(R⁸), —N(R⁷)(R⁸) and C₁-C₆alkylN(R⁷)(R⁸).

In certain embodiments, the cycloalkyl is substituted with —OH.

In certain embodiments, the cycloalkyl is substituted with four substituents independently selected from the group consisting of halogen, —CN, —OH, C₁-C₆alkoxy, C₁-C₆alkylOC₁-C₆alkyl, C₁-C₆alkylCOOH, —COOH, oxo, —COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, —C₁-C₆alkylOhaloC₁-C₆alkyl, haloC₁-C₆alkyl, C₁-C₆alkylOH, —CON(R⁷)(R⁸), —N(R⁷)(R⁸) and C₁-C₆alkylN(R⁷)(R⁸).

In certain embodiments, R^{b} is taken with R^{a} and forms a heterocycloalkyl, wherein the heterocycloalkyl is unsubstituted or substituted with one to four substituents independently selected from the group consisting of halogen, —CN, —OH, C₁-C₆alkoxy, C₁-C₆alkylOC₁-C₆alkyl, C₁-C₆alkylCOOH, —COOH, oxo, —COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, —C₁-C₆alkylOhaloC₁-C₆alkyl, haloC₁-C₆alkyl, C₁-C₆alkylOH, —CON(R⁷)(R⁸), —N(R⁷)(R⁸) and C₁-C₆alkylN(R⁷)(R⁸).

Non-limiting examples of monocyclic heterocycloalkyl groups include piperidyl, oxetanyl, pyrrolyl, piperazinyl, morpholinyl, thiomorpholinyl, thiazolidinyl, 1,4-dioxanyl, tetrahydrofuranyl, tetrahydrothiophenyl, beta lactam, gamma lactam, delta lactam, beta lactone, gamma lactone, delta lactone, and pyrrolidinone, and oxides thereof. Non-limiting examples of heterocycloalkyl groups include, but are not limited to,

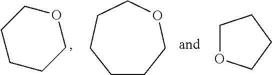

Non-limiting examples of bicyclic heterocycloalkyl groups include, but are not limited to,

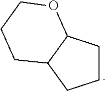

In certain embodiments, R^{b} is taken with R^{a} and forms:

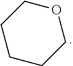

In certain embodiments, the heterocycloalkyl is unsubstituted. In certain embodiments, the heterocycloalkyl is substituted with one to four substituents independently selected from the group consisting of halogen, —CN, —OH, C₁-C₆alkoxy, C₁-C₆alkylOC₁-C₆alkyl, C₁-C₆alkylCOOH, —COOH, oxo, —COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, —C₁-C₆alkylOhaloC₁-C₆alkyl, haloC₁-C₆alkyl, C₁-C₆alkylOH, —CON(R⁷)(R⁸), —N(R⁷)(R⁸) and C₁-C₆alkylN(R⁷)(R⁸).

In certain embodiments, the heterocycloalkyl is substituted with one substituent selected from the group consisting of halogen, —CN, —OH, C₁-C₆alkoxy, C₁-C₆alkylOC₁-C₆alkyl, C₁-C₆alkylCOOH, —COOH, oxo, —COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, —C₁-C₆alkylOhaloC₁-C₆alkyl, haloC₁-C₆alkyl, C₁-C₆alkylOH, —CON(R⁷)(R⁸), —N(R⁷)(R⁸) and C₁-C₆alkylN(R⁷)(R⁸).

In certain embodiments, the heterocycloalkyl is substituted with two substituents independently selected from the group consisting of halogen, —CN, —OH, C₁-C₆alkoxy, C₁-C₆alkylOC₁-C₆alkyl, C₁-C₆alkylCOOH, —COOH, oxo, —COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, —C₁-C₆alkylOhaloC₁-C₆alkyl, haloC₁-C₆alkyl, C₁-C₆alkylOH, —CON(R⁷)(R⁸), —N(R⁷)(R⁸) and C₁-C₆alkylN(R⁷)(R⁸).

In certain embodiments, the heterocycloalkyl is substituted with two C₁-C₆alkyl substituents.

In certain embodiments, the heterocycloalkyl is substituted with three substituents independently selected from the group consisting of halogen, —CN, —OH, C₁-C₆alkoxy, C₁-C₆alkylOC₁-C₆alkyl, C₁-C₆alkylCOOH, —COOH, oxo, —COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, —C₁-C₆alkylOhaloC₁-C₆alkyl, haloC₁-C₆alkyl, C₁-C₆alkylOH, —CON(R⁷)(R⁸), —N(R⁷)(R⁸) and C₁-C₆alkylN(R⁷)(R⁸).

In certain embodiments, the heterocycloalkyl is substituted with three substituents independently selected from the group consisting of C₁-C₆alkyl and —OH.

In certain embodiments, the heterocycloalkyl is substituted with four substituents independently selected from the group consisting of halogen, —CN, —OH, C₁-C₆alkoxy, C₁-C₆alkylOC₁-C₆alkyl, C₁-C₆alkylCOOH, —COOH, oxo, —COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, —C₁-C₆alkylOhaloC₁-C₆alkyl, haloC₁-C₆alkyl, C₁-C₆alkylOH, —CON(R⁷)(R⁸), —N(R⁷)(R⁸) and C₁-C₆alkylN(R⁷)(R⁸).

In the embodiments described herein, R³ is hydrogen, halogen, —CN, —OH, C₁-C₆alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸), —N(R⁷)(R⁸), C₁-C₆alkylN(R⁷)(R⁸), C₁-C₆alkyl(OCH₂CH₂)ₙN(R⁷)(R⁸) or C₁-C₆alkylOhaloC₁-C₆alkyl or when taken with R⁴ forms a C₃-C₆cycloalkyl or C₃-C₆heterocycloalkyl.

In certain embodiments of the compounds described herein, R³ is hydrogen, halogen, —CN, —OH, C₁-C₆alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸), —N(R⁷)(R⁸) or C₁-C₆alkylN(R⁷)(R⁸) or when taken with R⁴ forms a C₃-C₆cycloalkyl or C₃-C₆heterocycloalkyl. In certain embodiments, R³ is hydrogen. In certain embodiments, R³ is halogen. Suitable halogens include fluorine, chlorine, bromine, and iodine. In certain embodiments, R³ is —CN. In certain embodiments, R³ is —OH.

In certain embodiments, R³ is C₁-C₆alkoxy. Suitable alkoxys include, but are not limited to, methoxy, ethoxy, n-propoxy, isopropoxy and n-butoxy. In certain embodiments, R³ is C₁-C₆alkylOC₁-C₆alkyl. In certain embodiments, R³ is —COOH. In certain embodiments, R³ is C₁-C₆alkylCOOH. In certain embodiments, R³ is C₃-C₆cycloalkyl. Suitable examples of cycloalkyls include, but are not limited to, cyclopropyl, cyclobutyl, cyclopentyl and cyclohexyl. In certain embodiments, R³ is C₁-C₆alkyl. Examples of C₁-C₆alkyl groups include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R³ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R³ is C₁-C₆alkylOH. Examples of suitable alcohols include, but are not limited to, methanol, ethanol, propanol, butanol and iso-butanol. In certain embodiments, R³ is —CON(R⁷)(R⁸). Suitable examples of —CON(R⁷)(R⁸) include, but are not limited to, —CONH₂ and —CON(CH₃)₂. In certain embodiments, R³ is N(R⁷)(R⁸). Suitable examples of N(R⁷)(R⁸) include, but are not limited to, NH₂ and N(CH₃)₂. In certain embodiments, R³ is C₁-C₆alkylN(R⁷)(R⁸). Suitable examples of C₁-C₆alkylN(R⁷)(R⁸) include, but are not limited to,

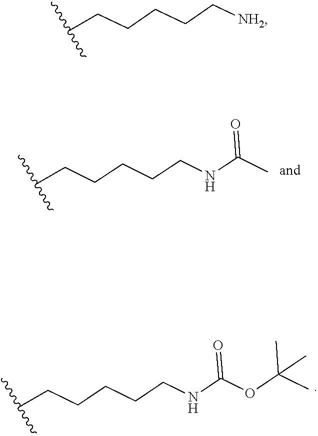

R⁷ and R⁸ are discussed in further detail below.

In certain embodiments, R³ is C₁-C₆alkylOhaloC₁-C₆alkyl. Suitable examples of C₁-C₆alkylOhaloalkyls include, but are not limited to,

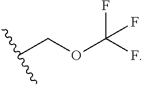 [SMILES: *COC(F)(F)F]

In certain embodiments, R³ is C₁-C₆alkyl(OCH₂CH₂)ₙN(R⁷)(R⁸). R⁷, R⁸ and n are discussed in detail below. Suitable examples of C₁-C₆alkyl(OCH₂CH₂)ₙN(R⁷)(R⁸) include, but are not limited to,

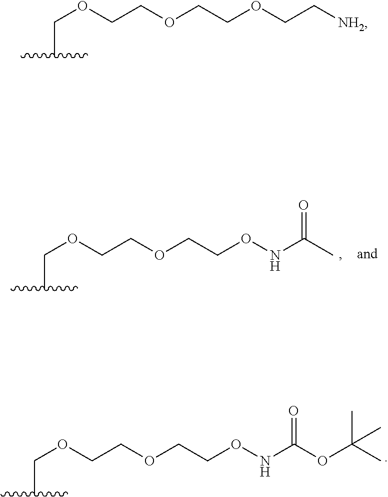 [SMILES: *COCCOCCOCCN ; *COCCOCCONC(C)=O ; *COCCOCCONC(=O)OC(C)(C)C]

With regard to the compounds described herein, n is 1, 2, 3 or 4. In certain embodiments, n is 1. In certain embodiments, n is 2. In certain embodiments, n is 3. In certain embodiments, n is 4.

In certain embodiments, R³ is taken with R⁴ and forms a C₃-C₆cycloalkyl or C₃-C₆heterocycloalkyl. In certain embodiments, R³ is taken with R⁴ and forms a C₃-C₆cycloalkyl. Suitable examples of cycloalkyls include, but are not limited to, cyclopropyl, cyclobutyl, cyclopentyl and cyclohexyl. In certain embodiments, R³ is taken with R⁴ and forms a C₃-C₆heterocycloalkyl. Suitable examples of heterocycloalkyls include, but are not limited to, piperidyl, oxetanyl, pyrrolyl, piperazinyl, morpholinyl, thiomorpholinyl, thiazolidinyl, 1,4-dioxanyl, tetrahydrofuranyl, tetrahydrothiophenyl, beta lactam, gamma lactam, delta lactam, beta lactone, gamma lactone, delta lactone, and pyrrolidinone, and oxides thereof.

In certain embodiments, R³ is hydrogen, fluorine, methyl, ethyl, —OH, methoxy,

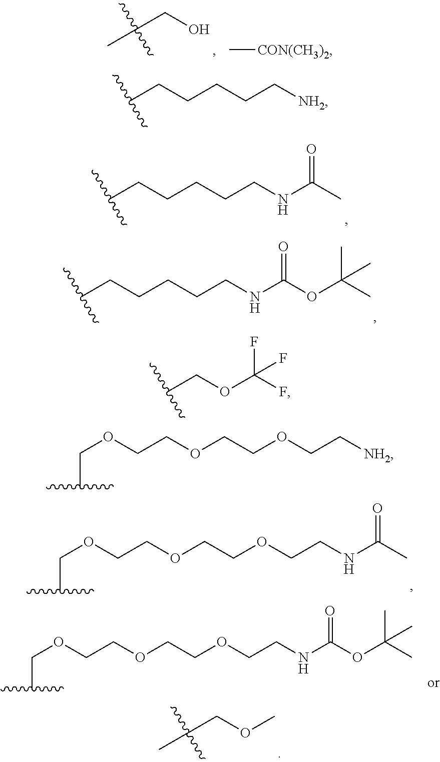

In certain embodiments, R³ is hydrogen, methyl, ethyl or

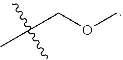 [SMILES: *COC]

In certain embodiments, R³ is taken with R⁴ to form oxetanyl.

In certain embodiments described herein, R⁴ is hydrogen, halogen, —CN, —OH, C₁-C₆alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸), —N(R⁷)(R⁸), C₁-C₆alkylN(R⁷)(R⁸), C₁-C₆alkyl(OCH₂CH₂)ₙN(R⁷)(R⁸) or C₁-C₆alkylOhaloC₁-C₆alkyl or when taken with R³ forms a C₃-C₆cycloalkyl or C₃-C₆heterocycloalkyl. In certain embodiments of the compounds described herein, R⁴ is hydrogen, halogen, —CN, —OH, C₁-C₆alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸), —N(R⁷)(R⁸) or C₁-C₆alkylN(R⁷)(R⁸) or when taken with R³ forms a C₃-C₆cycloalkyl or C₃-C₆heterocycloalkyl. In certain embodiments, R⁴ is hydrogen. In certain embodiments, R⁴ is halogen. Suitable halogens include fluorine, chlorine, bromine, or iodine. In certain embodiments, R⁴ is —CN. In certain embodiments, R⁴ is —OH.

In certain embodiments, R⁴ is C₁-C₆alkoxy. Suitable alkoxys include, but are not limited to, methoxy, ethoxy, n-propoxy, isopropoxy and n-butoxy. In certain embodiments, R⁴ is C₁-C₆alkylOC₁-C₆alkyl. In certain embodiments, R⁴ is —COOH. In certain embodiments, R⁴ is C₁-C₆alkylCOOH. In certain embodiments, R⁴ is C₃-C₆cycloalkyl. Suitable examples of cycloalkyls include, but are not limited to, cyclopropyl, cyclobutyl, cyclopentyl and cyclohexyl. In certain embodiments, R⁴ is C₁-C₆alkyl. Examples of C₁-C₆alkyl groups can include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R⁴ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R⁴ is C₁-C₆alkylOH. Examples of suitable alcohols include, but are not limited to, methanol, ethanol, propanol, butanol and iso-butanol. In certain embodiments, R⁴ is —CON(R⁷)(R⁸). Suitable examples of —CON(R⁷)(R⁸) include, but are not limited to, —CONH₂ and —CON(CH₃)₂. In certain embodiments, R⁴ is N(R⁷)(R⁸). Suitable examples of N(R⁷)(R⁸) include, but are not limited to, NH₂ and N(CH₃)₂. In certain embodiments, R⁴ is C₁-C₆alkylN(R⁷)(R⁸). Suitable examples of C₁-C₆alkylN(R⁷)(R⁸) include, but are not limited to,

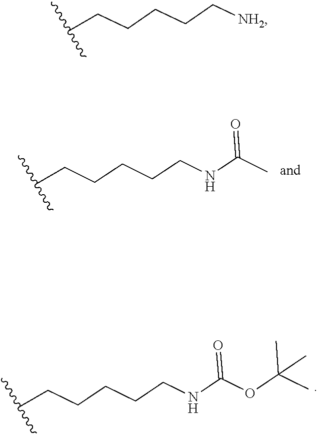

R⁷ and R⁸ are discussed in further detail below.

In certain embodiments, R⁴ is C₁-C₆alkylOhaloC₁-C₆alkyl. Suitable examples of haloalkyls include, but are not limited to,

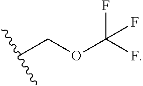 [SMILES: *COC(F)(F)F]

In certain embodiments, R⁴ is C₁-C₆alkyl(OCH₂CH₂)ₙN(R⁷)(R⁸). R⁷, R⁸ are discussed in detail below and n is discussed above. Suitable examples of C₁-C₆alkyl(OCH₂CH₂)ₙN(R⁷)(R⁸) include, but are not limited to,

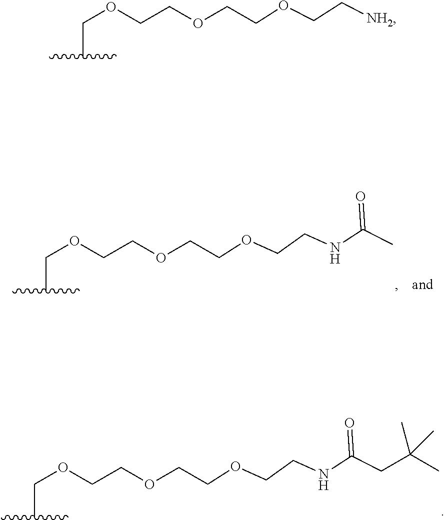 [SMILES: *COCCOCCOCCN ; *COCCOCCOCCNC(C)=O ; *COCCOCCOCCNC(=O)CC(C)(C)C]

In certain embodiments, R⁴ is taken with R³ and forms a C₃-C₆cycloalkyl or C₃-C₆heterocycloalkyl. In certain embodiments, R⁴ is taken with R³ and forms a C₃-C₆cycloalkyl. Suitable examples of cycloalkyls include, but are not limited to, cyclopropyl, cyclobutyl, cyclopentyl and cyclohexyl. In certain embodiments, R⁴ is taken with R³ and forms a C₃-C₆heterocycloalkyl. Suitable examples of heterocycloalkyls include, but are not limited to, piperidyl, oxetanyl, pyrrolyl, piperazinyl, morpholinyl, thiomorpholinyl, thiazolidinyl, 1,4-dioxanyl, tetrahydrofuranyl, tetrahydrothiophenyl, beta lactam, gamma lactam, delta lactam, beta lactone, gamma lactone, delta lactone, and pyrrolidinone, and oxides thereof.

In certain embodiments, R⁴ is hydrogen or methyl. In certain embodiments, R⁴ is hydrogen, methyl, ethyl or

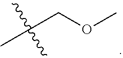 [SMILES: *COC]

In certain embodiments, R⁴ is taken with R³ to form oxetanyl. In certain embodiments, R³ and R⁴ are both hydrogen, methyl or ethyl.

In certain embodiments, R³ is hydrogen and R⁴ is hydrogen.

In the embodiments described herein, R⁵ is hydrogen, halogen, —CN, —OH, C₁-C₆alkoxy, C₁-C₆alkylOC₁-C₆alkyl, C₁-C₆alkylCOOH, —COOH, oxo, —COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, —C₁-C₆alkylOhaloC₁-C₆alkyl, haloC₁-C₆alkyl, C₁-C₆alkylOH, —CON(R⁷)(R⁸), —N(R⁷)(R⁸) or C₁-C₆alkylN(R⁷)(R⁸).

In certain embodiments described herein, R⁵ is hydrogen.

In certain embodiments, R⁵ is halogen. Examples of suitable halogens include chlorine, bromine, fluorine and iodine.

In certain embodiments, R⁵ is —CN.

In certain embodiments, R⁵ is —OH.

In certain embodiments, R⁵ is C₁-C₆alkoxy. Suitable alkoxys include, but are not limited to, methoxy, ethoxy, n-propoxy, isopropoxy and n-butoxy.

In certain embodiments, R⁵ is C₁-C₆alkylOC₁-C₆alkyl.

In certain embodiments, R⁵ is C₁-C₆alkylCOOH.

In certain embodiments, R⁵ is —COOH.

In certain embodiments, R⁵ is an oxo group.

In certain embodiments, R⁵ is —COOC₁-C₆alkyl.

In certain embodiments, R⁵ is C₁-C₆alkylCOOC₁-C₆alkyl.

In certain embodiments, R⁵ is C₃-C₆cycloalkyl. Suitable examples of cycloalkyls include, but are not limited to, cyclopropyl, cyclobutyl, cyclopentyl and cyclohexyl.

In certain embodiments, R⁵ is C₁-C₆alkylC₃-C₆cycloalkyl. Suitable examples of cycloalkyls include, but are not limited to

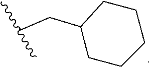 [SMILES: *CC1CCCCC1]

In certain embodiments, R⁵ is C₁-C₆alkyl. Examples of C₁-C₆alkyl groups can include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R⁵ is methyl.

In certain embodiments, R⁵ is C₁-C₆alkylOhaloC₁-C₆alkyl. Suitable examples of C₁-C₆alkylOhaloC₁-C₆alkyls include, but are not limited to,

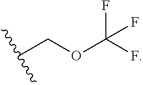 [SMILES: *COC(F)(F)F]

In certain embodiments, R⁵ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl.

In certain embodiments, R⁵ is C₁-C₆alkylOH. Suitable alcohols include, but are not limited to, methanol, ethanol, propanol, butanol and iso-butanol.

In certain embodiments, R⁵ is —CON(R⁷)(R⁸). In certain embodiments, R⁵ is N(R⁷)(R⁸). In certain embodiments, R⁵ is C₁-C₆alkylN(R⁷)(R⁸), wherein R⁷ and R⁸ will be described in detail below.

In certain embodiments, R⁵ is hydrogen, methyl, ethyl or t-butyl.

In the embodiments described herein, R⁶ is hydrogen, halogen, —CN, —OH, C₁-C₆alkoxy, C₁-C₆alkylOC₁-C₆alkyl, C₁-C₆alkylCOOH, —COOH, oxo, —COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, —C₁-C₆alkylOhaloC₁-C₆alkyl, haloC₁-C₆alkyl, C₁-C₆alkylOH, —CON(R⁷)(R⁸), —N(R⁷)(R⁸) or C₁-C₆alkylN(R⁷)(R⁸).

In certain embodiments described herein, R⁶ is hydrogen.

In certain embodiments, R⁶ is halogen. Examples of suitable halogens include chlorine, bromine, fluorine and iodine.

In certain embodiments, R⁶ is —CN.

In certain embodiments, R⁶ is —OH.

In certain embodiments, R⁶ is C₁-C₆alkoxy. Suitable alkoxys include, but are not limited to, methoxy, ethoxy, n-propoxy, isopropoxy and n-butoxy.

In certain embodiments, R⁶ is C₁-C₆alkylOC₁-C₆alkyl.

In certain embodiments, R⁶ is C₁-C₆alkylCOOH.

In certain embodiments, R⁶ is —COOH.

In certain embodiments, R⁶ is an oxo group.

In certain embodiments, R⁶ is —COOC₁-C₆alkyl.

In certain embodiments, R⁶ is C₁-C₆alkylCOOC₁-C₆alkyl.

In certain embodiments, R⁶ is C₃-C₆cycloalkyl. Suitable examples of cycloalkyls include, but are not limited to, cyclopropyl, cyclobutyl, cyclopentyl and cyclohexyl.

In certain embodiments, R⁶ is C₁-C₆alkylC₃-C₆cycloalkyl. Suitable examples of C₁-C₆alkylC₃-C₆cycloalkyls include, but are not limited to

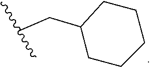 [SMILES: *CC1CCCCC1]

In certain embodiments, R⁶ is C₁-C₆alkyl. Examples of C₁-C₆alkyl groups include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R⁶ is methyl.

In certain embodiments, R⁶ is C₁-C₆alkylOhaloC₁-C₆alkyl. Suitable examples of C₁-C₆alkylOhaloC₁-C₆alkyls include, but are not limited to,

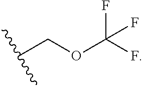 [SMILES: *COC(F)(F)F]

In certain embodiments, R⁶ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl.

In certain embodiments, R⁶ is C₁-C₆alkylOH. Suitable alcohols include, but are not limited to, methanol, ethanol, propanol, butanol and iso-butanol.

In certain embodiments, R⁶ is —CON(R⁷)(R⁸). In certain embodiments, R⁶ is N(R⁷)(R⁸). In certain embodiments, R⁶ is C₁-C₆alkylN(R⁷)(R⁸), wherein R⁷ and R⁸ will be described in detail below.

In certain embodiments, R⁶ is hydrogen, methyl, ethyl or t-butyl.

In the embodiments described herein, R⁷ is hydrogen, C₁-C₆alkylCOOH, —COOH, C₃-C₆cycloalkyl, C₁-C₆alkyl, haloC₁-C₆alkyl, C₁-C₆alkylOH, —COC₁-C₆alkyl or —COOC₁-C₆alkyl. In certain embodiments, R⁷ is hydrogen, C₁-C₆alkylCOOH, —COOH, C₃-C₆cycloalkyl, C₁-C₆alkyl, haloC₁-C₆alkyl or C₁-C₆alkylOH.

In certain embodiments, R⁷ is hydrogen. In certain embodiments, R⁷ is C₁-C₆alkylCOOH. In certain embodiments, R⁷ is —COOH. In certain embodiments, R⁷ is C₃-C₆cycloalkyl. Suitable examples of cycloalkyls include, but are not limited to, cyclopropyl, cyclobutyl, cyclopentyl and cyclohexyl. In certain embodiments, R⁷ is C₁-C₆alkyl. Examples of C₁-C₆alkyl groups can include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R⁷ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R⁷ is C₁-C₆alkylOH. Examples of suitable alcohols include, but are not limited to, methanol, ethanol, propanol, butanol and iso-butanol.

In certain embodiments, R⁷ is —COC₁-C₆alkyl. Suitable examples include, but are not limited to, —COCH₃. In certain embodiments, R⁷ is —COOC₁-C₆alkyl. Suitable examples include, but are not limited to, —COOCH₃.

In the embodiments described herein, R⁸ is hydrogen, C₁-C₆alkylCOOH, —COOH, C₃-C₆cycloalkyl, C₁-C₆alkyl, haloC₁-C₆alkyl, C₁-C₆alkylOH, —COC₁-C₆alkyl or —COOC₁-C₆alkyl. In certain embodiments, R⁸ is hydrogen, C₁-C₆alkylCOOH, —COOH, C₃-C₆cycloalkyl, C₁-C₆alkyl, haloC₁-C₆alkyl or C₁-C₆alkylOH.

In certain embodiments, R⁸ is hydrogen. In certain embodiments, R⁸ is C₁-C₆alkylCOOH. In certain embodiments, R⁸ is —COOH. In certain embodiments, R⁸ is C₃-C₆cycloalkyl. Suitable examples of cycloalkyls include, but are not limited to, cyclopropyl, cyclobutyl, cyclopentyl and cyclohexyl. In certain embodiments, R⁸ is C₁-C₆alkyl. Examples of C₁-C₆alkyl groups can include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R⁸ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R⁸ is C₁-C₆alkylOH. Examples of suitable alcohols include, but are not limited to, methanol, ethanol, propanol, butanol and iso-butanol.

In certain embodiments, R⁸ is —COC₁-C₆alkyl. Suitable examples include, but are not limited to, —COCH₃. In certain embodiments, R⁸ is —COOC₁-C₆alkyl. Suitable examples include, but are not limited to, —COOCH₃.

In the embodiments described herein, R⁹ is hydrogen, halogen, —CN, —OH, C₁-C₆alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸) and N(R⁷)(R⁸). In certain embodiments, R⁹ is hydrogen. In certain embodiments, R⁹ is halogen. Suitable halogens include fluorine, chlorine, bromine, or iodine. In certain embodiments, R⁹ is —CN. In certain embodiments, R⁹ is —OH.

In certain embodiments, R⁹ is C₁-C₆alkoxy. Suitable alkoxys include, but are not limited to, methoxy, ethoxy, n-propoxy, isopropoxy and n-butoxy. In certain embodiments, R⁹ is C₁-C₆alkylOC₁-C₆alkyl. In certain embodiments, R⁹ is —COOH. In certain embodiments, R⁹ is C₁-C₆alkylCOOH. In certain embodiments, R⁹ is C₃-C₆cycloalkyl. Suitable examples of cycloalkyls include, but are not limited to, cyclopropyl, cyclobutyl, cyclopentyl and cyclohexyl. In certain embodiments, R⁹ is C₁-C₆alkyl. Examples of C₁-C₆alkyl groups can include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R⁹ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R⁹ is C₁-C₆alkylOH. Examples of suitable alcohols include, but are not limited to, methanol, ethanol, propanol, butanol and iso-butanol. In certain embodiments, R⁹ is —CON(R⁷)(R⁸). In certain embodiments, R⁹ is N(R⁷)(R⁸). In certain embodiments, R⁹ is C₁-C₆alkylN(R⁷)(R⁸).

With regard to the compounds described herein, R¹⁰ is hydrogen, halogen, —CN, —OH, C₁-C₆alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸) or N(R⁷)(R⁸). In certain embodiments, R¹⁰ is hydrogen. In certain embodiments, R¹⁰ is halogen. Suitable halogens include fluorine, chlorine, bromine, or iodine. In certain embodiments, R¹⁰ is —CN. In certain embodiments, R¹⁰ is —OH.

In certain embodiments, R¹⁰ is C₁-C₆alkoxy. Suitable alkoxys include, but are not limited to, methoxy, ethoxy, n-propoxy, isopropoxy and n-butoxy. In certain embodiments, R¹⁰ is C₁-C₆alkylOC₁-C₆alkyl. In certain embodiments, R¹⁰ is —COOH. In certain embodiments, R¹⁰ is C₁-C₆alkylCOOH. In certain embodiments, R¹⁰ is C₃-C₆cycloalkyl. Suitable examples of cycloalkyls include, but are not limited to, cyclopropyl, cyclobutyl, cyclopentyl and cyclohexyl. In certain embodiments, R¹⁰ is C₁-C₆alkyl. Examples of C₁-C₆alkyl groups can include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R¹⁰ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R¹⁰ is C₁-C₆alkylOH. Examples of suitable alcohols include, but are not limited to, methanol, ethanol, propanol, butanol and iso-butanol. In certain embodiments, R¹⁰ is —CON(R⁷)(R⁸). In certain embodiments, R¹⁰ is N(R⁷)(R⁸). In certain embodiments, R¹⁰ is C₁-C₆alkylN(R⁷)(R⁸).

In the embodiments, described herein, R¹¹ is hydrogen, halogen, —CN, —OH, C₁-C₆alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸) or N(R⁷)(R⁸). In certain embodiments, R¹¹ is hydrogen. In certain embodiments, R¹¹ is halogen. Suitable halogens include fluorine, chlorine, bromine, or iodine. In certain embodiments, R¹¹ is —CN. In certain embodiments, R¹¹ is —OH.

In certain embodiments, R¹¹ is C₁-C₆alkoxy. Suitable alkoxys include, but are not limited to, methoxy, ethoxy, n-propoxy, isopropoxy and n-butoxy. In certain embodiments, R¹¹ is C₁-C₆alkylOC₁-C₆alkyl. In certain embodiments, R¹¹ is —COOH. In certain embodiments, R¹¹ is C₁-C₆alkylCOOH. In certain embodiments, R¹¹ is C₃-C₆cycloalkyl. Suitable examples of cycloalkyls include, but are not limited to, cyclopropyl, cyclobutyl, cyclopentyl and cyclohexyl. In certain embodiments, R¹¹ is C₁-C₆alkyl. Examples of C₁-C₆alkyl groups include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R¹¹ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R¹¹ is C₁-C₆alkylOH. Examples of suitable alcohols include, but are not limited to, methanol, ethanol, propanol, butanol and iso-butanol. In certain embodiments, R¹¹ is —CON(R⁷)(R⁸). In certain embodiments, R¹¹ is N(R⁷)(R⁸). In certain embodiments, R¹¹ is C₁-C₆alkylN(R⁷)(R⁸).

In the embodiments described herein, R¹² is hydrogen, halogen, —CN, —OH, C₁-C₆alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸), —N(R⁷)(R⁸) or C₁-C₆alkylN(R⁷)(R⁸). In certain embodiments, R¹² is hydrogen. In certain embodiments, R¹² is halogen. Suitable halogens include fluorine, chlorine, bromine, or iodine. In certain embodiments, R² is —CN. In certain embodiments, R² is —OH.

In certain embodiments, R¹² is C₁-C₆alkoxy. Suitable alkoxy groups include, but are not limited to, methoxy, ethoxy, n-propoxy, isopropoxy and n-butoxy. In certain embodiments, R¹² is C₁-C₆alkylOC₁-C₆alkyl. In certain embodiments, R¹² is —COOH. In certain embodiments, R¹² is C₁-C₆alkylCOOH. In certain embodiments, R¹² is C₃-C₆cycloalkyl. Suitable examples of cycloalkyls include, but are not limited to, cyclopropyl, cyclobutyl, cyclopentyl and cyclohexyl. In certain embodiments, R¹² is C₁-C₆alkyl. Examples of C₁-C₆alkyl groups include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R² is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R¹² is C₁-C₆alkylOH. Examples of suitable alcohols include, but are not limited to, methanol, ethanol, propanol, butanol and iso-butanol. In certain embodiments, R¹² is —CON(R⁷)(R⁸). In certain embodiments, R¹² is N(R⁷)(R⁸). In certain embodiments, R¹² is C₁-C₆alkylN(R⁷)(R⁸).

In certain embodiments, R¹² is hydrogen, methyl, ethyl, methoxy, —OH or

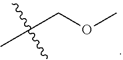 [SMILES: *COC]

In certain embodiments, R¹² is hydrogen or

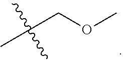 [SMILES: *COC]

In the embodiments described herein, R¹³ is hydrogen, halogen, —CN, —OH, C₁-C₆alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸), —N(R⁷)(R⁸) or C₁-C₆alkylN(R⁷)(R⁸). In certain embodiments, R¹³ is hydrogen. In certain embodiments, R¹³ is halogen. Suitable halogens include fluorine, chlorine, bromine, or iodine. In certain embodiments, R¹³ is —CN. In certain embodiments, R¹³ is —OH.

In certain embodiments, R¹³ is C₁-C₆alkoxy. Suitable alkoxy groups include, but are not limited to, methoxy, ethoxy, n-propoxy, isopropoxy and n-butoxy. In certain embodiments, R¹³ is C₁-C₆alkylOC₁-C₆alkyl. In certain embodiments, R¹³ is —COOH. In certain embodiments, R¹³ is C₁-C₆alkylCOOH. In certain embodiments, R¹³ is C₃-C₆cycloalkyl. Suitable examples of cycloalkyls include, but are not limited to, cyclopropyl, cyclobutyl, cyclopentyl and cyclohexyl. In certain embodiments, R¹³ is C₁-C₆alkyl. Examples of C₁-C₆alkyl groups include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R¹³ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R¹³ is C₁-C₆alkylOH. Examples of suitable alcohols include, but are not limited to, methanol, ethanol, propanol, butanol and iso-butanol. In certain embodiments, R¹³ is —CON(R⁷)(R⁸). In certain embodiments, R¹³ is N(R⁷)(R⁸). In certain embodiments, R¹³ is C₁-C₆alkylN(R⁷)(R⁸).

In certain embodiments, R¹³ is hydrogen, methyl, ethyl, methoxy, —OH or

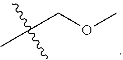 [SMILES: *COC]

In certain embodiments, R¹³ is hydrogen or

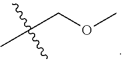 [SMILES: *COC]

In certain embodiments, R¹² and R¹³ are independently selected from the group consisting of hydrogen, C₁-C₆alkylOC₁-C₆alkyl, and C₁-C₆alkyl.

In certain embodiments described herein, each occurrence of R¹⁴ is independently selected from the group consisting of hydrogen, halogen, —CN, —OH, C₁-C₆alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸), —N(R⁷)(R⁸) and C₁-C₆alkylN(R⁷)(R⁸). In certain embodiments, R¹⁴ is hydrogen. In certain embodiments, R¹⁴ is halogen. Suitable halogens include fluorine, chlorine, bromine, or iodine. In certain embodiments, R¹⁴ is —CN. In certain embodiments, R¹⁴ is —OH.

In certain embodiments, R¹⁴ is C₁-C₆alkoxy. Suitable alkoxy groups include, but are not limited to, methoxy, ethoxy, n-propoxy, isopropoxy and n-butoxy. In certain embodiments, R¹⁴ is C₁-C₆alkylOC₁-C₆alkyl. In certain embodiments, R¹⁴ is —COOH. In certain embodiments, R¹⁴ is C₁-C₆alkylCOOH. In certain embodiments, R¹⁴ is C₃-C₆cycloalkyl. Suitable examples of cycloalkyls include, but are not limited to, cyclopropyl, cyclobutyl, cyclopentyl and cyclohexyl. In certain embodiments, R¹⁴ is C₁-C₆alkyl. Examples of C₁-C₆alkyl groups include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R¹⁴ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R¹⁴ is C₁-C₆alkylOH. Examples of suitable alcohols include, but are not limited to, methanol, ethanol, propanol, butanol and iso-butanol. In certain embodiments, R¹⁴ is —CON(R⁷)(R⁸). In certain embodiments, R¹⁴ is N(R⁷)(R⁸). In certain embodiments, R¹⁴ is C₁-C₆alkylN(R⁷)(R⁸).

In certain embodiments, wherein X is C(R¹⁴)₂, R¹⁴ is independently selected from the group consisting of hydrogen, halogen, —OH, C₁-C₆alkylOH, C₁-C₆alkylalkoxy, C₁-C₆alkylOC₁-C₆alkyl and C₁-C₆alkyl.

In certain embodiments, R¹⁴ is hydrogen, methyl, ethyl, methoxy, —OH or

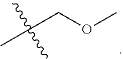 [SMILES: *COC]

In the embodiments described herein, R¹⁵ is hydrogen, halogen, —CN, —OH, C₁-C₆alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸) or N(R⁷)(R⁸). In certain embodiments, R¹⁵ is hydrogen. In certain embodiments, R⁵ is halogen. Suitable halogens include fluorine, chlorine, bromine, or iodine. In certain embodiments, R¹⁵ is —CN. In certain embodiments, R¹⁵ is —OH.

In certain embodiments, R¹⁵ is C₁-C₆alkoxy. Suitable alkoxys include, but are not limited to, methoxy, ethoxy, n-propoxy, isopropoxy and n-butoxy. In certain embodiments, R¹⁵ is C₁-C₆alkylOC₁-C₆alkyl. In certain embodiments, R¹⁵ is —COOH. In certain embodiments, R¹⁵ is C₁-C₆alkylCOOH. In certain embodiments, R¹⁵ is C₃-C₆cycloalkyl. Suitable examples of cycloalkyls include, but are not limited to, cyclopropyl, cyclobutyl, cyclopentyl and cyclohexyl. In certain embodiments, R¹⁵ is C₁-C₆alkyl. Examples of C₁-C₆alkyl groups include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R¹⁵ is ethyl.

In certain embodiments, R¹⁵ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R¹⁵ is C₁-C₆alkylOH. Examples of suitable alcohols include, but are not limited to, methanol, ethanol, propanol, butanol and iso-butanol. In certain embodiments, R¹⁵ is —CON(R⁷)(R⁸). In certain embodiments, R¹⁵ is N(R⁷)(R⁸). In certain embodiments, R¹⁵ is C₁-C₆alkylN(R⁷)(R⁸).

In certain embodiments, R¹⁵ is methyl or ethyl.

In the embodiments described herein, R¹⁶ is hydrogen, halogen, —CN, —OH, C₁-C₆alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸) or N(R⁷)(R⁸). In certain embodiments, R¹⁶ is hydrogen. In certain embodiments, R¹⁶ is halogen. Suitable halogens include fluorine, chlorine, bromine, or iodine. In certain embodiments, R¹⁶ is —CN. In certain embodiments, R¹⁶ is —OH.

In certain embodiments, R¹⁶ is C₁-C₆alkoxy. Suitable alkoxys include, but are not limited to, methoxy, ethoxy, n-propoxy, isopropoxy and n-butoxy. In certain embodiments, R¹⁶ is C₁-C₆alkylOC₁-C₆alkyl. In certain embodiments, R¹⁶ is —COOH. In certain embodiments, R¹⁶ is C₁-C₆alkylCOOH. In certain embodiments, R¹⁶ is C₃-C₆cycloalkyl. Suitable examples of cycloalkyls include, but are not limited to, cyclopropyl, cyclobutyl, cyclopentyl and cyclohexyl. In certain embodiments, R¹⁶ is C₁-C₆alkyl. Examples of C₁-C₆alkyl groups include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R¹⁶ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R¹⁶ is C₁-C₆alkylOH. Examples of suitable alcohols include, but are not limited to, methanol, ethanol, propanol, butanol and iso-butanol. In certain embodiments, R¹⁶ is —CON(R⁷)(R⁸). In certain embodiments, R¹⁶ is N(R⁷)(R⁸). In certain embodiments, R¹⁶ is C₁-C₆alkylN(R⁷)(R⁸).

In the embodiments of the compounds described herein, m is 0 or 1. In certain embodiments, m is 0. In certain embodiments, m is 1.

In the embodiments of the compounds described herein, p is 0 or 1. In certain embodiments, p is 0. In certain embodiments, p is 1.

In the embodiments described herein, A is a straight or branched, saturated or unsaturated (C₃-C₁₀)alkylene, phenyl(C₃-C₁₀)alkylene or cycloalkyl(C₃-C₁₀)alkylene comprising at least one —CH₂— group, wherein one or more additional —CH₂— groups in A are optionally and independently replaced with a moiety selected from the group consisting of O, S, NR, —CONR, NRCO, SO₂, and SO₂NR and wherein one or more of the hydrogens along A can be replaced with a halogen. In certain embodiments, A is a straight or branched, saturated or unsaturated (C₃-C₁₀)alkylene or cycloalkyl(C₃-C₁₀)alkylene, wherein one or more —CH₂— groups in A are optionally and independently replaced with a moiety selected from the group consisting of O, S and NH. In certain embodiment, A will always have at least one —CH₂— group.

In certain embodiments, A is a straight (C₃-C₁₀)alkylene. Examples of straight (C₃-C₁₀)alkylenes include,

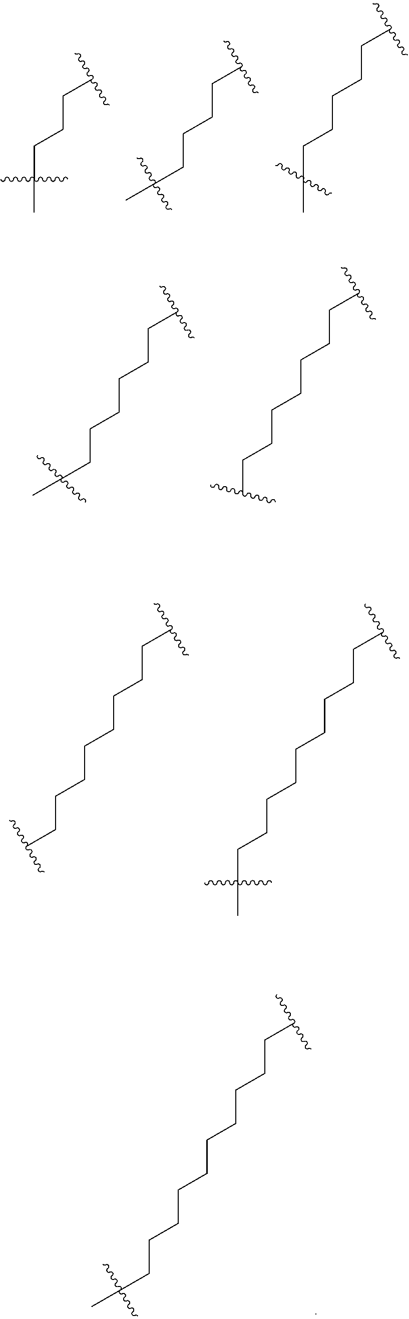

In certain embodiments, A is a branched (C₃-C₁₀)alkylene. Suitable branched (C₃-C₁₀)alkylenes include but are not limited to:

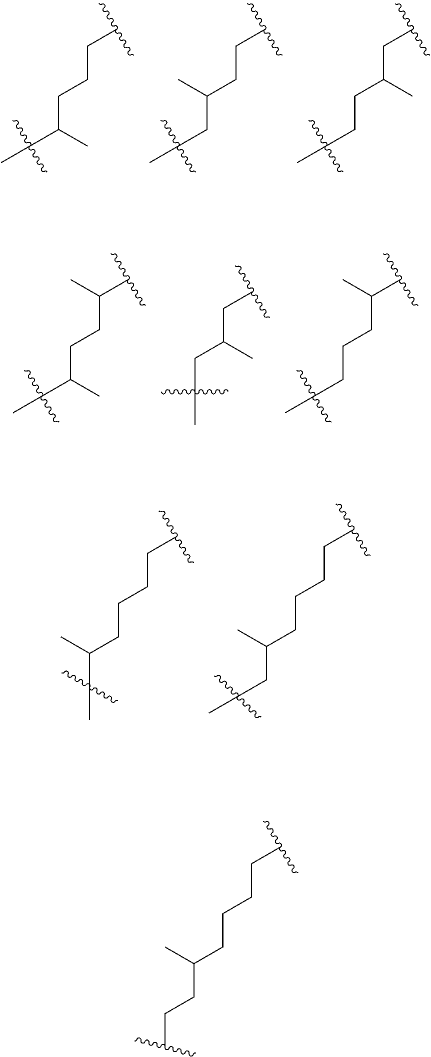

In certain embodiments, A is a saturated (C₃-C₁₀)alkylene. Examples include,

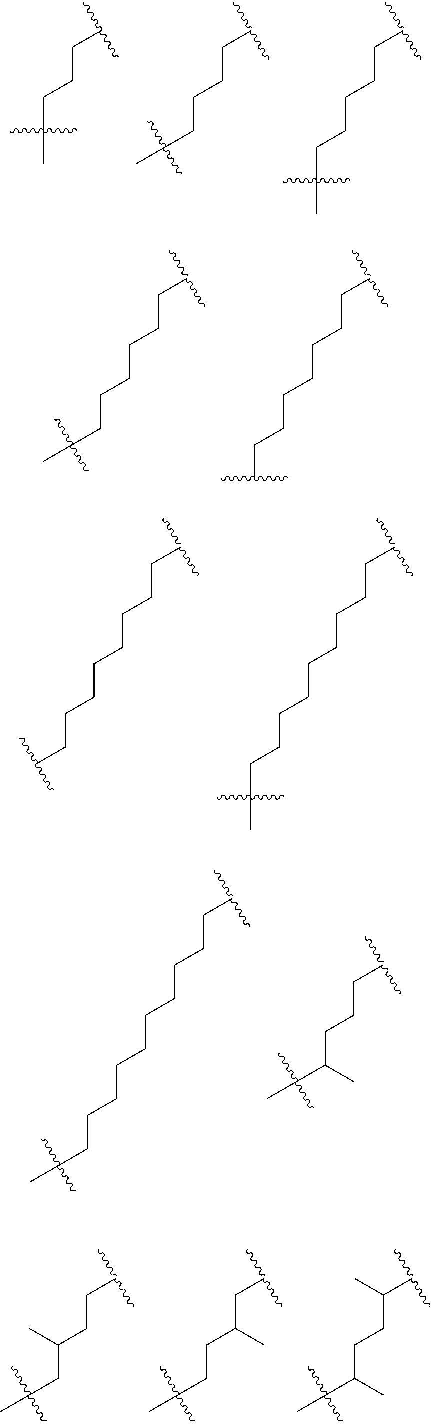

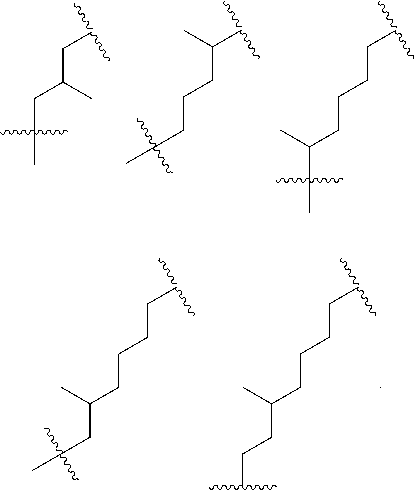

In certain embodiments, A is an unsaturated (C₃-C₁₀)alkylene. Suitable unsaturated (C₃-C₁₀)alkylenes include any of the saturated (C₃-C₁₀)alkylene, wherein hydrogens have been removed and one or more double or triple covalent bonds exist between adjacent carbon atoms.

Examples of unsaturated (C₃-C₁₀)alkylenes include, but are not limited to,

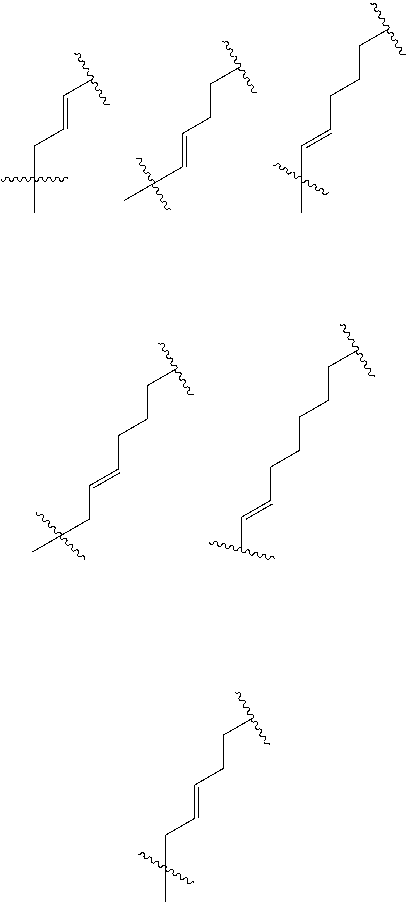 [SMILES: */C=C/C* ; */C=C/CC* ; */C=C/CCC* ; *C/C=C/CCC* ; */C=C/CCCCC* ; *C/C=C/CC*]

In other embodiments, A is a straight cycloalkyl(C₃-C₁₀)alkylene. Suitable straight cycloalkyl(C₃-C₁₀)alkylenes include a cycloalkyl(C₃-C₁₀)alkylene wherein two carbons in a chain are included in a (C₃-C₁₀)cycloalkyl. Examples of straight cycloalkyl(C₃-C₁₀)alkylenes include, but are not limited to,

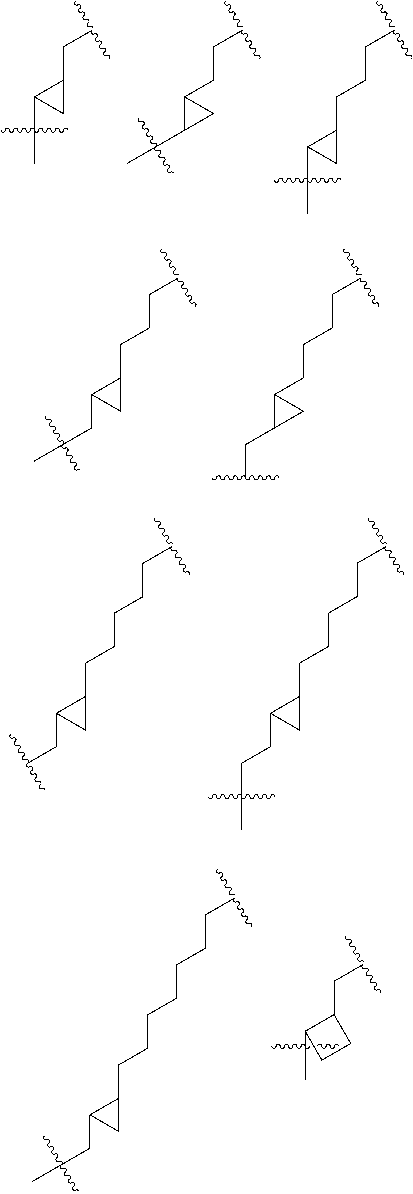

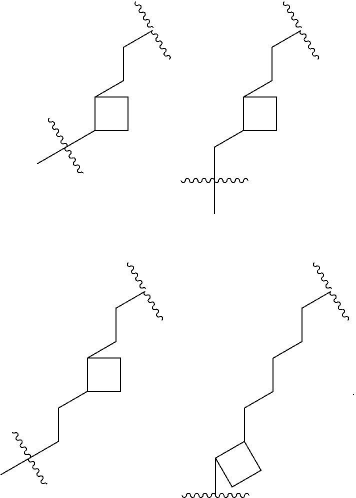 [SMILES: *CCC1CCC1* ; *CCC1CCC1C* ; *CCC1CCC1CC* ; *CCCCCC1CCC1*]

In certain embodiments, A is a branched cycloalkyl(C₃-C₁₀)alkylene. Suitable branched cycloalkyl(C₃-C₁₀)alkylenes include a branched (C₃-C₁₀)alkylene wherein two carbons in a chain are included in a (C₃-C₁₀)cycloalkyl. Examples of cycloalkyl(C₃-C₁₀)alkylenes include, but are not limited to,

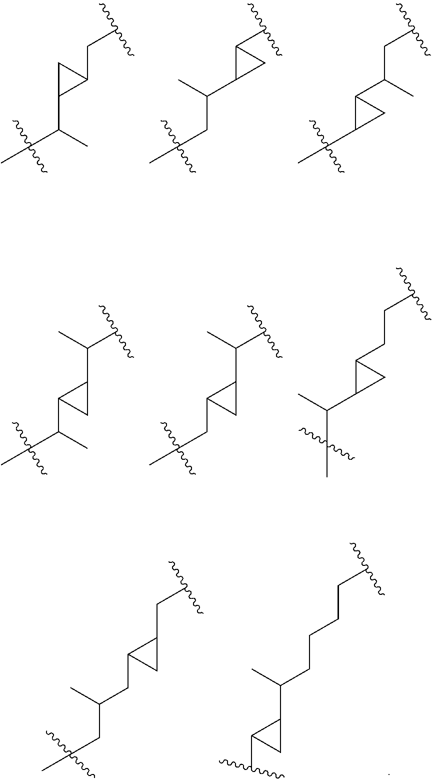

In certain embodiments, A is a saturated cycloalkyl(C₃-C₁₀)alkylene. Examples of saturated cycloalkyl(C₃-C₁₀)alkylenes include, but are not limited to,

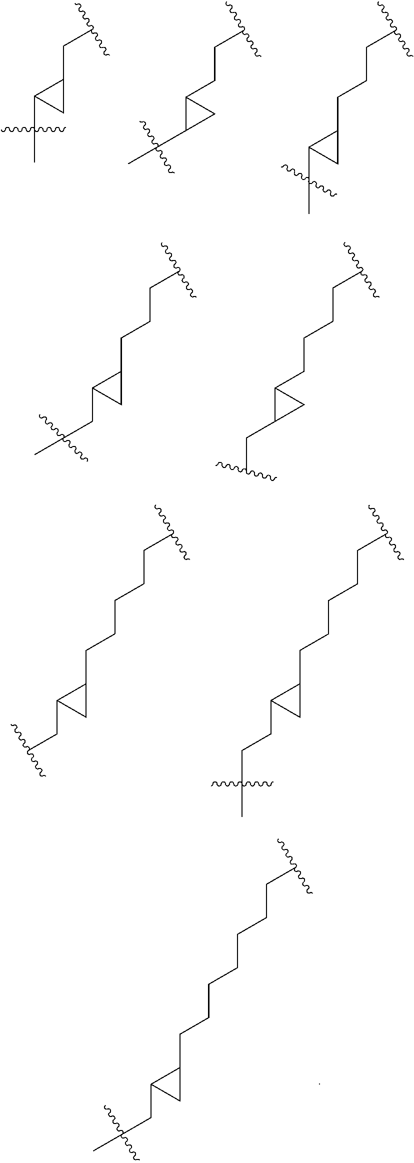

In certain embodiments, A is an unsaturated cycloalkyl(C₃-C₁₀)alkylene. Examples of unsaturated cyclo(C₃-C₁₀)alkylenes include, but are not limited to,

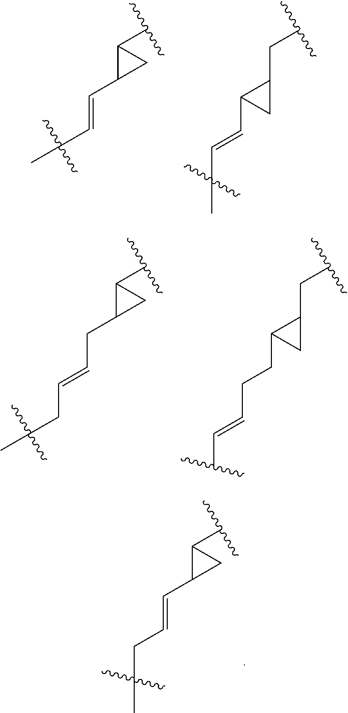 [SMILES: */C=C/C1CC1* ; */C=C/C1CC1C* ; *C/C=C/CC1CC1* ; */C=C/CCC1CC1C* ; *C/C=C/C1CC1*]

In certain embodiments, A is an unsaturated or saturated phenyl(C₃-C₁₀)alkylene. Examples of unsaturated and saturated phenyl(C₃-C₁₀)alkylenes include, but are not limited to,

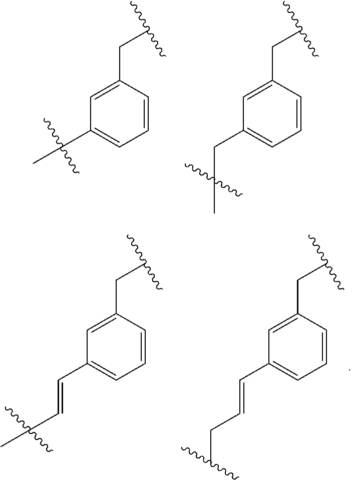 [SMILES: *Cc1cccc(*)c1 ; *Cc1cccc(C*)c1 ; */C=C/c1cccc(C*)c1 ; *C/C=C/c1cccc(C*)c1]

In other embodiments, one or more —CH₂— groups in A are optionally and independently replaced with a moiety selected from the group consisting of O, S, NR, —CONR, NRCO, SO₂, and SO₂NR. In other embodiments, one or more —CH₂— groups in A are optionally and independently replaced with a moiety selected from the group consisting of O, S and NH. In other embodiments, one or more —CH₂— groups in A are optionally and independently replaced with O. In other embodiments, one or more —CH₂— groups in A are optionally and independently replaced with S. In other embodiments, one or more —CH₂— groups in A are optionally and independently replaced with NR. In other embodiments, one or more —CH₂— groups in A are optionally and independently replaced with —CONR. In other embodiments, one or more —CH₂— groups in A are optionally and independently replaced with NRCO. In other embodiments, one or more —CH₂— groups in A are optionally and independently replaced with SO₂. In other embodiments, one or more —CH₂— groups in A are optionally and independently replaced with and SO₂NR. R will be described in further detail below.

In the embodiments described herein, R is hydrogen, C₁-C₆alkylCOOH, —COOH, C₃-C₆cycloalkyl, C₁-C₆alkyl, haloC₁-C₆alkyl, C₁-C₆alkylOH, —COC₁-C₆alkyl or —COOC₁-C₆alkyl. In certain embodiments, R is hydrogen, C₁-C₆alkylCOOH, —COOH, C₃-C₆cycloalkyl, C₁-C₆alkyl, haloC₁-C₆alkyl or C₁-C₆alkylOH.

In certain embodiments, R is hydrogen. In certain embodiments, R is C₁-C₆alkylCOOH. In certain embodiments, R is —COOH. In certain embodiments, R is C₃-C₆cycloalkyl. Suitable examples of cycloalkyls include, but are not limited to, cyclopropyl, cyclobutyl, cyclopentyl and cyclohexyl. In certain embodiments, R is C₁-C₆alkyl. Examples of C₁-C₆alkyl groups include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R is C₁-C₆alkylOH. Examples of suitable alcohols include, but are not limited to, methanol, ethanol, propanol, butanol and iso-butanol.

In certain embodiments, R is —COC₁-C₆alkyl. Suitable examples include, but are not limited to, —COCH₃. In certain embodiments, R is —COOC₁-C₆alkyl. Suitable examples include, but are not limited to, —COOCH₃.

Examples of such embodiments include, but are not limited to,

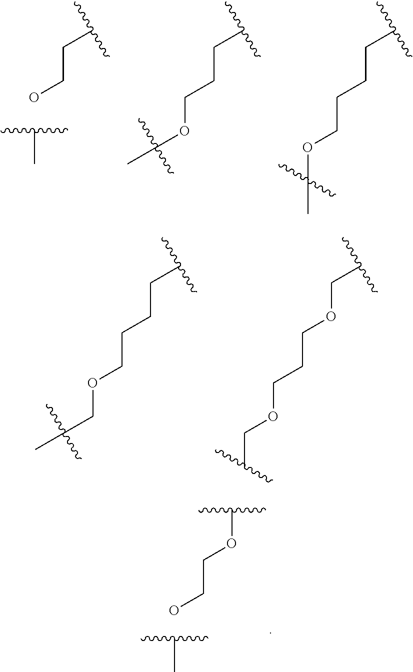

In certain embodiments, A is

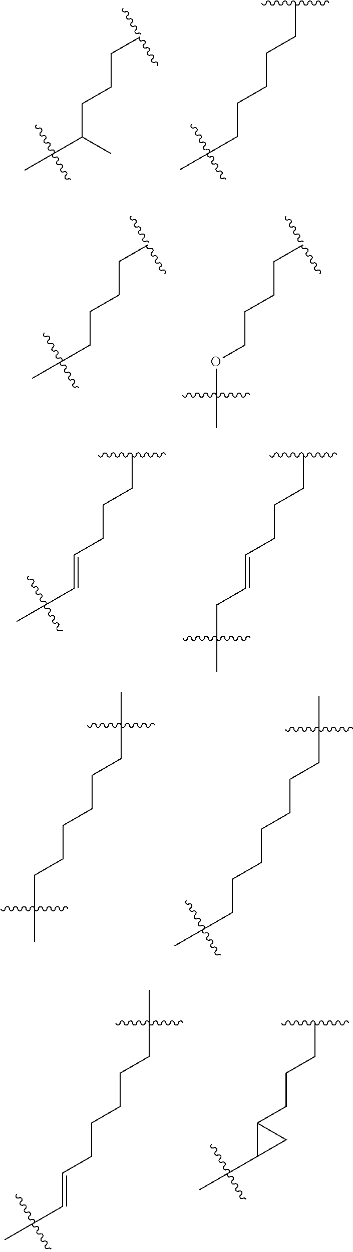

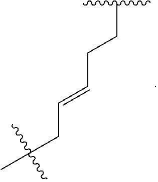 [SMILES: *C/C=C/CC*]

In certain embodiments, one or more of the hydrogens along A is replaced with a halogen. Examples of suitable halogens include chlorine, bromine, fluorine and iodine. In certain embodiments, A is

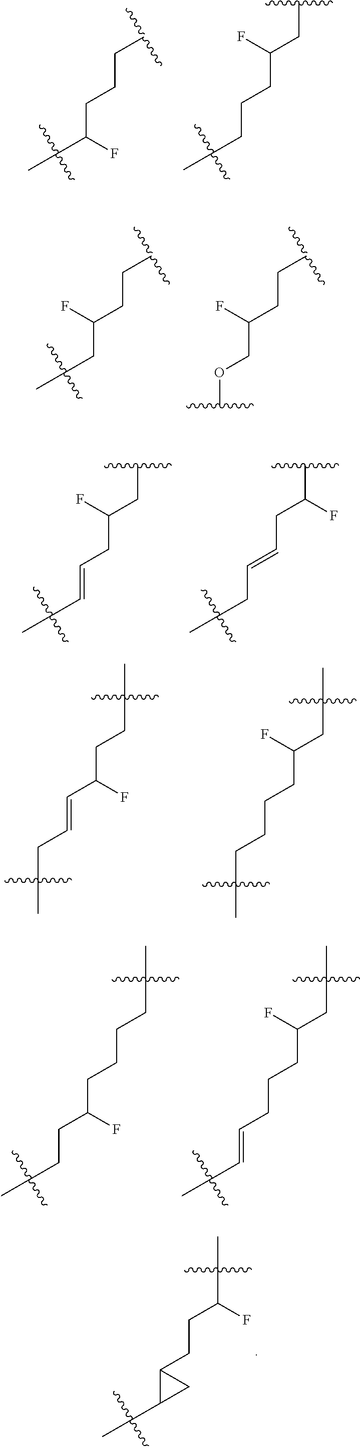

Also described herein are compounds having the structural Formula (III):

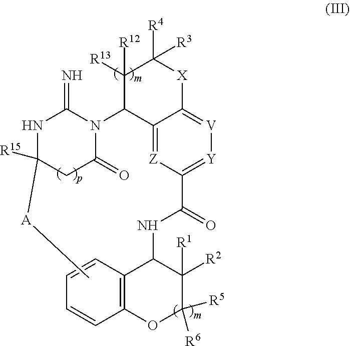

wherein, A is a straight or branched, saturated or unsaturated (C₃-C₁₀)alkylene, phenyl(C₃-C₁₀)alkylene or cycloalkyl(C₃-C₁₀)alkylene comprising at least one —CH₂— group, wherein one or more additional —CH₂— groups in A are optionally and independently replaced with a moiety selected from the group consisting of O, S, NR, —CONR, NRCO, SO₂, and SO₂NR and wherein one or more of the hydrogens along A can be replaced with a halogen;
Q is C(R¹⁶)₂, O, S, SO, SO₂ or NH;
X is a bond, C(R¹⁴)₂, O, S, SO, SO₂ or NH;
Y is CR⁹ or N, wherein when Y is N, Z is CR¹¹ and V is CR¹⁰;
V is CR¹⁰ or N, wherein when V is N, Z is CR¹¹ and Y is CR⁹;
Z is CR¹¹ or N, wherein when Z is N, V is CR¹⁰ and Y is CR⁹;
each occurrence of R is independently hydrogen, C₁-C₆alkylCOOH, —COOH, C₃-C₆cycloalkyl, C₁-C₆alkyl, haloC₁-C₆alkyl, C₁-C₆alkylOH, —COC₁-C₆alkyl or —COOC₁-C₆alkyl;
R¹ is hydrogen, halogen, —CN, —OH, C₁-C₆alkoxy, C₁-C₆alkylOC₁-C₆alkyl, C₁-C₆alkylCOOH, —COOH, oxo, —COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, —C₁-C₆alkylOhaloC₁-C₆alkyl, haloC₁-C₆alkyl, C₁-C₆alkylOH, —CON(R⁷)(R⁸), —N(R⁷)(R⁸) or C₁-C₆alkylN(R⁷)(R⁸);
R² is hydrogen, halogen, —CN, —OH, C₁-C₆alkoxy, C₁-C₆alkylOC₁-C₆alkyl, C₁-C₆alkylCOOH, —COOH, oxo, —COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, —C₁-C₆alkylOhaloC₁-C₆alkyl, haloC₁-C₆alkyl, C₁-C₆alkylOH, —CON(R⁷)(R⁸), —N(R⁷)(R⁸) or C₁-C₆alkylN(R⁷)(R⁸);
R³ is hydrogen, halogen, —CN, —OH, C₁-C₆alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸), —N(R⁷)(R⁸), C₁-C₆alkylN(R⁷)(R⁸), C₁-C₆alkyl(OCH₂CH₂)ₙN(R⁷)(R⁸) or C₁-C₆alkylOhaloC₁-C₆alkyl or when taken with R⁴ forms a C₃-C₆cycloalkyl or C₃-C₆heterocycloalkyl;
R⁴ is hydrogen, halogen, —CN, —OH, C₁-C₆alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸), —N(R⁷)(R⁸) C₁-C₆alkylN(R⁷)(R⁸), C₁-C₆alkyl(OCH₂CH₂)ₙN(R⁷)(R⁸) or C₁-C₆alkylOhaloC₁-C₆alkyl or when taken with R³ forms a C₃-C₆cycloalkyl or C₃-C₆heterocycloalkyl;
R⁵ is hydrogen, halogen, —CN, —OH, C₁-C₆alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸), —N(R⁷)(R⁸) or C₁-C₆alkylN(R⁷)(R⁸);
R⁶ is hydrogen, halogen, —CN, —OH, C₁-C₆alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸), —N(R⁷)(R⁸) or C₁-C₆alkylN(R⁷)(R⁸);
each occurrence of R⁷ is independently hydrogen, C₁-C₆alkylCOOH, —COOH, C₃-C₆cycloalkyl, C₁-C₆alkyl, haloC₁-C₆alkyl, C₁-C₆alkylOH, —COC₁-C₆alkyl or —COOC₁-C₆alkyl;
each occurrence of R⁸ is independently hydrogen, C₁-C₆alkylCOOH, —COOH, C₃-C₆cycloalkyl, C₁-C₆alkyl, haloC₁-C₆alkyl, C₁-C₆alkylOH, —COC₁-C₆alkyl or —COOC₁-C₆alkyl;
R⁹ is hydrogen, halogen, —CN, —OH, C₁-C₆alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸), —N(R⁷)(R⁸) or C₁-C₆alkylN(R⁷)(R⁸);
R¹⁰ is hydrogen, halogen, —CN, —OH, C₁-C₆alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸), —N(R⁷)(R⁸) or C₁-C₆alkylN(R⁷)(R⁸);
R¹¹ is hydrogen, halogen, —CN, —OH, C₁-C₆alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸), —N(R⁷)(R⁸) or C₁-C₆alkylN(R⁷)(R⁸);
R¹² is hydrogen, halogen, —CN, —OH, C₁-C₆alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸), —N(R⁷)(R⁸) or C₁-C₆alkylN(R⁷)(R⁸);
R¹³ is hydrogen, halogen, —CN, —OH, C₁-C₆alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸), —N(R⁷)(R⁸) or C₁-C₆alkylN(R⁷)(R⁸);
each occurrence of R¹⁴ is independently selected from the group consisting of hydrogen, halogen, —CN, —OH, C₁-C₆alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸), —N(R⁷)(R⁸) and C₁-C₆alkylN(R⁷)(R⁸);
R¹⁵ is hydrogen, halogen, —CN, —OH, C₁-C₆alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸), —N(R⁷)(R⁸) or C₁-C₆alkylN(R⁷)(R⁸);
each occurrence of R¹⁶ is independently selected from the group consisting of hydrogen, halogen, —CN, —OH, C₁-C₆alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸), —N(R⁷)(R⁸) and C₁-C₆alkylN(R⁷)(R⁸);
l is 0 or 1;
m is 0 or 1;
n is 1, 2, 3 or 4; and
p is 0 or 1.

In the embodiments described herein, Q is C(R¹⁶)₂, O, S, SO, SO₂ or NH. In certain embodiments, Q is C(R¹⁶)₂, wherein R¹⁶ is discussed in further detail below. In certain embodiments, Q is CH₂, CH(CH₃), C(CH₃)₂, O, CH(OCH₃), SO₂ or CF₂. In other embodiments Q is CH₂, O, S, SO, SO₂ or NH. In certain embodiments, Q is CH₂. In certain embodiments described herein, Q is O. In certain embodiments described herein, Q is S. In other embodiments described herein, Q is SO. In other embodiments described herein, Q is SO₂. In certain embodiments described herein, Q is NH. In other embodiments described herein, Q is O or SO₂. In still other embodiments described herein, Q is O or CH₂.

In the embodiments described herein, X is a bond, C(R¹⁴)₂, O, S, SO, SO₂ or NH. In certain embodiments described herein X is a bond. In certain embodiments, X is C(R¹⁴)₂, wherein R¹⁴ is discussed in further detail below. In certain embodiments, X is a bond, CH₂, CH(CH₃), C(CH₃)₂, O, CH(OCH₃), SO₂ or CF₂. In other embodiments X is CH₂, O, S, SO, SO₂ or NH. In certain embodiments, X is CH₂. In certain embodiments described herein, X is O. In certain embodiments described herein, X is S. In certain embodiments described herein, X is SO. In other embodiments described herein, X is SO₂. In certain embodiments described herein, X is NH.

In the embodiments described herein, Y is CR⁹ or N. In certain embodiments, Y is CR⁹, wherein R⁹ is discussed in detail below. In certain embodiments, Y is N. In certain embodiments, Y is CH. In certain embodiments, wherein when Y is N, Z is CR¹¹ and V is CR¹⁰.

In the embodiments described herein, V is CR¹⁰ or N. In certain embodiments, V is CR¹⁰, wherein R¹⁰ is discussed in detail below. In certain embodiments, V is N. In certain embodiments, V is CH. In certain embodiments, wherein when V is N, Z is CR¹¹ and Y is CR⁹.

In the embodiments described herein, Z is CR¹¹ or N. In certain embodiments, Z is CR¹¹, wherein R¹¹ is discussed in detail below. In certain embodiments, Z is CH. In certain embodiments, Z is N. In certain embodiments, wherein when Z is N, V is CR¹⁰ and Y is CR⁹.

In certain embodiments, X is O, Y and V are each CH and Z is N. In certain embodiments, X is O, Y and Z are each CH and V is N. In certain embodiments, X is O and V, Y and Z are all simultaneously CH.

Certain embodiments are represented as Formulae IV-VI:

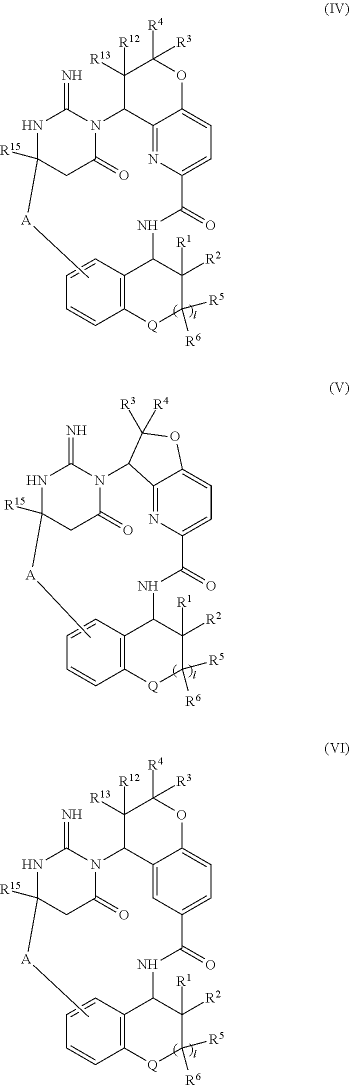

wherein A, Q, R¹, R², R³, R⁴, R⁵, R⁶, R¹², R¹³, R¹⁵ and l are as described above for Formula III.

In the embodiments described herein, R¹ is hydrogen, halogen, —CN, —OH, C₁-C₆alkoxy, C₁-C₆alkylOC₁-C₆alkyl, C₁-C₆alkylCOOH, —COOH, oxo, —COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, —C₁-C₆alkylOhaloC₁-C₆alkyl, haloC₁-C₆alkyl, C₁-C₆alkylOH, —CON(R⁷)(R⁸), —N(R⁷)(R⁸) or C₁-C₆alkylN(R⁷)(R⁸).

In certain embodiments described herein, R¹ is hydrogen.

In certain embodiments, R¹ is halogen. Examples of suitable halogens include chlorine, bromine, fluorine and iodine.

In certain embodiments, R¹ is —CN.

In certain embodiments, R¹ is —OH.

In certain embodiments, R¹ is C₁-C₆alkoxy. Suitable alkoxys include, but are not limited to, methoxy, ethoxy, n-propoxy, isopropoxy and n-butoxy.

In certain embodiments, R¹ is C₁-C₆alkylOC₁-C₆alkyl.

In certain embodiments, R¹ is C₁-C₆alkylCOOH.

In certain embodiments, R¹ is —COOH.

In certain embodiments, R¹ is an oxo group.

In certain embodiments, R¹ is —COOC₁-C₆alkyl.

In certain embodiments, R¹ is C₁-C₆alkylCOOC₁-C₆alkyl.

In certain embodiments, R¹ is C₃-C₆cycloalkyl. Suitable examples of cycloalkyls include, but are not limited to, cyclopropyl, cyclobutyl, cyclopentyl and cyclohexyl.

In certain embodiments, R¹ is C₁-C₆alkylC₃-C₆cycloalkyl. Suitable examples of cycloalkyls include, but are not limited to

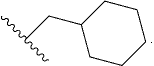 [SMILES: *CC1CCCCC1]

In certain embodiments, R¹ is C₁-C₆alkyl. Examples of C₁-C₆alkyl groups include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl.

In certain embodiments, R¹ is C₁-C₆alkylOhaloC₁-C₆alkyl. Suitable examples of C₁-C₆alkylOhaloC₁-C₆alkyls include, but are not limited to,

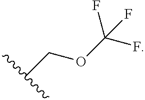 [SMILES: *COC(F)(F)F]

In certain embodiments, R¹ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl.

In certain embodiments, R¹ is C₁-C₆alkylOH. Suitable alcohols include, but are not limited to, methanol, ethanol, propanol, butanol and iso-butanol.

In certain embodiments, R¹ is —CON(R⁷)(R⁸). In certain embodiments, R¹ is N(R⁷)(R⁸). In certain embodiments, R¹ is C₁-C₆alkylN(R⁷)(R⁸), wherein R⁷ and R⁸ will be described in detail below.

In certain embodiments, R¹ is hydrogen, bromine, fluorine, chlorine, methyl, —OH, halogen, —CNoxo, methoxymethyl, —COOCH₂CH₃ or trifluoromethyl.

In the embodiments described herein, R² is hydrogen, halogen, —CN, —OH, C₁-C₆alkoxy, C₁-C₆alkylOC₁-C₆alkyl, C₁-C₆alkylCOOH, —COOH, oxo, —COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, —C₁-C₆alkylOhaloC₁-C₆alkyl, haloC₁-C₆alkyl, C₁-C₆alkylOH, —CON(R⁷)(R⁸), —N(R⁷)(R⁸) or C₁-C₆alkylN(R⁷)(R⁸).

In certain embodiments described herein, R² is hydrogen.

In certain embodiments, R² is halogen. Examples of suitable halogens include chlorine, bromine, fluorine and iodine.

In certain embodiments, R² is —CN.

In certain embodiments, R² is —OH.

In certain embodiments, R² is C₁-C₆alkoxy. Suitable alkoxys include, but are not limited to, methoxy, ethoxy, n-propoxy, isopropoxy and n-butoxy.

In certain embodiments, R² is C₁-C₆alkylOC₁-C₆alkyl.

In certain embodiments, R² is C₁-C₆alkylCOOH.

In certain embodiments, R² is —COOH.

In certain embodiments, R² is an oxo group.

In certain embodiments, R² is —COOC₁-C₆alkyl.

In certain embodiments, R² is C₁-C₆alkylCOOC₁-C₆alkyl.

In certain embodiments, R² is C₃-C₆cycloalkyl. Suitable examples of cycloalkyls include, but are not limited to, cyclopropyl, cyclobutyl, cyclopentyl and cyclohexyl.

In certain embodiments, R² is C₁-C₆alkylC₃-C₆cycloalkyl. Suitable examples of cycloalkyls include, but are not limited to

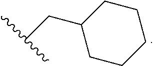 [SMILES: *CC1CCCCC1]

In certain embodiments, R² is C₁-C₆alkyl. Examples of C₁-C₆alkyl groups can include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl.

In certain embodiments, R² is C₁-C₆alkylOhaloC₁-C₆alkyl. Suitable examples of C₁-C₆alkylOhaloC₁-C₆alkyls include, but are not limited to,

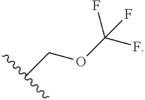 [SMILES: *COC(F)(F)F]

In certain embodiments, R² is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl.

In certain embodiments, R² is C₁-C₆alkylOH. Suitable alcohols include, but are not limited to, methanol, ethanol, propanol, butanol and iso-butanol.

In certain embodiments, R² is —CON(R⁷)(R⁸). In certain embodiments, R² is N(R⁷)(R⁸). In certain embodiments, R² is C₁-C₆alkylN(R⁷)(R⁸), wherein R⁷ and R⁸ will be described in detail below.

In certain embodiments, R² is hydrogen, bromine, fluorine, chlorine, methyl, —OH, halogen, —CN oxo, methoxymethyl, —COOCH₂CH₃ or trifluoromethyl.

In certain embodiments, R¹ and R² are both hydrogen. In certain embodiments, R¹ is —OH and R² is hydrogen.

In the embodiments described herein, R³ is hydrogen, halogen, —CN, —OH, C₁-C₆alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸), —N(R⁷)(R⁸), C₁-C₆alkylN(R⁷)(R⁸), C₁-C₆alkyl(OCH₂CH₂)ₙN(R⁷)(R⁸) or C₁-C₆alkylOhaloC₁-C₆alkyl or when taken with R⁴ forms a C₃-C₆cycloalkyl or C₃-C₆heterocycloalkyl.

In certain embodiments of the compounds described herein, R³ is hydrogen, halogen, —CN, —OH, C₁-C₆alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸), —N(R⁷)(R⁸) or C₁-C₆alkylN(R⁷)(R⁸) or when taken with R⁴ forms a C₃-C₆cycloalkyl or C₃-C₆heterocycloalkyl. In certain embodiments, R³ is hydrogen. In certain embodiments, R³ is halogen. Suitable halogens include fluorine, chlorine, bromine, and iodine. In certain embodiments, R³ is —CN. In certain embodiments, R³ is —OH.

In certain embodiments, R³ is C₁-C₆alkoxy. Suitable alkoxys include, but are not limited to, methoxy, ethoxy, n-propoxy, isopropoxy and n-butoxy. In certain embodiments, R³ is C₁-C₆alkylOC₁-C₆alkyl. In certain embodiments, R³ is —COOH. In certain embodiments, R³ is C₁-C₆alkylCOOH. In certain embodiments, R³ is C₃-C₆cycloalkyl. Suitable examples of cycloalkyls include, but are not limited to, cyclopropyl, cyclobutyl, cyclopentyl and cyclohexyl. In certain embodiments, R³ is C₁-C₆alkyl. Examples of C₁-C₆alkyl groups can include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R³ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R³ is C₁-C₆alkylOH. Examples of suitable alcohols include, but are not limited to, methanol, ethanol, propanol, butanol and iso-butanol. In certain embodiments, R³ is —CON(R⁷)(R⁸). Suitable examples of N(R⁷)(R⁸) include, but are not limited to, —CONH₂ and —CON(CH₃)₂. In certain embodiments, R³ is N(R⁷)(R⁸). Suitable examples of N(R⁷)(R⁸) include, but are not limited to, NH₂ and N(CH₃)₂. In certain embodiments, R³ is C₁-C₆alkylN(R⁷)(R⁸). Suitable examples of C₁-C₆alkylN(R⁷)(R⁸) include, but are not limited to,

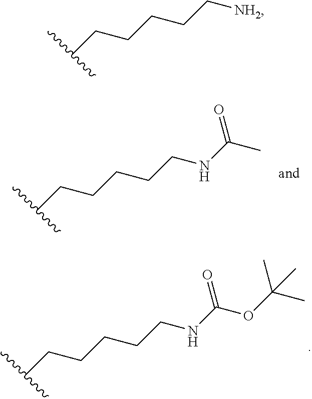

R⁷ and R⁸ are discussed in further detail below.

In certain embodiments, R³ is C₁-C₆alkylOhaloC₁-C₆alkyl. Suitable examples of haloalkyls include, but are not limited to,

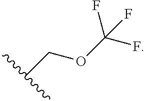 [SMILES: *COC(F)(F)F]

In certain embodiments, R³ is C₁-C₆alkyl(OCH₂CH₂)ₙN(R⁷)(R⁸). R⁷, R⁸ and n are discussed in detail below. Suitable examples of C₁-C₆alkyl(OCH₂CH₂)ₙN(R⁷)(R⁸) include, but are not limited to,

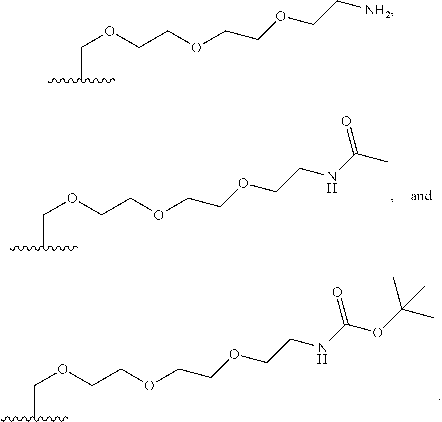 [SMILES: *COCCOCCOCCN ; *COCCOCCOCCNC(C)=O ; *COCCOCCOCCNC(=O)OC(C)(C)C]

With regard to the compounds described herein, n is 1, 2, 3 or 4. In certain embodiments, n is 1. In certain embodiments, n is 2. In certain embodiments, n is 3. In certain embodiments, n is 4.

In certain embodiments, R³ is taken with R⁴ and forms a C₃-C₆cycloalkyl or C₃-C₆heterocycloalkyl. In certain embodiments, R³ is taken with R⁴ and forms a C₃-C₆cycloalkyl. Suitable examples of cycloalkyls include, but are not limited to, cyclopropyl, cyclobutyl, cyclopentyl and cyclohexyl. In certain embodiments, R³ is taken with R⁴ and forms a C₃-C₆heterocycloalkyl. Suitable examples of heterocycloalkyls include, but are not limited to, piperidyl, oxetanyl, pyrrolyl, piperazinyl, morpholinyl, thiomorpholinyl, thiazolidinyl, 1,4-dioxanyl, tetrahydrofuranyl, tetrahydrothiophenyl, beta lactam, gamma lactam, delta lactam, beta lactone, gamma lactone, delta lactone, and pyrrolidinone, and oxides thereof.

In certain embodiments, R³ is hydrogen, fluorine, methyl, ethyl, —OH, methoxy,

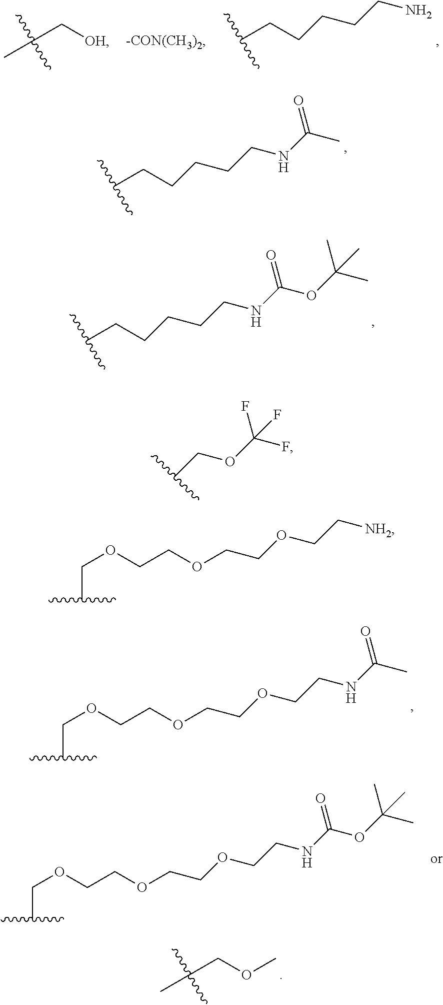

In certain embodiments, R³ is hydrogen, methyl, ethyl or

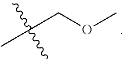 [SMILES: *COC]

In certain embodiments, R³ is taken with R⁴ to form oxetanyl.

In certain embodiments described herein, R⁴ is hydrogen, halogen, —CN, —OH, C₁-C₆alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸), —N(R⁷)(R⁸), C₁-C₆alkylN(R⁷)(R⁸), C₁-C₆alkyl(OCH₂CH₂)ₙN(R⁷)(R⁸) or C₁-C₆alkylOhaloC₁-C₆alkyl or when taken with R³ forms a C₃-C₆cycloalkyl or C₃-C₆heterocycloalkyl. In certain embodiments of the compounds described herein, R⁴ is hydrogen, halogen, —CN, —OH, C₁-C₆alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸), —N(R⁷)(R⁸) or C₁-C₆alkylN(R⁷)(R⁸) or when taken with R³ forms a C₃-C₆cycloalkyl or C₃-C₆heterocycloalkyl. In certain embodiments, R⁴ is hydrogen. In certain embodiments, R⁴ is halogen. Suitable halogens include fluorine, chlorine, bromine, or iodine. In certain embodiments, R⁴ is —CN. In certain embodiments, R⁴ is —OH.

In certain embodiments, R⁴ is C₁-C₆alkoxy. Suitable alkoxys include, but are not limited to, methoxy, ethoxy, n-propoxy, isopropoxy and n-butoxy. In certain embodiments, R⁴ is C₁-C₆alkylOC₁-C₆alkyl. In certain embodiments, R⁴ is —COOH. In certain embodiments, R⁴ is C₁-C₆alkylCOOH. In certain embodiments, R⁴ is C₃-C₆cycloalkyl. Suitable examples of cycloalkyls include, but are not limited to, cyclopropyl, cyclobutyl, cyclopentyl and cyclohexyl. In certain embodiments, R⁴ is C₁-C₆alkyl. Examples of C₁-C₆alkyl groups can include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R⁴ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R⁴ is C₁-C₆alkylOH. Examples of suitable alcohols include, but are not limited to, methanol, ethanol, propanol, butanol and iso-butanol. In certain embodiments, R⁴ is —CON(R⁷)(R⁸). Suitable examples of CON(R⁷)(R⁸) include, but are not limited to, —CONH₂ and —CON(CH₃)₂. In certain embodiments, R⁴ is N(R⁷)(R⁸). Suitable examples of N(R⁷)(R⁸) include, but are not limited to, NH₂ and N(CH₃)₂. In certain embodiments, R⁴ is C₁-C₆alkylN(R⁷)(R⁸). Suitable examples of C₁-C₆alkylN(R⁷)(R⁸) include, but are not limited to,

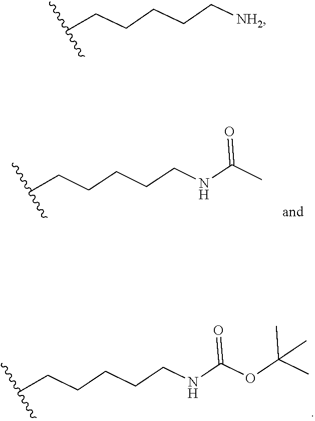

R⁷ and R⁸ are discussed in further detail below.

In certain embodiments, R⁴ is C₁-C₆alkylOhaloC₁-C₆alkyl. Suitable examples of haloalkyls include, but are not limited to,

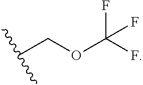 [SMILES: *COC(F)(F)F]

In certain embodiments, R⁴ is C₁-C₆alkyl(OCH₂CH₂)ₙN(R⁷)(R⁸). R⁷, R⁸ are discussed in detail below and n is discussed above. Suitable examples of C₁-C₆alkyl(OCH₂CH₂)ₙN(R⁷)(R⁸) include, but are not limited to,

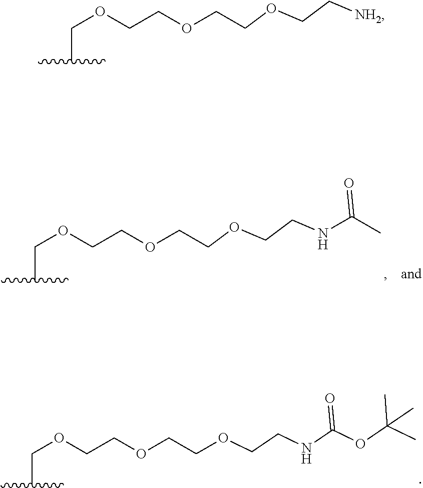 [SMILES: *COCCOCCOCCN ; *COCCOCCOCCNC(C)=O ; *COCCOCCOCCNC(=O)OC(C)(C)C]

In certain embodiments, R⁴ is taken with R³ and forms a C₃-C₆cycloalkyl or C₃-C₆heterocycloalkyl. In certain embodiments, R⁴ is taken with R³ and forms a C₃-C₆cycloalkyl. Suitable examples of cycloalkyls include, but are not limited to, cyclopropyl, cyclobutyl, cyclopentyl and cyclohexyl. In certain embodiments, R⁴ is taken with R³ and forms a C₃-C₆heterocycloalkyl. Suitable examples of heterocycloalkyls include, but are not limited to, piperidyl, oxetanyl, pyrrolyl, piperazinyl, morpholinyl, thiomorpholinyl, thiazolidinyl, 1,4-dioxanyl, tetrahydrofuranyl, tetrahydrothiophenyl, beta lactam, gamma lactam, delta lactam, beta lactone, gamma lactone, delta lactone, and pyrrolidinone, and oxides thereof.

In certain embodiments, R⁴ is hydrogen or methyl. In certain embodiments, R⁴ is hydrogen, methyl, ethyl or

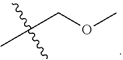 [SMILES: *COC]

In certain embodiments, R⁴ is taken with R³ to form oxetanyl. In certain embodiments, R³ and R⁴ are both hydrogen, methyl or ethyl.

In certain embodiments, R³ is hydrogen and R⁴ is hydrogen.

In the embodiments described herein, R⁵ is hydrogen, halogen, —CN, —OH, C₁-C₆alkoxy, C₁-C₆alkylOC₁-C₆alkyl, C₁-C₆alkylCOOH, —COOH, oxo, —COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, —C₁-C₆alkylOhaloC₁-C₆alkyl, haloC₁-C₆alkyl, C₁-C₆alkylOH, —CON(R⁷)(R⁸), —N(R⁷)(R⁸) or C₁-C₆alkylN(R⁷)(R⁸).

In certain embodiments described herein, R⁵ is hydrogen.

In certain embodiments, R⁵ is halogen. Examples of suitable halogens include chlorine, bromine, fluorine and iodine.

In certain embodiments, R⁵ is —CN.

In certain embodiments, R⁵ is —OH.

In certain embodiments, R⁵ is C₁-C₆alkoxy. Suitable alkoxys include, but are not limited to, methoxy, ethoxy, n-propoxy, isopropoxy and n-butoxy.

In certain embodiments, R⁵ is C₁-C₆alkylOC₁-C₆alkyl.

In certain embodiments, R⁵ is C₁-C₆alkylCOOH.

In certain embodiments, R⁵ is —COOH.

In certain embodiments, R⁵ is an oxo group.

In certain embodiments, R⁵ is —COOC₁-C₆alkyl.

In certain embodiments, R⁵ is C₁-C₆alkylCOOC₁-C₆alkyl.

In certain embodiments, R⁵ is C₃-C₆cycloalkyl. Suitable examples of cycloalkyls include, but are not limited to, cyclopropyl, cyclobutyl, cyclopentyl and cyclohexyl.

In certain embodiments, R⁵ is C₁-C₆alkylC₃-C₆cycloalkyl. Suitable examples of cycloalkyls include, but are not limited to

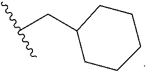 [SMILES: *CC1CCCCC1]

In certain embodiments, R⁵ is C₁-C₆alkyl. Examples of C₁-C₆alkyl groups can include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R⁵ is methyl.

In certain embodiments, R⁵ is C₁-C₆alkylOhaloC₁-C₆alkyl. Suitable examples of C₁-C₆alkylOhaloC₁-C₆alkyls include, but are not limited to,

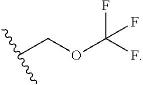 [SMILES: *COC(F)(F)F]

In certain embodiments, R⁵ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl.

In certain embodiments, R⁵ is C₁-C₆alkylOH. Suitable alcohols include, but are not limited to, methanol, ethanol, propanol, butanol and iso-butanol.

In certain embodiments, R⁵ is —CON(R⁷)(R⁸). In certain embodiments, R⁵ is N(R⁷)(R⁸). In certain embodiments, R⁵ is C₁-C₆alkylN(R⁷)(R⁸), wherein R⁷ and R⁸ will be described in detail below.

In certain embodiments, R⁵ is hydrogen, methyl, ethyl or t-butyl.

In the embodiments described herein, R⁶ is hydrogen, halogen, —CN, —OH, C₁-C₆alkoxy, C₁-C₆alkylOC₁-C₆alkyl, C₁-C₆alkylCOOH, —COOH, oxo, —COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, —C₁-C₆alkylOhaloC₁-C₆alkyl, haloC₁-C₆alkyl, C₁-C₆alkylOH, —CON(R⁷)(R⁸), —N(R⁷)(R⁸) or C₁-C₆alkylN(R⁷)(R⁸).

In certain embodiments described herein, R⁶ is hydrogen.

In certain embodiments, R⁶ is halogen. Examples of suitable halogens include chlorine, bromine, fluorine and iodine.

In certain embodiments, R⁶ is —CN.

In certain embodiments, R⁶ is —OH.

In certain embodiments, R⁶ is C₁-C₆alkoxy. Suitable alkoxys include, but are not limited to, methoxy, ethoxy, n-propoxy, isopropoxy and n-butoxy.

In certain embodiments, R⁶ is C₁-C₆alkylOC₁-C₆alkyl.

In certain embodiments, R⁶ is C₁-C₆alkylCOOH.

In certain embodiments, R⁶ is —COOH.

In certain embodiments, R⁶ is an oxo group.

In certain embodiments, R⁶ is —COOC₁-C₆alkyl.

In certain embodiments, R⁶ is C₁-C₆alkylCOOC₁-C₆alkyl.

In certain embodiments, R⁶ is C₃-C₆cycloalkyl. Suitable examples of cycloalkyls include, but are not limited to, cyclopropyl, cyclobutyl, cyclopentyl and cyclohexyl.

In certain embodiments, R⁶ is C₁-C₆alkylC₃-C₆cycloalkyl. Suitable examples of cycloalkyls include, but are not limited to

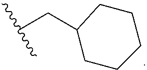 [SMILES: *CC1CCCCC1]

In certain embodiments, R⁶ is C₁-C₆alkyl. Examples of C₁-C₆alkyl groups include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R⁶ is methyl.

In certain embodiments, R⁶ is C₁-C₆alkylOhaloC₁-C₆alkyl. Suitable examples of C₁-C₆alkylOhaloC₁-C₆alkyls include, but are not limited to,

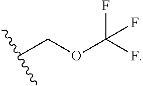 [SMILES: *COC(F)(F)F]

In certain embodiments, R⁶ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl.

In certain embodiments, R⁶ is C₁-C₆alkylOH. Suitable alcohols include, but are not limited to, methanol, ethanol, propanol, butanol and iso-butanol.

In certain embodiments, R⁶ is —CON(R⁷)(R⁸). In certain embodiments, R⁶ is N(R⁷)(R⁸). In certain embodiments, R⁶ is C₁-C₆alkylN(R⁷)(R⁸), wherein R⁷ and R⁸ will be described in detail below.

In certain embodiments, R⁶ is hydrogen, methyl, ethyl or t-butyl.

In the embodiments described herein, R⁷ is hydrogen, C₁-C₆alkylCOOH, —COOH, C₃-C₆cycloalkyl, C₁-C₆alkyl, haloC₁-C₆alkyl, C₁-C₆alkylOH, —COC₁-C₆alkyl or —COOC₁-C₆alkyl. In certain embodiments, R⁷ is hydrogen, C₁-C₆alkylCOOH, —COOH, C₃-C₆cycloalkyl, C₁-C₆alkyl, haloC₁-C₆alkyl or C₁-C₆alkylOH.

In certain embodiments, R⁷ is hydrogen. In certain embodiments, R⁷ is C₁-C₆alkylCOOH. In certain embodiments, R⁷ is —COOH. In certain embodiments, R⁷ is C₃-C₆cycloalkyl. Suitable examples of cycloalkyls include, but are not limited to, cyclopropyl, cyclobutyl, cyclopentyl and cyclohexyl. In certain embodiments, R⁷ is C₁-C₆alkyl. Examples of C₁-C₆alkyl groups include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R⁷ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R⁷ is C₁-C₆alkylOH. Examples of suitable alcohols include, but are not limited to, methanol, ethanol, propanol, butanol and iso-butanol.

In certain embodiments, R⁷ is —COC₁-C₆alkyl. Suitable examples include, but are not limited to, —COCH₃. In certain embodiments, R⁷ is —COOC₁-C₆alkyl. Suitable examples include, but are not limited to, —COOCH₃.

In the embodiments described herein, R⁸ is hydrogen, C₁-C₆alkylCOOH, —COOH, C₃-C₆cycloalkyl, C₁-C₆alkyl, haloC₁-C₆alkyl, C₁-C₆alkylOH, —COC₁-C₆alkyl or —COOC₁-C₆alkyl. In certain embodiments, R⁸ is hydrogen, C₁-C₆alkylCOOH, —COOH, C₃-C₆cycloalkyl, C₁-C₆alkyl, haloC₁-C₆alkyl or C₁-C₆alkylOH.

In certain embodiments, R⁸ is hydrogen. In certain embodiments, R⁸ is C₁-C₆alkylCOOH. In certain embodiments, R⁸ is —COOH. In certain embodiments, R⁸ is C₃-C₆cycloalkyl. Suitable examples of cycloalkyls include, but are not limited to, cyclopropyl, cyclobutyl, cyclopentyl and cyclohexyl. In certain embodiments, R⁸ is C₁-C₆alkyl. Examples of C₁-C₆alkyl groups include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R⁸ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R⁸ is C₁-C₆alkylOH. Examples of suitable alcohols include, but are not limited to, methanol, ethanol, propanol, butanol and iso-butanol.

In certain embodiments, R⁸ is —COC₁-C₆alkyl. Suitable examples include, but are not limited to, —COCH₃. In certain embodiments, R⁸ is —COOC₁-C₆alkyl. Suitable examples include, but are not limited to, —COOCH₃.

In the embodiments described herein, R⁹ is hydrogen, halogen, —CN, —OH, C₁-C₆alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸) or N(R⁷)(R⁸). In certain embodiments, R⁹ is hydrogen. In certain embodiments, R⁹ is halogen. Suitable halogens include fluorine, chlorine, bromine, or iodine. In certain embodiments, R⁹ is —CN. In certain embodiments, R⁹ is —OH.

In certain embodiments, R⁹ is C₁-C₆alkoxy. Suitable alkoxys include, but are not limited to, methoxy, ethoxy, n-propoxy, isopropoxy and n-butoxy. In certain embodiments, R⁹ is C₁-C₆alkylOC₁-C₆alkyl. In certain embodiments, R⁹ is —COOH. In certain embodiments, R⁹ is C₁-C₆alkylCOOH. In certain embodiments, R⁹ is C₃-C₆cycloalkyl. Suitable examples of cycloalkyls include, but are not limited to, cyclopropyl, cyclobutyl, cyclopentyl and cyclohexyl. In certain embodiments, R⁹ is C₁-C₆alkyl. Examples of C₁-C₆alkyl groups include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R⁹ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R⁹ is C₁-C₆alkylOH. Examples of suitable alcohols include, but are not limited to, methanol, ethanol, propanol, butanol and iso-butanol. In certain embodiments, R⁹ is —CON(R⁷)(R⁸). In certain embodiments, R⁹ is N(R⁷)(R⁸). In certain embodiments, R⁹ is C₁-C₆alkylN(R⁷)(R⁸).

With regard to the compounds described herein, R¹⁰ is hydrogen, halogen, —CN, —OH, C₁-C₆alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸) or N(R⁷)(R⁸). In certain embodiments, R¹⁰ is hydrogen. In certain embodiments, R¹⁰ is halogen. Suitable halogens include fluorine, chlorine, bromine, or iodine. In certain embodiments, R¹⁰ is —CN. In certain embodiments, R¹⁰ is —OH.

In certain embodiments, R¹⁰ is C₁-C₆alkoxy. Suitable alkoxys include, but are not limited to, methoxy, ethoxy, n-propoxy, isopropoxy and n-butoxy. In certain embodiments, R¹⁰ is C₁-C₆alkylOC₁-C₆alkyl. In certain embodiments, R¹⁰ is —COOH. In certain embodiments, R¹⁰ is C₁-C₆alkylCOOH. In certain embodiments, R¹⁰ is C₃-C₆cycloalkyl. Suitable examples of cycloalkyls include, but are not limited to, cyclopropyl, cyclobutyl, cyclopentyl and cyclohexyl. In certain embodiments, R¹⁰ is C₁-C₆alkyl. Examples of C₁-C₆alkyl groups include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R¹⁰ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R¹⁰ is C₁-C₆alkylOH. Examples of suitable alcohols include, but are not limited to, methanol, ethanol, propanol, butanol and iso-butanol. In certain embodiments, R¹⁰ is —CON(R⁷)(R⁸). In certain embodiments, R¹⁰ is N(R⁷)(R⁸). In certain embodiments, R¹⁰ is C₁-C₆alkylN(R⁷)(R⁸).

In the embodiments, described herein, R¹¹ is hydrogen, halogen, —CN, —OH, C₁-C₆alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸) or N(R⁷)(R⁸). In certain embodiments, R¹¹ is hydrogen. In certain embodiments, R¹¹ is halogen. Suitable halogens include fluorine, chlorine, bromine, or iodine. In certain embodiments, R¹¹ is —CN. In certain embodiments, R¹¹ is —OH.

In certain embodiments, R¹¹ is C₁-C₆alkoxy. Suitable alkoxys include, but are not limited to, methoxy, ethoxy, n-propoxy, isopropoxy and n-butoxy. In certain embodiments, R¹¹ is C₁-C₆alkylOC₁-C₆alkyl. In certain embodiments, R¹¹ is —COOH. In certain embodiments, R¹¹ is C₁-C₆alkylCOOH. In certain embodiments, R¹¹ is C₃-C₆cycloalkyl. Suitable examples of cycloalkyls include, but are not limited to, cyclopropyl, cyclobutyl, cyclopentyl and cyclohexyl. In certain embodiments, R¹¹ is C₁-C₆alkyl. Examples of C₁-C₆alkyl groups include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R¹¹ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R¹¹ is C₁-C₆alkylOH. Examples of suitable alcohols include, but are not limited to, methanol, ethanol, propanol, butanol and iso-butanol. In certain embodiments, R¹¹ is —CON(R⁷)(R⁸). In certain embodiments, R¹¹ is N(R⁷)(R⁸). In certain embodiments, R¹¹ is C₁-C₆alkylN(R⁷)(R⁸).

In the embodiments described herein, R¹² is hydrogen, halogen, —CN, —OH, C₁-C₆alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸), —N(R⁷)(R⁸) or C₁-C₆alkylN(R⁷)(R⁸). In certain embodiments, R¹² is hydrogen. In certain embodiments, R¹² is halogen. Suitable halogens include fluorine, chlorine, bromine, andiodine. In certain embodiments, R² is —CN. In certain embodiments, R² is —OH.

In certain embodiments, R² is C₁-C₆alkoxy. Suitable alkoxy groups include, but are not limited to, methoxy, ethoxy, n-propoxy, isopropoxy and n-butoxy. In certain embodiments, R¹² is C₁-C₆alkylOC₁-C₆alkyl. In certain embodiments, R¹² is —COOH. In certain embodiments, R¹² is C₁-C₆alkylCOOH. In certain embodiments, R² is C₃-C₆cycloalkyl. Suitable examples of cycloalkyls include, but are not limited to, cyclopropyl, cyclobutyl, cyclopentyl and cyclohexyl. In certain embodiments, R² is C₁-C₆alkyl. Examples of C₁-C₆alkyl groups include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R¹² is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R¹² is C₁-C₆alkylOH. Examples of suitable alcohols include, but are not limited to, methanol, ethanol, propanol, butanol and iso-butanol. In certain embodiments, R¹² is —CON(R⁷)(R⁸). In certain embodiments, R¹² is N(R⁷)(R⁸). In certain embodiments, R¹² is C₁-C₆alkylN(R⁷)(R⁸).

In certain embodiments, R¹² is hydrogen, methyl, ethyl, methoxy, —OH or

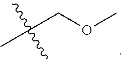 [SMILES: *COC]

In certain embodiments, R¹² is hydrogen or

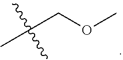 [SMILES: *COC]

In the embodiments described herein, R¹³ is hydrogen, halogen, —CN, —OH, C₁-C₆alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸), —N(R⁷)(R⁸) or C₁-C₆alkylN(R⁷)(R⁸). In certain embodiments, R¹³ is hydrogen. In certain embodiments, R¹³ is halogen. Suitable halogens include fluorine, chlorine, bromine, or iodine. In certain embodiments, R¹³ is —CN. In certain embodiments, R¹³ is —OH.

In certain embodiments, R¹³ is C₁-C₆alkoxy. Suitable alkoxy groups include, but are not limited to, methoxy, ethoxy, n-propoxy, isopropoxy and n-butoxy. In certain embodiments, R¹³ is C₁-C₆alkylOC₁-C₆alkyl. In certain embodiments, R¹³ is —COOH. In certain embodiments, R¹³ is C₁-C₆alkylCOOH. In certain embodiments, R¹³ is C₃-C₆cycloalkyl. Suitable examples of cycloalkyls include, but are not limited to, cyclopropyl, cyclobutyl, cyclopentyl and cyclohexyl. In certain embodiments, R¹³ is C₁-C₆alkyl. Examples of C₁-C₆alkyl groups include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R¹³ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R¹³ is C₁-C₆alkylOH. Examples of suitable alcohols include, but are not limited to, methanol, ethanol, propanol, butanol and iso-butanol. In certain embodiments, R¹³ is —CON(R⁷)(R⁸). In certain embodiments, R¹³ is N(R⁷)(R⁸). In certain embodiments, R¹³ is C₁-C₆alkylN(R⁷)(R⁸).

In certain embodiments, R¹³ is hydrogen, methyl, ethyl, methoxy, —OH or

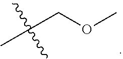 [SMILES: *COC]

In certain embodiments, R¹³ is hydrogen or

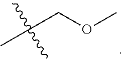 [SMILES: *COC]

In certain embodiments, R¹² and R¹³ are independently selected from the group consisting of hydrogen, C₁-C₆alkylOC₁-C₆alkyl, and C₁-C₆alkyl.

In certain embodiments described herein, each occurrence of R¹⁴ is independently selected from the group consisting of hydrogen, halogen, —CN, —OH, C₁-C₆alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸), —N(R⁷)(R⁸) and C₁-C₆alkylN(R⁷)(R⁸). In certain embodiments, R¹⁴ is hydrogen. In certain embodiments, R¹⁴ is halogen. Suitable halogens include fluorine, chlorine, bromine, or iodine. In certain embodiments, R¹⁴ is —CN. In certain embodiments, R¹⁴ is —OH.

In certain embodiments, R¹⁴ is C₁-C₆alkoxy. Suitable alkoxy groups include, but are not limited to, methoxy, ethoxy, n-propoxy, isopropoxy and n-butoxy. In certain embodiments, R¹⁴ is C₁-C₆alkylOC₁-C₆alkyl. In certain embodiments, R¹⁴ is —COOH. In certain embodiments, R¹⁴ is C₁-C₆alkylCOOH. In certain embodiments, R¹⁴ is C₃-C₆cycloalkyl. Suitable examples of cycloalkyls include, but are not limited to, cyclopropyl, cyclobutyl, cyclopentyl and cyclohexyl. In certain embodiments, R¹⁴ is C₁-C₆alkyl. Examples of C₁-C₆alkyl groups include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R¹⁴ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R¹⁴ is C₁-C₆alkylOH. Examples of suitable alcohols include, but are not limited to, methanol, ethanol, propanol, butanol and iso-butanol. In certain embodiments, R¹⁴ is —CON(R⁷)(R⁸). In certain embodiments, R¹⁴ is N(R⁷)(R⁸). In certain embodiments, R¹⁴ is C₁-C₆alkylN(R⁷)(R⁸).

In certain embodiments, wherein X is C(R¹⁴)₂, R¹⁴ is independently selected from the group consisting of hydrogen, halogen, —OH, C₁-C₆alkylOH, C₁-C₆alkylalkoxy, C₁-C₆alkylOC₁-C₆alkyl and C₁-C₆alkyl.

In certain embodiments, R¹⁴ is hydrogen, methyl, ethyl, methoxy, —OH or

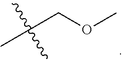 [SMILES: *COC]

In the embodiments described herein, R¹⁵ is hydrogen, halogen, —CN, —OH, C₁-C₆alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸) or N(R⁷)(R⁸). In certain embodiments, R¹⁵ is hydrogen. In certain embodiments, R⁵ is halogen. Suitable halogens include fluorine, chlorine, bromine, or iodine. In certain embodiments, R¹⁵ is —CN. In certain embodiments, R¹⁵ is —OH.

In certain embodiments, R¹⁵ is C₁-C₆alkoxy. Suitable alkoxys include, but are not limited to, methoxy, ethoxy, n-propoxy, isopropoxy and n-butoxy. In certain embodiments, R¹⁵ is C₁-C₆alkylOC₁-C₆alkyl. In certain embodiments, R¹⁵ is —COOH. In certain embodiments, R¹⁵ is C₁-C₆alkylCOOH. In certain embodiments, R¹⁵ is C₃-C₆cycloalkyl. Suitable examples of cycloalkyls include, but are not limited to, cyclopropyl, cyclobutyl, cyclopentyl and cyclohexyl. In certain embodiments, R¹⁵ is C₁-C₆alkyl. Examples of C₁-C₆alkyl groups include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R¹⁵ is ethyl.

In certain embodiments, R¹⁵ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R¹⁵ is C₁-C₆alkylOH. Examples of suitable alcohols include, but are not limited to, methanol, ethanol, propanol, butanol and iso-butanol. In certain embodiments, R¹⁵ is —CON(R⁷)(R⁸). In certain embodiments, R¹⁵ is N(R⁷)(R⁸). In certain embodiments, R¹⁵ is C₁-C₆alkylN(R⁷)(R⁸).

In certain embodiments, R¹⁵ is methyl or ethyl.

In the embodiments described herein, R¹⁶ is hydrogen, halogen, —CN, —OH, C₁-C₆alkoxy, C₁-C₆alkylOC₁-C₆alkyl, C₁-C₆alkylCOOH, —COOH, C₃-C₆cycloalkyl, C₁-C₆alkyl, haloC₁-C₆alkyl, C₁-C₆alkylOH, —CON(R⁷)(R⁸) or N(R⁷)(R⁸). In certain embodiments, R¹⁶ is hydrogen. In certain embodiments, R¹⁶ is halogen. Suitable halogens include fluorine, chlorine, bromine, or iodine. In certain embodiments, R¹⁶ is —CN. In certain embodiments, R¹⁶ is —OH.

In certain embodiments, R¹⁶ is C₁-C₆alkoxy. Suitable alkoxys include, but are not limited to, methoxy, ethoxy, n-propoxy, isopropoxy and n-butoxy. In certain embodiments, R¹⁶ is C₁-C₆alkylOC₁-C₆alkyl. In certain embodiments, R¹⁶ is —COOH. In certain embodiments, R¹⁶ is C₁-C₆alkylCOOH. In certain embodiments, R¹⁶ is C₃-C₆cycloalkyl. Suitable examples of cycloalkyls include, but are not limited to, cyclopropyl, cyclobutyl, cyclopentyl and cyclohexyl. In certain embodiments, R¹⁶ is C₁-C₆alkyl. Examples of C₁-C₆alkyl groups include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R¹⁶ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R¹⁶ is C₁-C₆alkylOH. Examples of suitable alcohols include, but are not limited to, methanol, ethanol, propanol, butanol and iso-butanol. In certain embodiments, R¹⁶ is —CON(R⁷)(R⁸). In certain embodiments, R¹⁶ is N(R⁷)(R⁸). In certain embodiments, R¹⁶ is C₁-C₆alkylN(R⁷)(R⁸).

In the embodiments of the compounds described herein, l is 0 or 1. In certain embodiments, l is 0. In certain embodiments, l is 1.

In the embodiments of the compounds described herein, m is 0 or 1. In certain embodiments, m is 0. In certain embodiments, m is 1.

In the embodiments of the compounds described herein, p is 0 or 1. In certain embodiments, p is 0. In certain embodiments, p is 1.

In certain embodiments, m and p are 1 and X is O.

In certain embodiments, m and p are 1 and X is CH₂.

In certain embodiments, m is 0, p is 1 and X is O.

In certain embodiments, m and p are 1 and X is SO₂.

In certain embodiments, m is 0, p is 1 and X is C(R¹⁴)₂, wherein each occurrence of R¹⁴ is independently selected from the group consisting of hydrogen, halogen, —OH, C₁-C₆alkoxy and C₁-C₆alkyl.

In certain embodiments, m is 1 and X is C(R¹⁴)₂, wherein each occurrence of R¹⁴ is independently selected from the group consisting of hydrogen, halogen, —OH, C₁-C₆alkoxy and C₁-C₆alkyl.

For example, in certain embodiments of Formula (I), l is 0; m is 1; p is 1; X is O; V, Y and Z are CH; and Q is CH₂ as shown in Formula (VII).

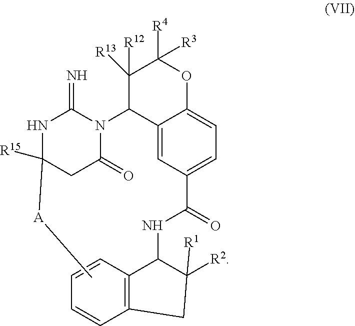

For example, in certain embodiments of Formula (I), l, m and p are 1; X is O; V, Y and Z are CH; and Q is O as shown in Formula (VIII).

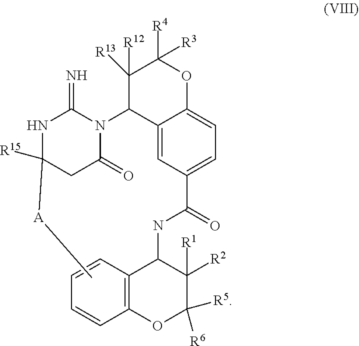

In the embodiments described herein, A is a straight or branched, saturated or unsaturated (C₃-C₁₀)alkylene, phenyl(C₃-C₁₀)alkylene or cycloalkyl(C₃-C₁₀)alkylene comprising at least one —CH₂— group, wherein one or more additional —CH₂— groups in A are optionally and independently replaced with a moiety selected from the group consisting of O, S, NR, —CONR, NRCO, SO₂, and SO₂NR and wherein one or more of the hydrogens along A can be replaced with a halogen. In certain embodiments, A is a straight or branched, saturated or unsaturated (C₃—C₁₀)alkylene or cycloalkyl(C₃-C₁₀)alkylene, wherein one or more —CH₂— groups in A are optionally and independently replaced with a moiety selected from the group consisting of O, S and NH. In certain embodiments, A will always have at least one —CH₂— group.

In certain embodiments, A is a straight (C₃-C₁₀)alkylene. Examples of straight (C₃-C₁₀)alkylenes include,

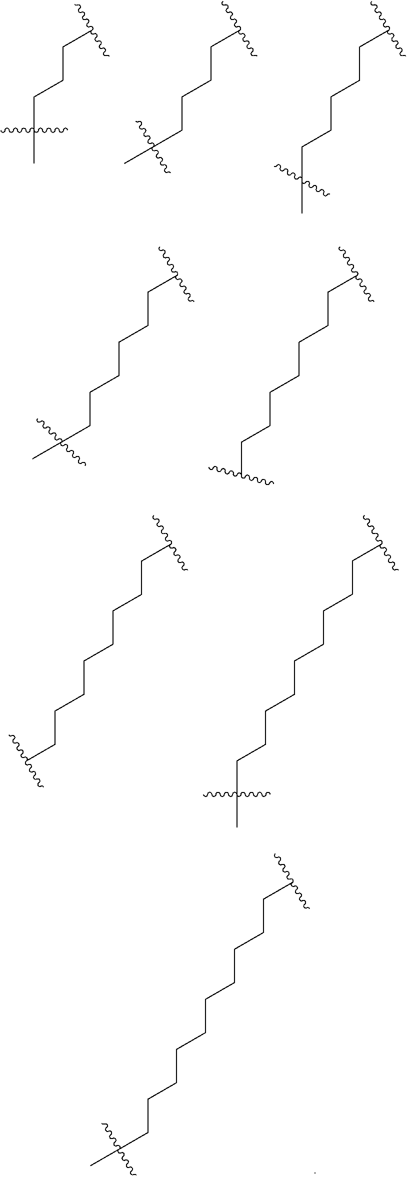

In certain embodiments, A is a branched (C₃-C₁₀)alkylene. Suitable branched (C₃-C₁₀)alkylenes include but are not limited to:

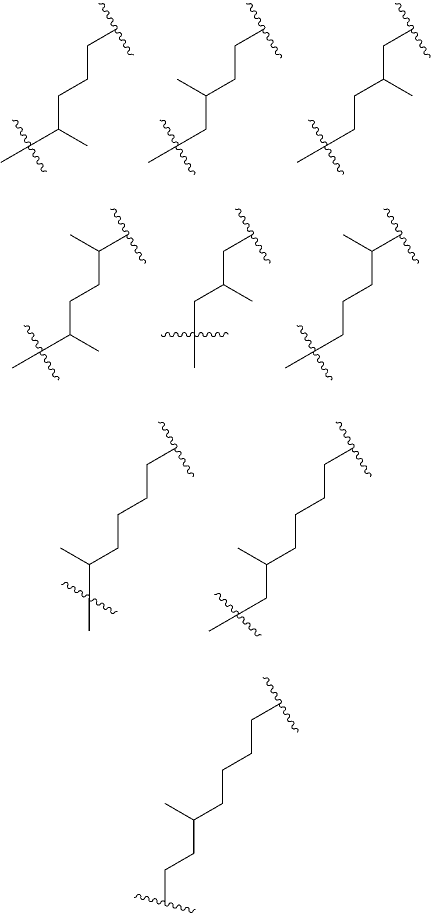

In certain embodiments, A is a saturated (C₃-C₁₀)alkylene. Examples include,

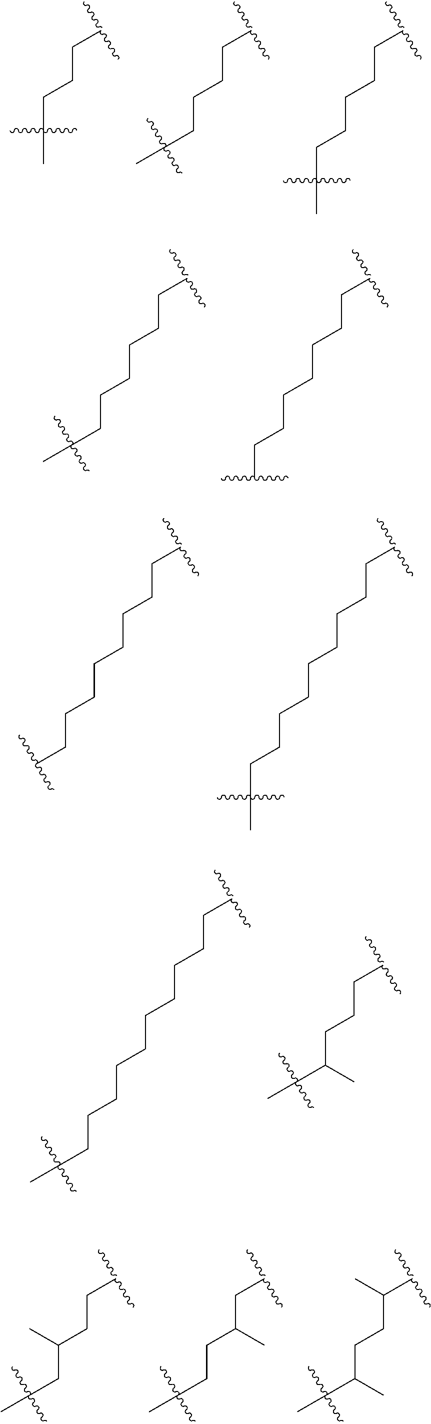

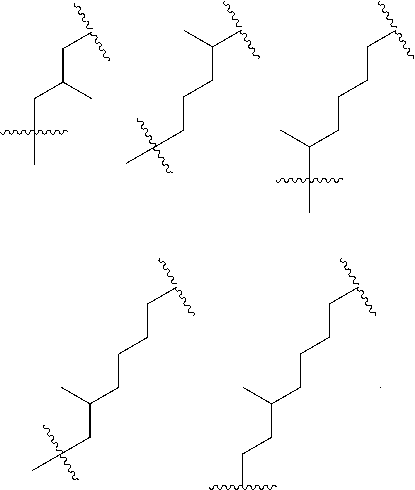

In certain embodiments, A is an unsaturated (C₃-C₁₀)alkylene. Suitable unsaturated (C₃-C₁₀)alkylenes include any of the saturated (C₃-C₁₀)alkylene, wherein hydrogens have been removed and one or more double or triple covalent bonds exist between adjacent carbon atoms. Examples of unsaturated (C₃-C₁₀)alkylenes include, but are not limited to,

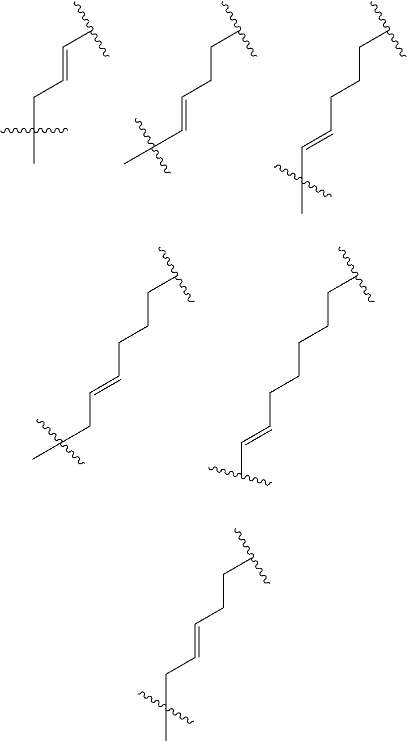 [SMILES: */C=C/C* ; */C=C/CC* ; */C=C/CCC* ; *C/C=C/CCC* ; */C=C/CCCCC* ; *C/C=C/CC*]

In other embodiments, A is a straight cycloalkyl(C₃-C₁₀)alkylene. Suitable straight cycloalkyl(C₃-C₁₀)alkylenes include a cycloalkyl(C₃-C₁₀)alkylene wherein two carbons in a chain are included in a (C₃-C₁₀)cycloalkyl. Examples of straight cycloalkyl(C₃-C₁₀)alkylenes include, but are not limited to,

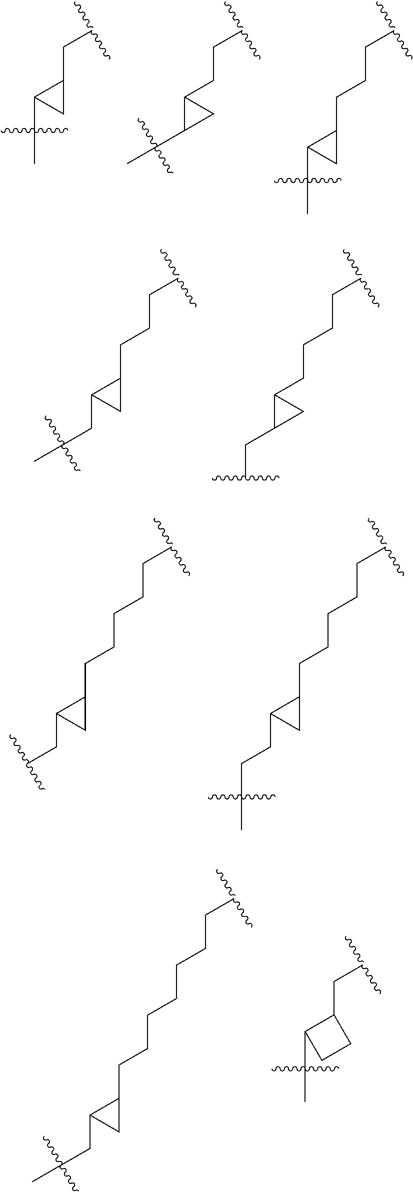

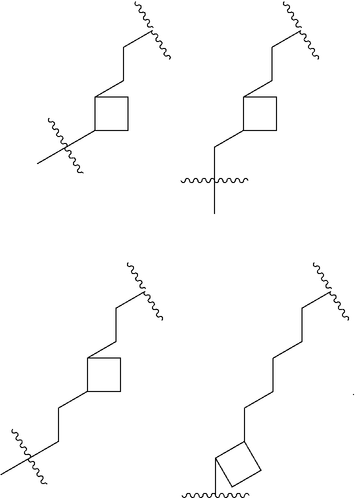 [SMILES: *CCC1CCC1* ; *CCC1CCC1C* ; *CCC1CCC1CC* ; *CCCCCC1CCC1*]

In certain embodiments, A is a branched cycloalkyl(C₃-C₁₀)alkylene. Suitable branched cycloalkyl(C₃-C₁₀)alkylenes include a branched (C₃-C₁₀)alkylene wherein two carbons in a chain are included in a (C₃-C₁₀)cycloalkyl. Examples of cycloalkyl(C₃-C₁₀)alkylenes include, but are not limited to,

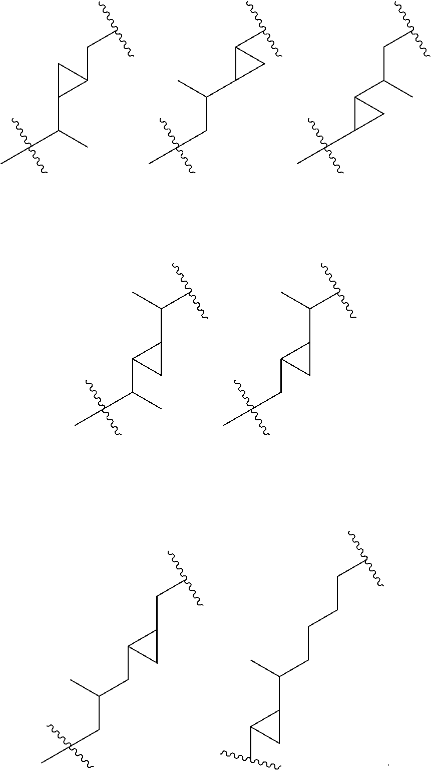

In certain embodiments, A is a saturated cycloalkyl(C₃-C₁₀)alkylene. Examples of saturated cycloalkyl(C₃-C₁₀)alkylenes include, but are not limited to,

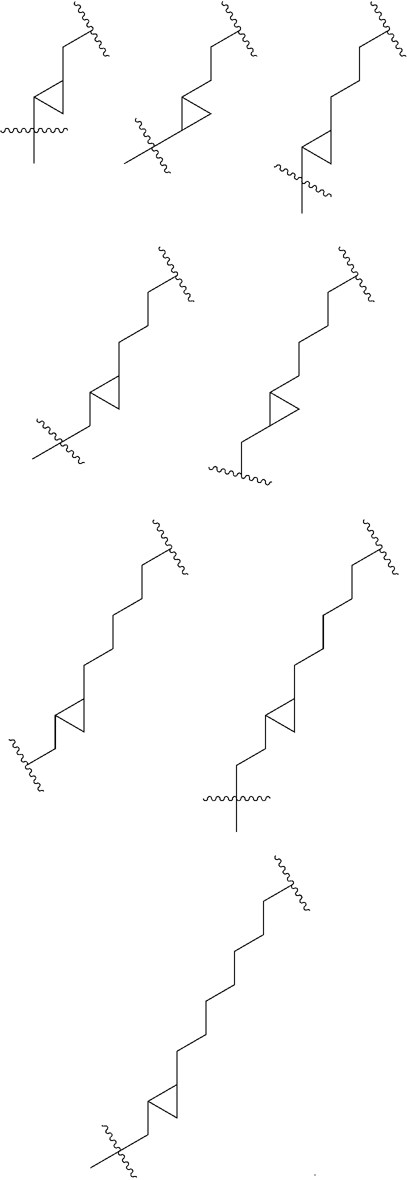

In certain embodiments, A is an unsaturated cycloalkyl(C₃-C₁₀)alkylene. Examples of unsaturated cycloalkyl(C₃-C₁₀)alkylenes include, but are not limited to,

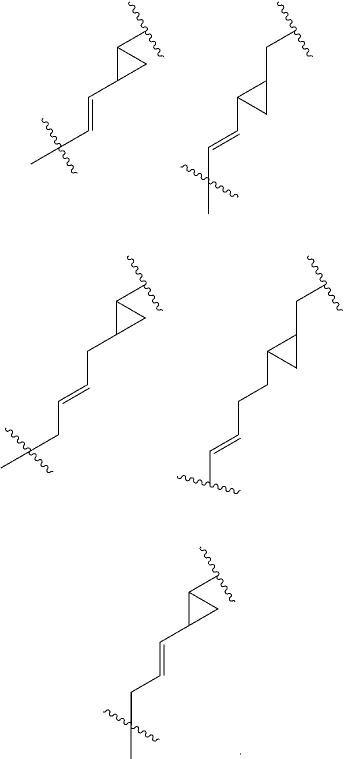 [SMILES: */C=C/C1CC1* ; */C=C/C1CC1C* ; *C/C=C/CC1CC1* ; */C=C/CCC1CC1C* ; *C/C=C/C1CC1*]

In certain embodiments, A is an unsaturated or saturated phenyl(C₃-C₁₀)alkylene. Examples of unsaturated and saturated phenyl(C₃-C₁₀)alkylenes include, but are not limited to

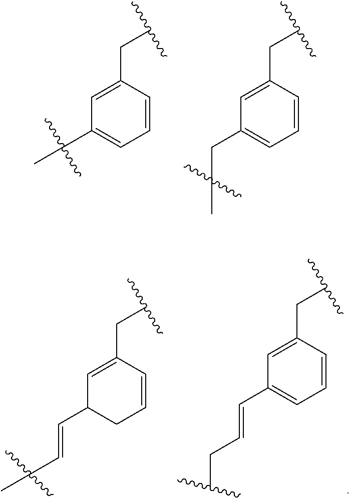 [SMILES: *Cc1cccc(*)c1 ; *Cc1cccc(C*)c1 ; */C=C/C1C=C(C*)C=CC1 ; *C/C=C/c1cccc(C*)c1]

In other embodiments, one or more —CH₂— groups in A are optionally and independently replaced with a moiety selected from the group consisting of O, S, NR, —CONR, NRCO, SO₂, and SO₂NR. In other embodiments, one or more —CH₂— groups in A are optionally and independently replaced with a moiety selected from the group consisting of O, S and NH. In other embodiments, one or more —CH₂— groups in A are optionally and independently replaced with O. In other embodiments, one or more —CH₂— groups in A are optionally and independently replaced with S. In other embodiments, one or more —CH₂— groups in A are optionally and independently replaced with NR. In other embodiments, one or more —CH₂— groups in A are optionally and independently replaced with —CONR. In other embodiments, one or more —CH₂— groups in A are optionally and independently replaced with NRCO. In other embodiments, one or more —CH₂— groups in A are optionally and independently replaced with SO₂. In other embodiments, one or more —CH₂— groups in A are optionally and independently replaced with and SO₂NR. R will be described in further detail below.

In the embodiments described herein, R is hydrogen, C₁-C₆alkylCOOH, —COOH, C₃-C₆cycloalkyl, C₁-C₆alkyl, haloC₁-C₆alkyl, C₁-C₆alkylOH, —COC₁-C₆alkyl or —COOC₁-C₆alkyl. In certain embodiments, R is hydrogen, C₁-C₆alkylCOOH, —COOH, C₃-C₆cycloalkyl, C₁-C₆alkyl, haloC₁-C₆alkyl or C₁-C₆alkylOH.

In certain embodiments, R is hydrogen. In certain embodiments, R is C₁-C₆alkylCOOH. In certain embodiments, R is —COOH. In certain embodiments, R is C₃-C₆cycloalkyl. Suitable examples of cycloalkyls include, but are not limited to, cyclopropyl, cyclobutyl, cyclopentyl and cyclohexyl. In certain embodiments, R is C₁-C₆alkyl. Examples of C₁-C₆alkyl groups include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R is C₁-C₆alkylOH. Examples of suitable alcohols include, but are not limited to, methanol, ethanol, propanol, butanol and iso-butanol.

In certain embodiments, R is —COC₁-C₆alkyl. Suitable examples include, but are not limited to, —COCH₃. In certain embodiments, R is —COOC₁-C₆alkyl. Suitable examples include, but are not limited to, —COOCH₃.

Examples of such embodiments include, but are not limited to,

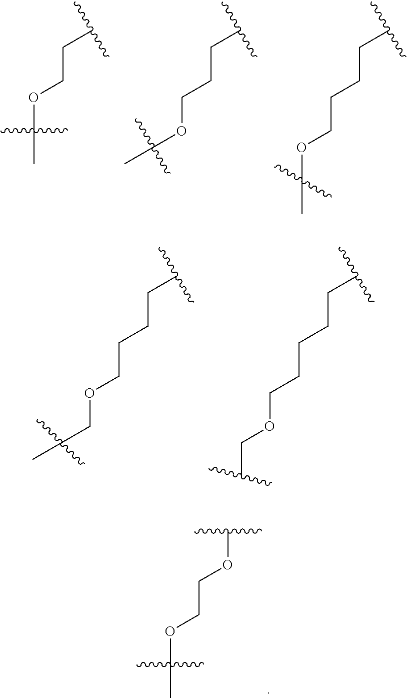 [SMILES: *CCO* ; *CCCO* ; *CCCCO* ; *CCCCOC* ; *CCCCCOC* ; *OCCO*]

In certain embodiments, A is

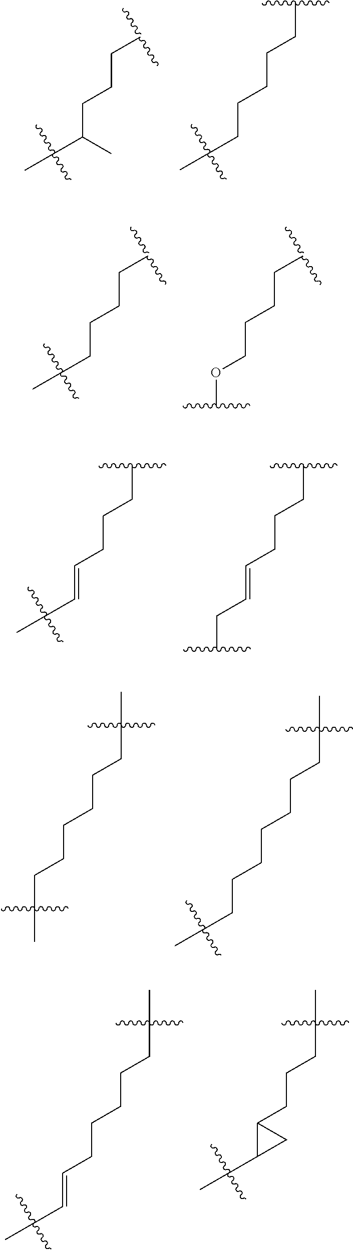

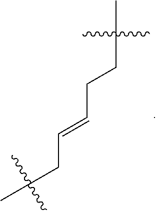 [SMILES: *C/C=C/CC*]

In certain embodiments, one or more of the hydrogens along A can be replaced with a halogen. Examples of suitable halogens include chlorine, bromine, fluorine and iodine. In certain embodiments, A is

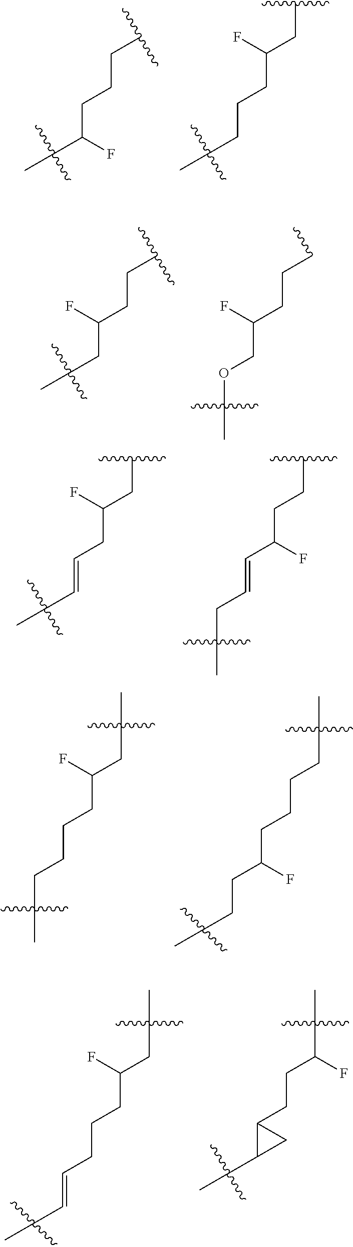

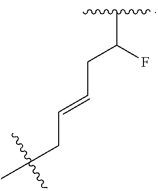 [SMILES: *C/C=C/CC(*)F]

In each of the various embodiments of the invention, in the compounds used in the methods herein, each variable (including those in each of Formulae (I)-(VIII), and the various embodiments thereof) it shall be understood that each variable is to be selected independently of the others unless otherwise indicated.

In each of the various embodiments of the invention, the compounds described herein, including those in each of Formulae (I)-(VIII) and the various embodiments thereof, may exit in different forms of the compounds such as, for example, any solvates, hydrates, stereoisomers, and tautomers of said compounds and of any pharmaceutically acceptable salts thereof.

In certain embodiments, compounds described herein include:

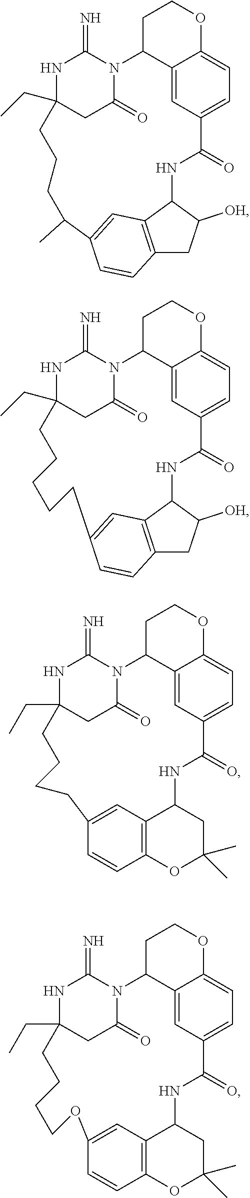

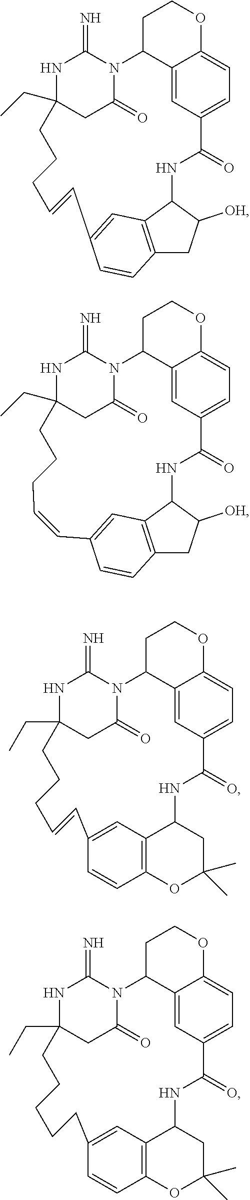

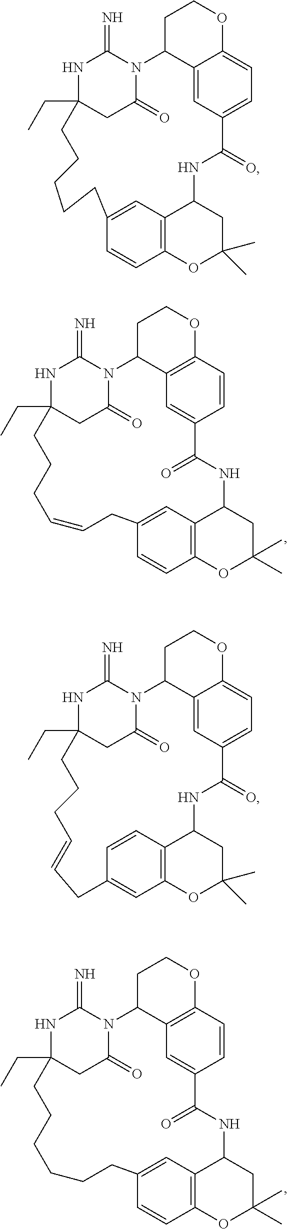

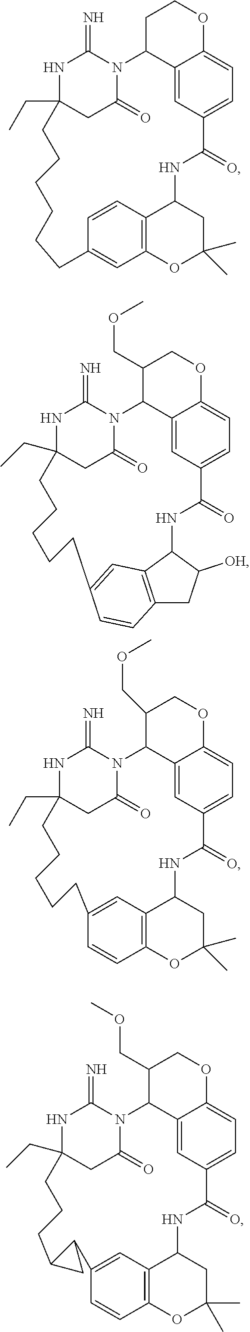

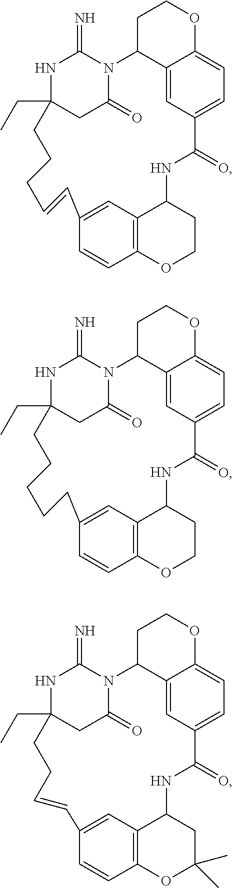

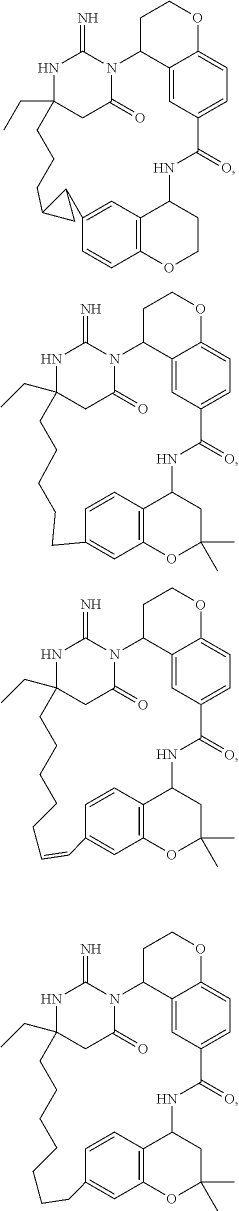

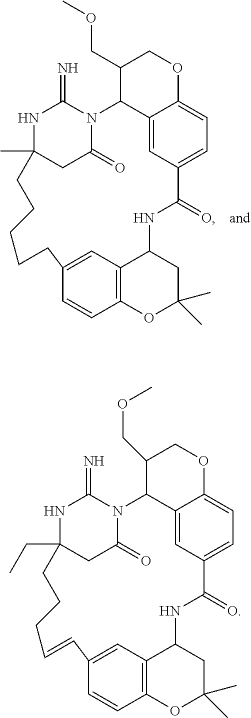

In certain embodiments, compounds described herein include:

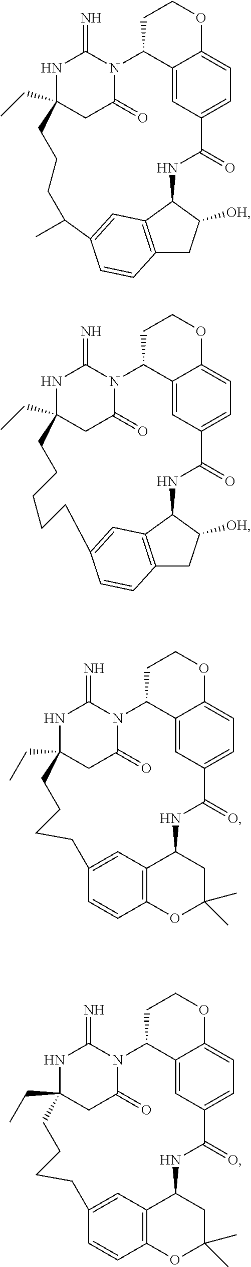

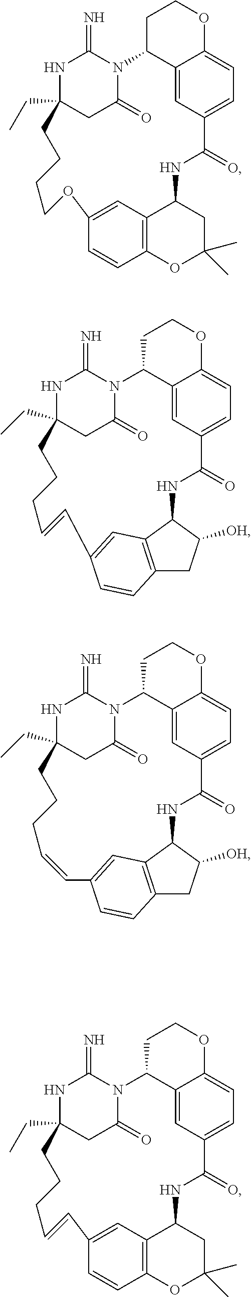

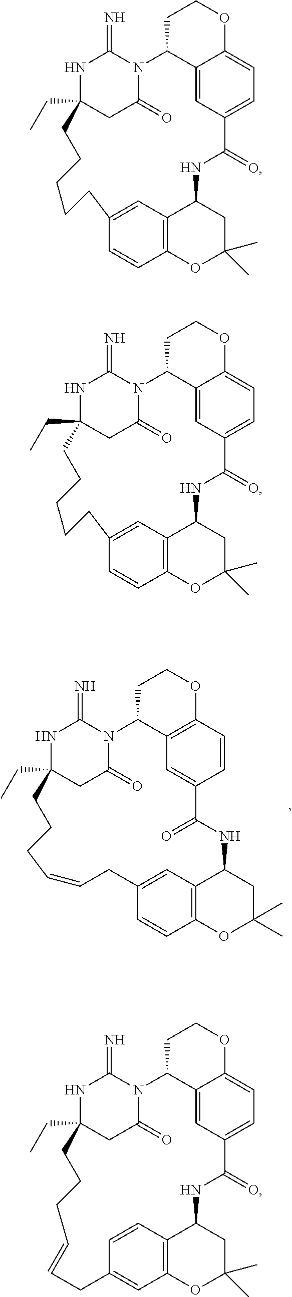

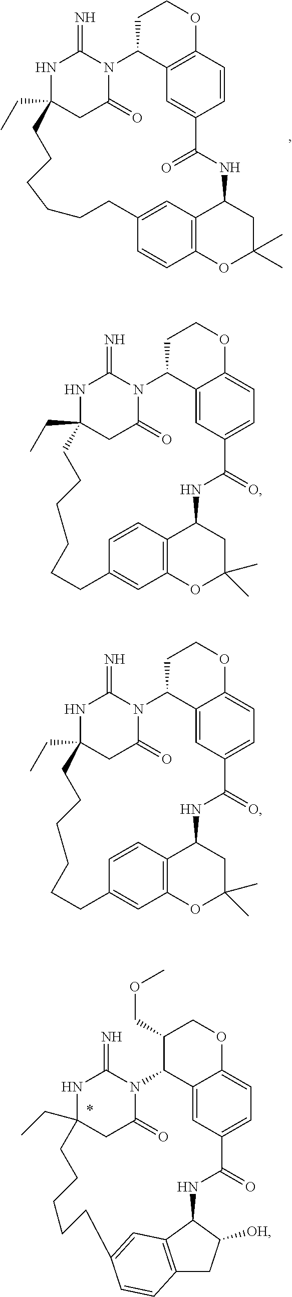

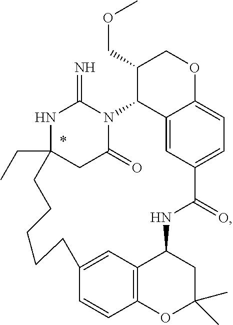 [SMILES: CCC12CCCCCc3ccc4c(c3)[C@H](CC(C)(C)O4)NC(=O)c3ccc4c(c3)[C@@H]([C@H](COC)CO4)N(C(=N)N1)C(=O)C2]

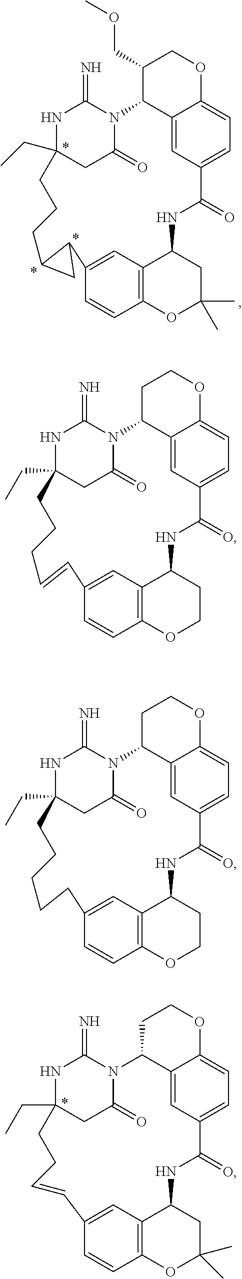

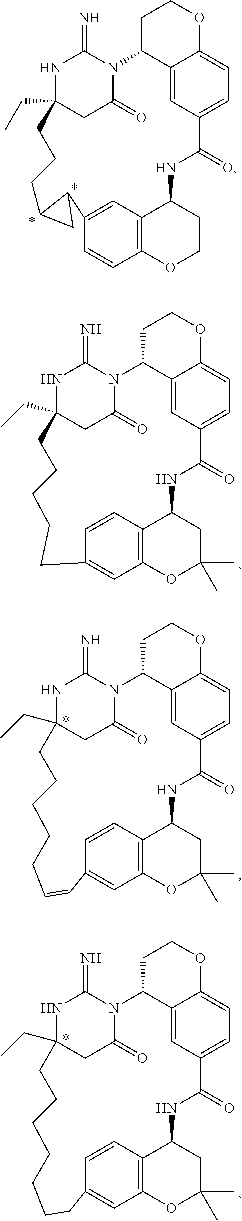

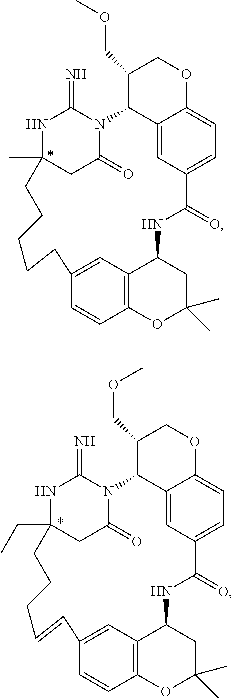

or a pharmaceutically acceptable salt thereof.

### Definitions and Abbreviations

The terms used herein have their ordinary meaning and the meaning of such terms is independent at each occurrence thereof. That notwithstanding and except where stated otherwise, the following definitions apply throughout the specification and claims. Chemical names, common names and chemical structures may be used interchangeably to describe that same structure. These definitions apply regardless of whether a term is used by itself or in combination with other terms, unless otherwise indicated. Hence the definition of “alkyl” applies to “alkyl” as well as the “alkyl” portion of “hydroxyalkyl”, “haloalkyl”, “arylalkyl-”, “alkylaryl-”, “alkoxy” etc.

It shall be understood that, in the various embodiments of the invention described herein, any variable not explicitly defined in the context of the embodiment is as defined in Formula (I).

In the various embodiments described herein, each variable is selected independently of the others unless otherwise indicated.

“Drug resistant” means, in connection with a Plasmodium parasite strain, a Plasmodium species which is no longer susceptible to at least one previously effective drug; which has developed the ability to withstand attack by at least one previously effective drug. A drug resistant strain may relay that ability to withstand to its progeny. Said resistance may be due to random genetic mutations in the bacterial cell that alters its sensitivity to a single drug or to different drugs.

“Patient” includes both human and non-human animals. Non-human animals include those research animals and companion animals such as mice, rats, primates, monkeys, chimpanzees, great apes, dogs, and house cats.

“Pharmaceutical composition” (or “pharmaceutically acceptable composition”) means a composition suitable for administration to a patient. Such compositions may contain the compound (or compounds) of the invention or mixtures thereof, or salts, solvates, prodrugs, isomers, or tautomers thereof, and one or more pharmaceutically acceptable carriers or diluents. The term “pharmaceutical composition” is also intended to encompass both the bulk composition and individual dosage units comprised of one or more (e.g., two) pharmaceutically active agents such as, for example, a compound of the present invention and an additional agent selected from the lists of the additional agents described herein, along with any pharmaceutically inactive excipients. The bulk composition and each individual dosage unit can contain fixed amounts of the afore-said “more than one pharmaceutically active agents”. The bulk composition is material that has not yet been formed into individual dosage units. An illustrative dosage unit is an oral dosage unit such as tablets, pills and the like. Similarly, the herein-described method of treating a patient by administering a pharmaceutical composition of the present invention is also intended to encompass the administration of the afore-said bulk composition and individual dosage units.

“Halogen” and “halo” mean fluorine, chlorine, bromine, or iodine. Preferred are fluorine, chlorine and bromine.

“Alkylene,” by itself or as part of another substituent means a divalent hydrocarbon chain radical having the stated number of carbon atoms. For example, —(C₁-C₅)alkylene, would include, e.g., —CH₂—, —CH₂CH₂—, —CH₂CH₂CH₂—, —CH₂CH₂CH₂CH₂—, —CH₂CH(CH₃)CH₂— or —CH₂CH₂CH₂CH₂CH₂—. A straight alkylene means a divalent straight hydrocarbon chain radical having the stated number of carbon atoms. A branched alkylene means a divalent branched hydrocarbon chain radical having the stated number of carbon atoms. A saturated alkylene means a divalent saturated hydrocarbon chain radical having the stated number of carbon atoms. An unsaturated alkylene means a divalent hydrocarbon chain radical having the stated number of carbon atoms and one or more double or triple covalent bonds within the chain. A cycloalkylalkylene means a divalent hydrocarbon chain radical having the stated number of carbon atoms and a cycloalkyl moiety within the chain. A phenylalkylene means a divalent hydrocarbon chain radical having the stated number of carbon atoms and a phenyl group within the chain.

“Alkyl” means an aliphatic hydrocarbon group which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more lower alkyl groups such as methyl, ethyl or propyl, are attached to a linear alkyl chain. “Lower alkyl” means a group having about 1 to about 6 carbon atoms in the chain which may be straight or branched. Non-limiting examples of suitable alkyl groups include methyl, ethyl, n-propyl, isopropyl and t-butyl.

“Haloalkyl” means an alkyl as defined above wherein one or more hydrogen atoms on the alkyl is replaced by a halo group defined above.

“Aryl” means an aromatic monocyclic or multicyclic ring system comprising about 6 to about 14 carbon atoms, preferably about 6 to about 10 carbon atoms. The aryl group can be optionally substituted with one or more “ring system substituents” which may be the same or different, and are as defined herein. Non-limiting examples of suitable aryl groups include phenyl and naphthyl. “Monocyclic aryl” means phenyl.

“Cycloalkyl” means a non-aromatic mono- or multicyclic ring system comprising about 3 to about 12 carbon atoms, preferably about 3 to about 10 carbon atoms. Preferred cycloalkyl rings contain about 5 to about 10 ring atoms. The cycloalkyl can be optionally substituted with one or more substituents, which may be the same or different, as described herein. Monocyclic cycloalkyl refers to monocyclic versions of the cycloalkyl moieties described herein. Non-limiting examples of suitable monocyclic cycloalkyls include cyclopropyl, cyclopentyl, cyclohexyl, cycloheptyl and the like. Multicyclic cycloalkyls refers to multicyclic, including bicyclic, rings that include a non-aromatic ring. Non-limiting examples of suitable multicyclic cycloalkyls include 1-decalinyl, norbornyl, adamantyl and the like. In certain embodiments, a non-aromatic ring is fused to an aromatic ring.

“Heterocycloalkyl” (or “heterocyclyl”) means a non-aromatic, saturated or partially saturated monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. Preferred heterocyclyls contain about 5 to about 6 ring atoms. The prefix aza, oxa or thia before the heterocyclyl root name means that at least a nitrogen, oxygen or sulfur atom respectively is present as a ring atom. Any —NH in a heterocyclyl ring may exist protected such as, for example, as an —N(Boc), —N(CBz), —N(Tos) group and the like; such protections are also considered part of this invention. The heterocyclyl can be optionally substituted by one or more substituents, which may be the same or different, as described herein. The nitrogen or sulfur atom of the heterocyclyl can be optionally oxidized to the corresponding N-oxide, S-oxide or S,S-dioxide. Thus, the term “oxide,” when it appears in a definition of a variable in a general structure described herein, refers to the corresponding N-oxide, S-oxide, or S,S-dioxide. “Heterocyclyl” also includes rings wherein ═O replaces two available hydrogens on the same carbon atom (i.e., heterocyclyl includes rings having a carbonyl group in the ring). Such ═O groups may be referred to herein as “oxo.” An example of such a moiety is pyrrolidinone (or pyrrolidone):

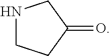

As used herein, the term “monocyclic heterocycloalkyl” refers monocyclic versions of the heterocycloalkyl moieties described herein and include a 4- to 7-membered monocyclic heterocycloalkyl groups comprising from 1 to 4 ring heteroatoms, said ring heteroatoms being independently selected from the group consisting of N,N-oxide, O, S, S-oxide, S(O), and S(O)₂. The point of attachment to the parent moiety is to any available ring carbon or ring heteroatom. Non-limiting examples of monocyclic heterocycloalkyl groups include piperidyl, oxetanyl, pyrrolyl, piperazinyl, morpholinyl, thiomorpholinyl, thiazolidinyl, 1,4-dioxanyl, tetrahydrofuranyl, tetrahydrothiophenyl, beta lactam, gamma lactam, delta lactam, beta lactone, gamma lactone, delta lactone, and pyrrolidinone, and oxides thereof. A non-limiting example of a monocyclic heterocycloalkyl group include the moiety:

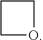

Non-limiting examples of multicyclic heterocycloalkyl groups include, bicyclic heterocycloalkyl groups. Specific examples include, but are not limited to,

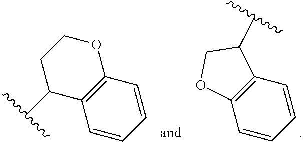 [SMILES: *C1CCOc2ccccc21 ; *C1COc2ccccc21]

“Alkoxy” means an alkyl-O— group in which the alkyl group is as previously described. Non-limiting examples of suitable alkoxy groups include methoxy, ethoxy, n-propoxy, isopropoxy and n-butoxy. The bond to the parent moiety is through the ether oxygen.

The term “substituted” means that one or more hydrogens on the designated atom is replaced with a selection from the indicated group, provided that the designated atom's normal valency under the existing circumstances is not exceeded, and that the substitution results in a stable compound. combinations of substituents and/or variables are permissible only if such combinations result in stable compounds. By “stable compound” or “stable structure” is meant a compound that is sufficiently robust to survive isolation to a useful degree of purity from a reaction mixture, and formulation into an efficacious therapeutic agent.

The term “optionally substituted” means optional substitution with the specified groups, radicals or moieties.

When a variable appears more than once in a group, e.g., R⁸ in —N(R⁸)₂, or a variable appears more than once in a structure presented herein, the variables can be the same or different.

A solid line —, as a bond generally indicates a mixture of, or either of, the possible isomers, e.g., containing (R)- and (S)-stereochemistry. For example:

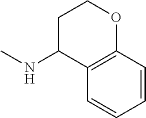

means containing either one of or both

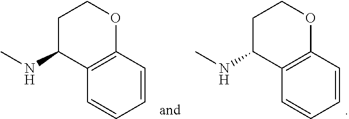

The wavy line [symbol] , as used herein shown crossing a line representing a chemical bond, indicates a point of attachment to the rest of the compound. Lines drawn into the ring systems, such as, for example

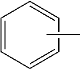

indicates that the indicated line (bond) may be attached to any of the substitutable ring atoms.

“Oxo” is defined as an oxygen atom that is double bonded to a ring carbon in a cycloalkyl, cycloalkenyl, heterocycloalkyl, heterocycloalkenyl, or another ring described herein, e.g.,

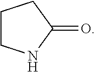

In this specification, where there are multiple oxygen and/or sulfur atoms in a ring system, there cannot be any adjacent oxygen and/or sulfur atoms present in said ring system.

As well known in the art, a bond drawn from a particular atom wherein no moiety is depicted at the terminal end of the bond indicates a methyl group bound through that bond to the atom, unless stated otherwise. For example:

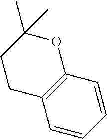

represents

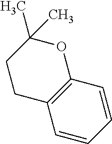

In another embodiment, the compounds useful in the methods of the invention, and/or compositions comprising them useful in said methods, are present in isolated and/or purified form. The term “purified”, “in purified form” or “in isolated and purified form” for a compound refers to the physical state of said compound after being isolated from a synthetic process (e.g. from a reaction mixture), or natural source or combination thereof. Thus, the term “purified”, “in purified form” or “in isolated and purified form” for a compound refers to the physical state of said compound (or a tautomer or stereoisomer thereof, or pharmaceutically acceptable salt or solvate of said compound, said stereoisomer, or said tautomer) after being obtained from a purification process or processes described herein or well known to the skilled artisan (e.g., chromatography, recrystallization and the like), in sufficient purity to be suitable for in vivo or medicinal use and/or characterizable by standard analytical techniques described herein or well known to the skilled artisan.

It shall be understood that any carbon as well as heteroatom with unsatisfied valences in the text, schemes, examples and tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in Organic Synthesis (1991), Wiley, New York.

Another embodiment provides prodrugs and/or solvates of the compounds of the invention. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g., a drug precursor) that is transformed in vivo to yield a compound of the invention or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a compound useful in the methods of the invention or a pharmaceutically acceptable salt thereof, contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl, and the like.

Similarly, if a compound used in the methods of the invention contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a compound used in the methods of the invention incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇) cycloalkyl, benzyl, or R-carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl, carboxy (C₁-C₆)alkyl, amino(C₁-C₄)alkyl or mono-N— or di-N,N—(C₁-C₆)alkylaminoalkyl, —C(Y⁴)Y⁵ wherein Y⁴ is H or methyl and Y⁵ is mono-N— or di-N,N—(C₁-C₆)alkylamino morpholino, piperidin-1-yl or pyrrolidin-1-yl, and the like.

One or more compounds used in the methods of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of the invention with one or more solvent molecules.

This physical association involves varying degrees of ionic and covalent bonding, including hydrogen bonding. In certain instances, the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

One or more compounds used in the methods of the invention may optionally be converted to a solvate. Preparation of solvates is generally known. Thus, for example M. Caira et al, J. Pharmaceutical Sci., 1993, 3, 601-611, describe the preparation of the solvates of the antifungal fluconazole in ethyl acetate as well as from water. Similar preparations of solvates, hemisolvate, hydrates and the like are described by E. C. van Tonder et al, AAPS PharmSciTech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

“Effective amount” or “therapeutically effective amount” is meant to describe an amount of compound or a composition used in the methods of the present invention effective in inhibiting the above-noted diseases or enzyme activity and thus producing the desired therapeutic, ameliorative, inhibitory or preventative effect.

Another embodiment provides pharmaceutically acceptable salts of the compounds to be used in the methods of the invention. Thus, reference to a compound used in the methods of the invention herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of the invention contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds used in the methods of the invention may be formed, for example, by reacting a compound of the invention with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates) and the like.

Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use, (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Another embodiment provides pharmaceutically acceptable esters of the compounds used in the methods of the invention. Such esters include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di(C₆₋₂₄)acyl glycerol.

As mentioned herein, another embodiment provides tautomers of the compounds of the invention to be used in the methods herein, and salts, solvates, esters and prodrugs of said tautomers. It shall be understood that all tautomeric forms of such compounds are within the scope of the compounds used in the methods of the invention. For example, all keto-enol and imine-enamine forms of the compounds, when present, are included in the invention.

The compounds used in the methods of the invention may contain asymmetric or chiral centers, and, therefore, exist in different stereoisomeric forms. It is intended that all stereoisomeric forms of the compounds used in the methods of the invention as well as mixtures thereof, including racemic mixtures, form part of the present invention. In addition, the present invention embraces use of all geometric and positional isomers. For example, if a compound used in the methods of the invention incorporates a double bond or a fused ring, both the cis- and trans-forms, (E) and (Z) forms, as well as mixtures, are embraced within the scope of the invention.

Another embodiment provides for diastereomeric mixtures and individual enantiomers of the compounds used in the methods of the invention. Diastereomeric mixtures can be separated into their individual diastereomers based on their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds used in the methods of the invention may be atropisomers (e.g., substituted biaryls) and are considered as part of this invention. Enantiomers can also be separated by use of chiral HPLCcolumn.

All stereoisomers (for example, geometric isomers, optical isomers and the like) of the compounds used in the methods of the invention (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated as embodiments within the scope of this invention, as are positional isomers (such as, for example, 4-pyridyl and 3-pyridyl). For example, if a compound of the invention incorporates a double bond or a fused ring, both the cis- and trans-forms, as well as mixtures, are embraced within the scope of the invention. Also, for example, all keto-enol and imine-enamine forms of the compounds are included in the methods of the invention.

Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to equally apply to the salt, solvate, ester and prodrug of enantiomers, stereoisomers, rotamers, tautomers, positional isomers, racemates or prodrugs of the inventive compounds.

Another embodiment provides isotopically-labelled compounds to be used in the methods the invention. Such compounds are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P ³²P, ³⁵S, ¹⁸F, and ³⁶Cl, respectively.

Certain isotopically-labelled compounds of the invention (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled compounds of the invention can generally be prepared by following procedures analogous to those disclosed in the Schemes and/or in the Examples hereinbelow, by substituting an appropriate isotopically labelled reagent for a non-isotopically labelled reagent.

In the compounds used in the methods of the invention, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of the invention. For example, different isotopic forms of hydrogen (H) include protium (¹H) and deuterium (²H). The presence of deuterium in the compounds of the invention is indicated by “D”. Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds of the invention can be prepared without undue experimentation by conventional techniques well known to those skilled in the art or by processes analogous to those described in the schemes and examples herein using appropriate isotopically-enriched reagents and/or intermediates.

Polymorphic forms of the compounds used in the methods of the invention, and of the salts, solvates, esters and prodrugs of the compounds of the invention, are intended to be included in the present invention.

### Methods of Treatment

The present invention is directed to methods of treatment of Plasmodium infections comprising administering to a subject in need thereof a compound described herein, or a pharmaceutically acceptable salt thereof. More specifically, the methods of the invention comprise administration of a compound of any of Formulas (I)-(VIII), or a pharmaceutically acceptable salt thereof. In certain embodiments, the compounds of any of Formulas (I)-(VIII), or a pharmaceutically acceptable salt thereof, are administered in the form of a pharmaceutical composition, further comprising a pharmaceutically acceptable carrier or excipient.

The present invention provides a method for treating a Plasmodium infection, or for treating malaria, or for inhibiting plasmepsin X which comprises administering to a subject in need of such treatment a therapeutically effective amount of a compound, or a pharmaceutically acceptable salt thereof, said compound having any of structural Formulas (I)-(VIII) described in the Summary of the Invention. In some embodiments, the compounds of any of Formulas (I)-(VIII) or pharmaceutically acceptable salts thereof, are administered with a pharmaceutically acceptable carrier, as a pharmaceutical composition. Also provided herein are various embodiments of these methods, as described, infra.

The invention also relates to the use of a compound of Formulae (I)-(VIII) or a pharmaceutically acceptable salt thereof for inhibiting plasmepsin X activity, for treating a Plasmodium infection, or for treating malaria. The invention further relates to the use of a compound of Formulae (I)-(VIII) or a pharmaceutically acceptable salt thereof in the manufacture of a medicament for inhibiting plasmepsin X activity, for treating a Plasmodium infection, or for treating malaria. The compounds of Formulae (I)-(VIII) or pharmaceutically acceptable salts thereof described in any of the embodiments of the invention herein are useful for any of the uses above.

The present invention provides a method for treating a Plasmodium infection, or for treating malaria, or for inhibiting plasmepsin IX which comprises administering to a subject in need of such treatment a therapeutically effective amount of a compound, or a pharmaceutically acceptable salt thereof, said compound having the structural Formula (I) described in the Summary of the Invention. In some embodiments, the compounds of Formula (I), or pharmaceutically acceptable salts thereof, are administered with a pharmaceutically acceptable carrier, as a pharmaceutical composition. Also provided herein are various embodiments of these methods, as described, infra.

The invention also relates to the use of a compound of Formulae (I)-(VIII) or a pharmaceutically acceptable salt thereof for inhibiting plasmepsin IX activity, for treating a Plasmodium infection, or for treating malaria. The invention further relates to the use of a compound of Formulae (I)-(VIII) or a pharmaceutically acceptable salt thereof in the manufacture of a medicament for inhibiting plasmepsin IX activity, for treating a Plasmodium infection, or for treating malaria. The compounds of Formulae (I)-(VIII) or pharmaceutically acceptable salts thereof described in any of the embodiments of the invention herein are useful for any of the uses above.

The present invention provides a method for treating a Plasmodium infection, or for treating malaria, or for inhibiting plasmepsin X and plasmepsin IX which comprises administering to a subject in need of such treatment a therapeutically effective amount of a compound, or a pharmaceutically acceptable salt thereof, said compound having the structural Formula (I) described in the Summary of the Invention. In some embodiments, the compounds of Formula (I), or pharmaceutically acceptable salts thereof, are administered with a pharmaceutically acceptable carrier, as a pharmaceutical composition. Also provided herein are various embodiments of these methods, as described, infra.

The invention also relates to the use of a compound of Formulae (I)-(VIII) or a pharmaceutically acceptable salt thereof for inhibiting plasmepsin X and plasmepsin IX activity, for treating a Plasmodium infection, or for treating malaria. The invention further relates to the use of a compound of Formulae (I)-(VIII) or a pharmaceutically acceptable salt thereof in the manufacture of a medicament for inhibiting plasmepsin X and plasmepsin IX activity, for treating a Plasmodium infection, or for treating malaria. The compounds of Formulae (I)-(VIII) or pharmaceutically acceptable salts thereof described in any of the embodiments of the invention herein are useful for any of the uses above.

The methods of the present invention are useful for treating malaria in that they inhibit the onset, growth, or progression of the condition, ameliorate the symptoms of the condition, cause regression of the condition, cure the condition, or otherwise improve the general well-being of a subject afflicted with, or at risk of, contracting the condition. Thus, in accordance with the presently disclosed subject matter, the terms “treat”, “treating”, and grammatical variations thereof, as well as the phrase “method of treating”, are meant to encompass any desired therapeutic intervention, including but not limited to a method for treating an existing infection in a subject of infection, such as in a subject that has been exposed to a parasite as disclosed herein.

Embodiments of the invention also include one or more of the compounds of Formulae (I)-(VIII) or a pharmaceutically acceptable salt thereof (i) for use in, (ii) for use as a medicament or composition for, or (iii) for use in the preparation of a medicament for: (a) therapy (e.g., of the human body); (b) medicine; (c) inhibition of parasite/Plasmodium growth, (d) treatment or prophylaxis of infection by Plasmodium species; (e) reduction of the progression, onset or severity of pathological symptoms associated with Plasmodium infection and/or reduction of the likelihood of severe Plasmodium infection or, (f) treatment, prophylaxis of, or delay in the onset, severity, or progression of Plasmodium-associated disease(s), including, but not limited to: malaria.

Accordingly, another embodiment provides methods for the treatment of malaria or for the treatment of Plasmodium infection, comprising administration of combinations comprising an amount of at least one compound of Formulae (I)-(VIII), or a pharmaceutically acceptable salt, solvate, ester or prodrug thereof, and an effective amount of one or more additional agents described below. In certain embodiments, described herein are methods for the treatment of malaria or for the treatment of Plasmodium infection, comprising administration of combinations comprising an amount of at least one compound of Formulae (I)-(VIII), or a pharmaceutically acceptable salt, solvate, ester or prodrug thereof, and an effective amount of one or more additional anti-malarial agents. In certain embodiments, described herein are methods for the treatment of malaria by inhibition of plasmepsin X, IX and at least one other mechanism, comprising administration of combinations comprising an amount of at least one compound of Formulae (I)-(VIII), or a pharmaceutically acceptable salt, solvate, ester or prodrug thereof, and an effective amount of one or more additional anti-malarial agents, wherein the additional anti-malarial agents act through a different mechanism than inhibiting plasmepsin IX or plasmepsin X. The pharmacological properties of the compounds of Formulae (I)-(VIII), or a pharmaceutically acceptable salt thereof may be confirmed by several pharmacological assays.

### Dosage and Administration

Another embodiment provides suitable dosages and dosage forms of the compounds used in the methods of the invention. Suitable doses for administering compounds used in the methods of the invention to patients may readily be determined by those skilled in the art, e.g., by an attending physician, pharmacist, or other skilled worker, and may vary according to patient health, age, weight, frequency of administration, use with other active ingredients, and/or indication for which the compounds are administered. Doses may range from about 0.001 to 500 mg/kg of body weight/day of the compound of the invention. In one embodiment, the dosage is from about 0.01 to about 25 mg/kg of body weight/day of a compound of the invention, or a pharmaceutically acceptable salt or solvate of said compound. In another embodiment, the quantity of active compound in a unit dose of preparation may be varied or adjusted from about 1 mg to about 100 mg, in specific embodiments from about 1 mg to about 50 mg, in specific embodiments from about 1 mg to about 25 mg, according to the particular application. In another embodiment, a typical recommended daily dosage regimen for oral administration can range from about 1 mg/day to about 500 mg/day, in specific embodiments 1 mg/day to 200 mg/day, in two to four divided doses.

As discussed above, the amount and frequency of administration of the compounds of the invention and/or the pharmaceutically acceptable salts thereof will be regulated according to the judgment of the attending clinician considering such factors as age, condition and size of the patient as well as severity of the symptoms being treated.

Liquid form preparations include solutions, suspensions and emulsions. As an example, may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations that are intended to be converted, shortly before use, to liquid form preparations for either oral or parenteral administration. Such liquid forms include solutions, suspensions and emulsions.

Another embodiment provides for use of compositions comprising a compound of Formulae (I)-(VIII), or a pharmaceutically acceptable salt thereof formulated for transdermal delivery. The transdermal compositions can take the form of creams, lotions, aerosols and/or emulsions and can be included in a transdermal patch of the matrix or reservoir type as are conventional in the art for this purpose.

Another embodiment provides for use of compositions comprising a compound of Formulae (I)-(VIII), or a pharmaceutically acceptable salt thereof formulated for subcutaneous delivery. Another embodiment provides for use of compositions suitable for oral delivery. In some embodiments, it may be advantageous for the pharmaceutical preparation comprising one or more compounds of Formulae (I)-(VIII), or a pharmaceutically acceptable salt thereof to be prepared in a unit dosage form. In such forms, the preparation is subdivided into suitably sized unit doses containing appropriate quantities of the active component, e.g., an effective amount to achieve the desired purpose. Each of the foregoing alternatives is considered as included in the various embodiments of the invention.

When used in combination with one or more additional therapeutic agents (“combination therapy”), the compounds used in the methods of this invention, i.e. the compounds of Formulae (I)-(VIII), may be administered together or sequentially. When administered sequentially, compounds of the invention may be administered before or after the one or more additional therapeutic agents, as determined by those skilled in the art or patient preference.

If formulated as a fixed dose, such combination products employ the compounds of Formulae (I)-(VIII), or a pharmaceutically acceptable salt thereof within the dosage range described herein and the other pharmaceutically active agent or treatment within its dosage range.

### Combination Therapy

Another embodiment provides for methods of treatment using pharmaceutically acceptable compositions comprising a compound of the invention, either as the neat chemical or optionally further comprising additional ingredients. Such compositions are contemplated for preparation and use alone or in combination therapy. For preparing pharmaceutical compositions from the compounds of the invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18^{th} Edition, (1990), Mack Publishing Co., Easton, Pennsylvania.

Non-limiting examples of additional drugs and active agents useful in combination therapies for the treatment of malaria, include the following: Coartem® (Novartis International AG, Basel, Switzerland; artemether+lumefantrine), Eurartesim® (Sigma-Tau Pharmaceuticals, Inc., Rome, Italy; dihydroartemisinin-piperaquine), Pyramax® (Shin Poong Pharmaceutical Co., Ltd., Seoul, Korea; pyronaridine-artesunate), ASAQ Winthrop® (Sanofi SA (Gentilly, France)/DNDi (Geneva, Switzerland); artesunate+amodiaquine), ASMQ (Cipla Limited (Mumbai, India)/DNDi, artesunate+mefloquine), SPAQ-CO™ (Guilin Pharmaceutical Co., Ltd. (Shanghai), amodiaquine+sulfadoxine, pyrimethamine), Artesun® (Guilin Pharmaceutical, artesunate), artemether, artesunate, dihydroartemisinin, lumefantrine, amodiaquine, mefloquine, piperaquine, quinine, chloroquine, atovaquone and proguanil and sulfadoxine-pyrimethamine, Tafenoquine (Glaxosmithkline), OZ439/PQP (Sanofi), OZ439/FQ (Sanofi), KAE609 (Novartis), KAF156 (Novartis), DSM265 (NIH/Takeda), and MK-4815 (Merck &Co., Inc., Powles et al., Antimicrobial Agents and Chemotherapy 56(5): 2414-2419(2012)). Selection of such additional active ingredients will be according to the diseases or disorders present for which treatment is desired, as determined by the attending physician or other health care provider.

Thus, the invention also provides methods of using the compounds of Formulae (I)-(VIII), or a pharmaceutically acceptable salt thereof to inhibit plasmepsin X, plasmepsin IX or plasmepsin X and IX, to treat Plasmodium infection or treat malaria wherein the method further comprises administering to a subject in need thereof, one or more additional anti-malarial agents. In some embodiments, the one or more additional anti-malarial agents are selected from the group consisting of: artemether, lumefantrine, dihydroartemisinin, piperaquine, pyronaridine, artesunate, amodiaquine, mefloquine, sulfadoxine, pyrimethamine, lumefantrine, quinine, chloroquine, atovaquone, and proguanil.

### EXAMPLES

The meanings of the abbreviations in Examples are shown below.
ACN=MeCN=CH₃CN=acetonitrile
AcOH=acetic acid
AIBN=Azobisisobutyronitrile
Ar=argon
BBr₃=Boron tribromide
BF₃Et₂O=Boron trifluoride etherate
Boc₂O=di-tert-butyl dicarbonate
Cbz=carboxybenzyl
CbzOSu=N-(Benzyloxycarbonyloxy)succinimide
CBr₄=Tetrabromomethane
CCl₄=carbontetrachloride
CELITE=diatomaceous earthConc.=concentrated
Cs₂CO₃=Cesium carbonate
DBU=1,8-Diazabicyclo[5.4.0]undec-7-ene
DCE=dichloroethane
DCM=dichloromethane
DDQ=2,3-Dichloro-5,6-dicyano-1,4-benzoquinone
DIBALH=diisobutylaluminum hydride
DIPEA=DIEA=N, N-Diisopropylethylamine, or Hünig's base
DMA=dimethylacetamide
DMAP=4-dimethylaminopyridine
DMF=N,N-Dimethylformamide
DMP=Dess-Martin periodinane
DMSO=dimethyl sulfoxide
DPPE=1,2-Bis(diphenylphosphino)ethane
DPPF=1,1′-Bis(diphenylphosphino)ferrocene
EDCI=EDC=1-ethyl-3-(3-dimethylaminopropyl)carbodiimide
Et₂O=diethyl ether
EtOAc=ethyl acetate
EtOH=ethanol
Et₃SiH=Triethylsilane
h=hours
H₂=hydrogen
HATU=1-[Bis(dimethylamino)methylene]-1H-1,2,3-triazolo[4,5-b]pyridinium 3-oxid hexafluorophosphate
HCl=hydrochloric acid
HFBA=Heptafluorobutyric acid
HOAc=acetic acid
I₂=iodine
IPA=isopropyl alcohol
[Ir(cod)Cl]₂=cyclooctadiene iridium chloride dimer
K₂CO₃=potassium carbonate
K₃PO₄=Tripotassium phosphate
KF=Potassium fluoride
KHMDS=Potassium bis(trimethylsilyl)amide
KOTMS=Potassium trimethylsilanolate
LCMS=Liquid chromatography-mass spectrometry
LDA=Lithium diisopropylamide
LHMDS=LiHMDS=lithium bis(trimethylsilyl)amide
LiAlH₄=lithium aluminum hydride
LiOH=lithium hydroxide
min=minutes
Me=methyl
MeCN=Acetonitrile
MeOH═CH₃OH=methanol
MgSO₄=Magnesium sulfate
MsCl=methanesulfonyl chloride
N₂=nitrogen
NaBH₄=sodium borohydrate
NaH=sodium hydride
NaHCO₃=Sodium hydrogencarbonate
NaIO₄=sodium periodate
NaOH=sodium hydroxide
Na₂CO₃=sodium carbonate
Na₂SO₃=sodium sulfite
Na₂SO₄=sodium sulfate
NH₄Cl=Ammonium chloride
NH₄OH=Ammonium hydroxide
NH₄OAc=Ammonium acetate
NaHMDS=sodium bis(trimethylsilyl)amide
OMs=mesylate
OTs=tosylate
OTf=trifluoromethanesulfonyl
Pd(OH)₂/C=Pearlman's Catalyst—Palladium hydroxide on carbon-C
Pd—C=Palladium on carbon-C
[Pd(C₃H₅)Cl₂]=Allylpalladium(II) chloride dimer
PdCl₂(dppf)-CH₂Cl₂=[1,1′-Bis(diphenylphosphino)ferrocene]dichloropalladium(II)
PPh₃=Triphenylphosphine
RuO₂·H₂O=ruthenium(IV)oxide hydrate
RP-HPLC=reverse phase high performance liquid chromatography
SFC=Supercritical Fluid Chromatography
TBDPS-Cl=TBSCl=tert-Butyl(chloro)diphenylsilane
TEA=Et₃N=triethylamine
TBAF=Tetra-n-butylammonium fluoride
TFA=trifluoroacetic acid
THF=tetrahydrofuran
Ti(EtO)₄=titanium ethoxide
TMS=Trimethylsilylb
TMSOTf=Trimethylsilyl trifluoromethanesulfonate
TsOH=p-Toluenesulfonic acid
CDCl₃=heavy chloroform
CD₃OD=heavy methanol
1 Standard atmosphere [atm]=101325 pascal [Pa]=14.6959488 psi

The meanings of the abbreviations in the nuclear magnetic resonance spectra are shown below:
s=singlet, d=doublet, dd=double doublet, dt=double triplet, ddd=double doublet, Sept=septet, t=triplet, m=multiplet, br=broad, brs=broad singlet, q=quartet J=coupling constant and Hz=hertz.

Several methods for preparing the compounds of this disclosure are described in the following Schemes and Examples. Starting materials and intermediates were purchased commercially from common catalog sources or were made using known procedures, or as otherwise illustrated. Some frequently applied routes to the compounds of Formula I are described in the Schemes that follow. In some cases, the order of carrying out the reaction steps in the schemes may be varied to facilitate the reaction or to avoid unwanted reaction products.

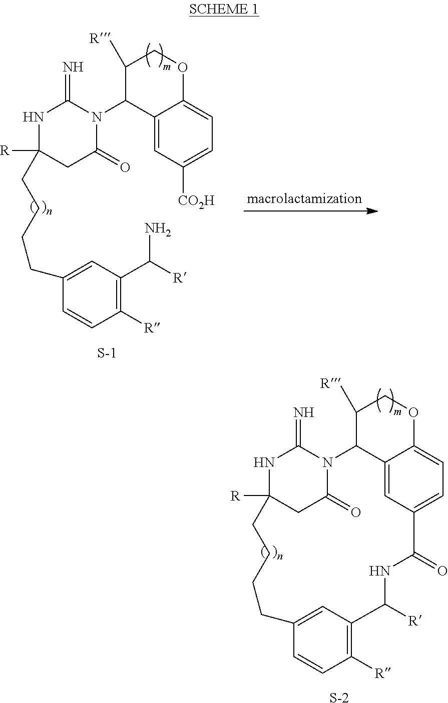

Compounds of Formula S-2 are prepared from S-1 by macrolactamization using amide coupling reagents.

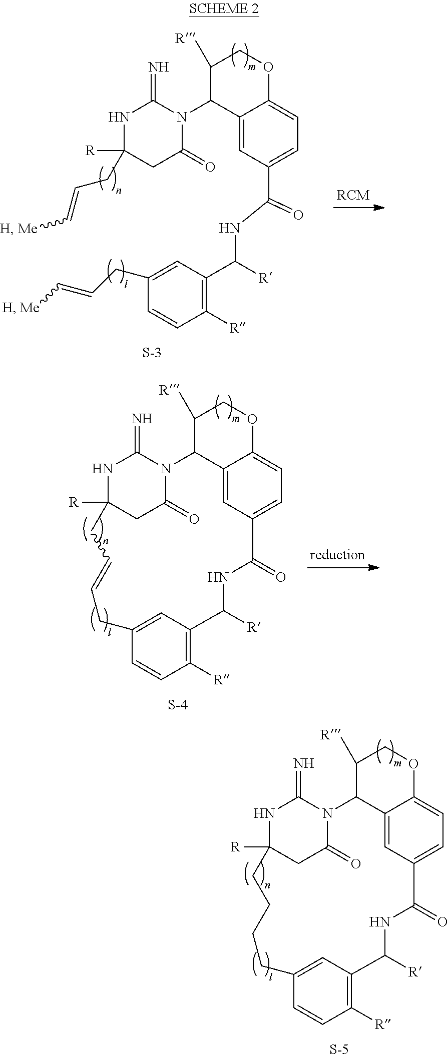

Intermediate compounds of Formula S-4 are prepared from S-3 after ring closing metathesis (RCM) reactions using catalysts such as the 2^{nd} generation Grubbs', Zhan's and Hoveyda/Grubbs' catalysts. Double bonds in S-4 can be reduced under for example hydrogenation conditions to yield the products of Formula S-5.

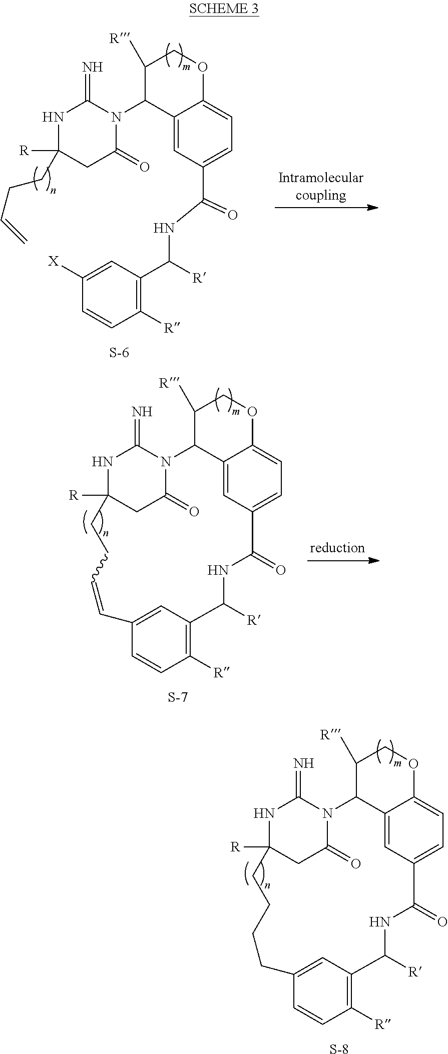

Intermediate compounds of Formula S-7 are prepared from S-6, in which X is a halogen such as Cl, Br and I, after transition metal catalyzed intramolecular cross coupling reactions such as Heck reactions. Resulting double bonds in S-7 can be reduced under for example hydrogenation conditions to yield the products of Formula S-8.

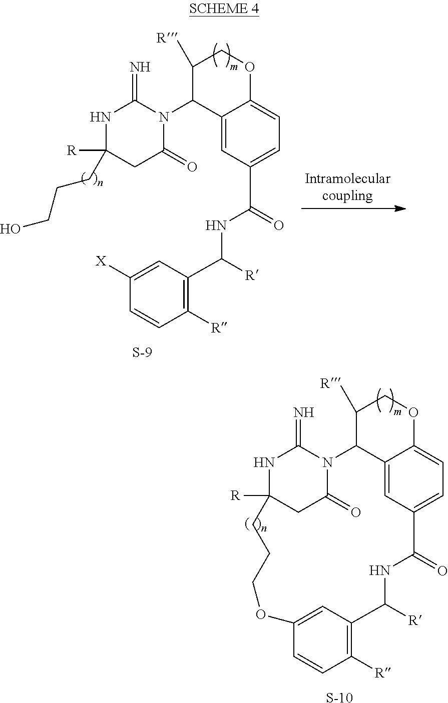

Products of Formula S-10 are prepared from S-9, in which X is a halogen such as Cl, Br and I, after transition metal catalyzed intramolecular cross coupling reactions such as palladium catalyzed C—O coupling reactions.

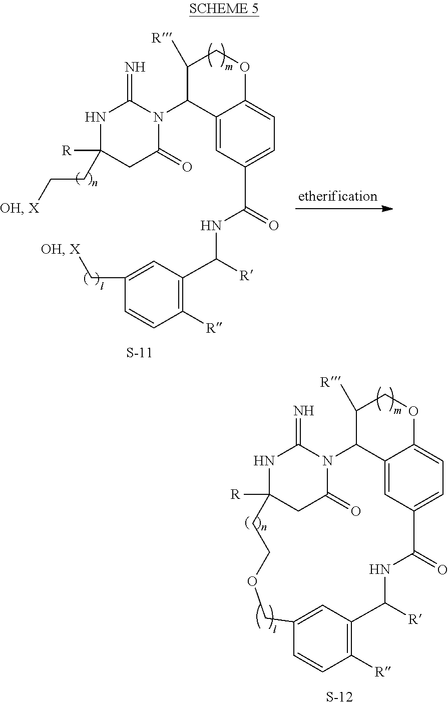

Products of Formula S-12 are prepared from S-11 after intramolecular S_{N}2 reactions between an alcohol and X, in which X is a leaving group such as Cl, Br, I, OMs, OTs or OTf Products of Formula S-12 are also prepared from S-11 diols after dehydration conditions using an acid or other dehydration reagents.

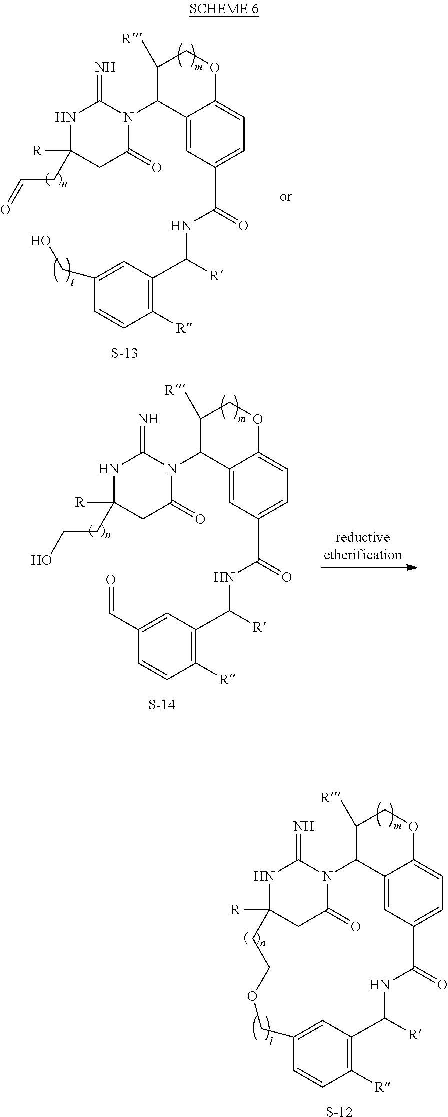

Products of Formula S-12 are prepared from intermediates S-13 or S-14 after intramolecular reductive etherification using conditions such as TMSOTf and Et₃SiH.

Reactions sensitive to moisture or air were performed inside a glove-box or under nitrogen or argon using anhydrous solvents and reagents. The progress of reactions was determined by either analytical thin layer chromatography (TLC) usually performed with E. Merck pre-coated TLC plates (Merck KGaA, Darmstadt, Germany), silica gel 60F-254, layer thickness 0.25 mm or liquid chromatography-mass spectrometry (LC/MS).

Typically, the analytical LC-MS system used consisted of a Waters ZQ™ platform with electrospray ionization in positive ion detection mode with an Agilent 1100 series HPLC with autosampler. The column was commonly a Waters Xterra MS C18, 3.0×50 mm, 5 μm or a Waters Acquity UPLC® BEH C18 1.0×50 mm, 1.7 μm. The flow rate was 1 mL/min, and the injection volume was 10 μL. UV detection was in the range 210-400 nm. The mobile phase consisted of solvent A (water plus 0.05% TFA) and solvent B (MeCN plus 0.05% TFA) with a gradient of 100% solvent A for 0.7 min changing to 100% solvent B over 3.75 min, maintained for 1.1 min, then reverting to 100% solvent A over 0.2 min.

Preparative HPLC purifications were usually performed using either a mass spectrometry directed system or a non-mass guided system. Usually they were performed on a Waters Chromatography Workstation configured with LC-MS System consisting of: Waters ZQ™ single quad MS system with Electrospray Ionization, Waters 2525 Gradient Pump, Waters 2767 Injecto/Collector, Waters 996 PDA Detector, the MS conditions of: 150-750 amu, Positive Electrospray, Collection Triggered by MS, and a Waters SUNFIRE® C-18 5-micron, 30 mm (id)×100 mm column. The mobile phases consisted of mixtures of acetonitrile (10-100%) in water containing 0.10% TFA. Flow rates were maintained at 50 mL/min, the injection volume was 1800 μL, and the UV detection range was 210-400 nm. An alternate preparative HPLC system used was a Gilson Workstation consisting of: Gilson GX-281 Injector/Collector, Gilson UV/VIS-155 Detector, Gilson 333 and 334 Pumps, and either a Phenomenex Gemini-NX C-18 5-micron, 50 mm (id)×250 mm column or a Waters XBridge™ C-18 5-micron OBD™, 30 mm (id)×250 mm column. The mobile phases consisted of mixtures of acetonitrile (0-75%) in water containing 5 mmol (NH₄)HCO₃. Flow rates were maintained at 50 mL/min for the Waters Xbridge™ column and 90 mL/min for the Phenomenex Gemini column. The injection volume ranged from 1000-8000 μL, and the UV detection range was 210-400 nm. Mobile phase gradients were optimized for the individual compounds. Reactions performed using microwave irradiation were normally carried out using an Emrys Optimizer manufactured by Personal Chemistry, or an Initiator manufactured by Biotage concentration of solutions was carried out on a rotary evaporator under reduced pressure. Flash chromatography was usually performed using either a Biotage® Flash Chromatography apparatus (DyaxCorp.), an ISCOCombiFlash® Rf apparatus, or an ISCOCombiFlash® companion XL on silica gel (32-63 μM, 60 Å pore size) in pre-packed cartridges of the size noted. ¹H NMR spectra were acquired at 500 MHz spectrometers in CDCl₃ solutions unless otherwise noted. Chemical shifts were reported in parts per million (ppm). Tetramethylsilane (TMS) was used as internal reference in CDCl₃ solutions, and residual CH₃OH peak or TMS was used as internal reference in CD₃OD solutions. Coupling constants (J) were reported in hertz (Hz). Chiral analytical chromatography was most commonly performed on one of CHIRALPAK® AS, CHIRALPAK®AD, CHIRALCEL® OD, CHIRALCEL®IA, or CHIRALCEL®OJ columns (250×4.6 mm) (Daicel Chemical Industries, Ltd.) with noted percentage of either ethanol in hexane (% Et/Hex) or isopropanol in heptane (% IPA/Hep) as isocratic solvent systems. Chiral preparative chromatography was conducted on one of CHIRALPAK AS, of CHIRALPAK AD, CHIRALCEL®OD, CHIRALCEL® IA, CHIRALCEL® OJ columns (20×250 mm) (Daicel Chemical Industries, Ltd.) with desired isocratic solvent systems identified on chiral analytical chromatography or by supercritical fluid (SFC) conditions.

It is understood that a chiral center in a compound may exist in the “S” or “R” stereo-configuration, or as a mixture of both. Within a molecule, each bond drawn as a straight line from a chiral center includes both the (R) and (S) stereoisomers as well as mixtures thereof.

### Intermediate 1

### Preparation of Intermediate 1-2

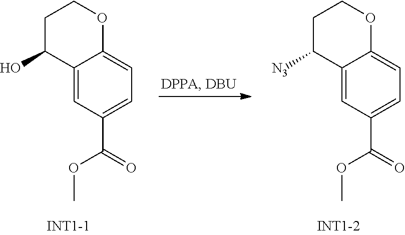 [SMILES: COC(=O)c1ccc2c(c1)[C@@H](O)CCO2 ; COC(=O)c1ccc2c(c1)[C@H](N=[N+]=[N-])CCO2]

DBU (21.72 mL, 144 mmol) and diphenylphosphinyl was added to a mixture of methyl (S)-4-hydroxychromane-6-carboxylate (INT1-1) (10 g, 48.0 mmol) in THF (80 mL). Azide (35.0 g, 144 mmol) was then added under N₂. The mixture was stirred at 50° C. for 12 h. The mixture was quenched with water (100 mL), and extracted with EtOAc (100 mL*3). The organic layers were washed with brine (80 mL), dried over Na₂SO₄, filtered and concentrated. The crude product was purified by flash silica gel chromatography (ISCO®; 330 g SepaFlash® Silica Flash column, Eluent of 15% EtOAc/Pet. ether gradient at 50 mL/min) to give methyl (R)-4-azidochromane-6-carboxylate (INT1-2).

MS (ESI) m/z 234.0 (M+H⁺)

¹H NMR (500 MHz, CHLOROFORM-d) δ 7.93-7.96 (m, 2H), 6.91 (d, J=8.5 Hz, 1H), 4.65 (t, J=3.5 Hz, 1H), 4.29-4.35 (m, 2H), 3.90 (s, 3H), 2.14-2.26 (m, 1H), 2.06-2.12 (m, 1H)

### Preparation of Intermediate 1-3

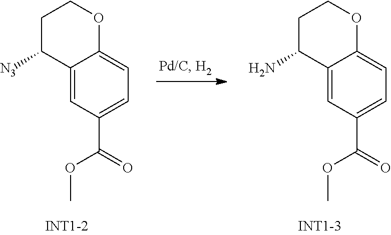 [SMILES: COC(=O)c1ccc2c(c1)[C@H](N=[N+]=[N-])CCO2 ; COC(=O)c1ccc2c(c1)[C@H](N)CCO2]

Pd—C (2.510 g, 4.72 mmol) was added to a solution of methyl (R)-4-azidochromane-6-carboxylate (INT1-2) (11 g, 47.2 mmol) in THF (200 mL) under N₂ atmosphere. The mixture was degassed and backfilled with H₂ (three times). The resulting mixture was stirred at 25° C. for 12 h under H₂ (15 psi) atmosphere. The catalyst was filtered off and the filtrate was concentrated under reduced pressure to give methyl (R)-4-aminochromane-6-carboxylate (INT1-3).

MS (ESI) m/z: 191.1 (M-17+H⁺)

### Preparation of Intermediate 1

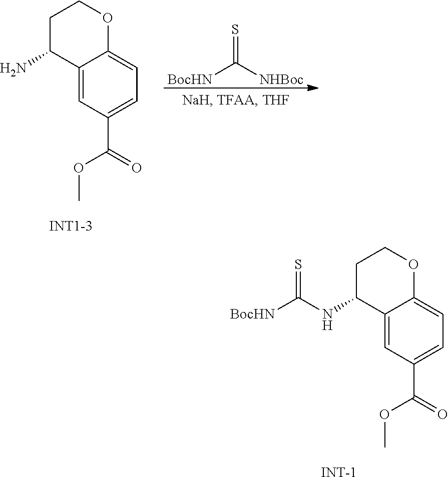 [SMILES: COC(=O)c1ccc2c(c1)[C@H](N)CCO2 ; CC(C)(C)OC(=O)NC(=S)NC(=O)OC(C)(C)C ; COC(=O)c1ccc2c(c1)[C@H](NC(=S)NC(=O)OC(C)(C)C)CCO2 ; "INT-1"]

Sodium hydride (3.77 g, 94 mmol) was added to a solution of N,N-bisboc-thiourea (16.94 g, 61.3 mmol) in THF (250 mL) at 0° C. in portions under N₂. After 1 hr at this temperature, 2,2,2-trifluoroacetic anhydride (8.82 mL, 61.3 mmol) was added dropwise. The mixture was stirred at 0° C. for 1 h. A solution of methyl (R)-4-aminochromane-6-carboxylate (INT1-3) (9.77 g, 47.1 mmol) in THF (50 mL) was added dropwise at 0° C. The mixture was stirred at 0° C. for 2 h. The mixture was quenched with water (80 mL), and extracted with EtOAc (50 mL*3). The organic layers were washed with brine (40 mL), dried over Na₂SO₄, filtered, and concentrated in vacuo. The crude was purified by flash silica gel chromatography (ISCO®; 220 g SepaFlash© Silica Flash column, Eluent of 20% EtOAc/Pet. ether gradient at 50 mL/min) to afford methyl (R)-4-(3-(tert-butoxycarbonyl)thioureido)chromane-6-carboxylate (INT-1).

MS (ESI) m/z 367.1 (M+H⁺)

¹H NMR (500 MHz, CHLOROFORM-d) δ 8.01 (d, J=1.5 Hz, 1H), 7.96 (s, 1H), 7.89 (dd, J=2.0, 9.0 Hz, 1H), 6.89 (d, J=9.0 Hz, 1H), 4.34-4.41 (m, 1H), 4.19-4.26 (m, 1H), 3.88 (s, 3H), 2.25-2.40 (m, 2H), 1.47 (s, 9H)

### Example 1

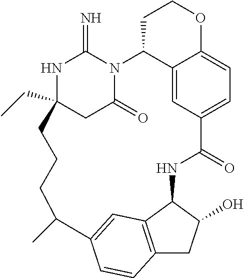 [SMILES: CC[C@]12CCCC(C)c3ccc4c(c3)[C@@H](NC(=O)c3ccc5c(c3)[C@@H](CCO5)N(C(=N)N1)C(=O)C2)[C@H](O)C4]

### (1R,5R,15R,16R)-5-ethyl-15-hydroxy-3-imino-9-methyl-23-oxa-2,4,17-triazahexacyclo[17.6.2.22,5.210,13.012,16.022,26]hentriaconta-10,12,19,21,26,28-hexaene-18,31-dione

### Preparation of Compound 1-2

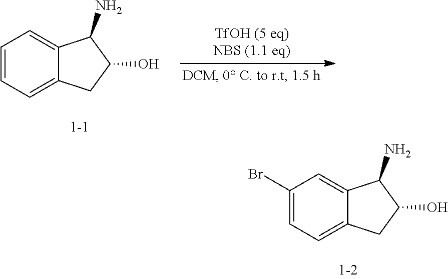 [SMILES: N[C@@H]1c2ccccc2C[C@H]1O ; N[C@@H]1c2cc(Br)ccc2C[C@H]1O]

Trifluoromethanesulfonic acid (503 mg, 3.35 mmol) and N-bromosuccinimide (596 mg, 3.35 mmol) was added to a solution of (1R,2R)-1-amino-2,3-dihydro-1H-inden-2-ol (1-1) (500 mg, 3.35 mmol) in DCM (10 mL) at 0° C. The reaction was stirred at 18° C. for 1 h under N₂ atmosphere. The mixture was quenched with saturated sodium bicarbonate solution (10 mL) at 0° C., and extracted with DCM (10 mL*1) then extracted with EtOAc (10 mL*3). The organic layers were washed with brine (10 mL), dried over Na₂SO₄, filtered and concentrated in vacuum, the crude was purified by purified by flash silica gel chromatography (ISCO®; 12 g SepaFlash® Silica Flash column, Eluent of 10% MeOH/DCM (added 1% ammonium hydroxide) gradient at 50 mL/min) with to afford (1R,2R)-1-amino-6-bromo-2,3-dihydro-1H-inden-2-ol (1-2).

MS (ESI) m/z: 228.1, 230.1 (M+H⁺)

¹H NMR (400 MHz, METHANOL-d₄) δ 7.50 (s, 1H), 7.33 (dd, J=1.6, 8.0 Hz, 1H), 7.10 (d, J=8.0 Hz, 1H), 4.10 (q, J=6.8 Hz, 1H), 4.01-4.05 (m, 1H), 3.15 (dd, J=6.8, 15.6 Hz, 1H), 2.68 (dd, J=7.2, 15.6 Hz, 1H)

### Preparation of Compound 1-3

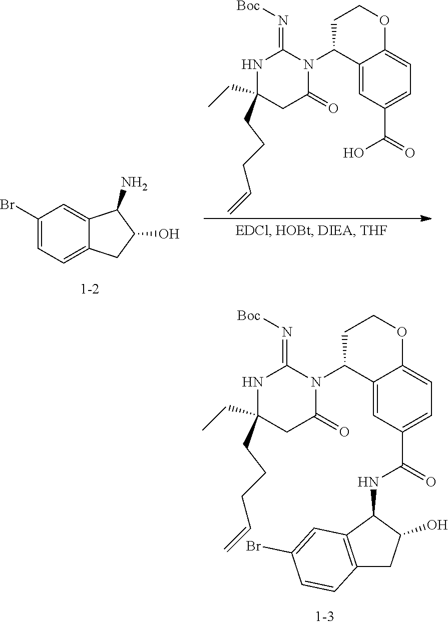 [SMILES: N[C@@H]1c2cc(Br)ccc2C[C@H]1O ; C=CCCC[C@]1(CC)CC(=O)N([C@@H]2CCOc3ccc(C(=O)O)cc32)/C(=N/C(=O)OC(C)(C)C)N1 ; C=CCCC[C@]1(CC)CC(=O)N([C@@H]2CCOc3ccc(C(=O)N[C@@H]4c5cc(Br)ccc5C[C@H]4O)cc32)/C(=N/C(=O)OC(C)(C)C)N1]

DIEA (0.276 mL, 1.582 mmol) was added to a solution of (R)-4-((R,E)-2-((tert-butoxycarbonyl)imino)-4-ethyl-6-oxo-4-(pent-4-en-1-yl)tetrahydropyrimidin-1(2H)-yl)chromane-6-carboxylic acid (1-2) (96 mg, 0.198 mmol), EDC (189 mg, 0.989 mmol), 1H-benzo[d][1,2,3]triazol-1-ol (134 mg, 0.989 mmol) and (1R,2R)-1-amino-6-bromo-2,3-dihydro-1H-inden-2-ol (45.1 mg, 0.198 mmol) in THF (5 mL). The reaction was stirred at 18° C. for 2 h. The mixture was quenched with water (5 mL), and extracted with EtOAc (5 mL*3). The organic layers were washed with brine (5 mL), dried over Na₂SO₄, filtered and concentrated in vacuum, and the crude was purified by prep-TLC (Pet. ether/EtOAc=1:1) to afford tert-butyl ((R,E)-1-((R)-6-(((1R,2R)-6-bromo-2-hydroxy-2,3-dihydro-1H-inden-1-yl)carbamoyl)chroman-4-yl)-4-ethyl-6-oxo-4-(pent-4-en-1-yl)tetrahydropyrimidin-2(1H)-ylidene)carbamate (1-3).

MS (ESI) m/z: 695.1, 697.1 (M+H⁺)

### Preparation of Compound 1-4A, 1-4B, and 1-4C

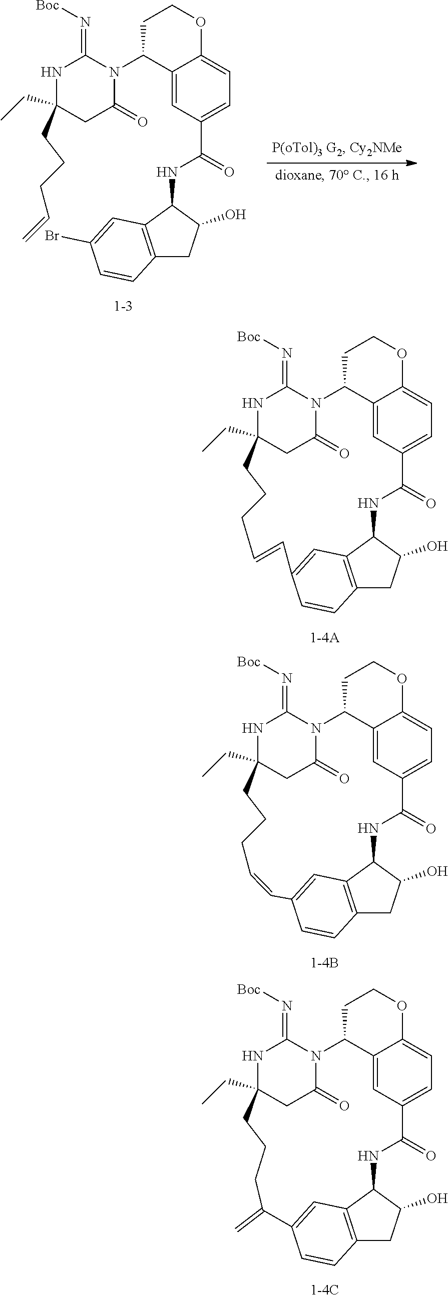 [SMILES: C=CCCC[C@]1(CC)CC(=O)N([C@@H]2CCOc3ccc(C(=O)N[C@@H]4c5cc(Br)ccc5C[C@H]4O)cc32)/C(=N/C(=O)OC(C)(C)C)N1 ; CC[C@@]12CCC/C=C/c3ccc4c(c3)[C@@H](NC(=O)c3ccc5c(c3)[C@@H](CCO5)N(C(=O)C1)/C(=N/C(=O)OC(C)(C)C)N2)[C@H](O)C4 ; CC[C@@]12CCC/C=C\c3ccc4c(c3)[C@@H](NC(=O)c3ccc5c(c3)[C@@H](CCO5)N(C(=O)C1)/C(=N/C(=O)OC(C)(C)C)N2)[C@H](O)C4 ; C=C1CCC[C@]2(CC)CC(=O)N(/C(=N/C(=O)OC(C)(C)C)N2)[C@@H]2CCOc3ccc(cc32)C(=O)N[C@@H]2c3cc1ccc3C[C@H]2O]

Chloro[tri(o-tolyl)phosphine][2-(2′-amino-1,1′-biphenyl)]palladium(II) (8.83 mg, 0.014 mmol) and N,N-dicyclohexylmethylamine (140 mg, 0.719 mmol) was added to a solution of tert-butyl ((R,E)-1-((R)-6-(((1R,2R)-6-bromo-2-hydroxy-2,3-dihydro-1H-inden-1-yl)carbamoyl)chroman-4-yl)-4-ethyl-6-oxo-4-(pent-4-en-1-yl)tetrahydropyrimidin-2(1H)-ylidene)carbamate (1-3) (100 mg, 0.144 mmol) in dioxane (1.5 mL) at 25° C. in a glove box. The reaction was stirred at 70° C. for 16 h. The mixture was quenched with water (3 mL), and extracted with EtOAc (5 mL*3). The organic layers were washed with brine (5 mL), dried over Na₂SO₄, filtered and concentrated in vacuum, and the crude was purified by reverse preparative HPLC (Instrument ED; Method column Boston Prime C18 150*30 mm*5 um; condition water (0.04% NH₃H₂O+10 mM NH₄HCO₃)-ACN Begin B 65; End B 95 Gradient Time (min) 10; 100% B Hold Time (min) 2 Flow Rate (mL/min) 25; Injections 5) to afford: tert-butyl ((4aR,8R,12E,18R,18aR,28E)-8-ethyl-18-hydroxy-6,20-dioxo-4,4a,7,8,9,10,11,17,18,18a,19,20-dodecahydro-3H,6H-1,21-(epiethane[1,2]diylidene)-8,5-(epiminomethano)-14,16-ethenocyclopenta[h]pyrano[4,3-b][1,7]diazacyclononadecin-28-ylidene)carbamate (1-4A); tert-butyl ((4aR,8R,12Z,18R,18aR,28E)-8-ethyl-18-hydroxy-6,20-dioxo-4,4a,7,8,9,10,11,17,18,18a,19,20-dodecahydro-3H,6H-1,21-(epiethane[1,2]diylidene)-8,5-(epiminomethano)-14,16-ethenocyclopenta[h]pyrano[4,3-b][1,7]diazacyclononadecin-28-ylidene)carbamate (1-4B); and tert-butyl ((4aR,8R,17R,17aR,E)-8-ethyl-17-hydroxy-12-methylene-6,19-dioxo-4,4a,6,7,8,9,10,11,12,16,17,17a,18,19-tetradecahydro-3H-1,20-(epiethane[1,2]diylidene)-8,5-(epiminomethano)-13,15-ethenocyclopenta[h]pyrano[4,3-b][1,7]diazacyclooctadecin-27-ylidene)carbamate (1-4C).

MS (ESI) m/z: 486.2 (M+H⁺)

1-4A: ¹H NMR (500 MHz, CHLOROFORM-d) δ 10.09 (s, 1H), 7.93 (dd, J=1.5, 8.50 Hz, 1H), 7.21-7.26 (m, 2H), 7.14 (d, J=8.0 Hz, 1H), 6.91-6.98 (m, 2H), 6.42-6.54 (m, 3H), 5.82-5.92 (m, 1H), 5.25 (t, J=5.5 Hz, 1H), 4.94 (s, 1H), 4.40-4.50 (m, 2H), 4.25-4.35 (m, 1H), 3.34 (dd, J=8.0, 15.5 Hz, 1H), 2.99-3.05 (m, 1H), 2.67-2.86 (m, 2H), 2.57 (d, J=13.5 Hz, 1H), 2.40 (d, J=15.5 Hz, 1H), 2.07 (s, 2H), 1.82 (d, J=11.0 Hz, 2H), 1.62-1.67 (m, 4H), 1.49 (s, 9H), 0.96 (t, J=7.5 Hz, 3H)

1-4B: ¹H NMR (500 MHz, CHLOROFORM-d) δ 10.13 (s, 1H), 7.90 (dd, J=2.0, 8.50 Hz, 1H), 7.37 (s, 1H), 7.25 (d, J=7.5 Hz, 1H), 7.17 (s, 1H), 7.12 (d, J=7.5 Hz, 1H), 6.96 (d, J=8.50 Hz, 1H), 6.41-6.48 (m, 2H), 6.26 (dd, J=7.0, 9.0 Hz, 1H), 5.60-5.70 (m, 1H), 5.34 (t, J=6.5 Hz, 1H), 4.60 (s, 1H), 4.46-4.54 (m, 1H), 4.42 (td, J=4.0, 11.0 Hz, 1H), 4.21 (dt, J=2.5, 11.0 Hz, 1H), 3.34 (dd, J=7.5, 16.0 Hz, 1H), 2.97 (dd, J=7.5, 16.0 Hz, 1H), 2.60-2.72 (m, 2H), 2.49-2.53 (m, 2H), 2.22-2.30 (m, 1H), 2.13-2.18 (m, 1H), 2.03-2.12 (m, 2H), 1.72-1.82 (m, 4H), 1.49 (s, 9H), 1.00 (t, J=7.5 Hz, 3H)

1-4C: ¹H NMR (500 MHz, CHLOROFORM-d) δ 10.04 (s, 1H), 7.81 (dd, J=2.0, 8.5 Hz, 1H), 7.52 (d, J=8.0 Hz, 1H), 7.24 (d, J=8.0 Hz, 1H), 6.99-7.16 (m, 2H), 6.91 (d, J=8.50 Hz, 1H), 6.38-6.50 (m, 2H), 5.44 (s, 1H), 5.31 (t, J=6.5 Hz, 1H), 5.13 (s, 1H), 4.41-4.57 (m, 2H), 4.36 (s, 1H), 4.23 (dt, J=2.0, 11.50 Hz, 1H), 3.34 (dd, J=8.0, 16.0 Hz, 1H), 2.98 (dd, J=8.0, 16.0 Hz, 1H), 2.69-2.79 (m, 1H), 2.59-2.63 (m, 1H), 2.45-2.52 (m, 2H), 1.93-2.09 (m, 2H), 1.86 (d, J=12.0 Hz, 2H), 1.53-1.65 (m, 4H), 1.45 (s, 9H), 0.98 (t, J=7.5 Hz, 3H)

### Preparation of Compound 1-5

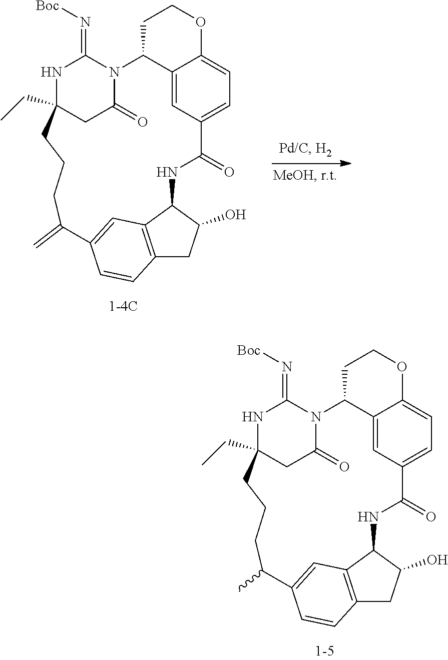 [SMILES: C=C1CCC[C@]2(CC)CC(=O)N(/C(=N/C(=O)OC(C)(C)C)N2)[C@@H]2CCOc3ccc(cc32)C(=O)N[C@@H]2c3cc1ccc3C[C@H]2O ; CC[C@@]12CCCC(C)c3ccc4c(c3)[C@@H](NC(=O)c3ccc5c(c3)[C@@H](CCO5)N(C(=O)C1)/C(=N/C(=O)OC(C)(C)C)N2)[C@H](O)C4]

Pd—C (1.731 mg, 3.25 μmol) was added to a solution of tert-butyl ((4aR,8R,17R,17aR,E)-8-ethyl-17-hydroxy-12-methylene-6,19-dioxo-4,4a,6,7,8,9,10,11,12,16,17,17a,18,19-tetradecahydro-3H-1,20-(epiethane[1,2]diylidene)-8,5-(epiminomethano)-13,15-ethenocyclopenta[h]pyrano[4,3-b][1,7]diazacyclooctadecin-27-ylidene)carbamate (1-4C) (10 mg, 0.016 mmol) in MeOH (2 ml) under N₂ atmosphere. The mixture was degassed and backfilled with H₂ (three times). The resulting mixture was stirred under H₂ (15 psi) at 25° C. for 12 h. The catalyst was filtered off and the filtrate was concentrated under reduced pressure to give tert-butyl ((4aR,8R,17R,17aR,E)-8-ethyl-17-hydroxy-12-methyl-6,19-dioxo-4,4a,6,7,8,9,10,11,12,16,17,17a,18,19-tetradecahydro-3H-1,20-(epiethane[1,2]diylidene)-8,5-(epiminomethano)-13,15-ethenocyclopenta[h]pyrano[4,3-b][1,7]diazacyclooctadecin-27-ylidene)carbamate (1-5).

MS (ESI) m/z: 617.3 (M+H⁺)

### Preparation of Example 1

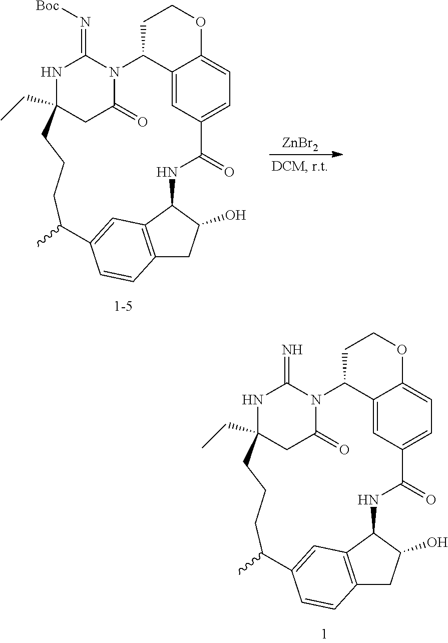

Zinc(II) bromide (29.2 mg, 0.130 mmol) was added to a solution of tert-butyl ((4aR,8R,17R,17aR,E)-8-ethyl-17-hydroxy-12-methyl-6,19-dioxo-4,4a,6,7,8,9,10,11,12,16,17,17a,18,19-tetradecahydro-3H-1,20-(epiethane[1,2]diylidene)-8,5-(epiminomethano)-13,15-ethenocyclopenta[h]pyrano[4,3-b][1,7]diazacyclooctadecin-27-ylidene)carbamate (1-5) (8 mg, 0.013 mmol) in DCM (3 ml), at 22° C. under N₂ atmosphere. The mixture was stirred at 22° C. for 16 h. The mixture was cooled, the solvent was evaporated under reduced pressure to give the crude product. The residue was purified by reverse preparative HPLC (Instrument EJ Method column Boston Green ODS 150*30 mm*5 um condition water (TFA)-ACN Begin B 22 End B 52 Gradient Time (min) 10 100% B Hold Time (min) 2 Flow Rate (ml/min) 25 Injections 1) to yield Example 1.

MS (ESI) m/z: 517.2 (M+H⁺)

¹H NMR (500 MHz, METHANOL-d₄) δ 8.49 (d, J=9.0 Hz, 1H), 7.72 (dd, J=2.0, 8.5 Hz, 1H), 7.39 (d, J=1.0 Hz, 1H), 7.19-7.24 (m, 1H), 7.14-7.19 (m, 1H), 6.91-6.96 (m, 2H), 5.30-5.39 (m, 2H), 4.38-4.53 (m, 2H), 4.13-4.15 (m, 1H), 3.18-3.29 (m, 1H), 2.75-2.94 (m, 3H), 2.61-2.72 (m, 2H), 2.20-2.30 (m, 1H), 1.74-1.85 (m, 3H), 1.58-1.68 (m, 2H), 1.50-1.57 (m, 1H), 1.31 (d, J=7.0 Hz, 3H), 1.24-1.29 (m, 1H), 1.11-1.20 (m, 1H), 0.97 (t, J=7.5 Hz, 3H)

### Example 2

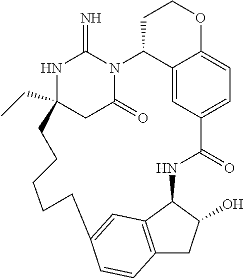 [SMILES: CC[C@]12CCCCCc3ccc4c(c3)[C@@H](NC(=O)c3ccc5c(c3)[C@@H](CCO5)N(C(=N)N1)C(=O)C2)[C@H](O)C4]

### (1R,5R,16R,17R)-5-ethyl-16-hydroxy-3-imino-24-oxa-2,4,18-triazahexacyclo[18.6.2.22,5.211,14.013,17.023,27]dotriaconta-11,13,20,22,27,29-hexaene-19,32-dione

### Preparation of Compound 2-1

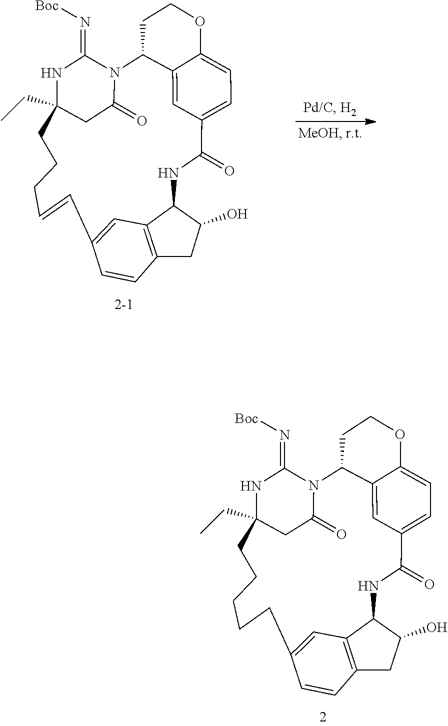 [SMILES: CC[C@@]12CCC/C=C/c3ccc4c(c3)[C@@H](NC(=O)c3ccc5c(c3)[C@@H](CCO5)N(C(=O)C1)/C(=N/C(=O)OC(C)(C)C)N2)[C@H](O)C4 ; CC[C@@]12CCCCCc3ccc4c(c3)[C@@H](NC(=O)c3ccc5c(c3)[C@@H](CCO5)N(C(=O)C1)/C(=N/C(=O)OC(C)(C)C)N2)[C@H](O)C4]

Pd—C (2.250 mg, 4.23 μmol) was added to a solution of tert-butyl ((4aR,8R,12E,18R,18aR,28E)-8-ethyl-18-hydroxy-6,20-dioxo-4,4a,7,8,9,10,11,17,18,18a,19,20-dodecahydro-3H,6H-1,21-(epiethane[1,2]diylidene)-8,5-(epiminomethano)-14,16-ethenocyclopenta[h]pyrano[4,3-b][1,7]diazacyclononadecin-28-ylidene)carbamate (2-1) (13 mg, 0.021 mmol) in MeOH (4 mL) under N₂ atmosphere. The mixture was degassed and backfilled with H₂ (three times). The resulting mixture was stirred under H₂ (15 psi) at 20° C. for 2 h. The catalyst was filtered off and the filtrate was concentrated under reduced pressure to give tert-butyl ((4aR,8R,18R,18aR,E)-8-ethyl-18-hydroxy-6,20-dioxo-4,4a,7,8,9,10,11,12,13,17,18,18a,19,20-tetradecahydro-3H,6H-1,21-(epiethane[1,2]diylidene)-8,5-(epiminomethano)-14,16-ethenocyclopenta[h]pyrano[4,3-b][1,7]diazacyclononadecin-28-ylidene)carbamate (2-1).

MS (ESI) m/z: 617.2 (M+H⁺)

### Preparation of Example 2

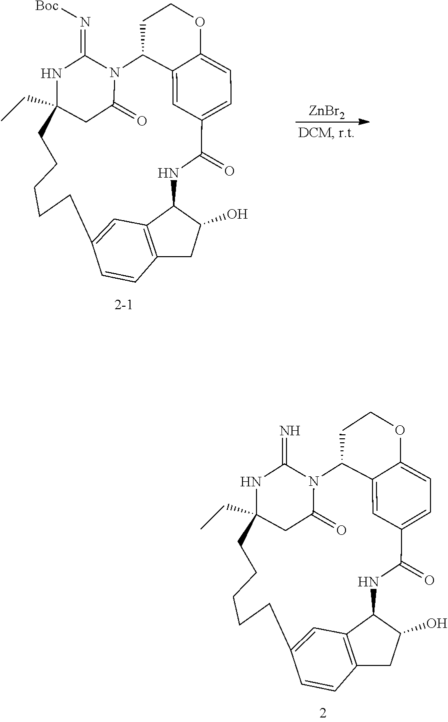 [SMILES: CC[C@@]12CCCCCc3ccc4c(c3)[C@@H](NC(=O)c3ccc5c(c3)[C@@H](CCO5)N(C(=O)C1)/C(=N/C(=O)OC(C)(C)C)N2)[C@H](O)C4 ; "ZnBr2" ; CC[C@]12CCCCCc3ccc4c(c3)[C@@H](NC(=O)c3ccc5c(c3)[C@@H](CCO5)N(C(=N)N1)C(=O)C2)[C@H](O)C4]

Zinc(II) bromide (47.5 mg, 0.211 mmol) was added to a solution of tert-butyl ((4aR,8R,18R,18aR,E)-8-ethyl-18-hydroxy-6,20-dioxo-4,4a,7,8,9,10,11,12,13,17,18,18a,19,20-tetradecahydro-3H,6H-1,21-(epiethane[1,2]diylidene)-8,5-(epiminomethano)-14,16-ethenocyclopenta[h]pyrano[4,3-b][1,7]diazacyclononadecin-28-ylidene)carbamate (2-1) (13 mg, 0.021 mmol) in DCM (3 mL), under N₂ atmosphere. The mixture was stirred at 22° C. for 16 h. The mixture was concentrated under reduced pressure to give the crude product. The residue was purified by reverse preparative HPLC (Instrument EJ; Method column Boston Green ODS 150*30 mm*5 um; Condition water (TFA)-ACN Begin B 22; End B 52 Gradient Time (min) 10; 100% B Hold Time (min) 2 Flow Rate (mL/min) 25; Injections 1) to give (4aR,8R,18R,18aR)-8-ethyl-18-hydroxy-28-imino-4,4a,8,9,10,11,12,13,17,18,18a,19-dodecahydro-3H,6H-1,21-(epiethane[1,2]diylidene)-8,5-(epiminomethano)-14,16-ethenocyclopenta[h]pyrano[4,3-b][1,7]diazacyclononadecine-6,20(7H)-dione Example 2.

MS (ESI) m/z: 517.2 (M+H⁺)

¹H NMR (500 MHz, METHANOL-d₄) δ 7.76 (dd, J=2.0, 8.5 Hz, 1H), 7.46 (d, J=1.5 Hz, 1H), 7.13 (d, J=8.0 Hz, 1H), 6.99-7.06 (m, 2H), 6.93 (d, J=8.5 Hz, 1H), 5.21 (t, J=7.5 Hz, 1H), 5.07 (d, J=5.5 Hz, 1H), 4.54-4.62 (m, 1H), 4.38-4.49 (m, 1H), 4.09-4.19 (m, 1H), 3.23-3.28 (m, 1H), 2.90 (d, J=16.5 Hz, 1H), 2.79 (dd, J=6.0, 16.0 Hz, 1H), 2.62-2.74 (m, 2H), 2.50-2.61 (m, 2H), 2.20-2.34 (m, 1H), 1.81 (s, 1H), 1.62-1.76 (m, 3H), 1.49 (t, J=11.5 Hz, 1H), 1.12-1.44 (m, 6H), 0.95 (t, J=7.5 Hz, 3H)

### Example 3A

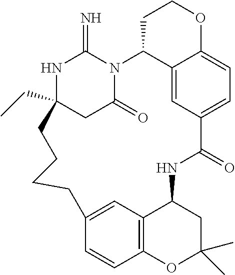 [SMILES: CC[C@]12CCCCc3ccc4c(c3)[C@H](CC(C)(C)O4)NC(=O)c3ccc4c(c3)[C@@H](CCO4)N(C(=N)N1)C(=O)C2]

### (1R,5R,17S)-5-ethyl-3-imino-15,15-dimethyl-14,24-dioxa-2,4,18-triazahexacyclo[18.6.2.22,5.210,13.012,17.023,27]dotriaconta-10,12,20,22,27,29-hexaene-19,32-dione

### Preparation of Compound 3-2

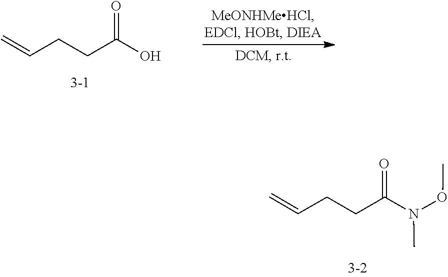

N,O-dimethylhydroxylamine hydrochloride (54.6 g, 559 mmol) was added to a solution of pent-4-enoic acid (40 g, 400 mmol), EDC (92 g, 479 mmol), 1H-benzo[d][1,2,3]triazol-1-ol (3-1) (64.8 g, 479 mmol) and N-ethyl-N-isopropylpropan-2-amine (279 mL, 1598 mmol) in DCM (400 mL). The reaction was stirred at 25° C. for 12 h under N₂ atmosphere. The mixture was quenched with water (300 mL), and extracted with DCM (100 mL*3). The organic layers were washed with brine (100 mL), dried over Na₂SO₄, filtered and concentrated in vacuo, and the crude was purified by flash silica gel chromatography (ISCO®; 220 g Agela Silica Flash column, eluent of 8% ethyl acetate/pet. ether gradient at 50 mL/min) to afford N-methoxy-N-methylpent-4-enamide (3-2).

MS (ESI) m/z 144.1 (M+H⁺)

¹H NMR (500 MHz, CHLOROFORM-d) δ 5.83-5.92 (m, 1H), 4.94-5.10 (m, 2H), 3.68 (s, 3H), 3.18 (s, 3H), 2.50-2.56 (m, 2H), 2.35-2.42 (m, 2H)

### Preparation of Compound 3-3

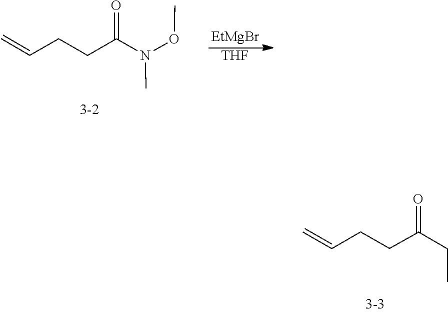

Ethylmagnesium bromide (69.8 mL, 210 mmol) was added dropwise to a solution of N-methoxy-N-methylpent-4-enamide (3-2) (20 g, 140 mmol) in THF (200 mL) under N₂ atmosphere at 0° C. The reaction was stirred at 25° C. for 1 h under N₂ atmosphere. The mixture was quenched with saturated NH₄Cl aqueous (100 mL) and water (100 mL), extracted with EtOAc (100 mL*3). The organic layers were washed with brine (50 mL), dried over Na₂SO₄, filtered and concentrated in vacuo, and the crude was purified by flash silica gel chromatography (ISCO®; 120 g Agela Silica Flash column, eluent of 5% ethyl acetate/pet. ether gradient at 40 mL/min) to afford hept-6-en-3-one (3-3).

¹H NMR (500 MHz, CHLOROFORM-d) δ 5.78-5.83 (m, 1H), 4.92-5.07 (m, 2H), 2.48-2.54 (m, 2H), 2.43 (q, J=7.0 Hz, 2H), 2.29-2.37 (m, 2H), 1.06 (t, J=7.0 Hz, 3H)

### Preparation of Compound 3-4

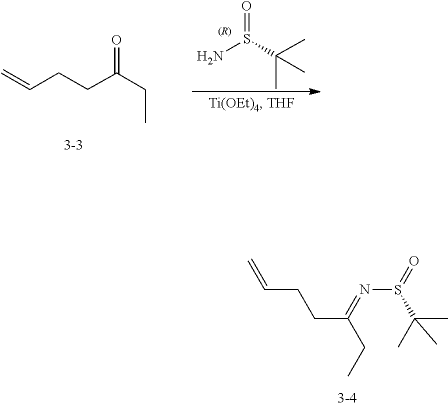

(R)-2-methylpropane-2-sulfinamide (12.97 g, 107 mmol) was added to a solution hept-6-en-3-one (3-3) (10 g, 89 mmol) in THF (100 mL). Ti(EtO)₄ (37.5 mL, 178 mmol) was also added and the reaction was stirred at 75° C. for 12 h under N₂ atmosphere. The mixture was cooled to room temperature, diluted with DCM (200 mL) and stirred for 15 min. Ice-cold-saturated aqueous sodium bicarbonate solution (50 mL) and Na₂SO₄ was added and the mixture was filtered and concentrated in vacuo. The crude was purified by flash silica gel chromatography (ISCO®; 120 g Agela Silica Flash column, eluent of 8% ethyl acetate/pet. ether gradient at 40 mL/min) to afford (R,E)-N-(hept-6-en-3-ylidene)-2-methylpropane-2-sulfinamide (3-4).

MS (ESI) m/z 216.2 (M+H⁺)

¹H NMR (500 MHz, CHLOROFORM-d) δ 5.73-5.88 (m, 1H), 4.94-5.10 (m, 2H), 2.64-2.87 (m, 2H), 2.31-2.58 (m, 4H), 1.22 (s, 9H), 1.05-1.20 (m, 3H)

### Preparation of Compound 3-5

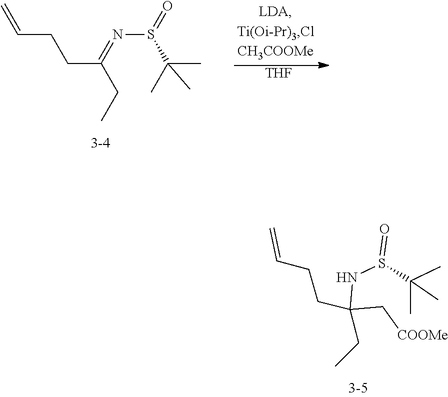 [SMILES: C=CCC/C(CC)=N/[S@](=O)C(C)(C)C ; C=CCCC(CC)(CC(=O)OC)N[S@](=O)C(C)(C)C ; "THF"]

Butyllithium (55.7 mL, 139 mmol), at −78° C., was added dropwise to a solution of diisopropylamine (19.64 mL, 139 mmol) in anhydrous THF (40 mL) under N₂ atmosphere. The reaction was stirred at 0° C. for 30 min to make LDA. The resulting LDA (76 mL, 93 mmol) was dropped to a mixture at −78° C. of ethyl acetate (7.48 mL, 93 mmol) and Ti(OiPr)₃Cl (116 mL, 116 mmol) in anhydrous THF (90 mL). After 1 h, a solution of (R,E)-N-(hept-6-en-3-ylidene)-2-methylpropane-2-sulfinamide (3-4) (10 g, 46.4 mmol) in anhydrous THF (20 mL) was then added dropwise and the mixture was stirred at −78° C. for 3 h. The mixture was quenched with ice-cold half-saturated aqueous ammonium chloride solution (60 mL). The slurry was diluted with EtOAc (200 mL) then filtered, rinsing with EtOAc and water. The organic layer was washed with brine (50 mL), dried over Na₂SO₄, filtered and concentrated in vacuo. The residue was purified by flash silica gel chromatography (ISCO®; 120 g Agela Silica Flash column, eluent of 25% ethyl acetate/pet. ether gradient at 40 mL/min) to afford methyl 3-(((R)-tert-butylsulfinyl)amino)-3-ethylhept-6-enoate (3-5).

MS (ESI) m/z 290.1 (M+H⁺)

¹H NMR (500 MHz, CHLOROFORM-d) δ 5.76-5.82 (m, 1H), 5.01-5.05 (m, 1H), 4.97 (dd, J=1.0, 10.0 Hz, 1H), 4.63 (br d, J=17.0 Hz, 1H), 3.68 (s, 3H), 2.72 (dd, J=5.0, 16.0 Hz, 1H), 2.52 (dd, J=2.5, 16.0 Hz, 1H), 2.07-2.13 (m, 1H), 1.82-1.91 (m, 1H), 1.76-1.81 (m, 1H), 1.71-1.75 (m, 1H), 1.26 (t, J=7.0 Hz, 2H), 1.24 (s, 9H), 0.87-0.95 (m, 3H)

### Preparation of Compound 3-6

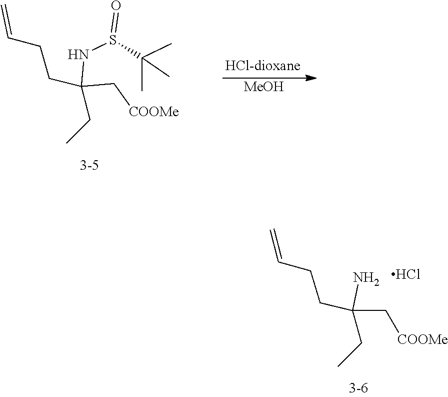 [SMILES: "MeOH" ; C=CCCC(CC)(CC(=O)OC)N[S@](=O)C(C)(C)C ; C=CCCC(N)(CC)CC(=O)OC ; Cl]

A solution of methyl 3-(((R)-tert-butylsulfinyl)amino)-3-ethylhept-6-enoate (3-5) (9 g, 31.1 mmol) in 4N HCl-dioxane (20 mL) and MeOH (20.00 mL) at 25° C. under N₂ atmosphere was stirred for 1 h. Solvent was evaporated under reduced pressure to give the crude product methyl 3-amino-3-ethylhept-6-enoate hydrochloride (3-6) used crude for the next step directly.

MS (ESI) m/z 186.2 (M+H⁺)

¹H NMR (500 MHz, CHLOROFORM-d) δ 5.75-5.80 (m, 1H), 5.08 (dd, J=1.0, 18.0 Hz, 1H), 4.98 (d, J=10.22 Hz, 1H), 3.72-3.75 (m, 3H), 2.78-2.85 (m, 2H), 2.21-2.29 (m, 2H), 1.84-2.00 (m, 4H), 1.06 (t, J=7.5 Hz, 3H)

### Preparation of Compound 3-7

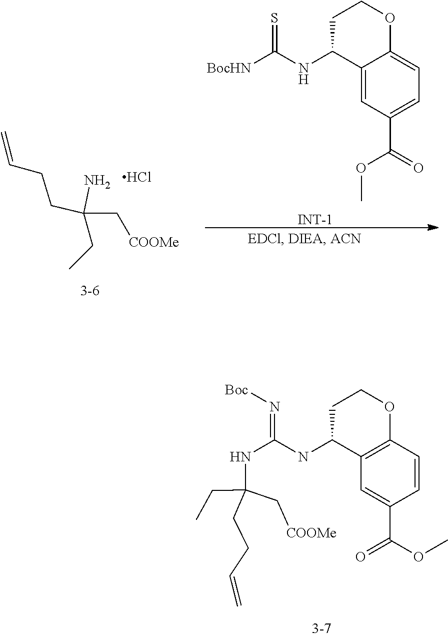

N-ethyl-N-isopropylpropan-2-amine (13.79 mL, 79 mmol) was added to a solution of EDC (9.08 g, 47.4 mmol), methyl 3-amino-3-ethylhept-6-enoate hydrochloride (3-6) (3.5 g, 15.79 mmol) and methyl (R)-4-(3-(tert-butoxycarbonyl)thioureido)chromane-6-carboxylate (INT-1) (5.78 g, 15.79 mmol) in ACN (30 mL). The reaction was stirred at 25° C. for 12 h under N₂ atmosphere. The mixture was quenched with water (50 mL), and extracted with EtOAc (50 mL*3). The organic layers were washed with brine (30 mL), dried over Na₂SO₄, filtered and concentrated in vacuo to afford methyl (4R)-4-((Z)-2-(tert-butoxycarbonyl)-3-(3-ethyl-1-methoxy-1-oxohept-6-en-3-yl)guanidino)chromane-6-carboxylate (3-7) used crude for the next step directly.

MS (ESI) m/z 518.2 (M+H⁺)

### Preparation of Compound 3-8

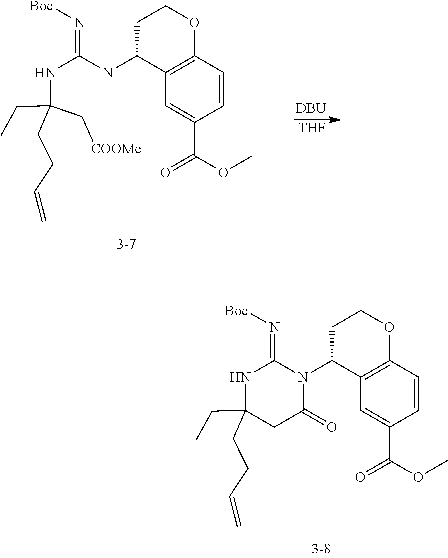

DBU (11.90 mL, 79 mmol) was added to a solution of methyl (4R)-4-((Z)-2-(tert-butoxycarbonyl)-3-(3-ethyl-1-methoxy-1-oxohept-6-en-3-yl)guanidino)chromane-6-carboxylate (3-7) (8.17 g, 15.78 mmol) in THF (50 mL). The mixture was stirred at 50° C. for 16 h under N₂ atmosphere. The mixture was quenched with water (50 mL), and extracted with EtOAc (50 mL*3). The organic layers were washed with brine (30 mL), dried over Na₂SO₄, filtered and concentrated in vacuo. The crude was purified by flash silica gel chromatography (ISCO®; 120 g Agela Silica Flash column, eluent of 25% ethyl acetate/pet. ether gradient at 40 mL/min) to afford methyl (4R)-4-((E)-4-(but-3-en-1-yl)-2-((tert-butoxycarbonyl)imino)-4-ethyl-6-oxotetrahydropyrimidin-1(2H)-yl)chromane-6-carboxylate (3-8).

MS (ESI) m/z 486.2 (M+H⁺)

¹H NMR (500 MHz, CHLOROFORM-d) δ 7.77 (td, J=2.0, 8.5 Hz, 1H), 7.62 (s, 1H), 6.84 (dd, J=2.0, 8.5 Hz, 1H), 6.33-6.42 (m, 1H), 5.76-5.90 (m, 1H), 5.00-5.14 (m, 2H), 4.42-4.49 (m, 1H), 4.22-4.23 (m, 1H), 3.81-3.86 (m, 3H), 2.70-2.81 (m, 1H), 2.51-2.60 (m, 2H), 2.03-2.16 (m, 4H), 1.75-1.84 (m, 2H), 1.65-1.73 (m, 4H), 1.52 (s, 9H), 0.98-1.04 (m, 3H)

### Preparation of Compounds 3-9A & 3-9B

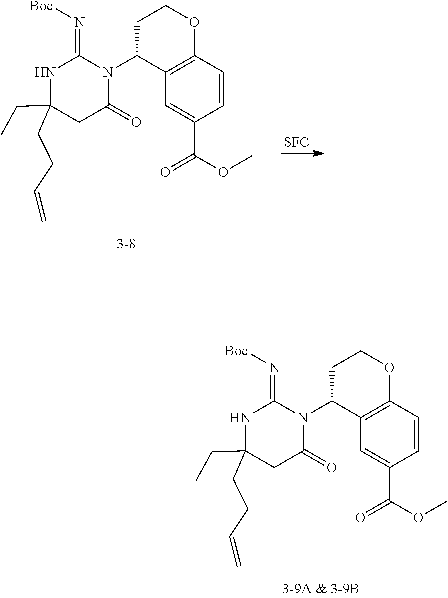

Methyl (4R)-4-((E)-4-(but-3-en-1-yl)-2-((tert-butoxycarbonyl)imino)-4-ethyl-6-oxotetrahydropyrimidin-1(2H)-yl)chromane-6-carboxylate (3-8) (4 g, 8.24 mmol) was separated by SFC (Column Boston Green ODS 150*30 mm*5 um condition water (TFA)-ACN Begin B 48 End B 78 Gradient Time (min) 10 100% B Hold Time (min) 2 Flow Rate (mL/min) 25 Injections 1) to give
P1 methyl (4R)-4-((E)-4-(but-3-en-1-yl)-2-((tert-butoxycarbonyl)imino)-4-ethyl-6-oxotetrahydropyrimidin-1(2H)-yl)chromane-6-carboxylate (3-9A) (t_{R}=2.105 min, UV=220 nm).
P2 methyl (4R)-4-((E)-4-(but-3-en-1-yl)-2-((tert-butoxycarbonyl)imino)-4-ethyl-6-oxotetrahydropyrimidin-1(2H)-yl)chromane-6-carboxylate (3-9B) (t_{R}=2.251 min, UV=220 nm).

MS (ESI) m/z 486.2 (M+H⁺)

3-9A: ¹H NMR (500 MHz, CHLOROFORM-d) δ 7.77 (dd, J=1.5, 8.5 Hz, 1H), 7.62 (s, 1H), 6.84 (d, J=9.0 Hz, 1H), 6.37 (br dd, J=7.0, 10.0 Hz, 1H), 5.81-5.86 (m, 1H), 5.00-5.12 (m, 2H), 4.42-4.45 (m, 2H), 4.21-4.24 (m, 1H), 3.83 (s, 3H), 2.68-2.78 (m, 1H), 2.07-2.16 (m, 3H), 1.66-1.83 (m, 5H), 1.51 (s, 9H), 0.96 (t, J=7.5 Hz, 3H)

3-9B: ¹H NMR (500 MHz, CHLOROFORM-d) δ 7.77 (br d, J=8.70 Hz, 1H), 7.62 (s, 1H), 6.84 (d, J=8.5 Hz, 1H), 6.39 (br dd, J=7.0, 10.0 Hz, 1H), 5.74-5.86 (m, 1H), 5.00-5.10 (m, 2H), 4.41-4.50 (m, 1H), 4.21-4.25 (m, 1H), 3.83 (s, 3H), 2.73-2.81 (m, 1H), 2.06-2.12 (m, 3H), 1.68-1.83 (m, 5H), 1.52 (s, 9H), 0.98-1.04 (m, 3H)

### Preparation of Compound 3-10

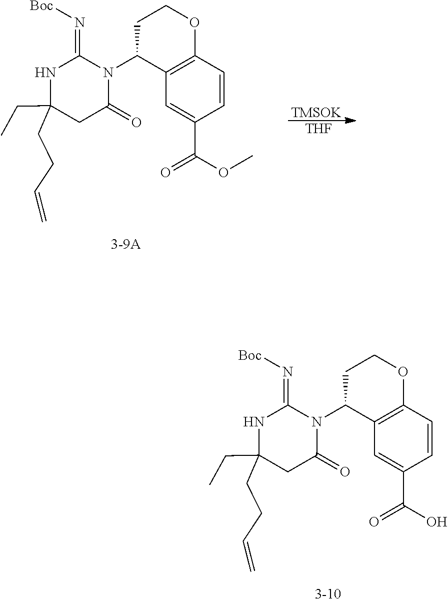

Potassium trimethylsilanolate (872 mg, 6.80 mmol) was added to a solution of methyl (R)-4-((R,E)-4-(but-3-en-1-yl)-2-((tert-butoxycarbonyl)imino)-4-ethyl-6-oxotetrahydropyrimidin-1(2H)-yl)chromane-6-carboxylate (3-9A) (550 mg, 1.133 mmol) in THF (5 mL). The reaction was stirred at 25° C. for 0.5 h. The solution of (R)-4-((R,E)-4-(but-3-en-1-yl)-2-((tert-butoxycarbonyl)imino)-4-ethyl-6-oxotetrahydropyrimidin-1(2H)-yl)chromane-6-carboxylic acid (3-10) was used for next step directly without any further manipulation or purification.

MS (ESI) m/z 472.2 (M+H⁺).

### Preparation of Compound 3-11

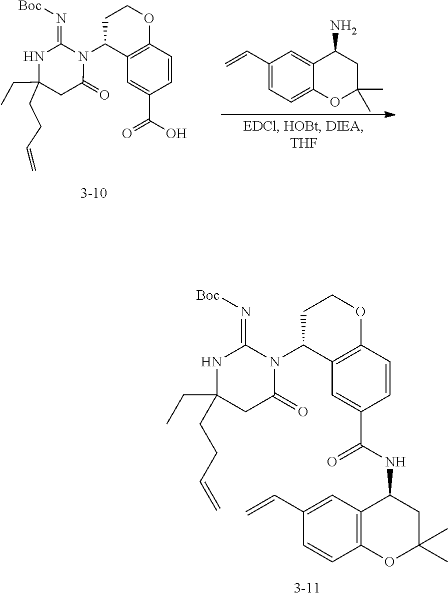

DIEA (1.582 mL, 9.06 mmol) was added to a solution of (R)-4-((R,E)-4-(but-3-en-1-yl)-2-((tert-butoxycarbonyl)imino)-4-ethyl-6-oxotetrahydropyrimidin-1(2H)-yl)chromane-6-carboxylic acid (3-10) (534 mg, 1.132 mmol), EDC (1085 mg, 5.66 mmol), 1H-benzo[d][1,2,3]triazol-1-ol (765 mg, 5.66 mmol) and (S)-2,2-dimethyl-6-vinylchroman-4-amine (253 mg, 1.246 mmol) in THF (50 mL). The reaction was stirred at 25° C. for 12 h. The mixture was quenched with water (20 mL), and extracted with EtOAc (20 mL*3). The organic layers were washed with brine (15 mL), dried over Na₂SO₄, filtered and concentrated in vacuo, and the crude was purified by prep-TLC (Pet. ether/EtOAc=2:1) to afford tert-butyl ((R,E)-4-(but-3-en-1-yl)-1-((R)-6-(((S)-2,2-dimethyl-6-vinylchroman-4-yl)carbamoyl)chroman-4-yl)-4-ethyl-6-oxotetrahydropyrimidin-2(1H)-ylidene)carbamate (3-11).

MS (ESI) m/z 657.3 (M+H⁺)

### Preparation of Compound 3-12

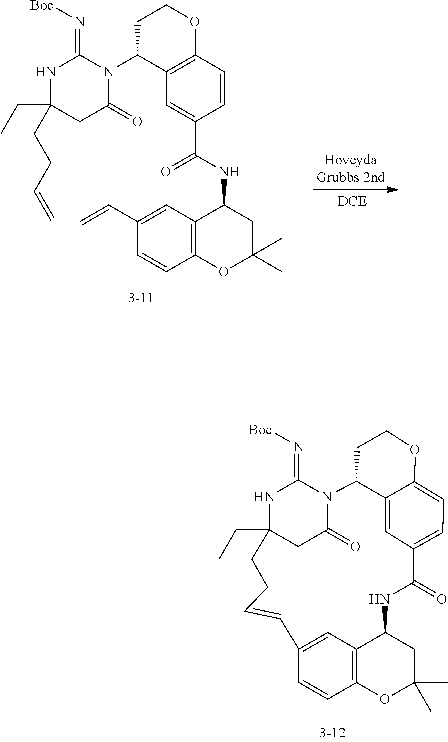

(1,3-Bis-(2,4,6-trimethylphenyl)-2-imidazolidinylidene)dichloro(o-isopropoxyphenylmethylene)ruthenium (95 mg, 0.152 mmol) was added to a solution of tert-butyl ((R,E)-4-(but-3-en-1-yl)-1-((R)-6-(((S)-2,2-dimethyl-6-vinylchroman-4-yl)carbamoyl)chroman-4-yl)-4-ethyl-6-oxotetrahydropyrimidin-2(1H)-ylidene)carbamate (3-11) (500 mg, 0.761 mmol) in DCE (500 mL). The reaction was stirred at 50° C. for 5 h under N₂ atmosphere. The mixture was concentrated in vacuo, and the crude was purified by flash column (Pet. ether/EtOAc/EtOH=8:3:1) to afford tert-butyl ((4aR,8R,11E,18aS,28E)-8-ethyl-17,17-dimethyl-6,20-dioxo-4,4a,7,8,9,10,18,18a,19,20-decahydro-3H,6H,17H-8,5-(epiminomethano)-1,21:13,15-diethenodipyrano[4,3-b:4′,3′-h][1,7]diazacyclooctadecin-28-ylidene)carbamate (3-12).

MS (ESI) m/z 629.3 (M+H⁺)

¹H NMR (500 MHz, CHLOROFORM-d) δ 7.67 (dd, J=1.5, 8.5 Hz, 1H), 7.36 (s, 1H), 7.19 (s, 1H), 7.05-7.10 (m, 1H), 6.91 (d, J=8.5 Hz, 1H), 6.69 (d, J=8.5 Hz, 1H), 6.37 (d, J=15.5 Hz, 1H), 6.09-6.22 (m, 2H), 5.92-5.93 (m, 1H), 5.40-5.42 (m, 1H), 4.49 (td, J=4.0, 11.5 Hz, 1H), 2.93-3.04 (m, 2H), 2.46-2.53 (m, 2H), 2.35-2.41 (m, 1H), 2.29 (dd, J=6.5, 13.0 Hz, 1H), 1.91-1.99 (m, 1H), 1.71-1.78 (m, 2H), 1.66-1.71 (m, 4H), 1.61-1.66 (m, 1H), 1.50-1.53 (m, 1H), 1.46-1.50 (m, 1H), 1.43 (s, 3H), 1.37 (s, 3H), 1.26 (s, 9H), 0.95 (t, J=7.48 Hz, 3H)

### Preparation of Compound 3-13

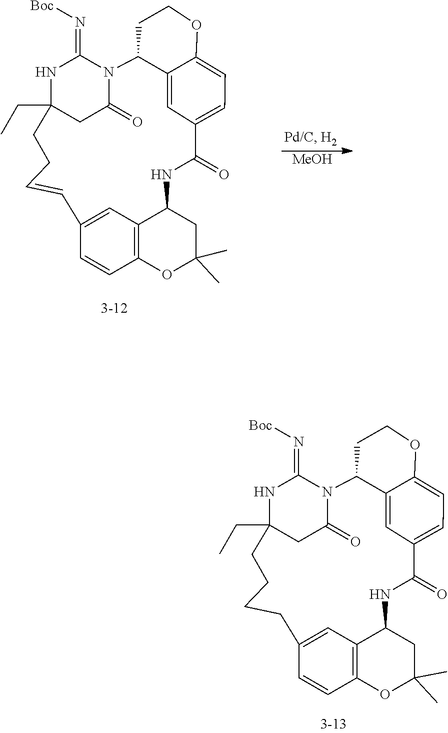 [SMILES: "MeOH" ; CCC12CC/C=C/c3ccc4c(c3)[C@H](CC(C)(C)O4)NC(=O)c3ccc4c(c3)[C@@H](CCO4)N(C(=O)C1)/C(=N/C(=O)OC(C)(C)C)N2 ; CCC12CCCCc3ccc4c(c3)[C@H](CC(C)(C)O4)NC(=O)c3ccc4c(c3)[C@@H](CCO4)N(C(=O)C1)/C(=N/C(=O)OC(C)(C)C)N2]

Pd—C (50.8 mg, 0.048 mmol) was added to a solution of tert-butyl ((4aR,8R,11E,18aS,28E)-8-ethyl-17,17-dimethyl-6,20-dioxo-4,4a,7,8,9,10,18,18a,19,20-decahydro-3H,6H,17H-8,5-(epiminomethano)-1,21:13,15-diethenodipyrano[4,3-b:4′,3′-h][1,7]diazacyclooctadecin-28-ylidene)carbamate (3-12) (300 mg, 0.477 mmol) in MeOH (10 mL), under N₂ atmosphere. The mixture was degassed and backfilled with H₂ (three times). The resulting mixture was stirred at 25° C. for 0.5 h under H₂ (15 psi). The catalyst was filtered off and filtrate was concentrated under reduced pressure to give tert-butyl ((4aR,8R,18aS,E)-8-ethyl-17,17-dimethyl-6,20-dioxo-4,4a,7,8,9,10,11,12,18,18a,19,20-dodecahydro-3H,6H,17H-8,5-(epiminomethano)-1,21:13,15-diethenodipyrano[4,3-b:4′,3′-h][1,7]diazacyclooctadecin-28-ylidene)carbamate (3-13) which was used for the next step directly.

MS (ESI) m/z 631.3 (M+H⁺)

### Preparation of Example 3A

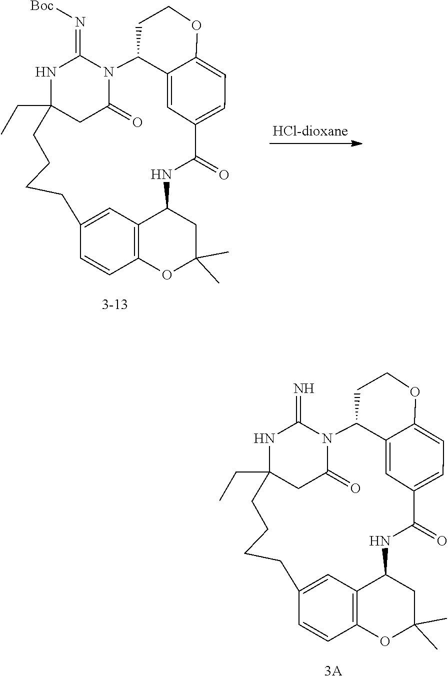 [SMILES: CCC12CCCCc3ccc4c(c3)[C@H](CC(C)(C)O4)NC(=O)c3ccc4c(c3)[C@@H](CCO4)N(C(=O)C1)/C(=N/C(=O)OC(C)(C)C)N2 ; CCC12CCCCc3ccc4c(c3)[C@H](CC(C)(C)O4)NC(=O)c3ccc4c(c3)[C@@H](CCO4)N(C(=N)N1)C(=O)C2]

A solution of tert-butyl ((4aR,8R,18aS,E)-8-ethyl-17,17-dimethyl-6,20-dioxo-4,4a,7,8,9,10,11,12,18,18a,19,20-dodecahydro-3H,6H,17H-8,5-(epiminomethano)-1,21:13,15-diethenodipyrano[4,3-b:4′,3′-h][1,7]diazacyclooctadecin-28-ylidene)carbamate (3-13) (280 mg, 0.444 mmol) in HCl-dioxane (4N) (30 mL) was stirred at 25° C. for 16 h. The solvent was evaporated under reduced pressure to give the crude product. The residue was purified by reverse preparative HPLC (Column Boston Green ODS 150*30 mm*5 um condition water (HCl)-ACN Begin B 30 End B 50 Gradient Time (min) 10 100% B Hold Time (min) 2 Flow Rate (mL/min) 25 Injections 6) to give (4aR,8R,18aS)-8-ethyl-28-imino-17,17-dimethyl-4,4a,7,8,9,10,11,12,17,18,18a,19-dodecahydro-3H,6H,20H-8,5-(epiminomethano)-1,21:13,15-diethenodipyrano[4,3-b:4′,3′-h][1,7]diazacyclooctadecine-6,20-dione Example 3A.

MS (ESI) m/z 531.2 (M+H⁺)

¹H NMR (500 MHz, METHANOL-d₄) δ 7.73 (dd, J=2.0, 8.5 Hz, 1H), 7.45 (s, 1H), 7.02 (s, 1H), 6.92-6.99 (m, 2H), 6.70 (d, J=8.5 Hz, 1H), 5.37-5.44 (m, 2H), 4.44-4.47 (m, 1H), 4.12-4.17 (m, 1H), 2.93 (d, J=16.5 Hz, 1H), 2.55-2.70 (m, 4H), 2.23-2.31 (m, 1H), 2.12 (dd, J=6.5, 13.0 Hz, 1H), 1.81-1.87 (m, 1H), 1.66-1.80 (m, 5H), 1.49-1.59 (m, 1H), 1.42 (s, 3H), 1.34-1.40 (m, 2H), 1.32 (s, 3H), 0.98 (t, J=7.5 Hz, 3H)

### Example 4

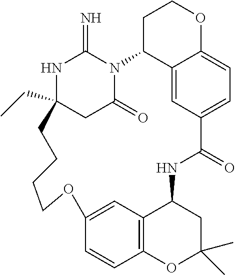 [SMILES: CC[C@]12CCCCOc3ccc4c(c3)[C@H](CC(C)(C)O4)NC(=O)c3ccc4c(c3)[C@@H](CCO4)N(C(=N)N1)C(=O)C2]

### (1R,5R,18S)-5-ethyl-3-imino-16,16-dimethyl-10,15,25-trioxa-2,4,19-triazahexacyclo[19.6.2.22,5.211,14.013,18.024,28]tritriaconta-11,13,21,23,28,30-hexaene-20,33-dione

### Preparation of Compound 4-2

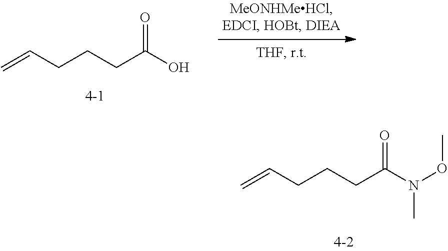

DIEA (36.7 mL, 210 mmol) was added to a solution of hex-5-enoic acid (4-1) (8 g, 70.1 mmol), EDC (20.15 g, 105 mmol), 1H-benzo[d][1,2,3]triazol-1-ol (14.21 g, 105 mmol) and N,O-dimethylhydroxylamine hydrochloride (7.52 g, 77 mmol) in THF (100 mL). The reaction was stirred at 25° C. for 5 h. The mixture was quenched with water (150 mL), and extracted with EtOAc (50 mL*3). The organic layers were washed with brine (50 mL), dried over Na₂SO₄, filtered and concentrated in vacuo. The crude product was purified by flash silica gel chromatography (ISCO®; 80 g SepaFlash® Silica Flash column, Eluent of 5% EtOAc/Pet. ether gradient at 80 mL/min) to give N-methoxy-N-methylhex-5-enamide (4-2).

MS (ESI) m/z 157.7 (M+H⁺)

### Preparation of Compound 4-3

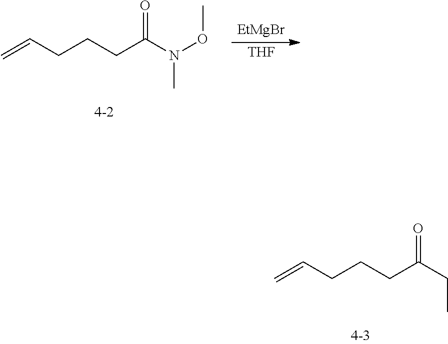

Ethylmagnesium bromide (38.2 mL, 114 mmol), at 0° C., was added dropwise to a solution of N-methoxy-N-methylhex-5-enamide (4-2) (9 g, 57.2 mmol) in THF (120 mL) under N₂ atmosphere. Then the mixture was stirred 20° C. for 2 h. The mixture was quenched with saturated aqueous NH₄Cl (25 mL). The mixture was quenched with water (150 ml) and extracted with EtOAc (100 mL*3). The combined organic layers were washed with brine (80 mL), dried over Na₂SO₄, filtered. Solvent was removed under reduced pressure to give crude product which was purified by column chromatography (SiO₂, Pet. ether:EtOAc=100:1˜10:1) to afford oct-7-en-3-one (4-3).

¹H NMR (500 MHz, CHLOROFORM-d) δ 5.76 (m, 1H), 4.93-5.04 (m, 2H), 2.38-2.44 (m, 4H), 2.05 (q, J=7.0 Hz, 2H), 1.68 (m, 2H), 1.04 (t, J=7.5 Hz, 3H).

### Preparation of Compound 4-4

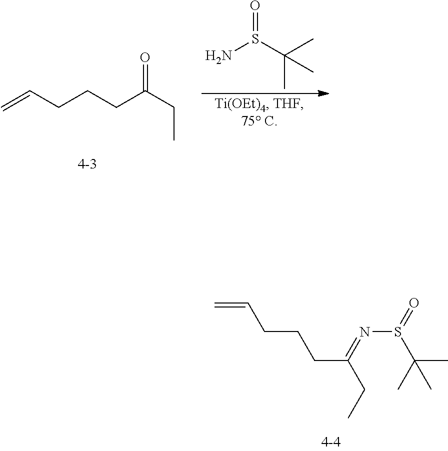

2-methylpropane-2-sulfinamide (6.34 g, 52.3 mmol) was added to a solution oct-7-en-3-one (4-3) (5.5 g, 43.6 mmol) in THF (80 mL), followed by Ti(EtO)₄ (18.31 ml, 87 mmol). The reaction was stirred at 75° C. for 16 h under N₂ atmosphere. The final mixture was cooled to 0° C., diluted with DCM (100 mL) and stirred for 15 min. Ice cold-saturated aqueous sodium bicarbonate solution (15 mL) was added and the solution was filtered and concentrated in vacuo. The crude was purified by flash column (SiO₂, Pet. ether/EtOAc=100:1 to 5:1) to afford ((E)-2-methyl-N-(oct-7-en-3-ylidene)propane-2-sulfinamide (4-4).

MS (ESI) m/z 230.2 (M+H⁺)

¹H NMR (500 MHz, CHLOROFORM-d) δ 5.78-5.80 (m, 1H), 4.96-5.07 (m, 2H), 2.62-2.76 (m, 2H), 2.39-2.50 (m, 2H), 2.04-2.17 (m, 2H), 1.67-1.76 (m, 2H), 1.23 (s, 9H), 1.06-1.21 (m, 3H)

### Preparation of Compound 4-5

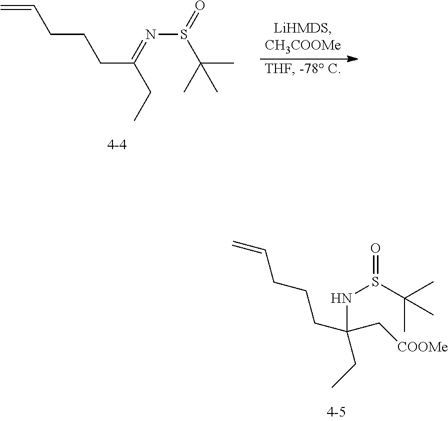

Methyl acetate (1.545 ml, 19.18 mmol) at −78° C. was added to a solution of LiHMDS (22.67 ml, 22.67 mmol) in anhydrous THF (35 mL) under N₂ atmosphere. The reaction was stirred at −78° C. for 15 min. (E)-2-methyl-N-(oct-7-en-3-ylidene)propane-2-sulfinamide (4-4) (4 g, 17.44 mmol) in THF (15 mL) was then added dropwise and the mixture was stirred at −78° C. for 3 h. The mixture was quenched with ice-cold half-saturated aqueous ammonium chloride solution (30 mL). The slurry was diluted with EtOAc (50 mL) then filtered, rinsing with EtOAc and water. The organic layer was washed with brine (50 mL), dried over Na₂SO₄, filtered and concentrated in vacuo. The residue was purified by flash column (SiO₂, Pet. ether/EtOAc=100:0 to 10:1) to afford methyl 3-((tert-butylsulfinyl)amino)-3-ethyloct-7-enoate (4-5).

MS (ESI) m/z 304.3 (M+H⁺).

¹H NMR (500 MHz, CHLOROFORM-d) δ 5.72-5.86 (m, 1H), 4.91-5.06 (m, 2H), 4.59-4.61 (m, 1H), 3.63-3.76 (m, 3H), 2.68-2.71 (m, 1H), 2.42-2.55 (m, 1H), 1.99-2.13 (m, 2H), 1.58-1.91 (m, 5H), 1.31-1.48 (m, 2H), 1.16-1.24 (m, 9H), 0.80-0.93 (m, 3H)

### Preparation of Compound 4-6

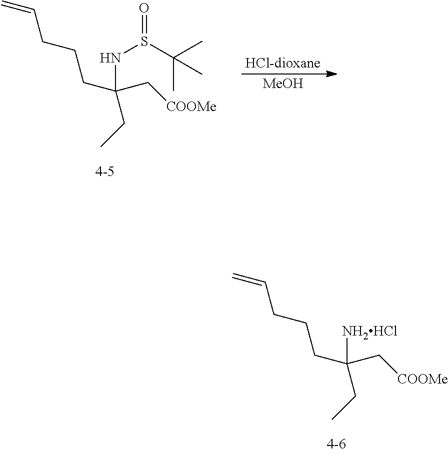

A solution of methyl 3-((tert-butylsulfinyl)amino)-3-ethyloct-7-enoate (4-5) (2 g, 6.59 mmol) in HCl-dioxane (4N) (5 mL) and MeOH (5 mL) at 15° C. under N₂ atmosphere was stirred at 15° C. for 1 h. The solution was concentrated in vacuo to give the crude product methyl 3-amino-3-ethyloct-7-enoate hydrochloride (4-6) which was used without any further purification.

¹H NMR (500 MHz, CHLOROFORM-d) δ 5.77 (m, 1H), 4.93-5.06 (m, 2H), 3.73 (s, 3H), 2.72-2.88 (m, 2H), 2.02-2.14 (m, 2H), 1.70-2.00 (m, 5H), 1.56 (m, 2H), 1.04 (t, J=7.50 Hz, 3H)

### Preparation of Compound 4-7

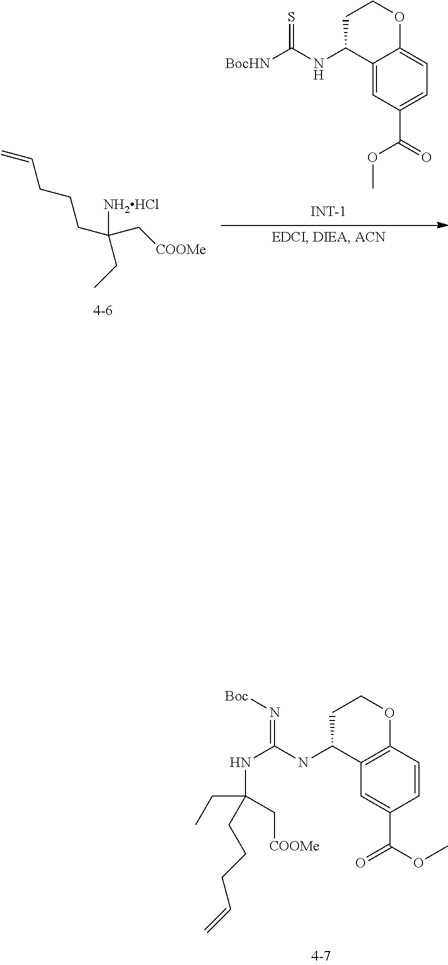

N-ethyl-N-isopropylpropan-2-amine (4.37 ml, 24.56 mmol) was added to a solution of methyl (R)-4-(3-(tert-butoxycarbonyl)thioureido)chromane-6-carboxylate (INT-1) (1.5 g, 4.09 mmol), methyl 3-amino-3-ethyloct-7-enoate hydrochloride (4-6) (1.448 g, 6.14 mmol) and N-(3-Dimethylaminopropyl)-N′-ethylcarbodiimide hydrochloride (2.354 g, 12.28 mmol) in acetonitrile (30 mL). The mixture was stirred at 15° C. for 10 h. The mixture was quenched with water (50 mL), and extracted with EtOAc (40 mL*3). The organic layers were washed with brine (20 mL), dried over Na₂SO₄, filtered, and concentrated in vacuo to afford methyl (4R)-4-((Z)-2-(tert-butoxycarbonyl)-3-(3-ethyl-1-methoxy-1-oxooct-7-en-3-yl)guanidino)chromane-6-carboxylate (4-7) which was used crude for the next step directly.

MS (ESI) m/z 532.3 (M+H⁺).

### Preparation of Compound 4-8

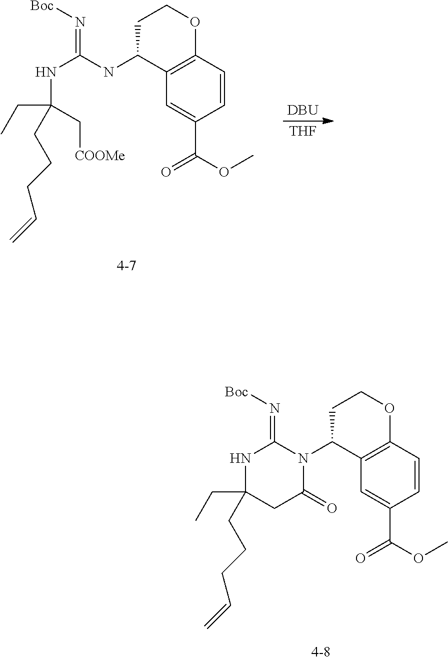

DBU (2.84 mL, 18.81 mmol) was added to a solution of methyl (4R)-4-((Z)-2-(tert-butoxycarbonyl)-3-(3-ethyl-1-methoxy-1-oxooct-7-en-3-yl)guanidino)chromane-6-carboxylate (4-7) (2.0 g, 3.76 mmol) in THF (20 mL). The mixture was stirred at 50° C. for 10 h. The mixture was quenched with water (40 mL), and extracted with EtOAc (40 mL*3). The organic layers were washed with brine (20 mL), dried over Na₂SO₄, filtered, and concentrated in vacuo. The crude was purified by flash silica gel chromatography (ISCO®; 20 g Agela Silica Flash column, eluent of 15% ethyl acetate/pet. ether gradient at 60 mL/min) to afford methyl (4R)-4-((E)-2-((tert-butoxycarbonyl)imino)-4-ethyl-6-oxo-4-(pent-4-en-1-yl)tetrahydropyrimidin-1(2H)-yl)chromane-6-carboxylate (4-8).

MS (ESI) m/z: 500.6 (M+H⁺).

¹H NMR (400 MHz, CHLOROFORM-d) δ 10.10 (s, 1H), 7.75 (d, J=8.8 Hz, 1H), 7.61 (s, 1H), 6.83 (dd, J=0.8, 8.8 Hz, 1H), 6.31-6.46 (m, 1H), 5.77 (m, 1H), 4.97-5.10 (m, 2H), 4.43 (d, J=11.2 Hz, 1H), 4.17-4.29 (m, 1H), 3.82 (s, 3H), 2.68-2.86 (m, 1H), 2.47-2.59 (m, 2H), 2.06-2.18 (m, 2H), 1.56-1.81 (m, 5H), 1.51 (s, 9H), 1.45 (s, 3H), 1.40 (s, 1H)

### Preparation of Compounds 4-9A & 4-9B

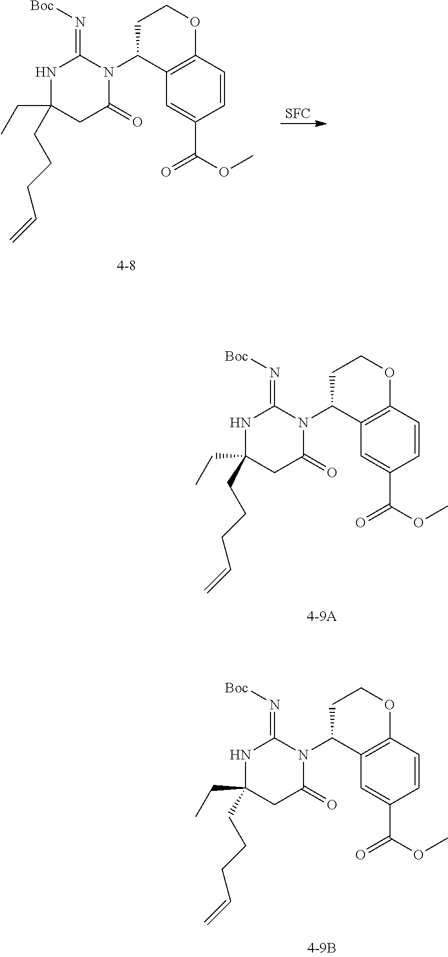

Methyl (E)-4-(2-((tert-butoxycarbonyl)imino)-4-ethyl-6-oxo-4-(pent-4-en-1-yl)tetrahydropyrimidin-1(2H)-yl)chromane-6-carboxylate (4-8) (1.8 g, 3.60 mmol) was separated by SFC (Instrument SFC-22 Method column DAICEL CHIRALPAK AD (250 mm*30 mm, 10 um) condition 0.1% NH₃H₂O IPA Begin B 10% End B 10% Gradient Time (min) 100% B Hold Time (min) Flow Rate (mL/min) 50 Injections 60) to afford product (methyl (R)-4-((R,E)-2-((tert-butoxycarbonyl)imino)-4-ethyl-6-oxo-4-(pent-4-en-1-yl)tetrahydropyrimidin-1(2H)-yl)chromane-6-carboxylate (4-9A) (peak1, Rt=0.816) and (methyl (E)-4-(2-((tert-butoxycarbonyl)imino)-4-ethyl-6-oxo-4-(pent-4-en-1-yl)tetrahydropyrimidin-1(2H)-yl)chromane-6-carboxylate (4-9B) (peak2, Rt=0.879).

MS (ESI) m/z: 500.2 (M+H⁺)

### Preparation of Compound 4-10

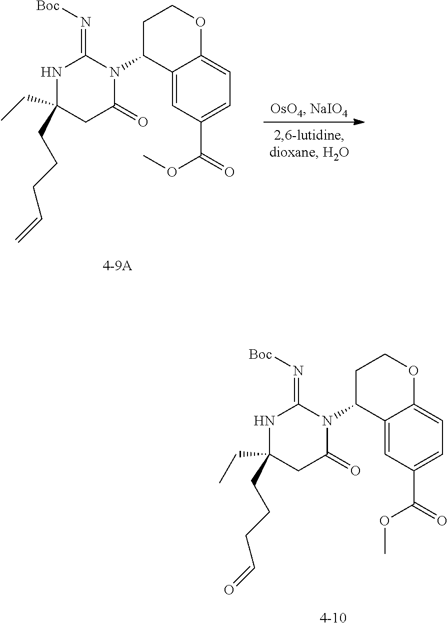

2,6-dimethylpyridine (42.9 mg, 0.400 mmol) and osmium (VIII) oxide (5.09 mg, 0.020 mmol) were added to a solution of methyl (R)-4-((R,E)-2-((tert-butoxycarbonyl)imino)-4-ethyl-6-oxo-4-(pent-4-en-1-yl)tetrahydropyrimidin-1(2H)-yl)chromane-6-carboxylate (4-9A) (100 mg, 0.200 mmol) in 1,4-dioxane (4 mL) and water (1 mL). The resulting solution was stirred for 0.2 h at 25° C. Sodium periodate (171 mg, 0.801 mmol) was added and the resulting solution was stirred for 2 h at 25° C. The mixture was quenched with Sat. Na₂SO₃ (10 mL) and water (5 mL), and extracted with EtOAc (10 mL*3). The organic layers were washed with brine (5 mL), dried over Na₂SO₄, filtered and concentrated in vacuo to afford methyl (R)-4-((R,E)-2-((tert-butoxycarbonyl)imino)-4-ethyl-6-oxo-4-(4-oxobutyl)tetrahydropyrimidin-1(2H)-yl)chromane-6-carboxylate (4-10) which was used crude for the next step directly.

MS (ESI) m/z: 502.3 (M+H⁺)

### Preparation of Compound 4-11

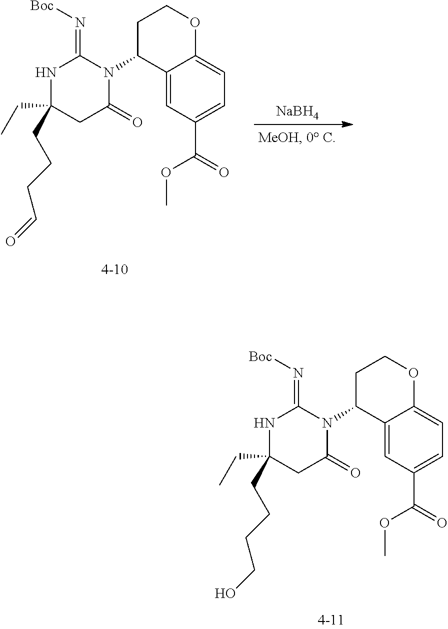

NaBH₄ (10.75 mg, 0.284 mmol) was added in portions to a solution of methyl (R)-4-((R,E)-2-((tert-butoxycarbonyl)imino)-4-ethyl-6-oxo-4-(4-oxobutyl)tetrahydropyrimidin-1(2H)-yl)chromane-6-carboxylate (4-10) (95 mg, 0.189 mmol) in MeOH (2 mL) at 0° C. The mixture was stirred at 0° C. for 1 h. LCMS showed major desired product mass. The mixture was quenched with water (5 mL), and extracted with EtOAc (5 mL*4). The combined organic layers were washed with brine (5 mL), dried over Na₂SO₄, filtered, and concentrated in vacuum, the crude was purified by prep-TLC (Pet. ether/EtOAc=3:1) to afford methyl (R)-4-((R,E)-2-((tert-butoxycarbonyl)imino)-4-ethyl-4-(4-hydroxybutyl)-6-oxotetrahydropyrimidin-1(2H)-yl)chromane-6-carboxylate (4-11).

MS (ESI) m/z: 504.4 (M+H⁺)

¹H NMR (400 MHz, CHLOROFORM-d) δ 10.14 (s, 1H), 7.76 (dd, J=2.0, 8.4 Hz, 1H), 7.65 (s, 1H), 6.84 (d, J=8.4 Hz, 1H), 6.36 (dd, J=7.2, 10.0 Hz, 1H), 4.43-4.46 (m, 1H), 4.22-4.26 (m, 1H), 3.84 (s, 3H), 3.68 (t, J=6.4 Hz, 2H), 2.69-2.78 (m, 1H), 2.51-2.62 (m, 2H), 1.95-2.24 (m, 2H), 1.64-1.69 (m, 6H), 1.52 (s, 9H), 1.43-1.49 (m, 2H), 0.97 (t, J=7.6 Hz, 3H)

### Preparation of Compound 4-12

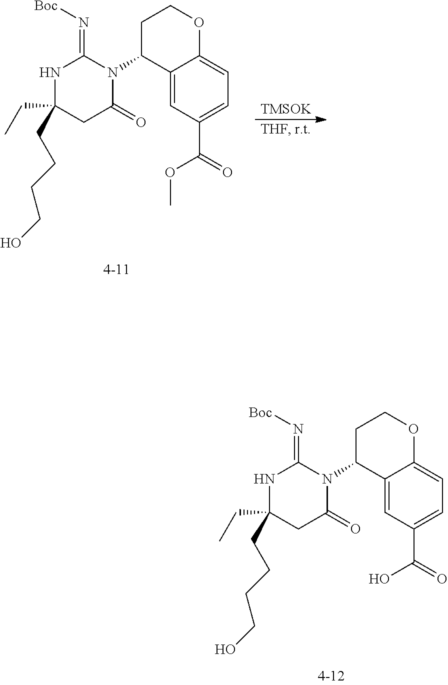

Potassium trimethylsilanolate (81 mg, 0.631 mmol) was added to a solution of methyl (R)-4-((R,E)-2-((tert-butoxycarbonyl)imino)-4-ethyl-4-(4-hydroxybutyl)-6-oxotetrahydropyrimidin-1(2H)-yl)chromane-6-carboxylate (4-11) (53 mg, 0.105 mmol) in THF (1 mL). The reaction was stirred at 25° C. for 0.5 h. LCMS showed major desired product mass. The solution of (R)-4-((R,E)-2-((tert-butoxycarbonyl)imino)-4-ethyl-4-(4-hydroxybutyl)-6-oxotetrahydropyrimidin-1(2H)-yl)chromane-6-carboxylic acid (4-12) was used in the next step without any further manipulation or purification.

MS (ESI) m/z: 490.2 (M+H⁺)

### Preparation of Compound 4-13

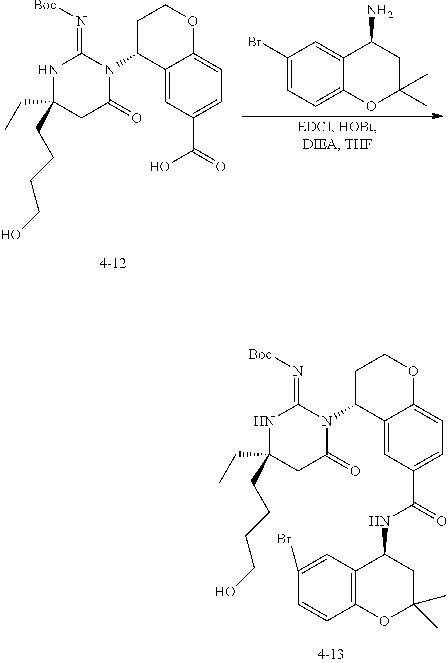

DIEA (0.143 mL, 0.817 mmol) was added to a solution of (R)-4-((R,E)-2-((tert-butoxycarbonyl)imino)-4-ethyl-4-(4-hydroxybutyl)-6-oxotetrahydropyrimidin-1(2H)-yl)chromane-6-carboxylic acid (4-12) (50 mg, 0.102 mmol), EDC (98 mg, 0.511 mmol), 1H-benzo[d][1,2,3]triazol-1-ol (69.0 mg, 0.511 mmol) and (S)-6-bromo-2,2-dimethylchroman-4-amine (28.8 mg, 0.112 mmol) in THF (5 mL). The reaction was stirred at 25° C. for 3 h. The mixture was quenched with water (5 mL), and extracted with EtOAc (5 mL*3). The organic layers were washed with brine (5 mL), dried over Na₂SO₄, filtered and concentrated in vacuum, the crude was purified by prep-TLC (Pet. ether/EtOAc=3:1) to afford tert-butyl ((R,E)-1-((R)-6-(((S)-6-bromo-2,2-dimethylchroman-4-yl)carbamoyl)chroman-4-yl)-4-ethyl-4-(4-hydroxybutyl)-6-oxotetrahydropyrimidin-2(1H)-ylidene)carbamate (4-13).

MS (ESI) m/z: 727.2, 729.2 (M+H⁺)

### Preparation of Compound 4-14

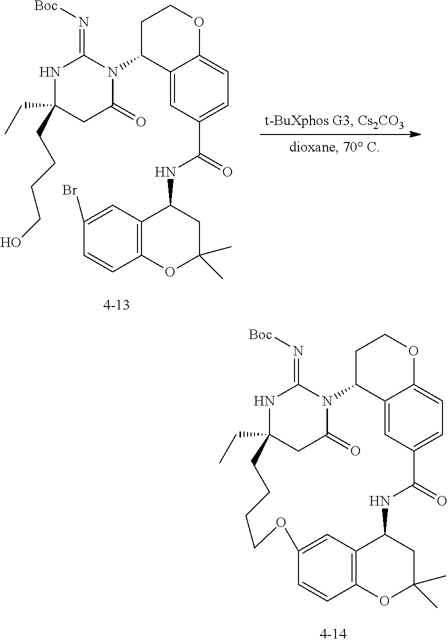

In a glove box, [(2-Di-tert-butylphosphino-2′,4′,6′-triisopropyl-1,1′-biphenyl)-2-(2′-amino-1,1′-biphenyl)]palladium(II) methanesulfonate (7.64 mg, 9.62 μmol) and CsCO₃ (37.1 mg, 0.192 mmol) was added to a mixture of tert-butyl ((R,E)-1-((R)-6-(((S)-6-bromo-2,2-dimethylchroman-4-yl)carbamoyl)chroman-4-yl)-4-ethyl-4-(4-hydroxybutyl)-6-oxotetrahydropyrimidin-2(1H)-ylidene)carbamate (4-13) (70 mg, 0.096 mmol) in dioxane (3 mL). The mixture was stirred at 70° C. for 3 h. The mixture was diluted with water (10 mL) and extracted with EtOAc (10 mL*3). The organic layers were washed with brine (5 mL), dried over Na₂SO₄, filtered and concentrated in vacuum. The crude was purified by prep-TLC (Pet. ether/EtOAc=2:1) to afford tert-butyl ((4aR,5R,8R,19aS,E)-8-ethyl-18,18-dimethyl-6,21-dioxo-4,4a,7,8,9,10,11,12,19,19a,20,21-dodecahydro-3H,6H,18H-1,22-(epiethane[1,2]diylidene)-8,5-(epiminomethano)-14,16-ethenodipyrano[3,4-d:3′,4′-j][1]oxa[6,12]diazacyclononadecin-29-ylidene)carbamate (4-14).

MS (ESI) m/z: 647.3 (M+H⁺)

¹H NMR (500 MHz, CHLOROFORM-d) δ 7.81 (dd, J=2.0, 8.5 Hz, 1H), 7.01 (s, 1H), 6.93 (d, J=8.5 Hz, 1H), 6.88 (d, J=1.0 Hz, 1H), 6.78 (s, 2H), 6.40 (dd, J=7.0, 10.0 Hz, 1H), 6.13 (d, J=8.50 Hz, 1H), 5.37-5.44 (m, 1H), 4.44 (td, J=3.5, 11.5 Hz, 1H), 4.17-4.23 (m, 2H), 3.82 (dt, J=5.0, 9.5 Hz, 1H), 2.71-2.79 (m, 2H), 2.53 (d, J=16.0 Hz, 1H), 2.35 (dd, J=6.5, 13.0 Hz, 1H), 2.07-2.14 (m, 2H), 1.78 (dd, J=7.50, 10.50 Hz, 4H), 1.52 (s, 2H), 1.49 (s, 9H), 1.42 (s, 3H), 1.37 (s, 3H), 0.95 (t, J=7.5 Hz, 3H)

### Preparation of Example 4

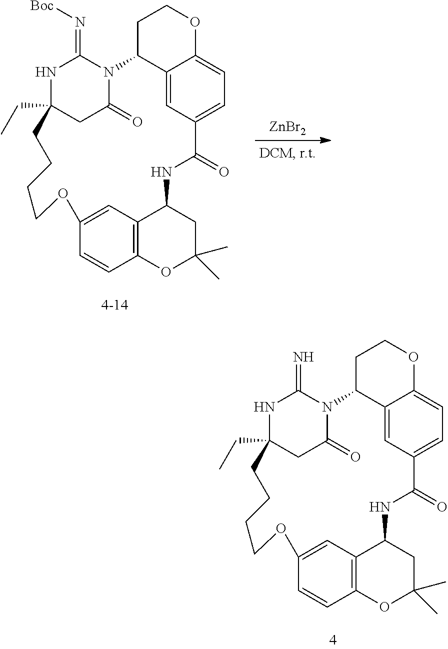

Zinc(II) bromide (104 mg, 0.464 mmol) and oxa[6,12]diazacyclononadecin-29-ylidene)carbamate (4-14) (30 mg, 0.046 mmol) in DCM (3 mL) at 25° C. were added to a solution of tert-butyl ((4aR,5R,8R,19aS,E)-8-ethyl-18,18-dimethyl-6,21-dioxo-4,4a,7,8,9,10,11,12,19,19a,20,21-dodecahydro-3H,6H,18H-1,22-(epiethane[1,2]diylidene)-8,5-(epiminomethano)-14,16-ethenodipyrano[3,4-d:3′,4′-j][1], under N₂ atmosphere. The mixture was stirred at 25° C. for 16 h. The solvent was evaporated under reduced pressure to give the crude product and the residue was purified by reverse preparative HPLC (Instrument EG; Method column Welch Xtimate C18 150*25 mm*5 um; condition water (TFA)-ACN Begin B 25; End B 55 Gradient Time (min) 11; 100% B Hold Time (min) 2 Flow Rate (mL/min) 25; Injections 1) to give (4aR,5R,8R,19aS)-8-ethyl-29-imino-18,18-dimethyl-4,4a,7,8,9,10,11,12,18,19,19a,20-dodecahydro-3H,6H,21H-1,22-(epiethane[1,2]diylidene)-8,5-(epiminomethano)-14,16-ethenodipyrano[3,4-d:3′,4′-j][1]oxa[6,12]diazacyclononadecine-6,21-dione Example 4.

MS (ESI) m/z: 547.2 (M+H⁺)

1H NMR (500 MHz, METHANOL-d4) δ 8.55 (d, J=8.5 Hz, 1H), 7.73 (dd, J=2.0, 8.5 Hz, 1H), 7.45 (d, J=1.5 Hz, 1H), 6.94 (d, J=8.50 Hz, 1H), 6.86 (d, J=2.5 Hz, 1H), 6.76-6.82 (m, 1H), 6.70 (d, J=9.0 Hz, 1H), 5.35-5.40 (m, 1H), 5.24-5.32 (m, 1H), 4.47 (td, J=4.5, 11.50 Hz, 1H), 4.13-4.21 (m, 2H), 3.90-3.92 (m, 1H), 2.75-2.88 (m, 2H), 2.60-2.72 (m, 1H), 2.22-2.31 (m, 1H), 2.15 (dd, J=6.5, 13.0 Hz, 1H), 1.66-1.85 (m, 7H), 1.47-1.60 (m, 2H), 1.40 (s, 3H), 1.33 (s, 3H), 0.97 (t, J=7.5 Hz, 3H)

The compounds in Table 1 were prepared in an analogous fashion to that described for Example 1. The isomers were separated by preparative HPLC or/and preparative chiral SFC.

An asterisk (*) may be used in a chemical structure drawing that indicates the location of a chiral center.

TABLE 1

| Example | Structure | MS Observed | Chemical Name |
|---|---|---|---|
| 3B | 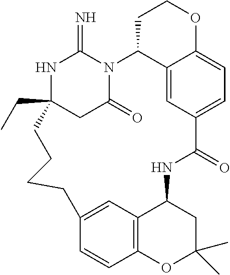 [SMILES: CC[C@@]12CCCCc3ccc4c(c3)[C@H](CC(C)(C)O4)NC(=O)c3ccc4c(c3)[C@@H](CCO4)N(C(=N)N1)C(=O)C2] | 531.2 | (1R,5S,17S)-5- ethyl-3-imino- 15,15-dimethyl- 14,24-dioxa- 2,4,18- triazahexacyclo[18. 6.2.22,5.210,13.012, 17.023,27] dotriaconta- 10,12,20,22,27,29- hexaene-19,32- dione |
| 5 | 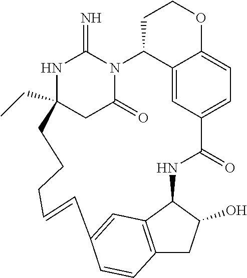 [SMILES: CC[C@]12CCC/C=C/c3ccc4c(c3)[C@@H](NC(=O)c3ccc5c(c3)[C@@H](CCO5)N(C(=N)N1)C(=O)C2)[C@H](O)C4] | 515.3 | (1R,5R,9E,16R, 17R)-5-ethyl-16- hydroxy-3-imino- 24-oxa-2,4,18- triazahexacyclo[18. 6.2.22,5.211,14.013, 17.023,27] dotriaconta- 9,11,13,20,22,27, 29-heptaene-19,32- dione |
| 6 | 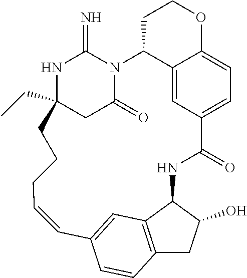 [SMILES: CC[C@]12CCC/C=C\c3ccc4c(c3)[C@@H](NC(=O)c3ccc5c(c3)[C@@H](CCO5)N(C(=N)N1)C(=O)C2)[C@H](O)C4] | 515.2 | (1R,5R,9Z,16R, 17R)-5-ethyl-16- hydroxy-3-imino- 24-oxa-2,4,18- triazahexacyclo[18. 6.2.22,5.211,14.013, 17.023,27] dotriaconta- 9,11,13,20,22,27, 29-heptaene-19,32- dione |
| 7 | 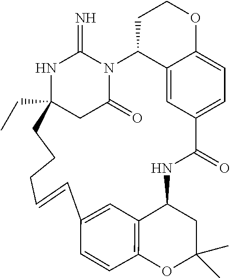 [SMILES: CC[C@]12CCC/C=C/c3ccc4c(c3)[C@H](CC(C)(C)O4)NC(=O)c3ccc4c(c3)[C@@H](CCO4)N(C(=N)N1)C(=O)C2] | 543.3 | (1R,5R,9E,18S)-5- ethyl-3-imino- 16,16-dimethyl- 15,25-dioxa- 2,4,19- triazahexacyclo[19. 6.2.22,5.211,14.013, 18.024,28] tritriaconta- 9,11,13,21,23,28, 30-heptaene-20,33- dione |
| 8 | 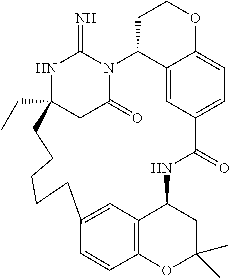 [SMILES: CC[C@]12CCCCCc3ccc4c(c3)[C@H](CC(C)(C)O4)NC(=O)c3ccc4c(c3)[C@@H](CCO4)N(C(=N)N1)C(=O)C2] | 545.3 | (1R,5R,18S)-5- ethyl-3-imino- 16,16-dimethyl- 15,25-dioxa- 2,4,19- triazahexacyclo[19. 6.2.22,5.211,14.013, 18.024,28] tritriaconta- 11,13,21,23,28,30- hexaene-20,33- dione |
| 9 | 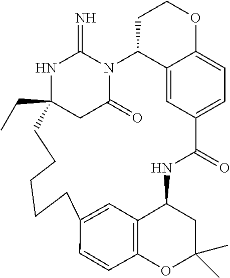 [SMILES: CC[C@@]12CCCCCc3ccc4c(c3)[C@H](CC(C)(C)O4)NC(=O)c3ccc4c(c3)[C@@H](CCO4)N(C(=N)N1)C(=O)C2] | 545.3 | (1R,5S,18S)-5- ethyl-3-imino- 16,16-dimethyl- 15,25-dioxa- 2,4,19- triazahexacyclo[19. 6.2.22,5.211,14.013, 18.024,28] tritriaconta- 11,13,21,23,28,30- hexaene-20,33- dione |
| 10 | 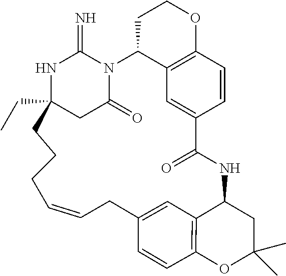 [SMILES: CC[C@]12CCC/C=C\Cc3ccc4c(c3)[C@H](CC(C)(C)O4)NC(=O)c3ccc4c(c3)[C@@H](CCO4)N(C(=N)N1)C(=O)C2] | 557.3 | (1R,5R,9Z,19S)-5- ethyl-3-imino- 17,17-dimethyl- 16,26-dioxa- 2,4,20- triazahexacyclo[20. 6.2.22,5.212,15.014, 19.025,29] tetratriaconta- 9,12,14,22(30),23, 25(29),31- heptaene-21,34- dione |
| 11 | 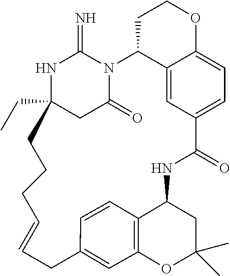 [SMILES: CC[C@]12CCC/C=C/Cc3ccc4c(c3)OC(C)(C)C[C@@H]4NC(=O)c3ccc4c(c3)[C@@H](CCO4)N(C(=N)N1)C(=O)C2] | 557.3 | (1R,5R,9E,20S)-5- ethyl-3-imino- 18,18-dimethyl- 17,27-dioxa- 2,4,21- triazahexacyclo[21. 6.2.22,5.112,16.015, 20.026,30] tetratriaconta- 9,12(32),13,15,23, 25,30-heptaene- 22,34-dione |
| 12 | 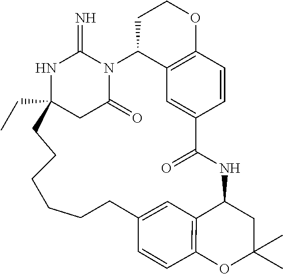 [SMILES: CC[C@]12CCCCCCc3ccc4c(c3)[C@H](CC(C)(C)O4)NC(=O)c3ccc4c(c3)[C@@H](CCO4)N(C(=N)N1)C(=O)C2] | 559.3 | (1R,5R,19S)-5- ethyl-3-imino- 17,17-dimethyl- 16,26-dioxa- 2,4,20- triazahexacyclo[20. 6.2.22,5.212,15.014, 19.025,29] tetratriaconta- 12,14,22(30),23, 25(29),31-hexaene- 21,34-dione |
| 13 | 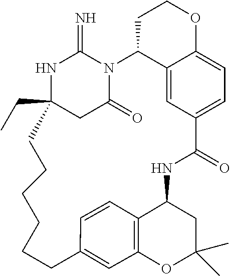 [SMILES: CC[C@@]12CCCCCCc3ccc4c(c3)OC(C)(C)C[C@@H]4NC(=O)c3ccc4c(c3)[C@@H](CCO4)N(C(=N)N1)C(=O)C2] | 559.3 | (1R,5S,20S)-5- ethyl-3-imino- 18,18-dimethyl- 17,27-dioxa- 2,4,21- triazahexacyclo[21. 6.2.22,5.112,16.015, 20.026,30] tetratriaconta- 12(32),13,15,23,25, 30-hexaene- 22,34-dione |
| 14 | 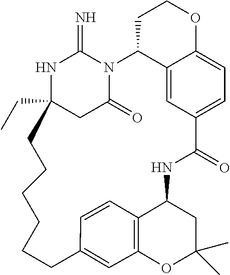 [SMILES: CC[C@]12CCCCCCc3ccc4c(c3)OC(C)(C)C[C@@H]4NC(=O)c3ccc4c(c3)[C@@H](CCO4)N(C(=N)N1)C(=O)C2] | 559.3 | (1R,5R,20S)-5- ethyl-3-imino- 18,18-dimethyl- 17,27-dioxa- 2,4,21- triazahexacyclo[21. 6.2.22,5.112,16.015, 20.026,30] tetratriaconta- 12(32),13,15,23, 25,30-hexaene- 22,34-dione |
| 15A | 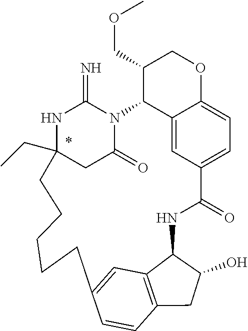 [SMILES: CCC12CCCCCc3ccc4c(c3)[C@@H](NC(=O)c3ccc5c(c3)[C@@H]([C@H](COC)CO5)N(C(=N)N1)C(=O)C2)[C@H](O)C4] | 561.2 | (1R,16R,17R,26R)- 5-ethyl-16- hydroxy-3-imino- 26- (methoxymethyl)- 24-oxa-2,4,18- triazahexacyclo[18. 6.2.22,5.211,14.013, 17.023,27] dotriaconta- 11,13,20,22,27,29- hexaene-19,32- dione |
| 15B | 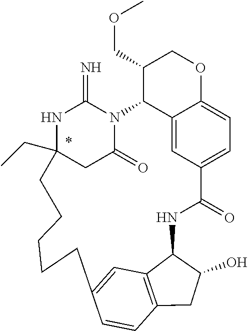 [SMILES: CCC12CCCCCc3ccc4c(c3)[C@@H](NC(=O)c3ccc5c(c3)[C@@H]([C@H](COC)CO5)N(C(=N)N1)C(=O)C2)[C@H](O)C4] | 561.2 | (1R,16R,17R,26R)- 5-ethyl-16- hydroxy-3-imino- 26- (methoxymethyl)- 24-oxa-2,4,18- triazahexacyclo[18. 6.2.22,5.211,14.013, 17.023,27] dotriaconta- 11,13,20,22,27,29- hexaene-19,32- dione |
| 16A | 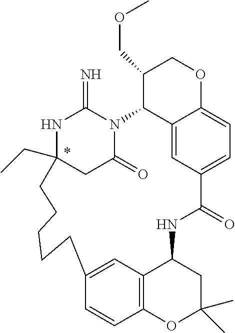 [SMILES: CCC12CCCCCc3ccc4c(c3)[C@H](CC(C)(C)O4)NC(=O)c3ccc4c(c3)[C@@H]([C@H](COC)CO4)N(C(=N)N1)C(=O)C2] | 589.3 | (1R,18S,27R)-5- ethyl-3-imino-27- (methoxymethyl)- 16,16-dimethyl- 15,25-dioxa- 2,4,19- triazahexacyclo[19. 6.2.22,5.211,14.013, 18.024,28] tritriaconta- 11,13,21,23,28,30- hexaene-20,33- dione |
| 16B | 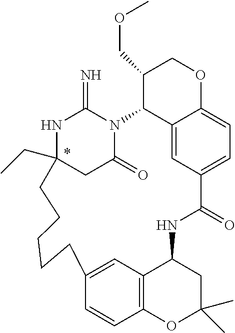 [SMILES: CCC12CCCCCc3ccc4c(c3)[C@H](CC(C)(C)O4)NC(=O)c3ccc4c(c3)[C@@H]([C@H](COC)CO4)N(C(=N)N1)C(=O)C2] | 589.3 | rac-(1R,18S,27R)- 5-ethyl-3-imino- 27- (methoxymethyl)- 16,16-dimethyl- 15,25-dioxa- 2,4,19- triazahexacyclo[19. 6.2.22,5.211,14.013, 18.024,28] tritriaconta- 11,13,21,23,28,30- hexaene-20,33- dione |
| 17 | 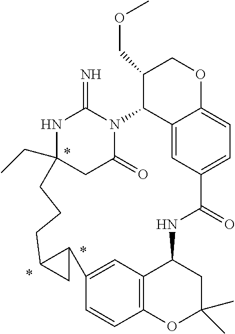 [SMILES: CCC12CCCC3CC3c3ccc4c(c3)[C@H](CC(C)(C)O4)NC(=O)c3ccc4c(c3)[C@@H]([C@H](COC)CO4)N(C(=N)N1)C(=O)C2] | 601.3 | rac- (12R,13R,22S)-8- ethyl-10-imino- 13- (methoxymethyl)- 24,24-dimethyl- 15,25-dioxa- 9,11,21- triazaheptacyclo[20. 6.2.28,11.216,19.02, 4.012,17.026,30] tetratriaconta- 1(29),16(32),17,19 (31),26(30),27- hexaene-20,33- dione |
| 18 | 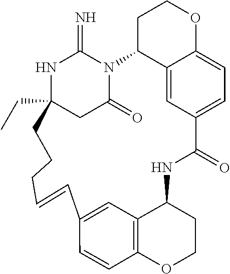 [SMILES: CC[C@]12CCC/C=C/c3ccc4c(c3)[C@H](CCO4)NC(=O)c3ccc4c(c3)[C@@H](CCO4)N(C(=N)N1)C(=O)C2] | 515.2 | rac- (1R,5R,9E,18S)-5- ethyl-3-imino- 15,25-dioxa- 2,4,19- triazahexacyclo[19. 6.2.22,5.211,14.013, 18.024,28] tritriaconta- 9,11,13,21,23,28,30- heptaene-20,33- dione |
| 19 | 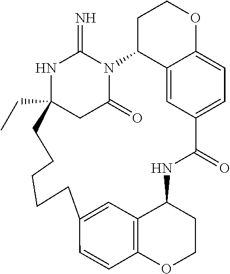 [SMILES: CC[C@]12CCCCCc3ccc4c(c3)[C@H](CCO4)NC(=O)c3ccc4c(c3)[C@@H](CCO4)N(C(=N)N1)C(=O)C2] | 517.2 | rac-(1R,5R,18S)- 5-ethyl-3-imino- 15,25-dioxa- 2,4,19- triazahexacyclo[19. 6.2.22,5.211,14.013, 18.024,28] tritriaconta- 11,13,21,23,28,30- hexaene-20,33- dione |
| 20 | 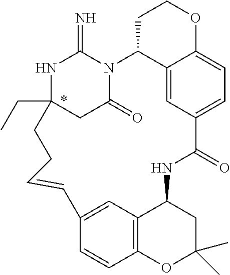 [SMILES: CCC12CC/C=C/c3ccc4c(c3)[C@H](CC(C)(C)O4)NC(=O)c3ccc4c(c3)[C@@H](CCO4)N(C(=N)N1)C(=O)C2] | 529.2 | rac-(1R,8E,17S)- 5-ethyl-3-imino- 15,15-dimethyl- 14,24-dioxa- 2,4,18- triazahexacyclo[18. 6.2.22,5.210,13.012, 17.023,27] dotriaconta- 8,10,12,20,22,27,29- heptaene-19,32- dione |
| 21 | 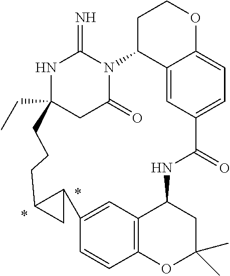 [SMILES: CC[C@]12CCCC3CC3c3ccc4c(c3)[C@H](CC(C)(C)O4)NC(=O)c3ccc4c(c3)[C@@H](CCO4)N(C(=N)N1)C(=O)C2] | 529.2 | rac-(8R,12R,22S)- 8-ethyl-10-imino- 15,25-dioxa- 9,11,21- triazaheptacyclo[20. 6.2.28,11.216,19.02, 4.012,17.026,30] tetratriaconta- 1(29),16(32),17,19 (31),26(30),27- hexaene-20,33- dione |
| 22 | 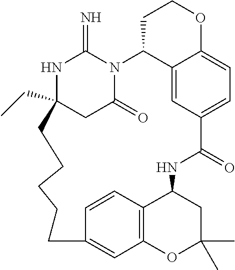 [SMILES: CC[C@]12CCCCCc3ccc4c(c3)OC(C)(C)C[C@@H]4NC(=O)c3ccc4c(c3)[C@@H](CCO4)N(C(=N)N1)C(=O)C2] | 545.2 | rac-(1R,5R,19S)- 5-ethyl-3-imino- 17,17-dimethyl- 16,26-dioxa- 2,4,20- triazahexacyclo[20. 6.2.22,5.111,15.014, 19.025,29] tritriaconta- 11(31),12,14,22,24, 29-hexaene- 21,33-dione |
| 23 | 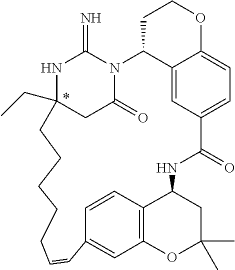 [SMILES: CCC12CCCCC/C=C\c3ccc4c(c3)OC(C)(C)C[C@@H]4NC(=O)c3ccc4c(c3)[C@@H](CCO4)N(C(=N)N1)C(=O)C2] | 571.3 | rac-(1R,11Z,21S)- 5-ethyl-3-imino- 19,19-dimethyl- 18,28-dioxa- 2,4,22- triazahexacyclo[22. 6.2.22,5.113,17.016, 21.027,31] pentatriaconta- 11,13(33),14,16,24, 26,31-heptaene- 23,35-dione |
| 24A | 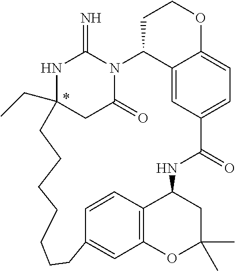 [SMILES: CCC12CCCCCCCc3ccc4c(c3)OC(C)(C)C[C@@H]4NC(=O)c3ccc4c(c3)[C@@H](CCO4)N(C(=N)N1)C(=O)C2] | 573.3 | rac-(1R,21S)-5- ethyl-3-imino- 19,19-dimethyl- 18,28-dioxa- 2,4,22- triazahexacyclo[22. 6.2.22,5.113,17.016, 21.027,31] pentatriaconta- 13(33),14,16,24,26, 31-hexaene- 23,35-dione |
| 24B | 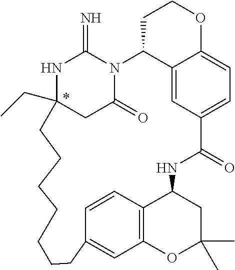 [SMILES: CCC12CCCCCCCc3ccc4c(c3)OC(C)(C)C[C@@H]4NC(=O)c3ccc4c(c3)[C@@H](CCO4)N(C(=N)N1)C(=O)C2] | 573.3 | rac-(1R,21S)-5- ethyl-3-imino- 19,19-dimethyl- 18,28-dioxa- 2,4,22- triazahexacyclo[22. 6.2.22,5.113,17.016, 21.027,31] pentatriaconta- 13(33),14,16,24,26, 31-hexaene- 23,35-dione |
| 25A | 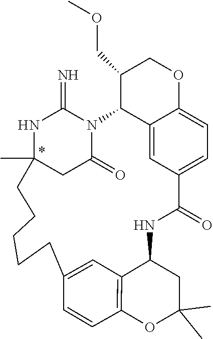 | 575.4 | rac-(1R,18S,27R)- 3-imino-27- (methoxymethyl)- 5,16,16-trimethyl- 15,25-dioxa- 2,4,19- triazahexacyclo[19. 6.2.22,5.211,14.013, 18.024,28] tritriaconta- 11,13,21,23,28,30- hexaene-20,33- dione |
| 25B | 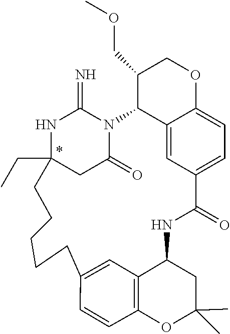 [SMILES: CCC12CCCCCc3ccc4c(c3)[C@H](CC(C)(C)O4)NC(=O)c3ccc4c(c3)[C@@H]([C@H](COC)CO4)N(C(=N)N1)C(=O)C2] | 575.3 | rac-(1R,18S,27R)- 3-imino-27- (methoxymethyl)- 5,16,16-trimethyl- 15,25-dioxa- 2,4,19- triazahexacyclo[19. 6.2.22,5.211,14.013, 18.024,28] tritriaconta- 11,13,21,23,28,30- hexaene-20,33- dione |
| 26A | 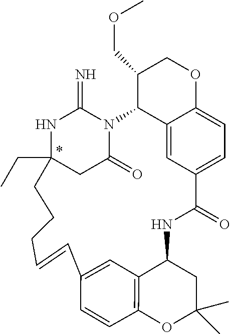 [SMILES: CCC12CCC/C=C/c3ccc4c(c3)[C@H](CC(C)(C)O4)NC(=O)c3ccc4c(c3)[C@@H]([C@H](COC)CO4)N(C(=N)N1)C(=O)C2] | 587.5 | rac- (1R,9E,18S,27R)- 5-ethyl-3-imino- 27- (methoxymethyl)- 16,16-dimethyl- 15,25-dioxa- 2,4,19- triazahexacyclo[19. 6.2.22,5.211,14.013, 18.024,28] tritriaconta- 9,11,13,21,23,28,30- heptaene-20,33- dione |
| 26B | 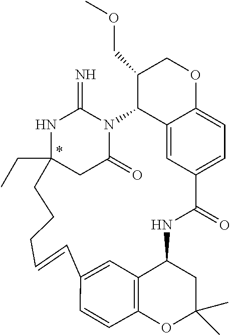 [SMILES: CCC12CCC/C=C/c3ccc4c(c3)[C@H](CC(C)(C)O4)NC(=O)c3ccc4c(c3)[C@@H]([C@H](COC)CO4)N(C(=N)N1)C(=O)C2] | 587.3 | rac- (1R,9E,18S,27R)- 5-ethy1-3-imino- 27- (methoxymethyl)- 16,16-dimethyl- 15,25-dioxa- 2,4,19- triazahexacyclo[19. 6.2.22,5.211,14.013, 18.024,28] tritriaconta- 9,11,13,21,23,28,30- heptaene-20,33- dione |

### Assessing Antiparasite Potency in a Parasite LDH Growth Assay (Parasite Assay)

The parasite stock was maintained at 4% haematocrit in RPMI-Hepes media 30 buffered with sodium bicarbonate and supplemented with 5% heat inactivated human serum and 0.5% albumax. Approximately 42 hours prior to the potency assay being set up, parasites were synchronized with 5% sorbitol to select for ring stage parasites. On the day of assay set up, a blood smear of the parasite culture was Giemsa stained and counted. The parasitemia was 35 adjusted to 0.7% rings and the haematocrit was diluted to 2% in RPMI-Hepes media buffered with sodium bicarbonate and supplemented with 5% heat inactivated human serum and 0.5% albumax. 30 ul of diluted parasites are then added into 10 μl of media+compound in pre-prepared Greiner TC assay plates. Parasite assay plates were placed in gassed humidified boxes in single layer and allowed to incubate at 37° C. for 72 hours. After 72 hours growth, assay plates are sealed with parafilm and frozen flat, in single file at −80° C. overnight. On the following day, assay plates were allowed to thaw at room temperature for 4 hours to which an LDH assay was performed to measure parasite growth.

Assay EC50 results are shown in Table 2.

TABLE 2

| Example | EC₅₀ (nM) |
|---|---|
| 1 | 2.7 |
| 2 | 11.0 |
| 3A | 0.2 |
| 3B | 269.8 |
| 4 | 2.0 |
| 5 | 0.9 |
| 6 | 2.8 |
| 7 | 0.9 |
| 8 | 1.1 |
| 9 | 76.5 |
| 10 | 2.5 |
| 11 | 4.6 |
| 12 | 1.0 |
| 13 | 22.0 |
| 14 | 34.0 |
| 15A | 9.2 |
| 15B | 81.0 |
| 16A | 61.5 |
| 16B | 3.4 |
| 17 | 18.9 |
| 18 | 6.0 |
| 19 | 4.8 |
| 20 | 3.6 |
| 21 | 5.1 |
| 22 | 57.4 |
| 23 | 7.5 |
| 24A | 5.4 |
| 24B | 2.0 |
| 25A | 23.0 |
| 25B | 0.7 |
| 26A | 189.7 |
| 26B | 7.4 |

1. A compound having the structural Formula (I): 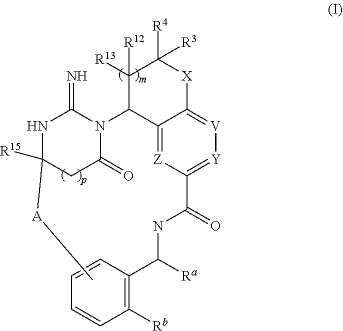
or a pharmaceutically acceptable salt thereof, wherein:
A is a straight or branched, saturated or unsaturated (C₃-C₁₀)alkylene, phenyl(C₃-C₁₀)alkylene or cycloalkyl(C₃-C₁₀)alkylene comprising at least one —CH₂— group, wherein one or more additional —CH₂— groups in A are optionally and independently replaced with a moiety selected from the group consisting of O, S, NR, CONR, NRCO, SO₂, and SO₂NR and wherein one or more of the hydrogens along A can be replaced with a group independently selected from hydroxyl, halogen and C₁₋₃ haloalkyl;
X is a bond, C(R¹⁴)₂, O, S, SO, SO₂ or NH;
Y is CR⁹ or N, wherein when Y is N, Z is CR¹¹ and V is CR¹⁰;
V is CR¹⁰ or N, wherein when V is N, Z is CR¹¹ and Y is CR⁹;
Z is CR¹¹ or N, wherein when Z is N, V is CR¹⁰ and Y is CR⁹;
R is hydrogen, C₁-C₆alkylCOOH, COOH, C₃-C₆cycloalkyl, C₁-C₆alkyl, haloC₁-C₆alkyl, C₁-C₆alkylOH, COC₁-C₆alkyl or COOC₁-C₆alkyl;
R^{a} is hydrogen, halogen, CN, OH, C₁-C₆alkoxy, C₁-C₆alkylOC₁-C₆alkyl, C₁-C₆alkylCOOH, COOH, oxo, COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, —C₁-C₆alkylOhaloC₁-C₆alkyl, haloC₁-C₆alkyl, C₁-C₆alkylOH, CON(R⁷)(R⁸), N(R⁷)(R⁸) or C₁-C₆alkylN(R⁷)(R⁸) or when taken with R^{b} forms a C₃-C₆cycloalkyl or heterocycloalkyl, wherein the C₃-C₆cycloalkyl or heterocycloalkyl is unsubstituted or substituted with one or two substituents selected from the group consisting of halogen, CN, OH, C₁-C₆alkoxy, C₁-C₆alkylOC₁-C₆alkyl, C₁-C₆alkylCOOH, COOH, oxo, COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, —C₁-C₆alkylOhaloC₁-C₆alkyl, haloC₁-C₆alkyl, C₁-C₆alkylOH, CON(R⁷)(R⁸), N(R⁷)(R⁸) and C₁-C₆alkylN(R⁷)(R⁸);
R^{b} is hydrogen, halogen, CN, OH, C₁-C₆alkoxy, C₁-C₆alkylOC₁-C₆alkyl, C₁-C₆alkylCOOH, COOH, oxo, COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, —C₁-C₆alkylOhaloC₁-C₆alkyl, haloC₁-C₆alkyl, C₁-C₆alkylOH, CON(R⁷)(R⁸), N(R⁷)(R⁸) or C₁-C₆alkylN(R⁷)(R⁸) or when taken with R^{a} forms a C₃-C₆cycloalkyl or heterocycloalkyl, wherein the C₃-C₆cycloalkyl or heterocycloalkyl is unsubstituted or substituted with one or three substituents selected from the group consisting of halogen, CN, OH, C₁-C₆alkoxy, C₁-C₆alkylOC₁-C₆alkyl, C₁-C₆alkylCOOH, COOH, oxo, COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, —C₁-C₆alkylOhaloC₁-C₆alkyl, haloC₁-C₆alkyl, C₁-C₆alkylOH, CON(R⁷)(R⁸), N(R⁷)(R⁸) and C₁-C₆alkylN(R⁷)(R⁸);
R³ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸), C₁-C₆alkylN(R⁷)(R⁸), C₁-C₆alkyl(OCH₂CH₂)ₙN(R⁷)(R⁸) or C₁-C₆alkylOhaloC₁-C₆alkyl or when taken with R⁴ forms a C₃-C₆cycloalkyl or C₃-C₆heterocycloalkyl;
R⁴ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸), C₁-C₆alkylN(R⁷)(R⁸), C₁-C₆alkyl(OCH₂CH₂)ₙN(R⁷)(R⁸) or C₁-C₆alkylOhaloC₁-C₆alkyl or when taken with R³ forms a C₃-C₆cycloalkyl or C₃-C₆heterocycloalkyl;
R⁷ is hydrogen, C₁-C₆alkylCOOH, COOH, C₃-C₆cycloalkyl, C₁-C₆alkyl, haloC₁-C₆alkyl, C₁-C₆alkylOH, COC₁-C₆alkyl or COOC₁-C₆alkyl;
R⁸ is hydrogen, C₁-C₆alkylCOOH, COOH, C₃-C₆cycloalkyl, C₁-C₆alkyl, haloC₁-C₆alkyl, C₁-C₆alkylOH, COC₁-C₆alkyl or COOC₁-C₆alkyl;
R⁹ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) or C₁-C₆alkylN(R⁷)(R⁸);
R¹⁰ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) or C₁-C₆alkylN(R⁷)(R⁸);
R¹¹ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) or C₁-C₆alkylN(R⁷)(R⁸);
R¹² is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) or C₁-C₆alkylN(R⁷)(R⁸);
R¹³ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) or C₁-C₆alkylN(R⁷)(R⁸);
each occurrence of R¹⁴ is independently selected from the group consisting of hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) and C₁-C₆alkylN(R⁷)(R⁸);
R¹⁵ is hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) or C₁-C₆alkylN(R⁷)(R⁸);
each occurrence of R¹⁶ is independently selected from the group consisting of hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) and C₁-C₆alkylN(R⁷)(R⁸);
m is 0 or 1;
n is 1, 2, 3 or 4; and
p is 0 or 1.

2. The compound of claim 1, or a pharmaceutically acceptable salt thereof, wherein m is 1, p is 1 and X is O.

3. The compound of claim 1, or a pharmaceutically acceptable salt thereof, wherein m is 1, p is 1 and X is a bond.

4. The compound of claim 1, or a pharmaceutically acceptable salt thereof, wherein R^{a} is taken with R^{b} and forms a heterocycloalkyl.

5. The compound of claim 1, or a pharmaceutically acceptable salt thereof, wherein R^{a} is taken with R^{b} and forms a heterocycloalkyl, wherein the heterocycloalkyl is 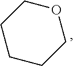
and wherein the heterocycloalkyl is substituted with two C₁-C₆alkyl groups.

6. The compound of claim 1, or a pharmaceutically acceptable salt thereof, wherein R^{a} is taken with R^{b} and forms a C₃-C₆cycloalkyl.

7. The compound of claim 1, or a pharmaceutically acceptable salt thereof, wherein R^{a} is taken with R^{b} and forms a C₃-C₆cycloalkyl, wherein the C₃-C₆cycloalkyl is 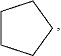
and wherein the C₃-C₆cycloalkyl is unsubstituted or substituted with OH.

8. The compound of claim 1, or a pharmaceutically acceptable salt thereof, wherein R³ is hydrogen, C₁-C₆alkylOC₁-C₆alkyl or C₁-C₆alkyl or when taken with R⁴ forms a C₃-C₆heterocycloalkyl.

9. The compound of claim 1, or a pharmaceutically acceptable salt thereof, wherein R³ is hydrogen or C₁-C₆alkyl or when taken with R⁴ forms a C₃-C₆heterocycloalkyl.

10. The compound of claim 1, or a pharmaceutically acceptable salt thereof, wherein R³ and R⁴ are both halogen.

11. The compound of claim 1, or a pharmaceutically acceptable salt thereof, wherein R¹⁵ is hydrogen or C₁-C₆alkyl.

12. The compound of claim 1, or a pharmaceutically acceptable salt thereof, wherein R³ and R⁴ are independently selected from the group consisting of hydrogen, halogen, OH, C₁-C₆alkylOH, C₁-C₆alkylalkoxy, C₁-C₆alkylOC₁-C₆alkyl, C₁-C₆alkyl, C₁-C₆alkylOhaloC₁-C₆alkyl, CON(C₁-C₆alkyl)₂, C₁-C₆alkylN(R⁷)(R⁸) and C₁-C₆alkyl(OCH₂CH₂)ₙN(R⁷)(R⁸);
R⁷ is hydrogen, C₁-C₆alkylCOOH, COOH, C₃-C₆cycloalkyl, C₁-C₆alkyl, haloC₁-C₆alkyl, C₁-C₆alkylOH, COC₁-C₆alkyl or COOC₁-C₆alkyl;
R⁸ is hydrogen, C₁-C₆alkylCOOH, COOH, C₃-C₆cycloalkyl, C₁-C₆alkyl, haloC₁-C₆alkyl, C₁-C₆alkylOH, COC₁-C₆alkyl or COOC₁-C₆alkyl; and
n is 3.

13. The compound of claim 1, or a pharmaceutically acceptable salt thereof, wherein R¹² and R¹³ are independently selected from the group consisting of hydrogen, halogen, OH, C₁-C₆alkylOH, C₁-C₆alkylalkoxy, and C₁-C₆alkylOC₁-C₆alkyl, and C₁-C₆alkyl.

14. The compound of claim 1, or a pharmaceutically acceptable salt thereof, wherein X is C(R¹⁴)₂, and R¹⁴ is independently selected from the group consisting of hydrogen, halogen, OH, C₁-C₆alkylOH, C₁-C₆alkylalkoxy, C₁-C₆alkylOC₁-C₆alkyl and C₁-C₆alkyl.

15. The compound of claim 1, or a pharmaceutically acceptable salt thereof, wherein Y is CH.

16. The compound of claim 1, or a pharmaceutically acceptable salt thereof, wherein Z is CH.

17. The compound of claim 1, or a pharmaceutically acceptable salt thereof, wherein V is CH.

18. The compound of claim 1, or a pharmaceutically acceptable salt thereof, wherein A is a straight, saturated or unsaturated (C₃-C₁₀)alkylene.

19. The compound of claim 1, or a pharmaceutically acceptable salt thereof, wherein A is a branched, saturated or unsaturated (C₃-C₁₀)alkylene.

20. The compound of claim 1, or a pharmaceutically acceptable salt thereof, wherein A is

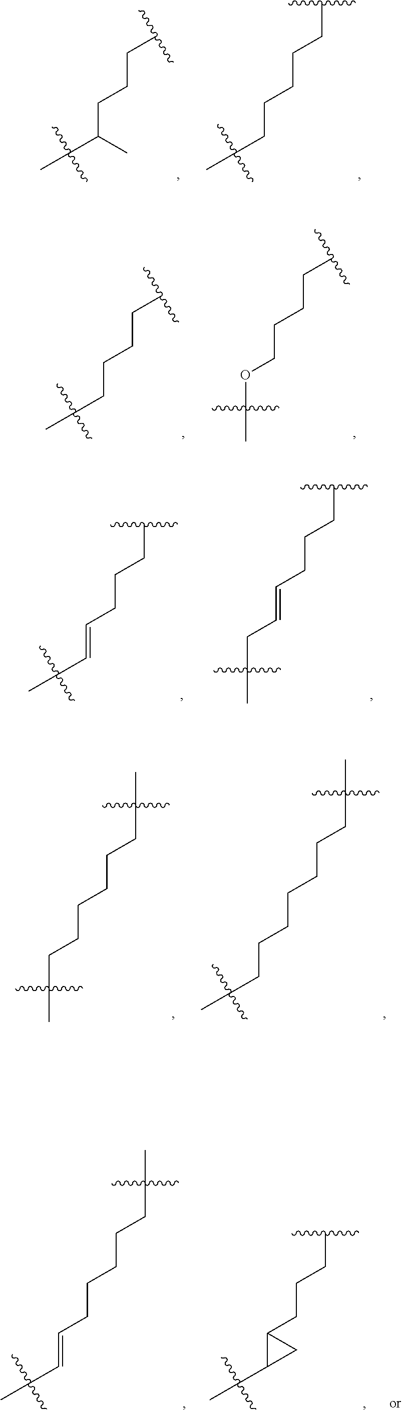

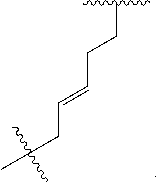 [SMILES: *C/C=C/CC*]

21. (canceled)

22. A compound of formula: 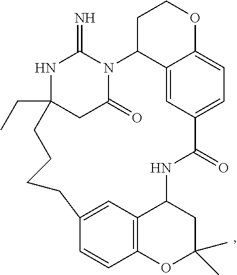 [SMILES: CCC12CCCCc3ccc4c(c3)C(CC(C)(C)O4)NC(=O)c3ccc4c(c3)C(CCO4)N(C(=N)N1)C(=O)C2]
or a pharmaceutically acceptable salt thereof.

23. A compound of formula: 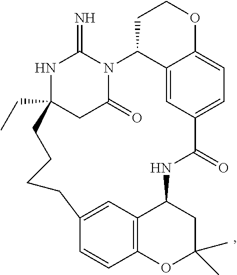 [SMILES: CC[C@]12CCCCc3ccc4c(c3)[C@H](CC(C)(C)O4)NC(=O)c3ccc4c(c3)[C@@H](CCO4)N(C(=N)N1)C(=O)C2]
or a pharmaceutically acceptable salt thereof.

24. A compound of formula: 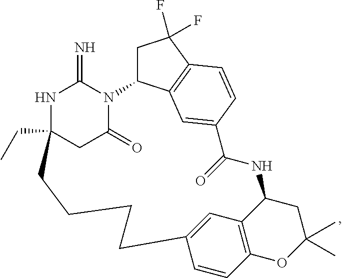 [SMILES: CC[C@]12CCCCc3ccc4c(c3)[C@H](CC(C)(C)O4)NC(=O)c3ccc4c(c3)[C@@H](CC4(F)F)N(C(=N)N1)C(=O)C2]
or a pharmaceutically acceptable salt thereof.

25. A method for treating a Plasmodium infection, or for treating malaria, which comprises administering to a subject in need of such treatment a therapeutically effective amount of claim 1, or a pharmaceutically acceptable salt thereof.

26. A method for inhibiting plasmepsin X which comprises administering to a subject in need of such treatment a therapeutically effective amount of a compound of claim 1, or a pharmaceutically acceptable salt thereof.

27. A method for inhibiting plasmepsin IX which comprises administering to a subject in need of such treatment a therapeutically effective amount of a compound of claim 1, or a pharmaceutically acceptable salt thereof.

28. A method for dual inhibition of plasmepsin X and plasmepsin X which comprises administering to a subject in need of such treatment a therapeutically effective amount of a compound of claim 1, or a pharmaceutically acceptable salt thereof.

29. (canceled)

30. (canceled)

31. (canceled)

32. (canceled)

33. A pharmaceutical composition comprising a compound of claim 1, or a pharmaceutically acceptable salt thereof, and a pharmaceutically acceptable carrier.

34. A pharmaceutical composition comprising a compound of claim 1 and a pharmaceutically acceptable carrier.

35. A method for treating a Plasmodium infection, or for treating malaria, comprising administration of a compound of claim 1, or a pharmaceutically acceptable salt thereof, and an effective amount of one or more additional anti-malarial agents.

36. A method for the treatment of malaria by inhibition of plasmepsin X, IX and at least one other mechanism, comprising administration of a compound of claim 1, or a pharmaceutically acceptable salt thereof, and an effective amount of one additional anti-malarial agent, wherein the additional anti-malarial agent acts through a different mechanism than inhibiting plasmepsin IX or plasmepsin X.

37. The compound of claim 1 represented by structural formula III: 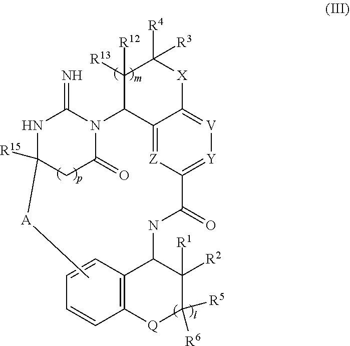
or a pharmaceutically acceptable salt thereof,
wherein R¹ and R² are independently selected from hydrogen, halogen, CN, OH, C₁-C₆alkoxy, C₁-C₆alkylOC₁-C₆alkyl, C₁-C₆alkylCOOH, COOH, oxo, COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, —C₁-C₆alkylOhaloC₁-C₆alkyl, haloC₁-C₆alkyl, C₁-C₆alkylOH, CON(R⁷)(R⁸), N(R⁷)(R⁸) and C₁-C₆alkylN(R⁷)(R⁸);
R³ and R⁴ are independently selected from hydrogen, C₁-C₆alkylOC₁-C₆alkyl or C₁-C₆alkyl, or R³ when taken with R⁴ forms a C₃-C₆heterocycloalkyl; or R³ and R⁴ are both hydrogen, methyl, ethyl, or halogen;
R⁵ and R⁶ are independently selected from hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) or C₁-C₆alkylN(R⁷)(R⁸);
Q is selected from the group consisting of CH₂, CH(CH₃), C(CH₃)₂, O, CH(OCH₃), SO₂, NH, and CF₂; A is a straight saturated or unsaturated (C₃-C₁₀)alkylene comprising at least one —CH₂— group, wherein one or more additional —CH₂— groups in A are optionally and independently replaced with a moiety selected from the group consisting of O, S, NR, CONR, NRCO, SO₂, and SO₂NR and wherein one or more of the hydrogens along A can be replaced with a group independently selected from hydroxyl, halogen and C₁₋₃ haloalkyl;
R¹² and R¹³ are independently selected from hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) and C₁-C₆alkylN(R⁷)(R⁸);
R¹⁵ is hydrogen or C₁-C₆alkyl;
X is a bond, C(R¹⁴)₂, or 0,
l is 0 or 1, and
Y, V, Z, R, R⁷, R⁸, R¹⁴, m and p are as defined in claim 1.

38. The compound of claim 37 wherein V, Y and Z are each CH; X is selected from a bond, C(R¹⁴)₂, and O, Q is selected from CH₂, CH(CH₃), C(CH₃)₂, O, CH(OCH₃), R¹ and R² are independently selected from hydrogen, bromine, fluorine, chlorine, methyl, OH, halogen, CN oxo, methoxymethyl, COOCH₂CH₃, and trifluoromethyl, and R⁵ and R⁶ are independently selected from hydrogen, methyl, ethyl and t-butyl.

39. The compound of claim 1 represented by structural formula IIIA: 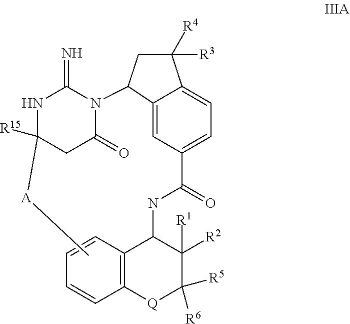
or a pharmaceutically acceptable salt thereof, wherein R¹ and R² are independently selected from hydrogen, halogen, CN, OH, C₁-C₆alkoxy, C₁-C₆alkylOC₁-C₆alkyl, C₁-C₆alkylCOOH, COOH, oxo, COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, —C₁-C₆alkylOhaloC₁-C₆alkyl, haloC₁-C₆alkyl, C₁-C₆alkylOH, CON(R⁷)(R⁸), N(R⁷)(R⁸) and C₁-C₆alkylN(R⁷)(R⁸);
R³ and R⁴ are independently selected from hydrogen, C₁-C₆alkylOC₁-C₆alkyl or C₁-C₆alkyl, or R³ when taken with R⁴ forms a C₃-C₆heterocycloalkyl; or R³ and R⁴ are both hydrogen, methyl, ethyl, or halogen; R⁵ and R⁶ are independently selected from hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) or C₁-C₆alkylN(R⁷)(R⁸);
Q is selected from the group consisting of CH₂, CH(CH₃), C(CH₃)₂, O, CH(OCH₃), SO₂, NH, and CF₂; A is a straight saturated or unsaturated (C₃-C₁₀)alkylene comprising at least one —CH₂— group, wherein one or more additional —CH₂— groups in A are optionally and independently replaced with a moiety selected from the group consisting of O, S, NR, CONR, NRCO, SO₂, and SO₂NR and wherein one or more of the hydrogens along A can be replaced with a group independently selected from hydroxyl, halogen and C₁₋₃ haloalkyl; and
R¹⁵ is hydrogen or C₁-C₆alkyl.

40. The compound according to claim 39, or a pharmaceutically acceptable salt thereof wherein Q is selected from CH₂, CH(CH₃), C(CH₃)₂, O, CH(OCH₃), R¹ and R² are independently selected from hydrogen, bromine, fluorine, chlorine, methyl, OH, halogen, CN oxo, methoxymethyl, COOCH₂CH₃, and trifluoromethyl, and R⁵ and R⁶ are independently selected from hydrogen, methyl, ethyl and t-butyl.

41. The compound of claim 1 represented by structural formula VI: 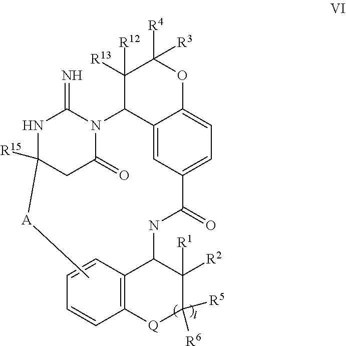
or a pharmaceutically acceptable salt thereof,
wherein R¹ and R² are independently selected from hydrogen, halogen, CN, OH, C₁-C₆alkoxy, C₁-C₆alkylOC₁-C₆alkyl, C₁-C₆alkylCOOH, COOH, oxo, COOC₁-C₆alkyl, C₁-C₆alkylCOOC₁-C₆alkyl, C₃-C₆cycloalkyl, C₁-C₆alkylC₃-C₆cycloalkyl, C₁-C₆alkyl, —C₁-C₆alkylOhaloC₁-C₆alkyl, haloC₁-C₆alkyl, C₁-C₆alkylOH, CON(R⁷)(R⁸), N(R⁷)(R⁸) and C₁-C₆alkylN(R⁷)(R⁸);
R³ and R⁴ are independently selected from hydrogen, C₁-C₆alkylOC₁-C₆alkyl or C₁-C₆alkyl, or R³ when taken with R⁴ forms a C₃-C₆heterocycloalkyl; or R³ and R⁴ are both hydrogen, methyl, ethyl, or halogen;
R⁵ and R⁶ are independently selected from hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) or C₁-C₆alkylN(R⁷)(R⁸);
Q is selected from the group consisting of CH₂, CH(CH₃), C(CH₃)₂, O, CH(OCH₃), SO₂, NH, and CF₂; A is a straight saturated or unsaturated (C₃-C₁₀)alkylene comprising at least one —CH₂— group, wherein one or more additional —CH₂— groups in A are optionally and independently replaced with a moiety selected from the group consisting of O, S, NR, CONR, NRCO, SO₂, and SO₂NR and wherein one or more of the hydrogens along A can be replaced with a group independently selected from hydroxyl, halogen and C₁₋₃ haloalkyl;
R¹² and R¹³ are independently selected from hydrogen, halogen, CN, OH, C₁-C₆alkoxy, C₁-C₆alkylOC₁-C₆alkyl, C₁-C₆alkylCOOH, COOH, C₃-C₆cycloalkyl, C₁-C₆alkyl, haloC₁-C₆alkyl, C₁-C₆alkylOH, CON(R⁷)(R⁸), N(R⁷)(R⁸) and C₁-C₆alkylN(R⁷)(R⁸);
R¹⁵ is hydrogen or C₁-C₆alkyl, and
l is 0 or 1.

42. The compound of claim 41 wherein Q is selected from the group consisting of CH₂, CH(CH₃), C(CH₃)₂, O, CH(OCH₃), R¹ and R² are independently selected from hydrogen, bromine, fluorine, chlorine, methyl, OH, halogen, CN oxo, methoxymethyl, COOCH₂CH₃, and trifluoromethyl, and R⁵ and R⁶ are independently selected from hydrogen, methyl, ethyl and t-butyl.

43. A compound selected from the group consisting of: 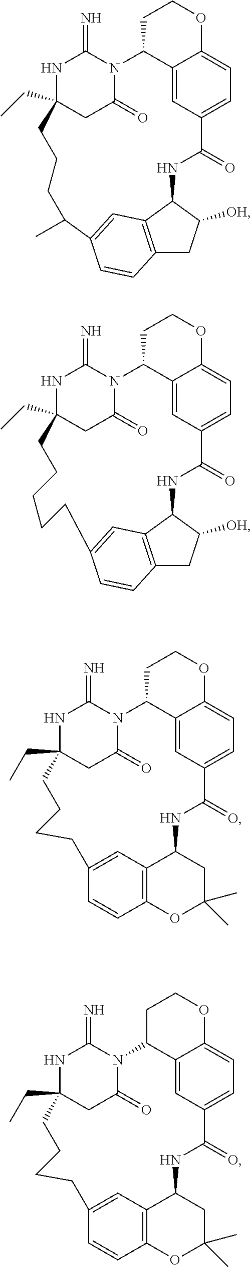 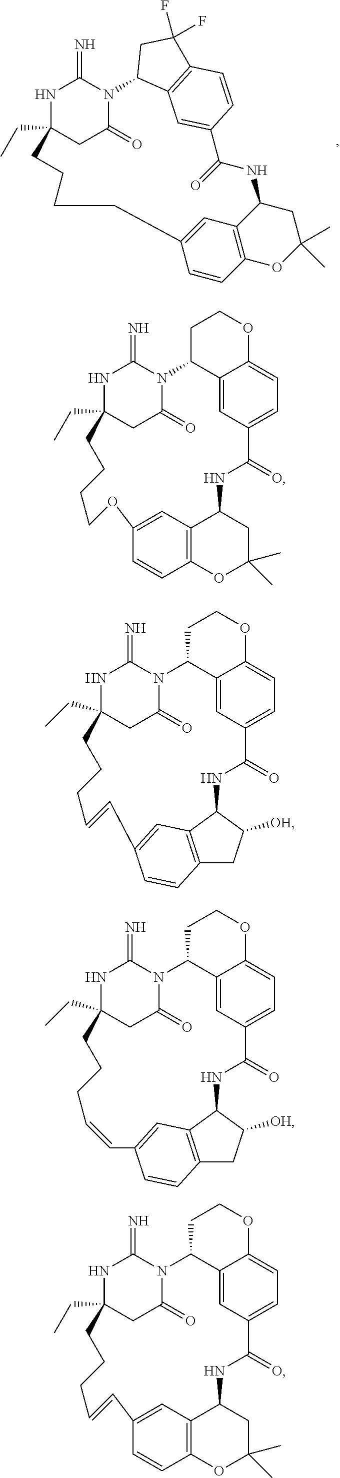 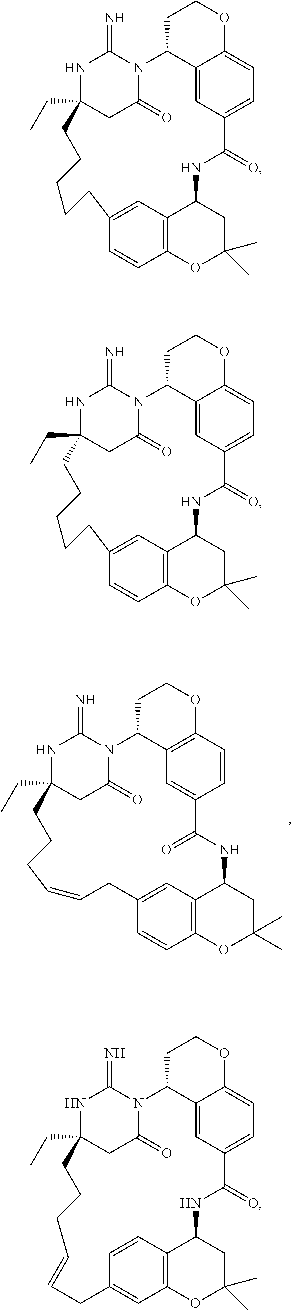 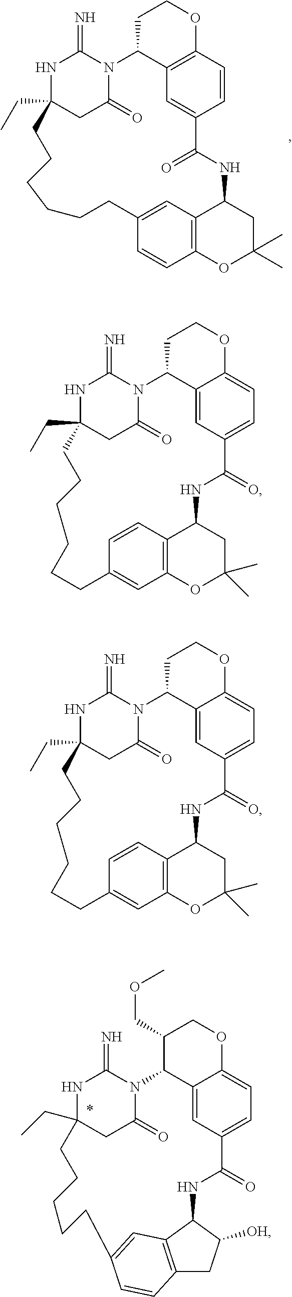 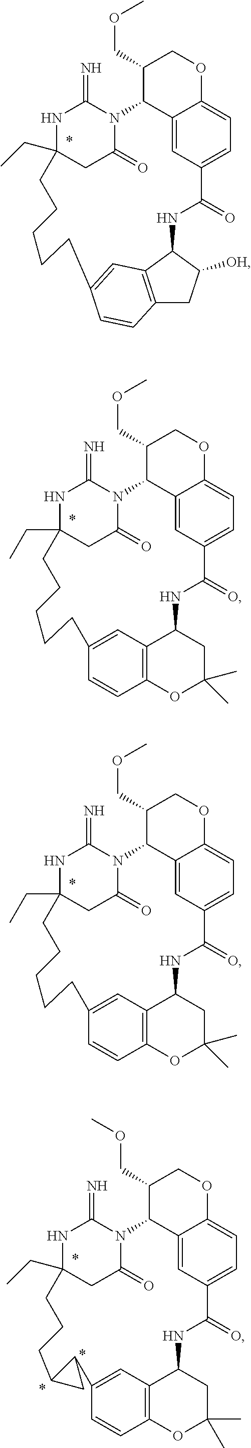 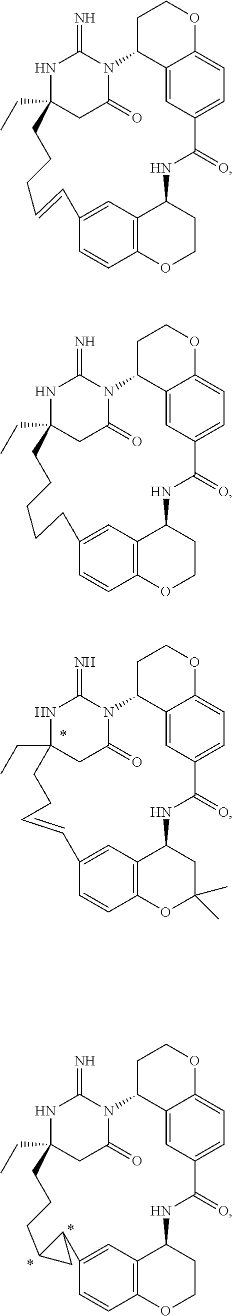 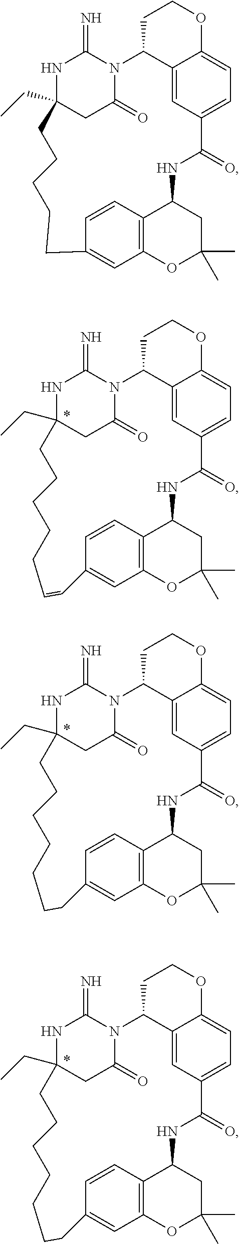 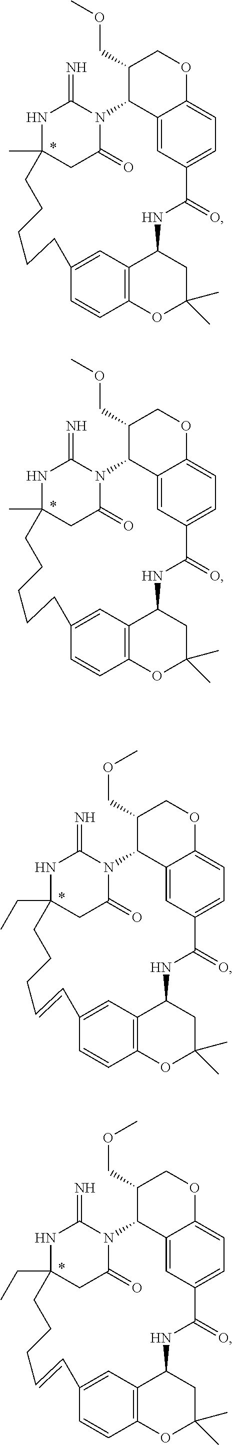 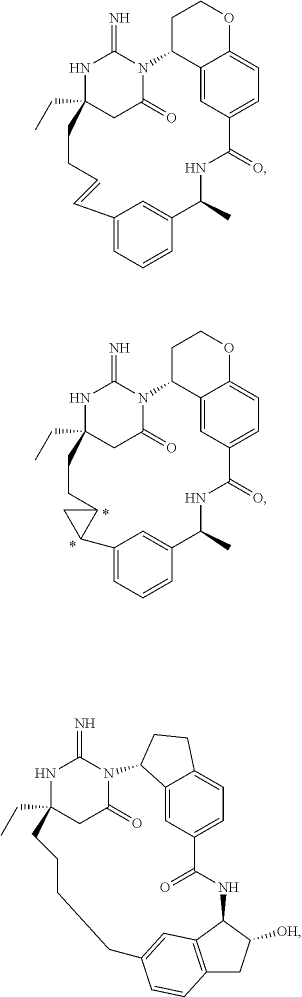 [SMILES: CC[C@]12CC/C=C/c3cccc(c3)[C@H](C)NC(=O)c3ccc4c(c3)[C@@H](CCO4)N(C(=N)N1)C(=O)C2 ; CC[C@]12CCC3CC3c3cccc(c3)[C@H](C)NC(=O)c3ccc4c(c3)[C@@H](CCO4)N(C(=N)N1)C(=O)C2 ; CC[C@]12CCCCc3ccc4c(c3)[C@@H](NC(=O)c3ccc5c(c3)[C@@H](CC5)N(C(=N)N1)C(=O)C2)[C@H](O)C4] 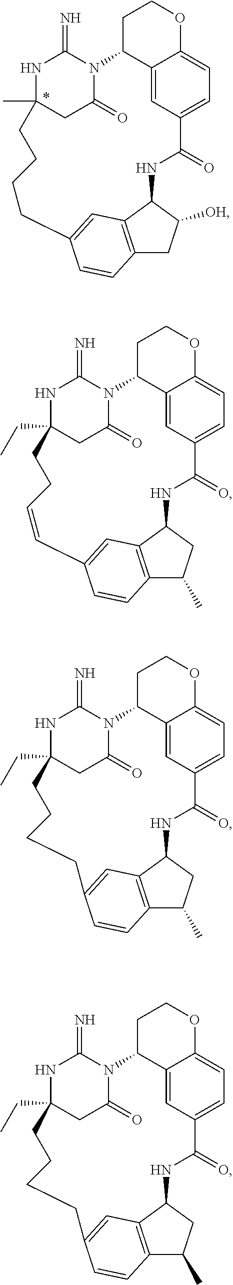 [SMILES: CC12CCCCc3ccc4c(c3)[C@@H](NC(=O)c3ccc5c(c3)[C@@H](CCO5)N(C(=N)N1)C(=O)C2)[C@H](O)C4 ; CC[C@]12CC/C=C\c3ccc4c(c3)[C@H](C[C@@H]4C)NC(=O)c3ccc4c(c3)[C@@H](CCO4)N(C(=N)N1)C(=O)C2 ; CC[C@]12CCCCc3ccc4c(c3)[C@H](C[C@@H]4C)NC(=O)c3ccc4c(c3)[C@@H](CCO4)N(C(=N)N1)C(=O)C2 ; CC[C@]12CCCCc3ccc4c(c3)[C@H](C[C@H]4C)NC(=O)c3ccc4c(c3)[C@@H](CCO4)N(C(=N)N1)C(=O)C2] 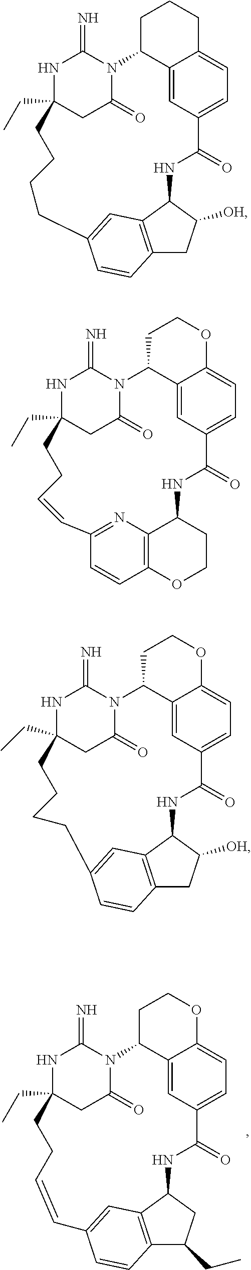 [SMILES: CC[C@]12CCCCc3ccc4c(c3)[C@@H](NC(=O)c3ccc5c(c3)[C@@H](CCC5)N(C(=N)N1)C(=O)C2)[C@H](O)C4 ; CC[C@]12CC/C=C\c3ccc4c(n3)[C@H](CCO4)NC(=O)c3ccc4c(c3)[C@@H](CCO4)N(C(=N)N1)C(=O)C2 ; CC[C@]12CCCCc3ccc4c(c3)[C@@H](NC(=O)c3ccc5c(c3)[C@@H](CCO5)N(C(=N)N1)C(=O)C2)[C@H](O)C4 ; CC[C@@H]1C[C@@H]2NC(=O)c3ccc4c(c3)[C@@H](CCO4)N3C(=N)N[C@](CC)(CC/C=C\c4ccc1c2c4)CC3=O] 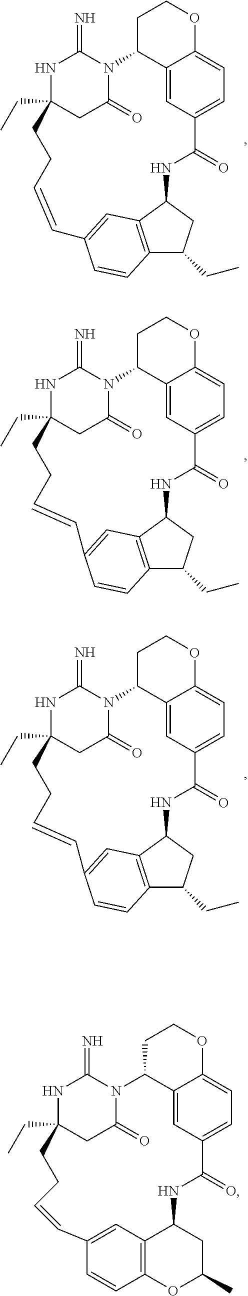 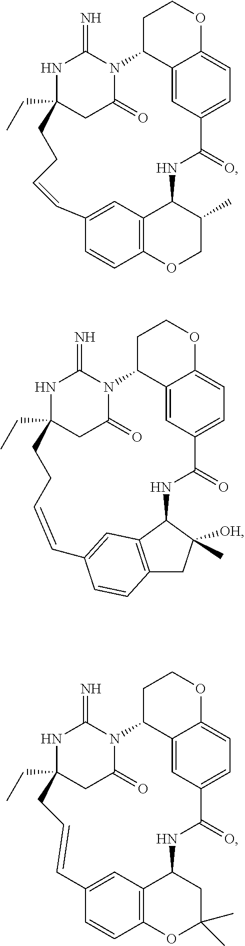 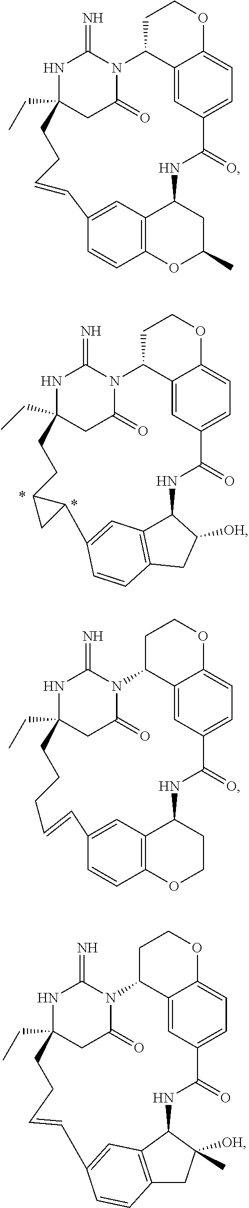 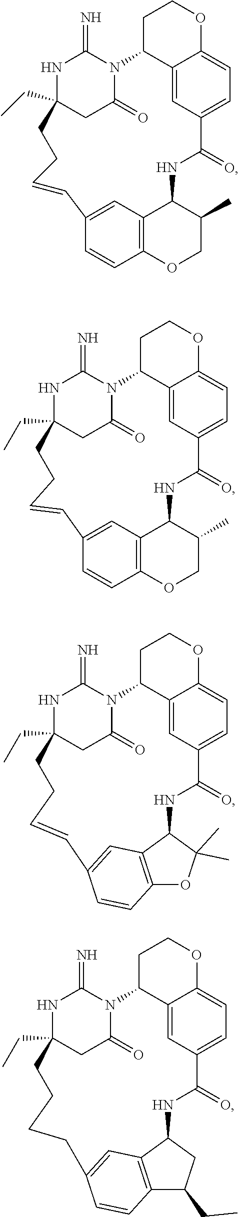 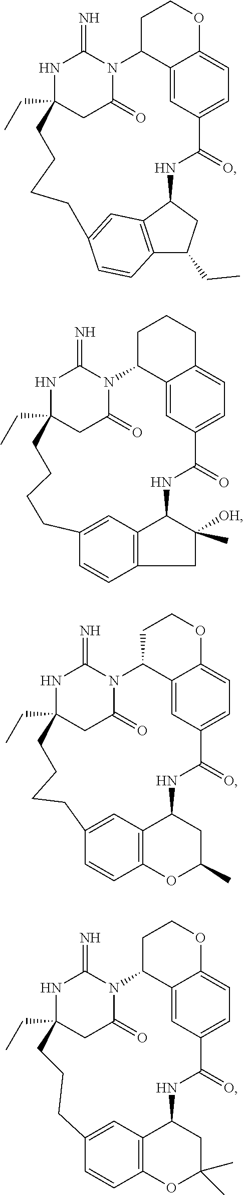 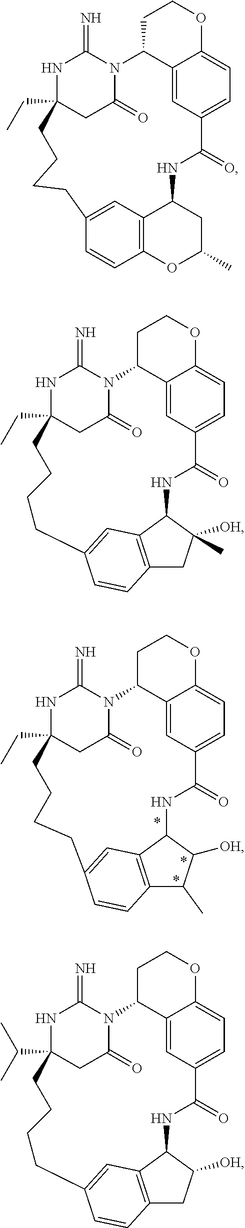 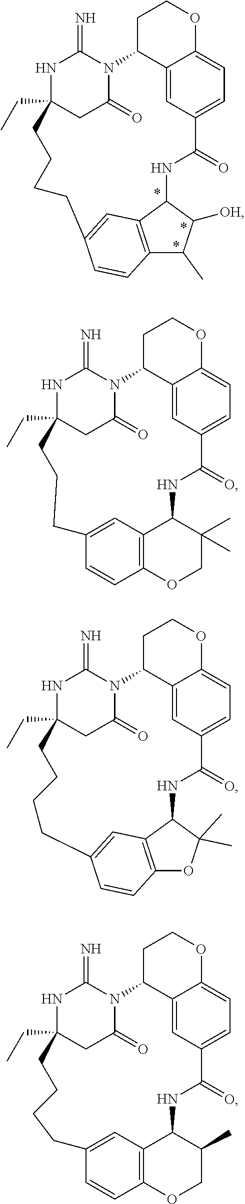 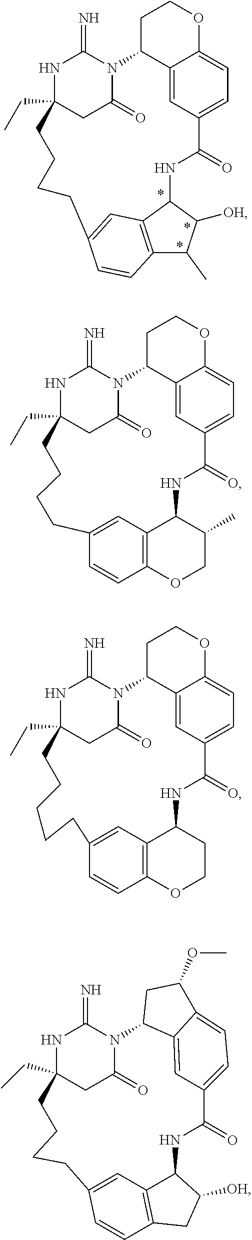 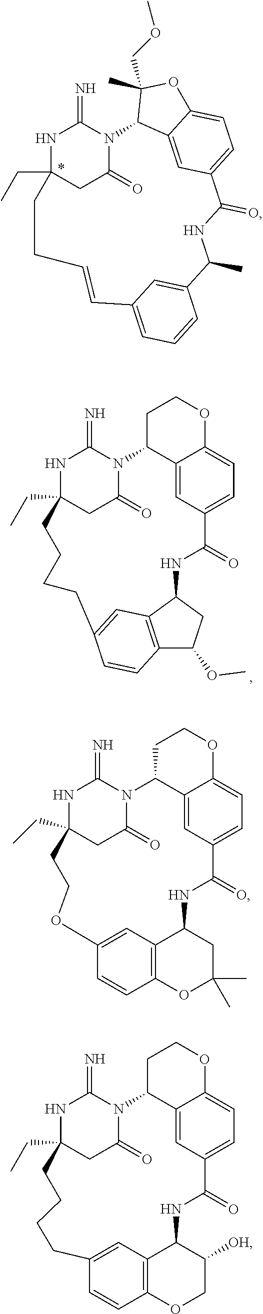 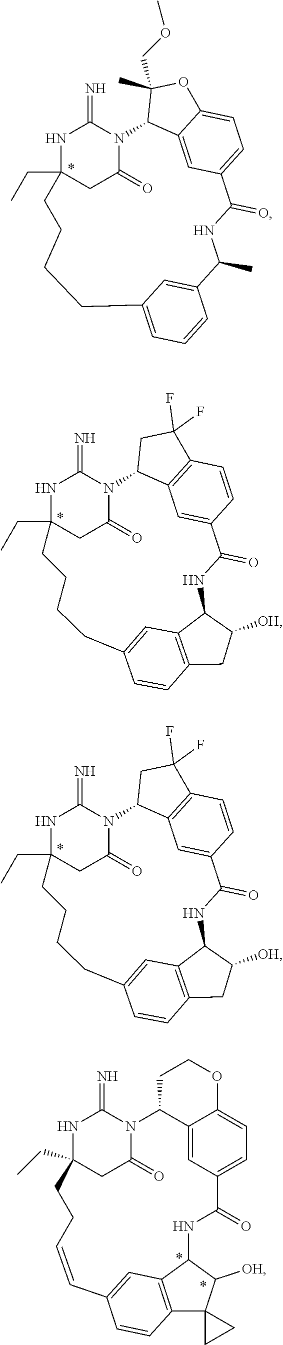 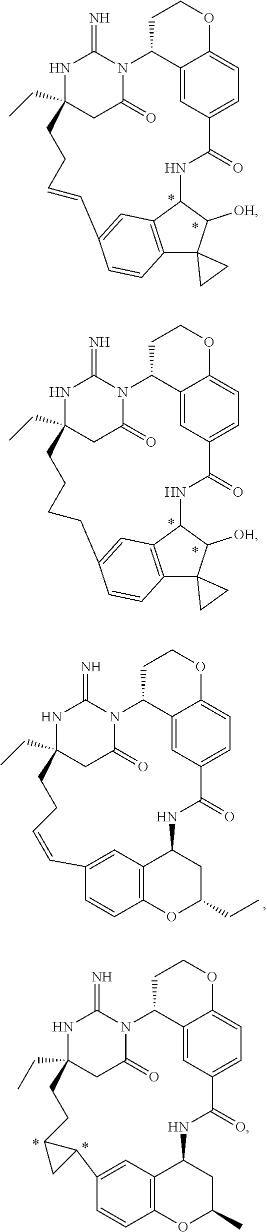 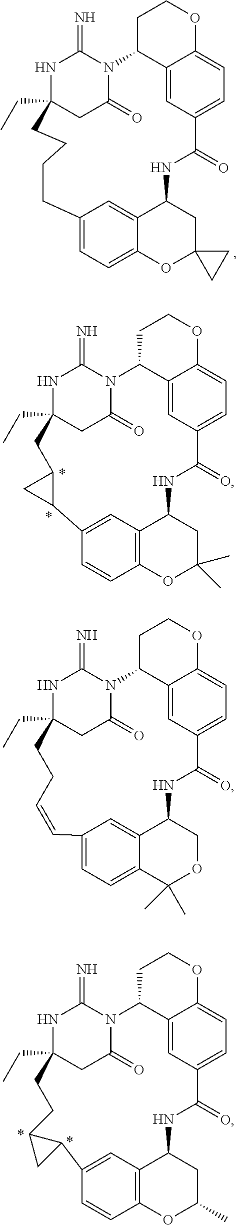 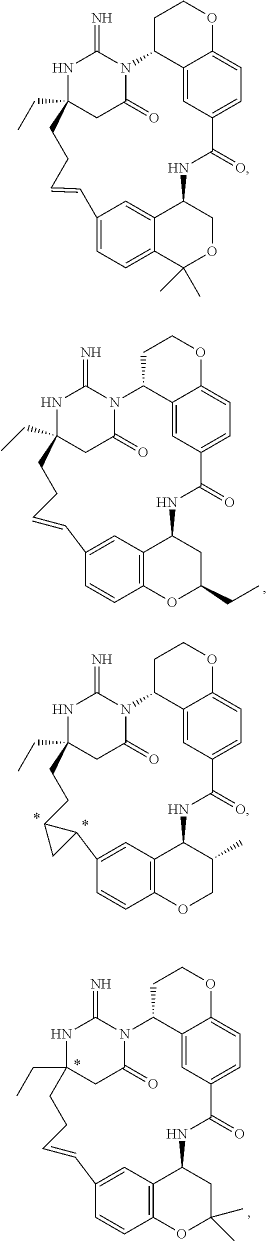 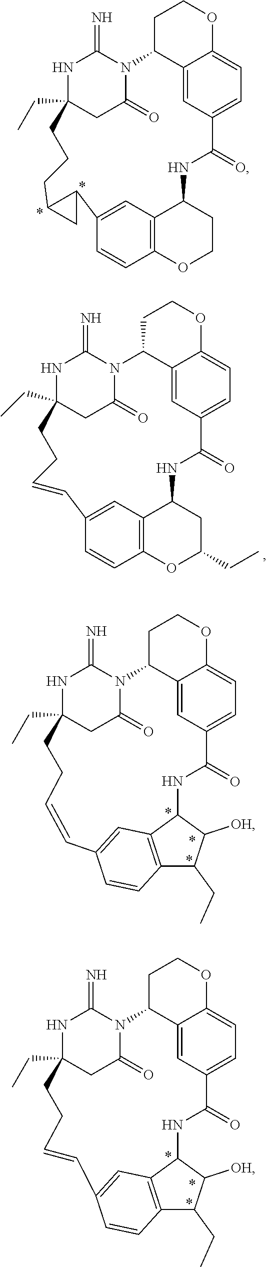 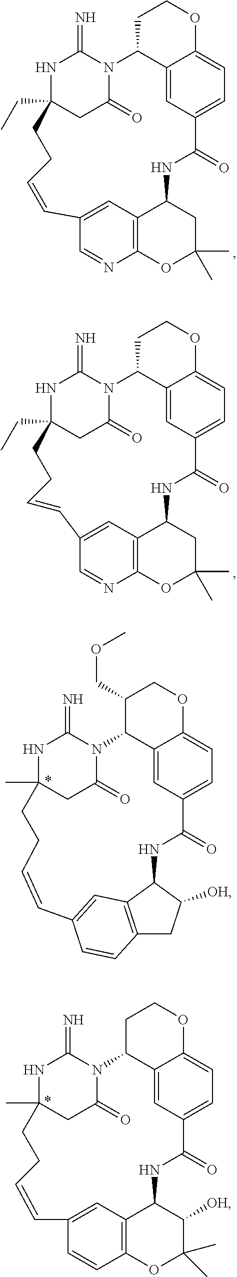 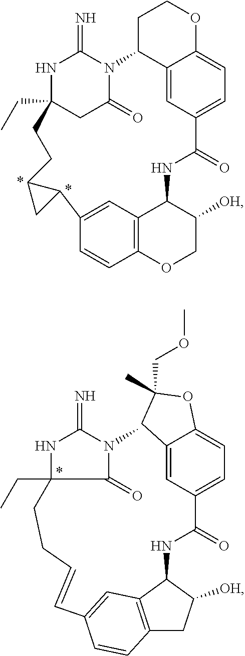 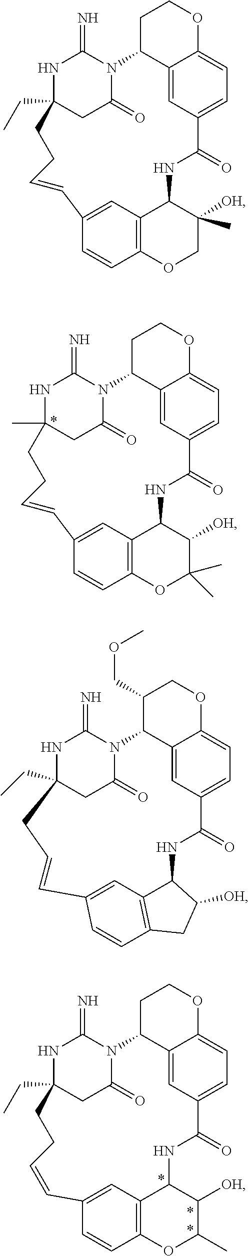 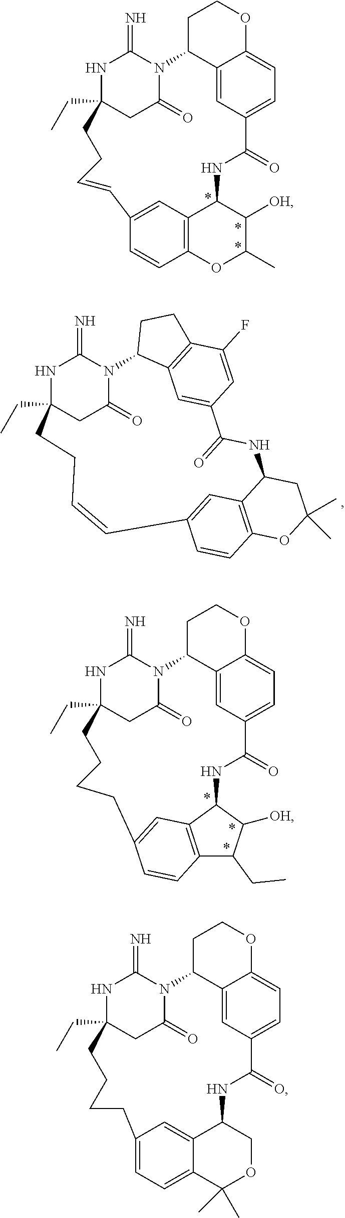 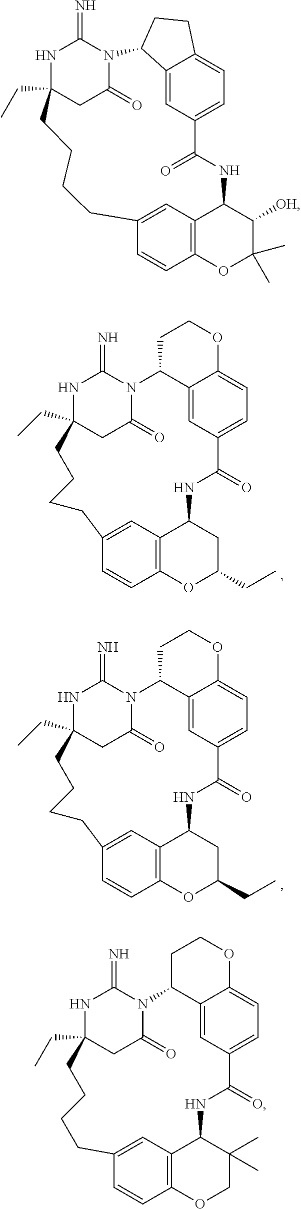 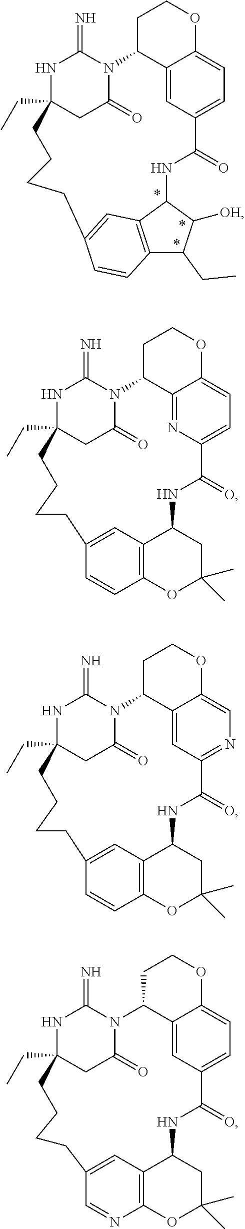 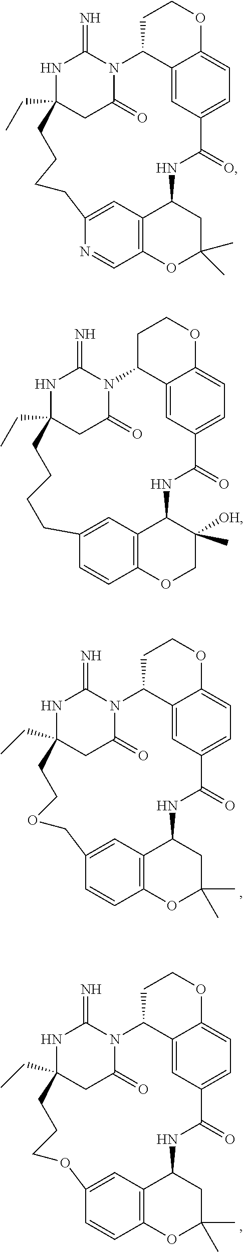 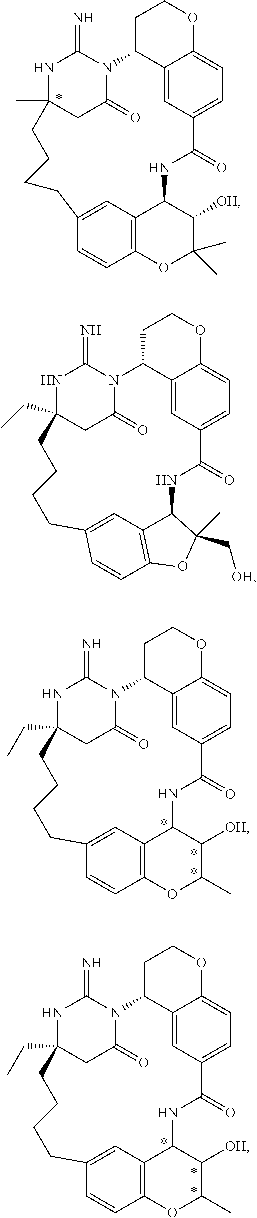 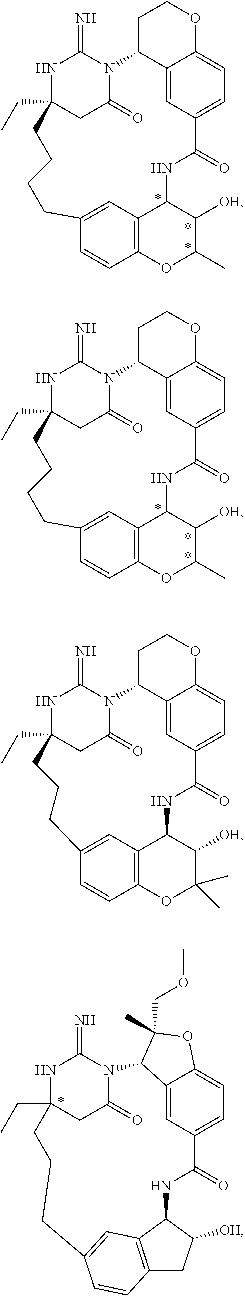 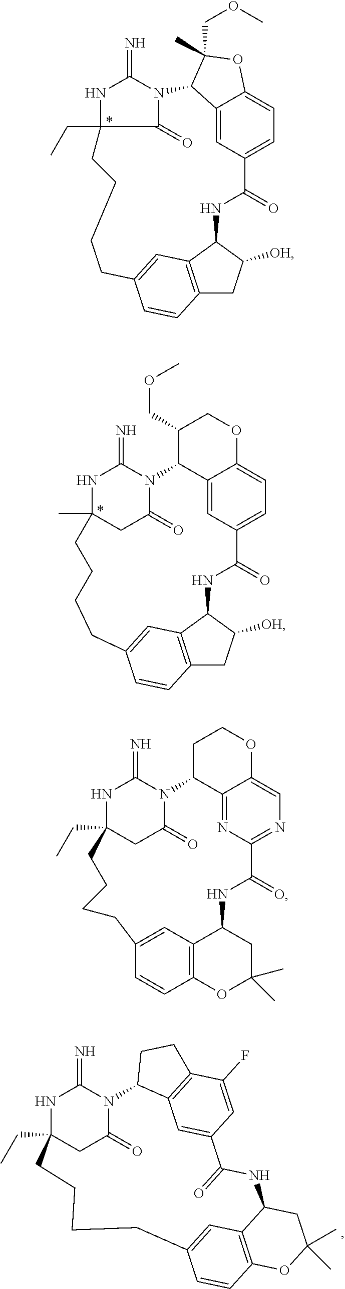 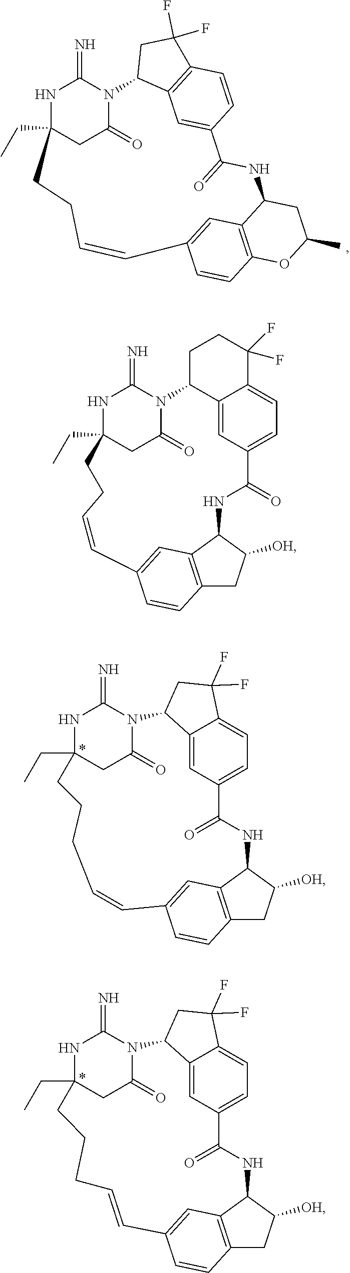 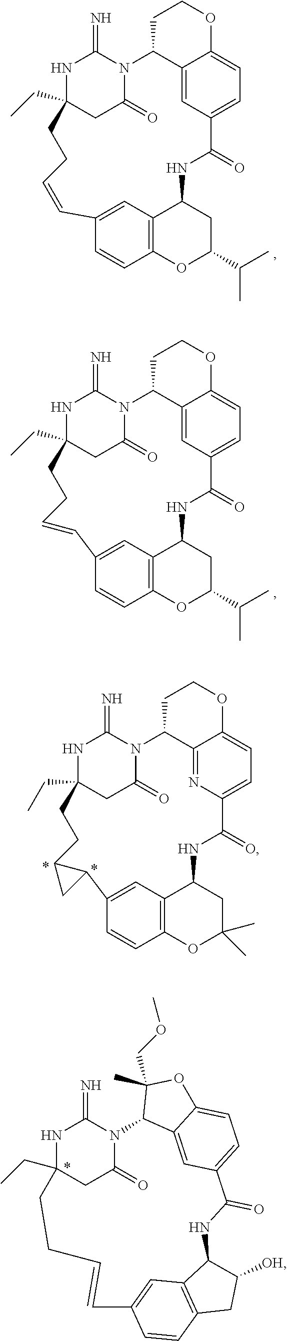 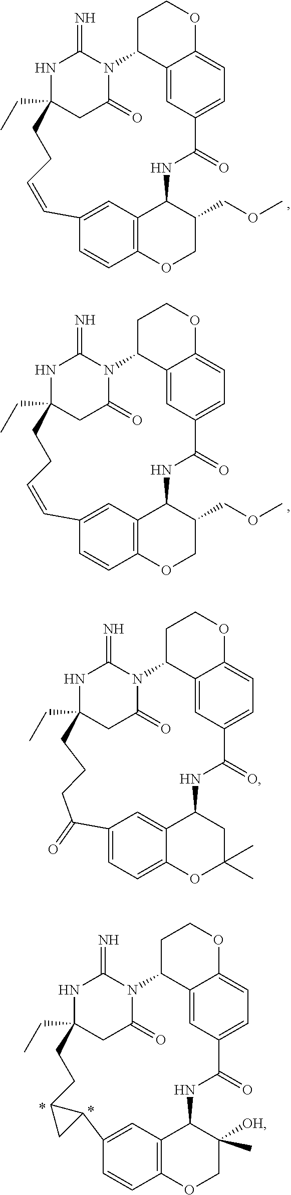 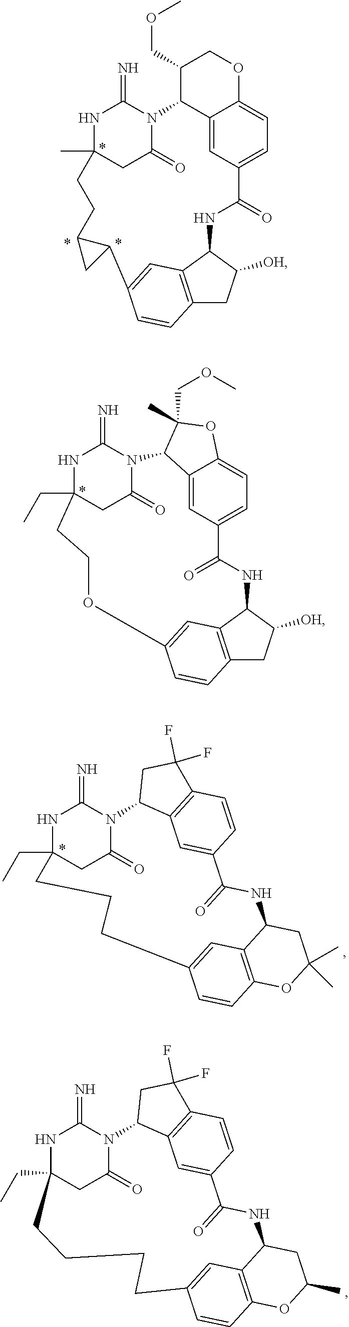 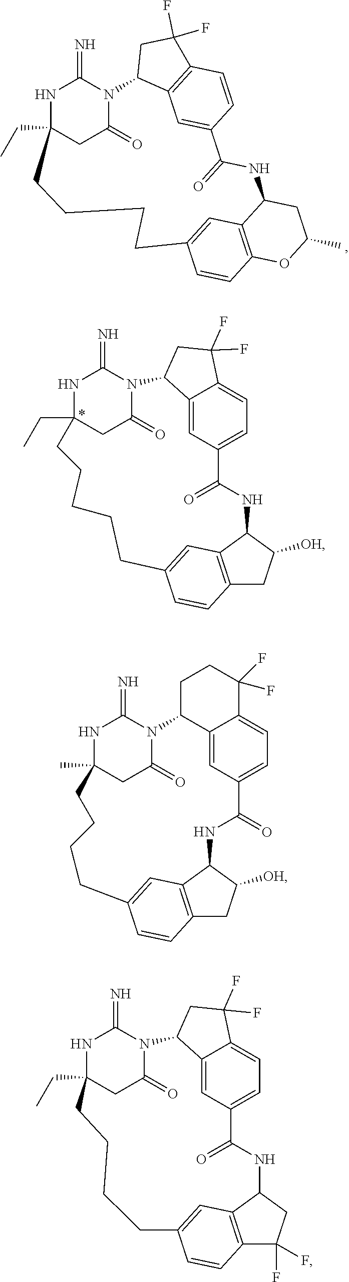 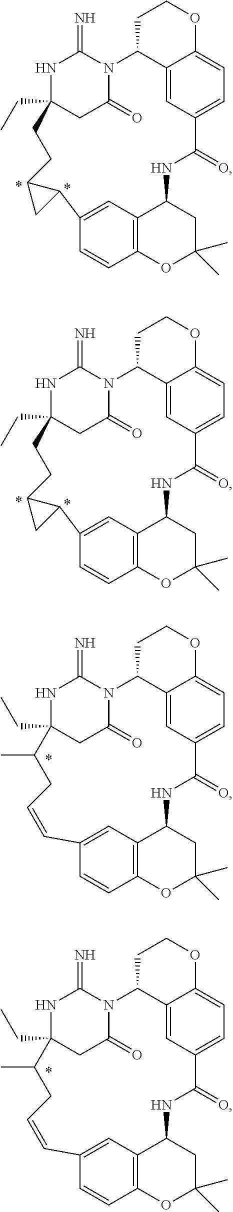 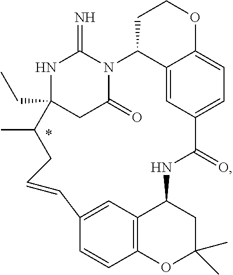 [SMILES: CC[C@@]12CC(=O)N(C(=N)N1)[C@@H]1CCOc3ccc(cc31)C(=O)N[C@H]1CC(C)(C)Oc3ccc(cc31)/C=C/CC2C] 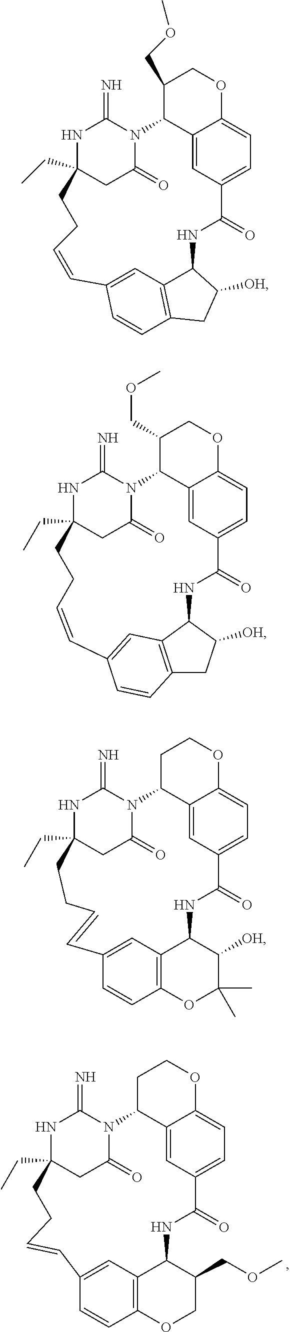 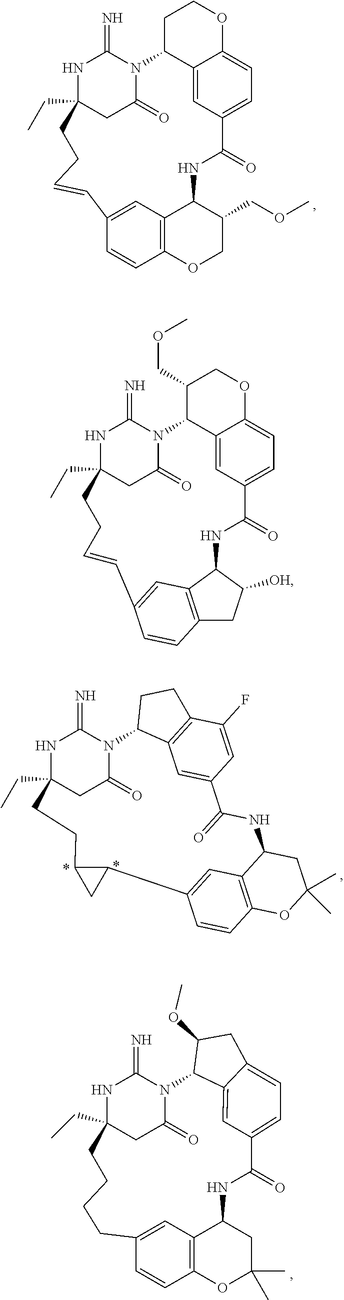 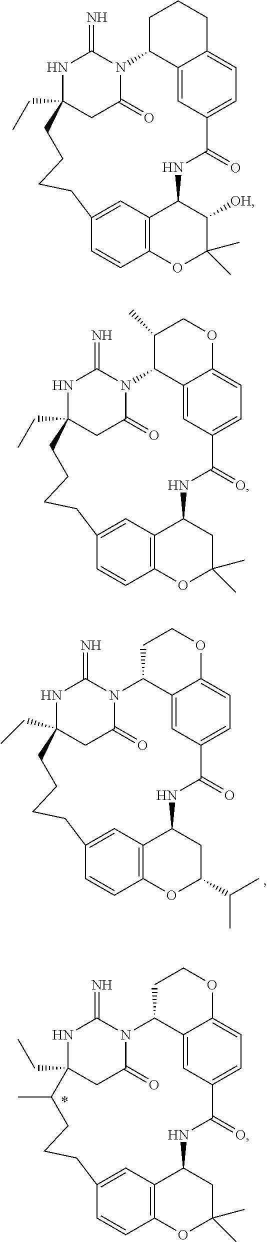 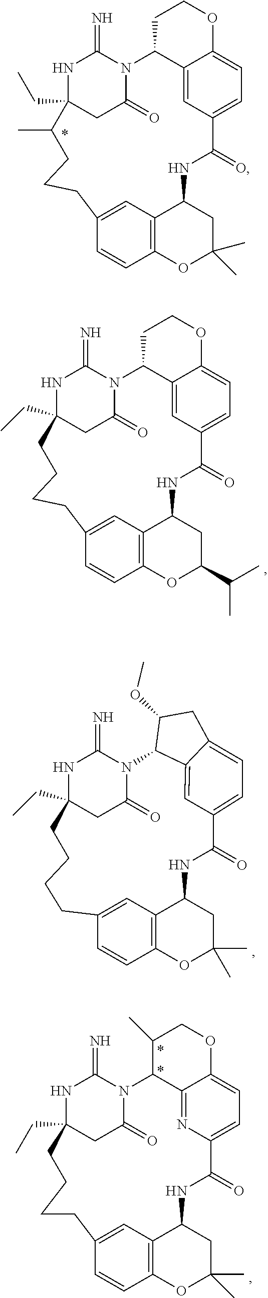 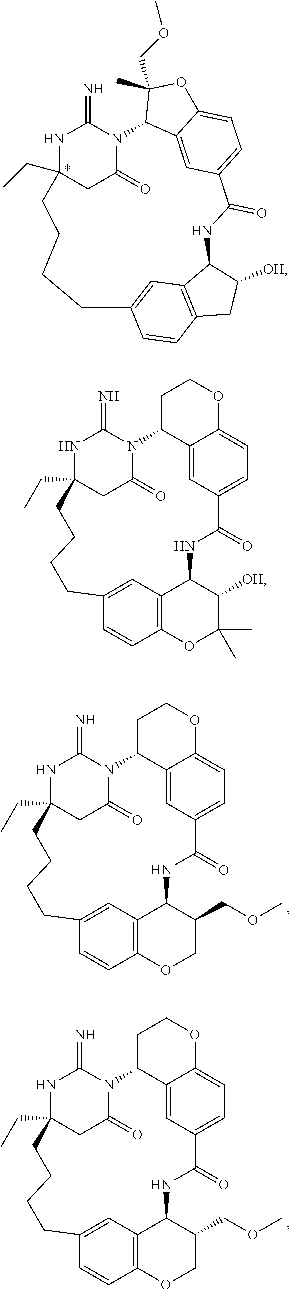 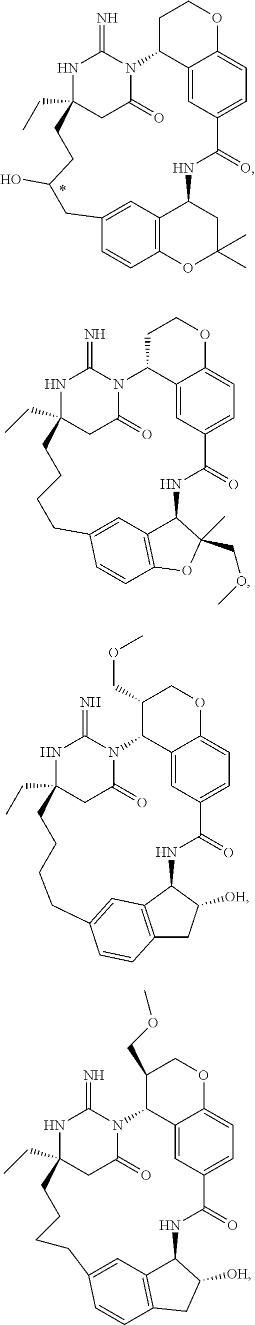 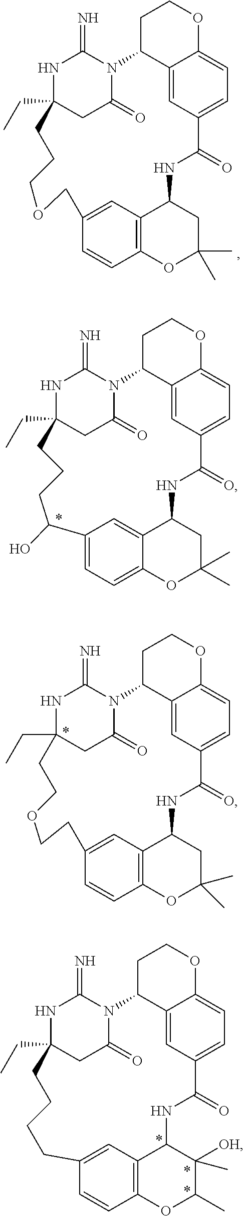 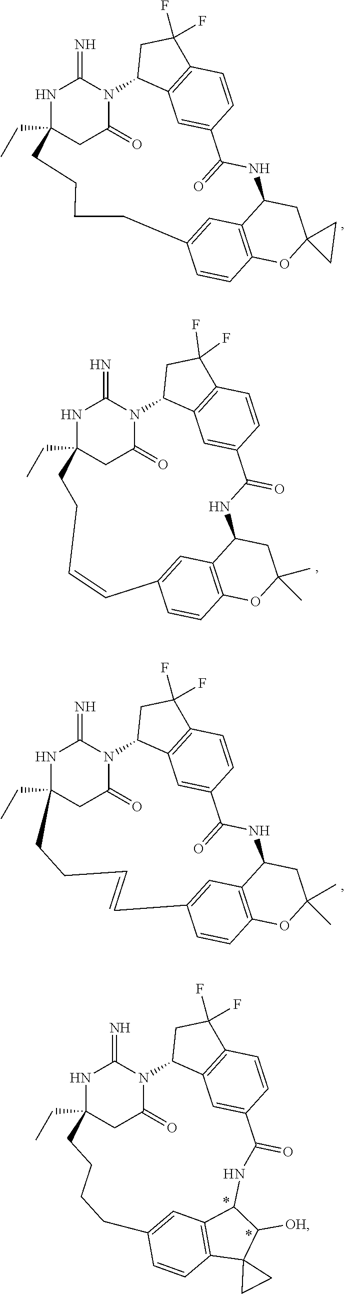 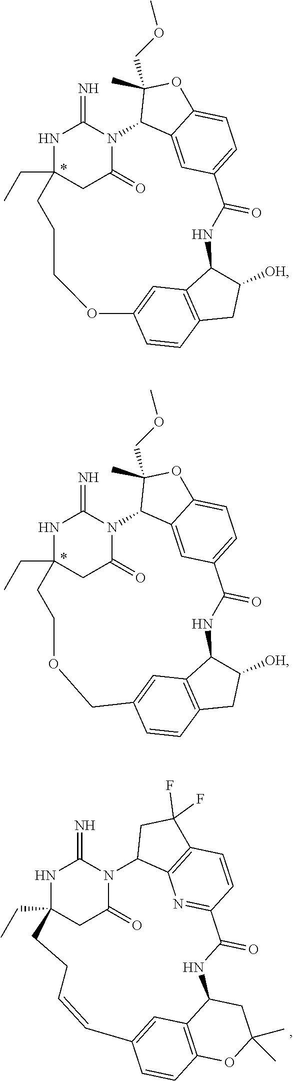 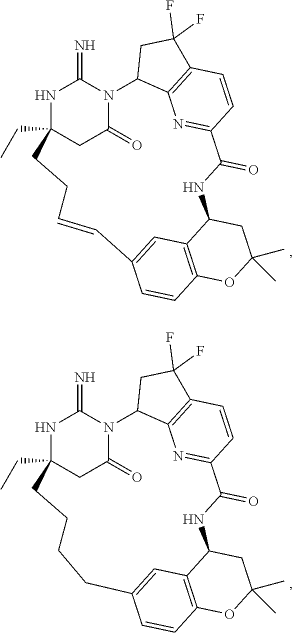 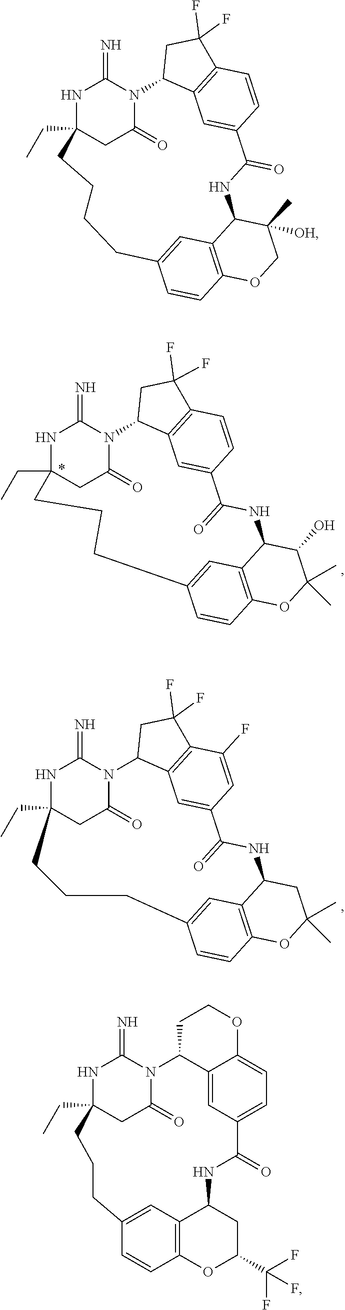 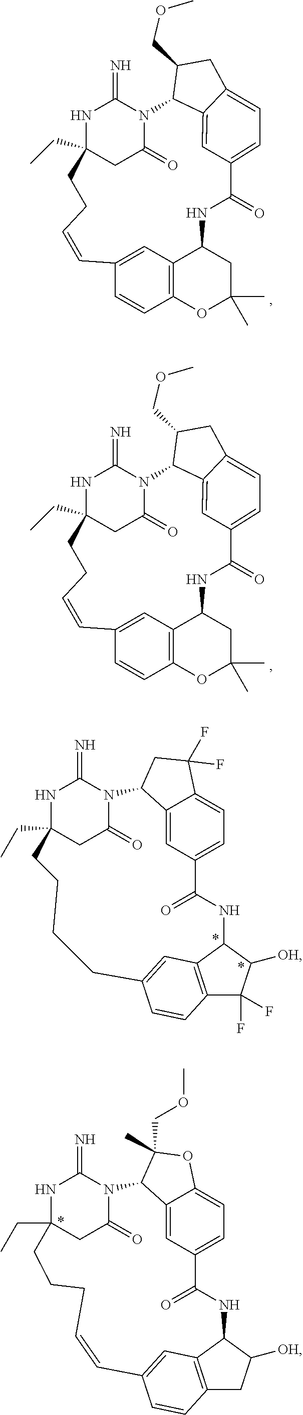 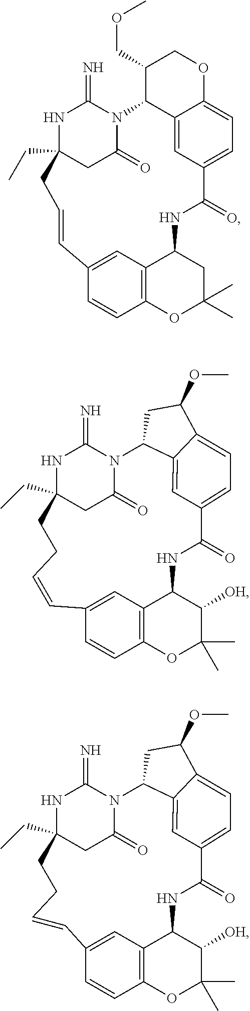 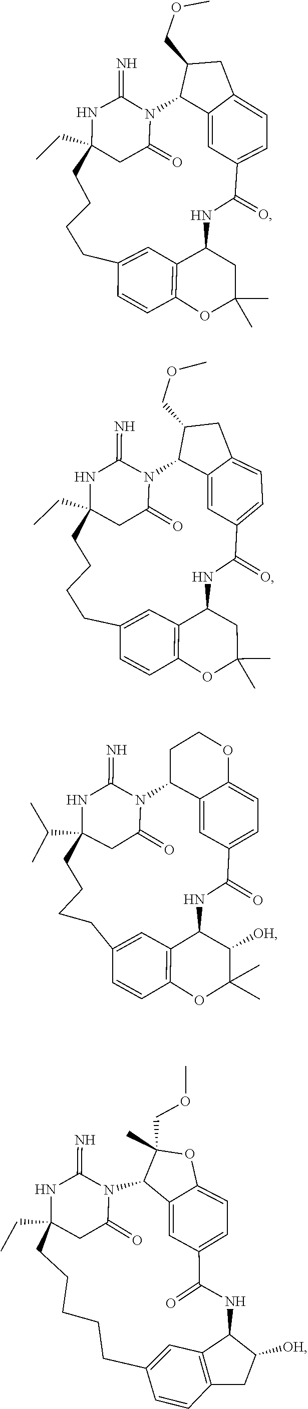 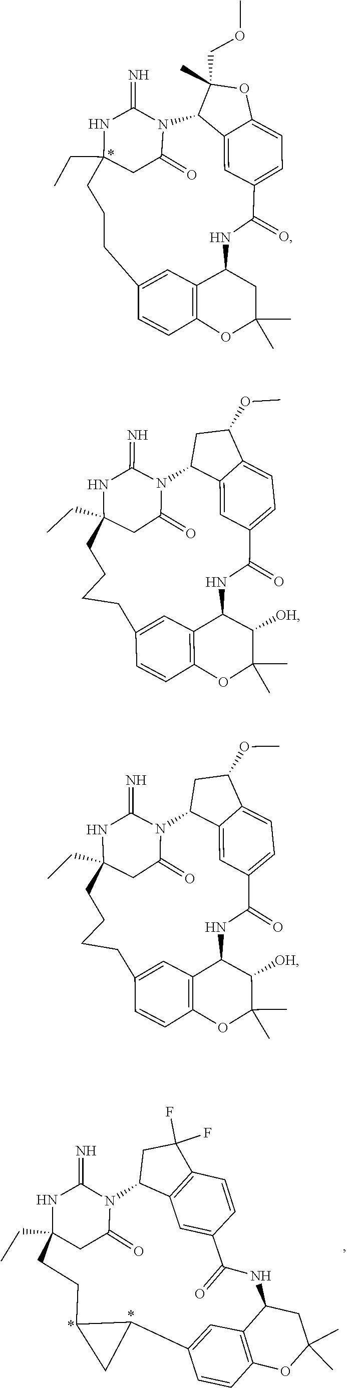 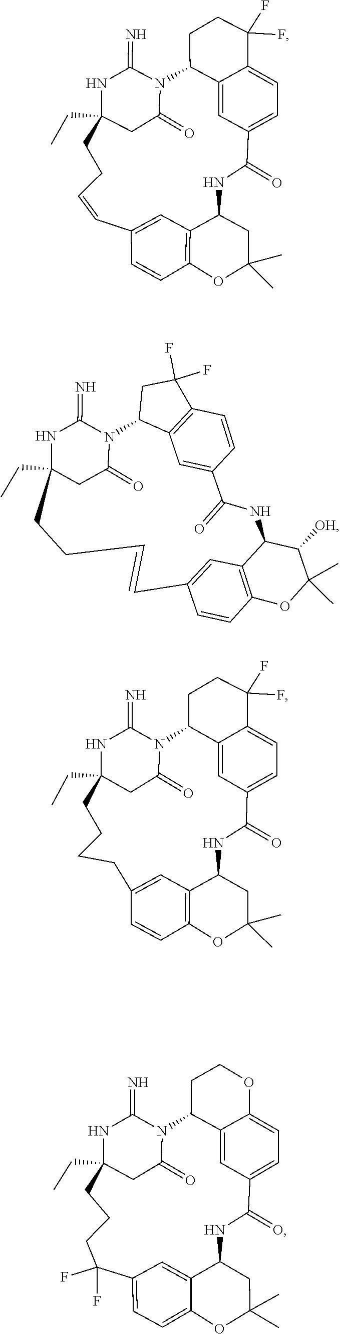 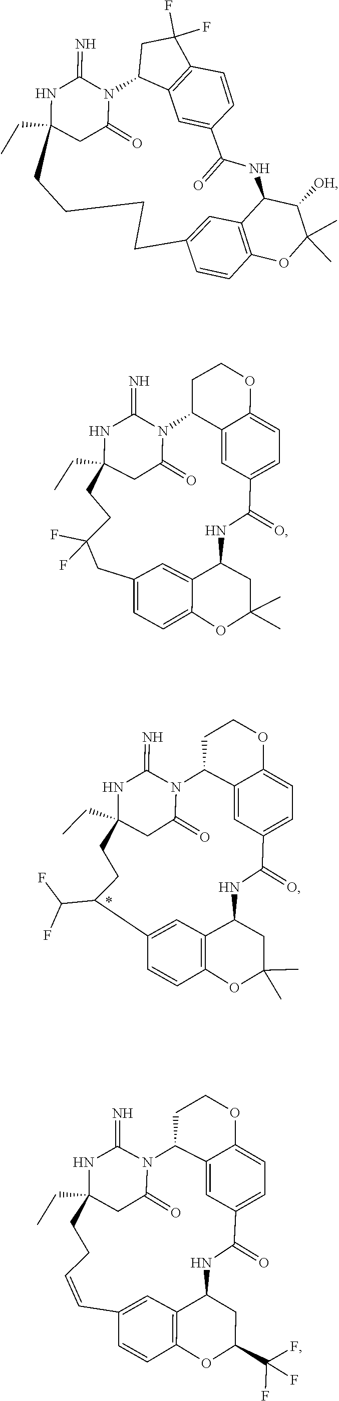 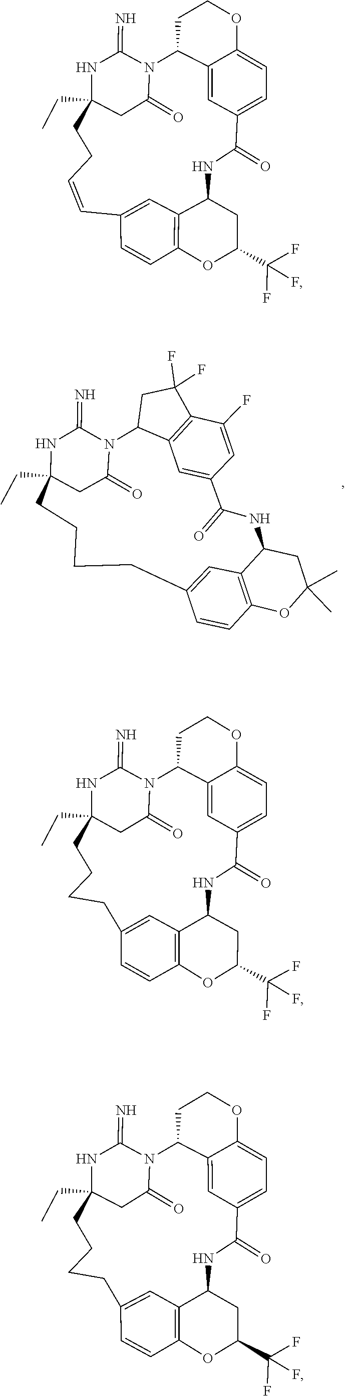 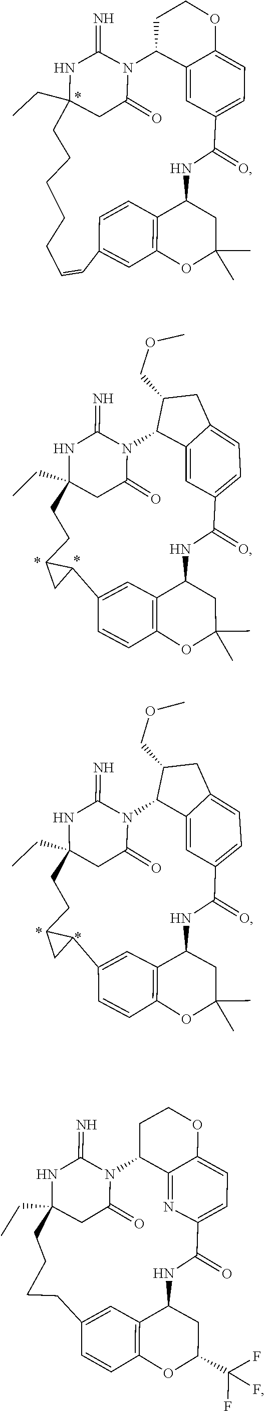 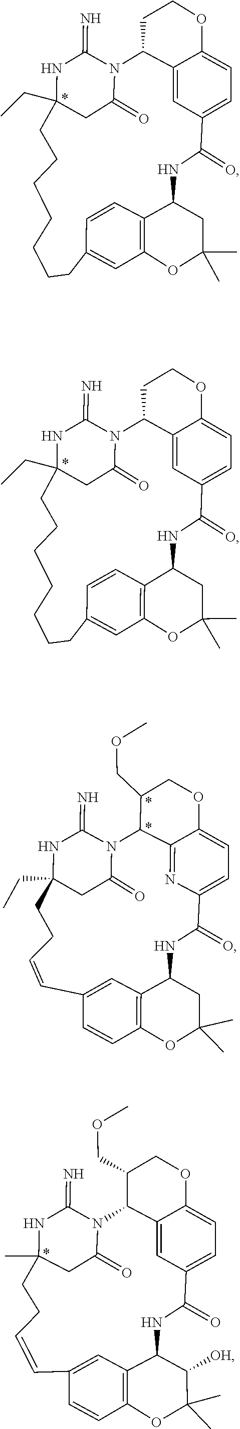 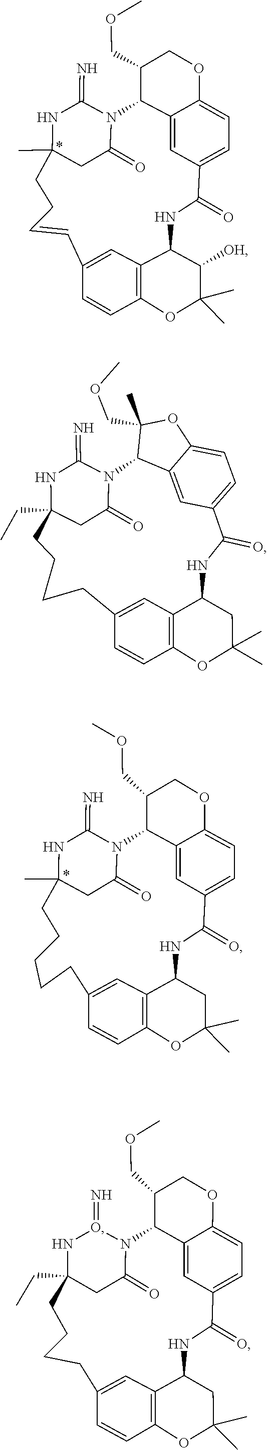 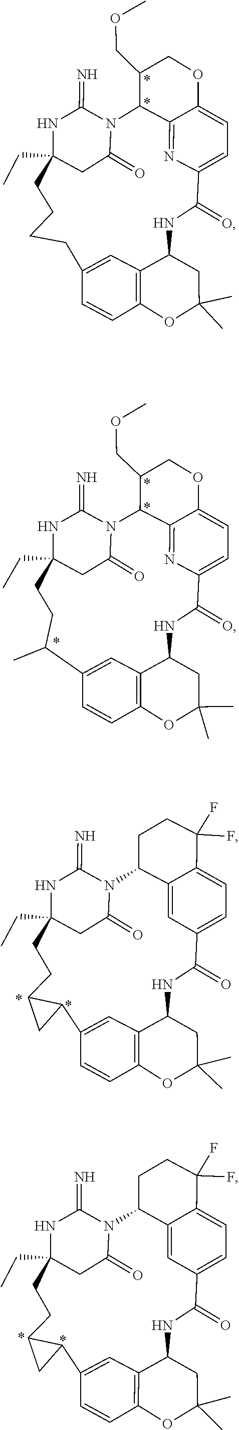 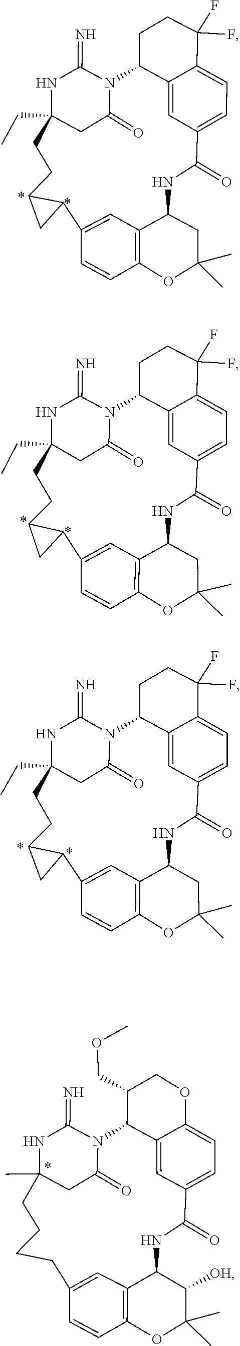 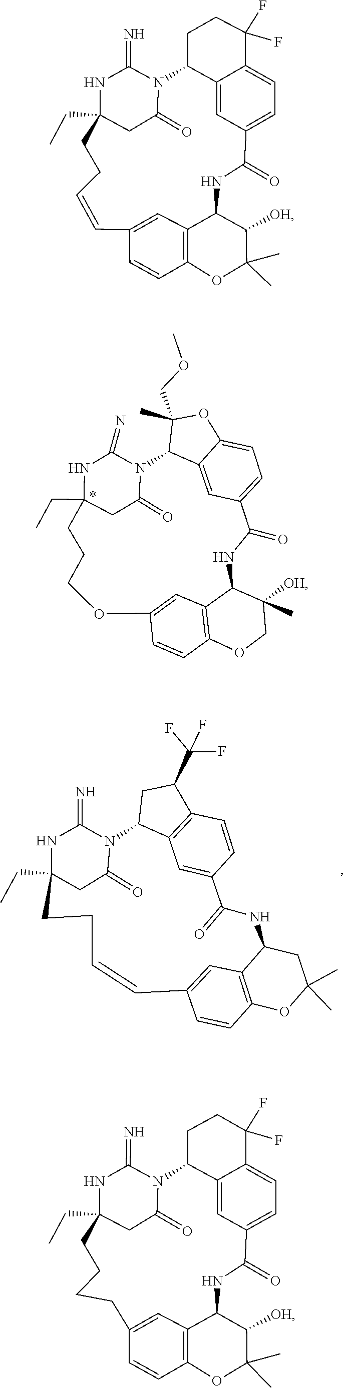 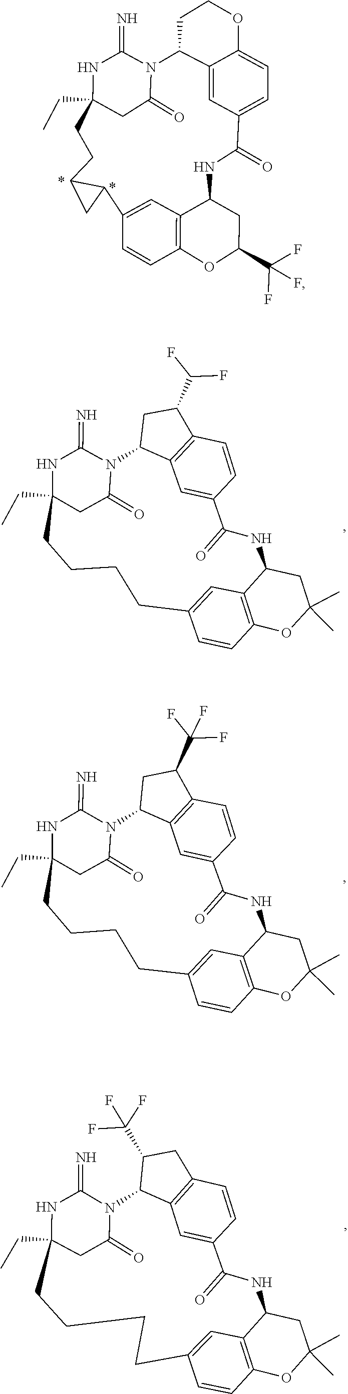 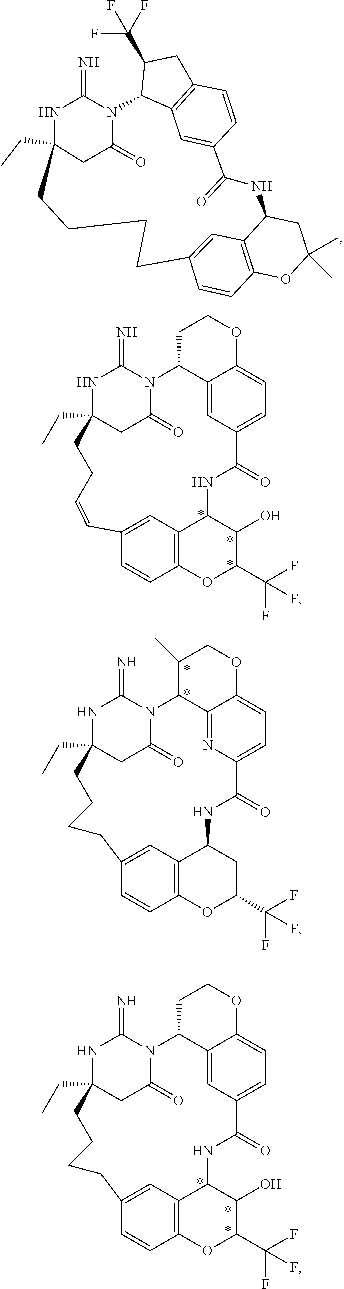 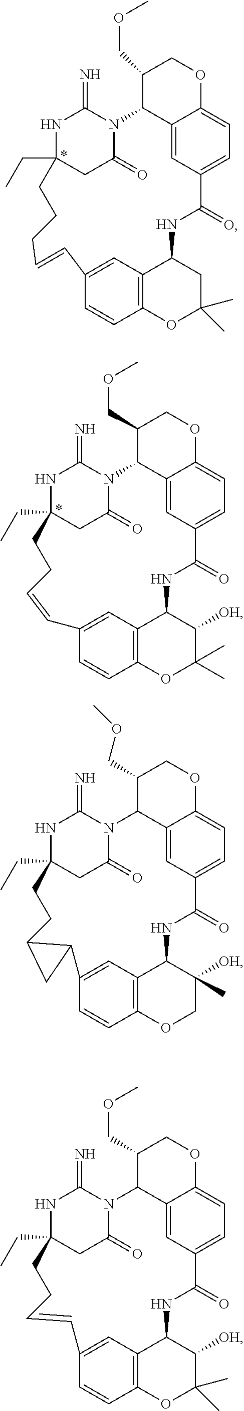 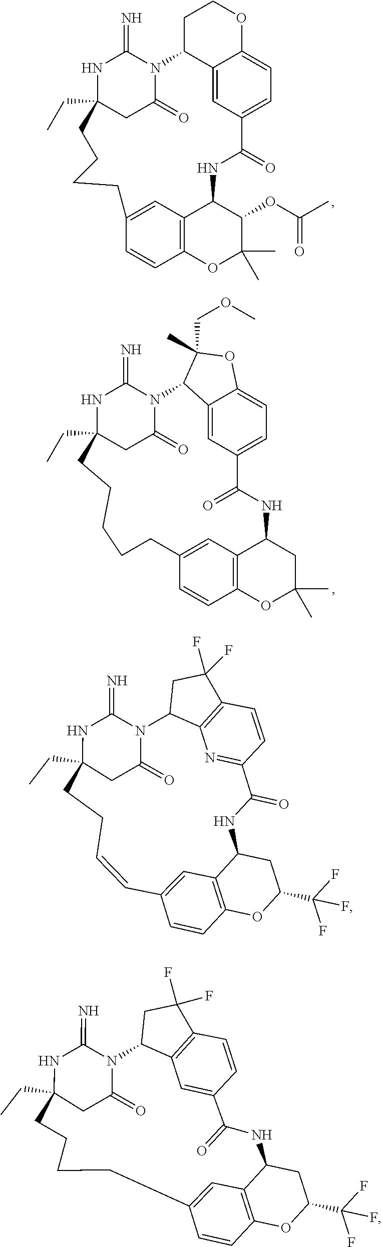 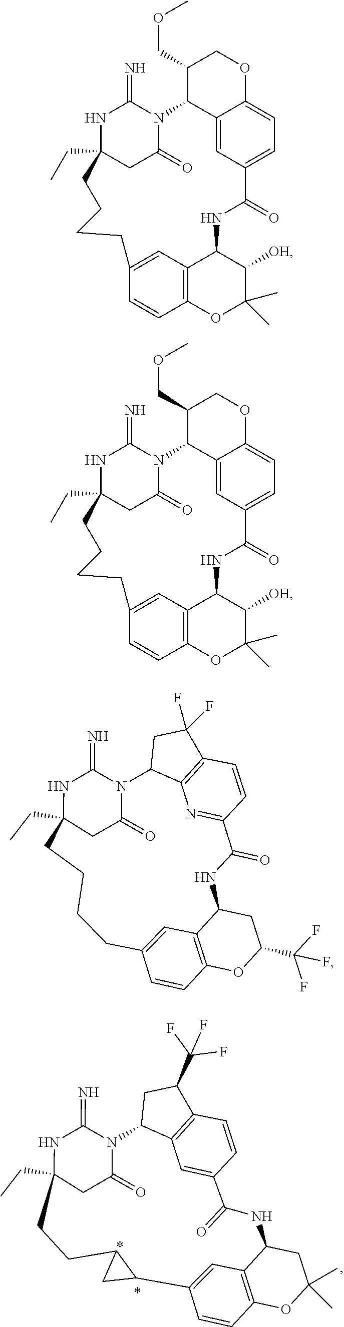 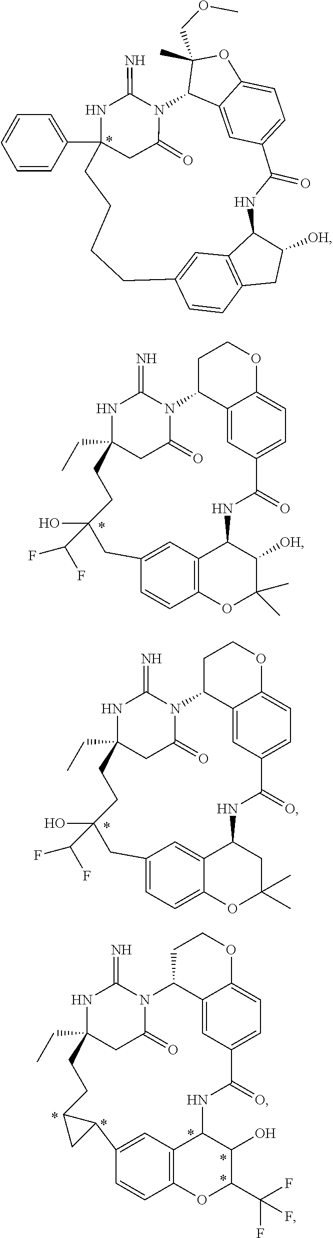 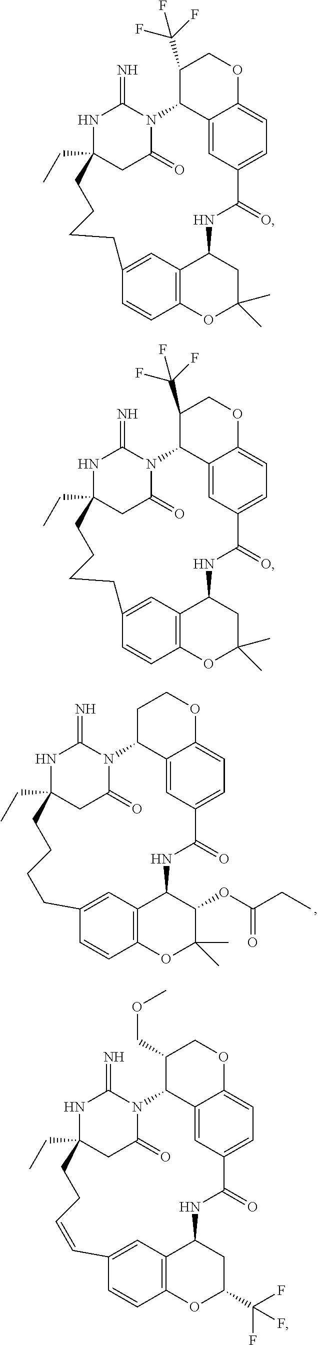 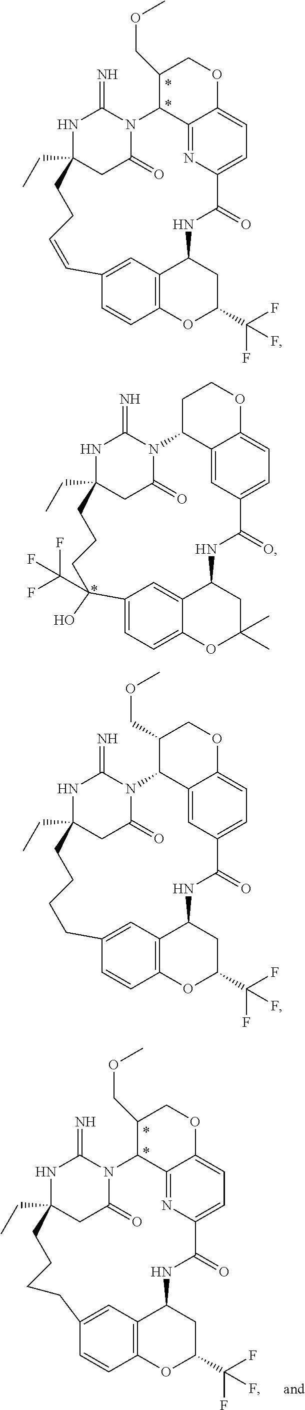 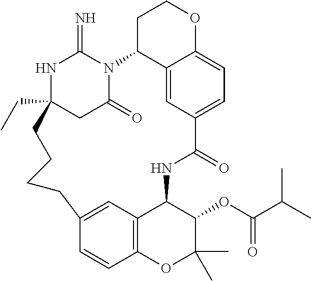 [SMILES: CC[C@]12CCCCc3ccc4c(c3)[C@@H](NC(=O)c3ccc5c(c3)[C@@H](CCO5)N(C(=N)N1)C(=O)C2)[C@H](OC(=O)C(C)C)C(C)(C)O4]
or a pharmaceutically acceptable salt thereof.
